(19) United States
(12) Patent Application Publication
(10) Pub. No.: US 2025/0352512 A2
(43) Pub. Date: Nov. 20, 2025

(54) C4-SUBSTITUTED TRYPTAMINE DERIVATIVES AND METHODS OF USING

(71) Applicant: ENVERIC BIOSCIENCES CANADA INC., Calgary (CA)

(72) Inventors: Kaveh MATINKHOO, Calgary (CA); David James PRESS, Calgary (CA); Ye CAI, Edmonton (CA); Glynnis Elizabeth JENSEN, Calgary (CA); Jessica Bik-Jing LEE, Calgary (CA); Jillian M. HAGEL, Calgary (CA); Peter J. FACCHINI, Calgary (CA)

(21) Appl. No.: 18/848,277

(22) PCT Filed: Mar. 17, 2023
(86) PCT No.: PCT/CA2023/050352

§ 371 Date: Sep. 18, 2024

Related U.S. Application Data
(60) Provisional application No. 63/321,440, filed on Mar. 18, 2022.
(60) Provisional application No. 63/347,835, filed on Jun. 1, 2022.

(30) Foreign Application Priority Data
Aug. 11, 2022 (WO) PCT/CA2022/051228
Aug. 22, 2022 (WO) PCT/CA2022/051266

(51) Int. Cl. C07D 209/16; A61K 31/4045; A61K 31/675; A61K 31/695; C07F 7/18; C07F 9/572

(52) U.S. Cl. CPC A61K 31/4045; A61K 31/4439; A61K 31/675; A61K 31/695; C07D 209/16; C07D 401/12; C07D 405/12; C07F 7/1804; C07F 9/5728

(57) ABSTRACT

Disclosed are novel C₄-substituted tryptamine derivative compounds and pharmaceutical and recreational drug formulations containing the same, including C₄-ether-substituted tryptamine derivative compounds, C₄-carbonic ester-substituted tryptamine derivative compounds, C₄-polyether substituted tryptamine derivative compounds, and C₄-phosphate substituted tryptamine derivative compounds. The pharmaceutical formulations may be used to treat psychiatric disorders.

### RELATED APPLICATIONS

This application is a National Phase Entry of PCT Application No. PCT/CA2023/050352 which claims the benefit of priority of U.S. Provisional Application No. 63/321,440, filed Mar. 18, 2022, U.S. Provisional Application No. 63/347,835, filed Jun. 1, 2022; PCT Patent Application No. PCT/CA2022/051228, filed on Aug. 11, 2022, and PCT Patent Application No. PCT/CA2022/051266, filed on Aug. 22, 2022, the entire contents of U.S. Provisional Patent Application Nos. 63/321,440 and 63/347,835 and of PCT Patent Application Nos. PCT/CA2022/051228 and PCT/CA2022/051266 are hereby incorporated by reference.

### FIELD OF THE DISCLOSURE

The compositions and methods disclosed herein relate to tryptamines. Furthermore, the compositions and methods disclosed herein relate in particular to C₄-substituted tryptamine derivatives.

### BACKGROUND OF THE DISCLOSURE

The following paragraphs are provided by way of background to the present disclosure. They are not however an admission that anything discussed therein is prior art or part of the knowledge of a person of skill in the art.

Tryptamines are a class of chemical compounds that share a common chemical structure (notably, a fused benzene and pyrrole ring, together known as an indole, and linked to the pyrrole ring, at the third carbon atom, a 2-aminoethyl group), and can be formulated as therapeutic drug compounds. For example, psilocybin has been evaluated as a drug for its clinical potential in the treatment of mental health conditions (Daniel, J. et al. Mental Health Clin/, 2017; 7(1): 24-28), including to treat anxiety in terminal cancer patients (Grob, C. et al. Arch. Gen. Psychiatry, 2011, 68(1) 71-78) and to alleviate symptoms of treatment-resistant depression (Cathart-Harris, R. L. et al. Lancet Psychiatry, 2016, 3: 619-627). Other known drug compounds within the tryptamine class of compounds include, for example, melatonin, serotonin, bufotenin, dimethyltryptamine (DMT), and psilocin.

It is commonly understood that tryptamine-based drugs can produce their in vivo therapeutic effects by molecular interaction with macromolecules present in human cells, known as receptors. In this respect, in broad terms, specific receptors can be thought of as being located in a relatively fixed anatomical space (e.g., a specific brain tissue). Following administration of a drug, the drug moves through the body to the receptor to interact therewith, and then back out of the body. It is generally desirable that when a tryptamine-based drug is administered, the drug is specifically active at the desired anatomical location within a patient's body, such as, for example, in a specific brain tissue and/or at a specific receptor, a 5-hydroxytryptamine (5-HT) receptor, for example. Moreover, it is generally desirable that the specific molecular interaction between the drug and a receptor, such as a 5-HT receptor, is such that the drug-receptor molecular interaction results in appropriate modulation of the target receptor.

In many instances the observed pharmacological effect of tryptamine-based drugs is suboptimal. Thus, administration of the drug may fall short of the desired therapeutic effect (e.g., the successful treatment of a psychotic disorder) and/or undesirable side effects may be observed.

The underlying causes for these observed shortcomings in pharmacological effects may be manifold. For example, the administered drug additionally may interact with receptors other than the target receptor, and/or the specific molecular interaction between drug and target may not lead to the desired receptor modulation, and/or the concentration of the drug at the receptor may be suboptimal. In this respect, known tryptamine-based drugs can be said to frequently display suboptimal pharmacodynamic (PD) characteristics, i.e., suboptimal characteristics with respect to the pharmacological effect exerted by the drug on the body. Thus, for example, the intensity of the drug's effect, the concentration of the drug at the receptor, and the molecular interactions between the drug, and receptor may not be as desired.

Furthermore, as is the case with many pharmaceutical compounds, tryptamine compounds when administered can penetrate multiple tissues by diffusion, resulting in broad bodily distribution of the drug compound (Bodor, N. et al., 2001, J. Pharmacy and Pharmacology, 53: 889-894). Thus, frequently a substantial proportion of the administered drug fails to reach the desired target receptor. This in turn may necessitate more frequent dosing of the drug. Such frequent dosing is less convenient to a patient, and, moreover, may negatively affect patient compliance with the prescribed drug therapy. In addition, generally toxicity associated with drug formulations tends to be more problematic as a result of broad bodily distribution of the drug throughout the patient's body since undesirable side effects may manifest themselves as a result of interaction of the drug with healthy organs.

Furthermore, it is generally desirable that drug compounds exert a pharmacological effect for an appropriate period of time. However, tryptamine-based drugs when systemically administered to a patient can exhibit a high blood plasma clearance, typically on the order of minutes (Vitale, A. et al., 2011, J. of Nucl. Med., 52(6), 970-977). Thus, rapid drug clearance can also necessitate more frequent dosing of tryptamine-based drug formulations. In this respect, known tryptamine containing drug formulations can be said to frequently display suboptimal pharmacokinetic (PK) characteristics, i.e., suboptimal characteristics with respect to movement of the drug through the body to and from the desired anatomical location, including, for example, suboptimal drug absorption, distribution, metabolism, and excretion.

There exists therefore a need in the art for improved tryptamine compounds.

### SUMMARY OF THE DISCLOSURE

The following paragraphs are intended to introduce the reader to the more detailed description, not to define or limit the claimed subject matter of the present disclosure.

In one aspect, the present disclosure relates to tryptamines and derivative compounds thereof.

In another aspect, the present disclosure relates to C₄-substituted tryptamine derivative compounds.

Accordingly, in one aspect, the present disclosure provides, in at least one embodiment, in accordance with the teachings herein, a chemical compound having chemical formula (I):

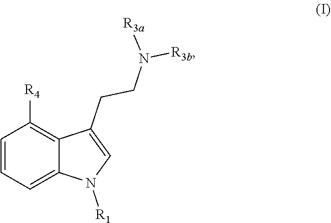

wherein R₄ is a substituent containing:
(A) an ether moiety or a derivative thereof;
(B) a carbonic ester moiety or a derivative thereof;
(C) a carboxylic acid moiety or a derivative thereof;
(D) a polyether or a derivative thereof;
(E) a carbonothioate moiety or a derivative thereof; or
(F) a phosphate moiety or a derivative thereof;
   wherein R₁ is a hydrogen atom or a moiety containing a carbonic ester or derivative thereof, or a ketone or ketone derivative; and wherein R₃ₐ and R_{3b} are each independently a hydrogen atom or an alkyl group.

In at least one embodiment, in an aspect, R₄ can be a substituent selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof.

In at least one embodiment, in an aspect, in the compound having chemical formula (I) wherein R₄ is an ether moiety or derivative thereof, the compound having formula (I) can have the chemical formula (XX):

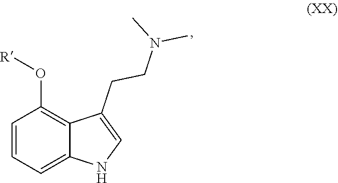

wherein
R′ is an aryl group, a heteroaryl group, a heterocyclic group, an alkyl-aryl group, an alkyl-heterocyclic group, a silyl group or an alkyl-heteroaryl group, wherein the aryl group, heteroaryl group, alkyl-aryl group, alkyl-heterocycle group, or alkyl-heteroaryl groups are substituted or unsubstituted, and wherein the optional substituents are a halo group, an amide group, an oxo group, or an alkyl group, wherein two or more alkyl groups optionally form an additional 5-7-membered ring.

In at least one embodiment, in an aspect, in the compound having formula (I) wherein R₄ is an ether moiety or derivative thereof, the ether moiety or derivative thereof can be an aryl ether group, a heteroaryl ether group, a heterocyclic ether group, an alkyl-aryl ether group, an alkyl-heterocyclic group, a silyl ether group or an alkyl-heteroaryl group, wherein the aryl group, heteroaryl group, alkyl-aryl group, alkyl-heterocycle group, or alkyl-heteroaryl groups are substituted or unsubstituted, and wherein the optional substituents are a halo group, an amide group, an oxo group, or an alkyl group, wherein two or more alkyl groups optionally form an additional 5-7-membered ring.

In at least one embodiment, in an aspect, the alkyl-aryl can be —CH₂-phenyl or —CH₂-naphthyl, in which the phenyl or naphthyl are optionally substituted.

In at least one embodiment, in an aspect, the aryl group can be a phenyl group or a naphthyl group.

In at least one embodiment, in an aspect, the additional ring can be a cyclopentyl or cyclohexyl group.

In at least one embodiment, in an aspect, the alkaryl ether or alkyl-heteroaryl group can be a C₁-C₆-alkylene-aryl group or a C₁-C₆-alkylene-heteroaryl group.

In at least one embodiment, in an aspect, the C₁-C₆-alkylene-aryl group or a C₁-C₆-alkylene-heteroaryl group can be a C₁-C₆-alkylene-phenyl or C₁-C₆-alkylene-naphthyl, respectively.

In at least one embodiment, in an aspect, the ether moiety or derivative can be a cycloalkyl ether, in which at least one carbon atom of the ring is optionally replaced with a heteroatom selected from O or N.

In at least one embodiment, in an aspect, the cycloalkyl ether can be a C₃-C₆-cycloalkyl group.

In at least one embodiment, in an aspect, the ether moiety or derivative thereof can be a silyl ether wherein the silicon atom is substituted with one or more alkyl groups or aryl groups.

In at least one embodiment, in an aspect, the alkyl group can be a C₁-C₆-alkyl group and the aryl group can be a phenyl group.

In at least one embodiment, in an aspect, the C₁-C₆-alkyl group can be a methyl, ethyl, propyl, n-butyl, s-butyl, or t-butyl.

In at least one embodiment, in an aspect, in the compound having chemical formula (I) wherein R₄ is an ether moiety or derivative thereof, the compound can be selected from the group consisting of A(I), A(II), A(III), A(IV), A(V), A(VI), A(VII), A(VIII), A(IX) and A(X):

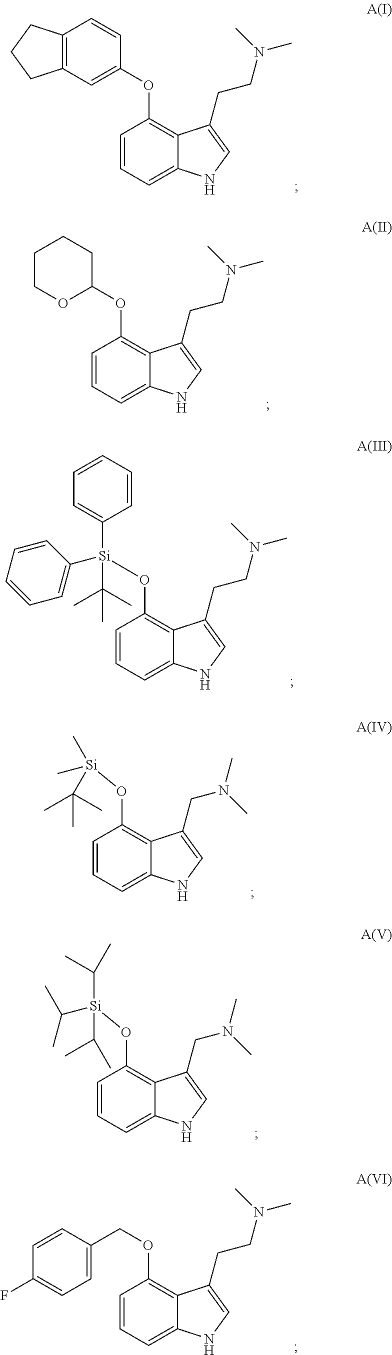

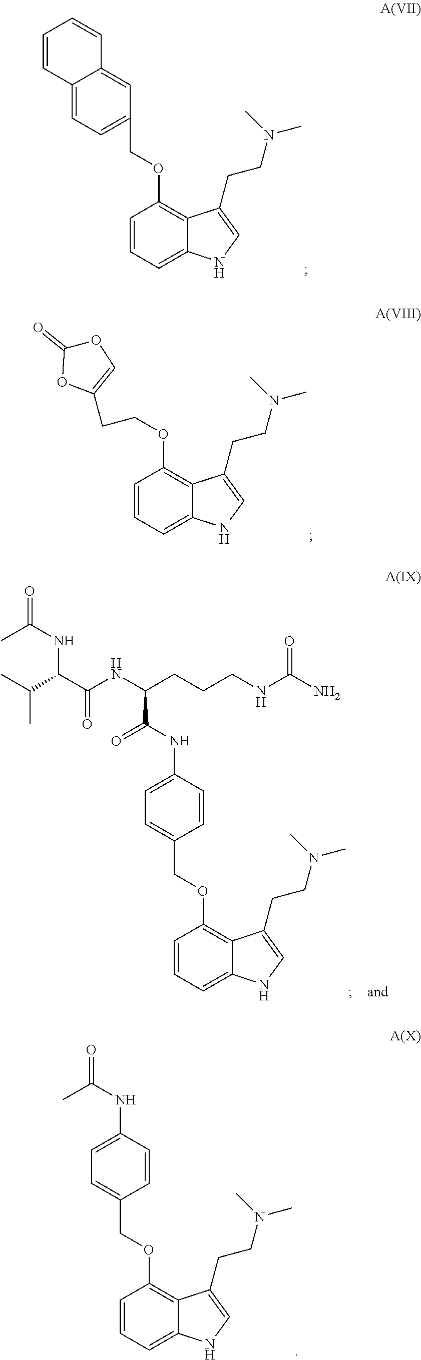

In at least one embodiment, in an aspect, in the compound having chemical formula (I) wherein R₄ is a carbonic ester moiety or derivative thereof, the compound having formula (I) can have the chemical formula (XIV):

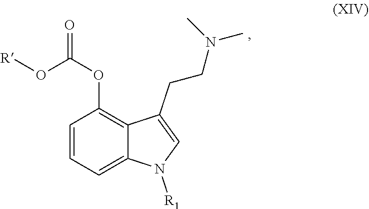

wherein
R₁ is a hydrogen atom or a carbonic ester moiety —O—C(═O)—O—(C₁-C₆)-alkyl, wherein the alkyl is optionally substituted with halo or C₆-C₁₀-aryl;
R′ is C₁-C₁₀-alkyl, wherein the C₁-C₁₀-alkyl group is optionally substituted with one or more of halo, C₆-C₁₀-aryl, C₃-C₆-cycloalkyl, or a polycyclic hydrocarbon.

In at least one embodiment, in an aspect, the optional substituent can be fluorenyl, phenyl, cyclopropyl, or chloro.

In at least one embodiment, R₁ can be O—C(═O)—O—CH₂-phenyl.

In at least one embodiment, in an aspect, in the compound having chemical formula (I) wherein R₄ is a carbonic ester moiety or derivative thereof, the compound can be selected from the group consisting of B(I), B(II), B(III), B(IV), B(V), and B(VI):

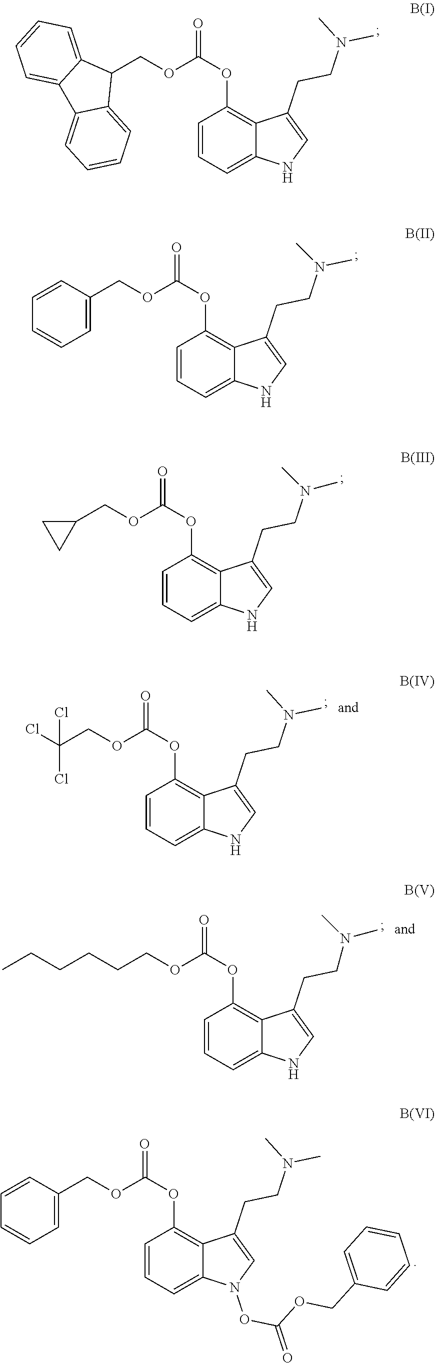

In at least one embodiment, in an aspect, in the compound having formula (I) wherein R₄ is a polyether moiety or derivative thereof, the polyether moiety or derivative thereof can include a carboxylic acid moiety or derivative thereof.

In at least one embodiment, in an aspect, the polyether moiety or derivative thereof can include an alkyl group, a cyclo-alkyl group, or an O-alkyl group.

In at least one embodiment, in an aspect, the polyether derivative can be a compound having the formula (XV):

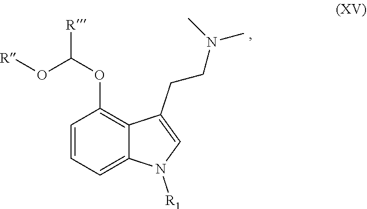

wherein
R₁ is a hydrogen atom; a (C₁-C₆)-alkyl group, in which two hydrogens are optionally replaced with an oxo group; a carbonic ester moiety —C—O—C(═O)—O—(C₁-C₆)-alkyl; or an acyl moiety —C(═O)—(C₁-C₆)-alkyl,
R″ is a polyether moiety or derivative thereof, and
R′″ is H or C₁-C₆-alkyl.

In at least one embodiment, in an aspect, R₁ can be —C(═O)CH₂CH₃.

In at least one embodiment, in an aspect, R′″ can be a methyl.

In at least one embodiment, in an aspect, R″ can be optionally substituted C₁-C₁₀-alkyl, in which the optional substituents can be alkyl, cycloalkyl, aryl, oxo, wherein two hydrogens on the alkyl group form an oxo group (═O), and in which one or more carbon atoms in the alkyl group are optionally replaced with an oxygen (O) atom.

In at least one embodiment, in an aspect, R″ can be alkyl substituted by phenyl (—CH₂-phenyl).

In at least one embodiment, in aspect, R″ can be methyl.

In at least one embodiment, in an aspect, R″ can be a C₁-C₆ alkyl group in which two geminal hydrogens on the alkyl group are joined together to form an oxo group (═O).

In at least one embodiment, in an aspect, the C₁-C₆-alkyl group substituted by an oxo group, can be further substituted by a C₃-C₆-cycloalkyl group or additional alkyl group.

In at least one embodiment, in an aspect, the C₃-C₆-cycloalkyl group can be a cyclopropyl group.

In at least one embodiment, in an aspect, the additional alkyl group can be a methyl group.

In at least one embodiment, in an aspect, R″ can be an optionally substituted C₁-C₁₀-alkyl group in which at least one of the carbon atoms in the alkyl group are optionally replaced with an oxygen (O) atom.

In at least one embodiment, in an aspect, R″ can be an optionally substituted C₁-C₆-alkyl group in which at least one of the carbon atoms in the alkyl group are optionally replaced with an oxygen (O) atom, and which is further substituted by an oxo group or an alkyl group.

In at least one embodiment, in an aspect, the polyether moiety or derivative thereof can be selected from the group of compounds consisting of D(I), D(II), D(III), D(IV), D(V), D(VI), D(VII), D(VIII), D(IX), D(X), D(XI), D(XII), D(XIII), D(XIV), D(XV), D(XVI), D(XVII), D(XVIII), and D(XIX):

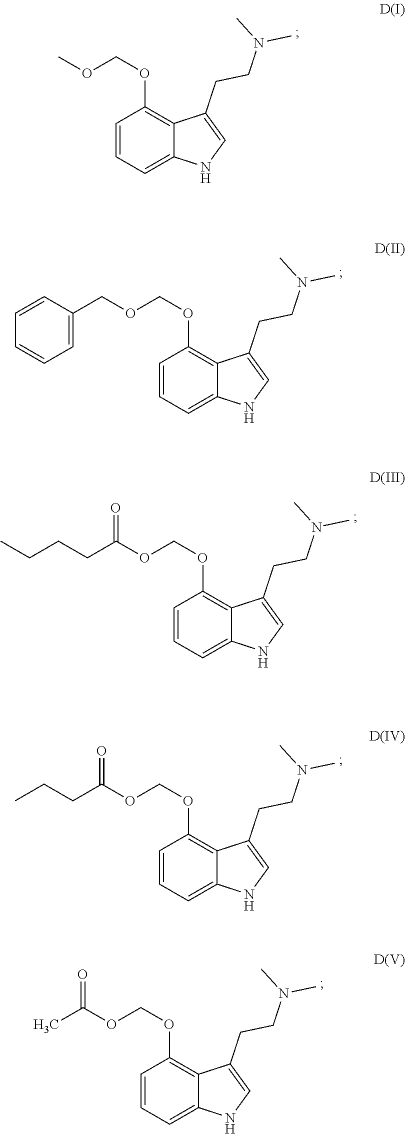

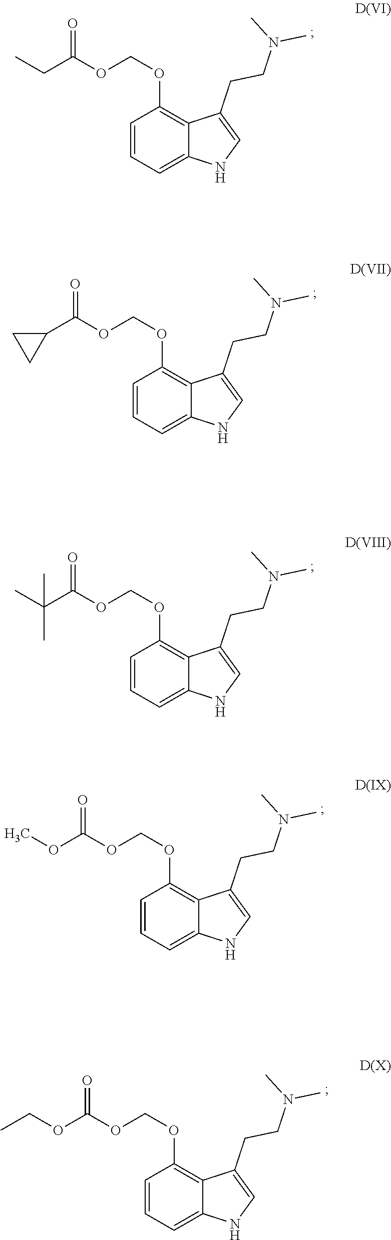

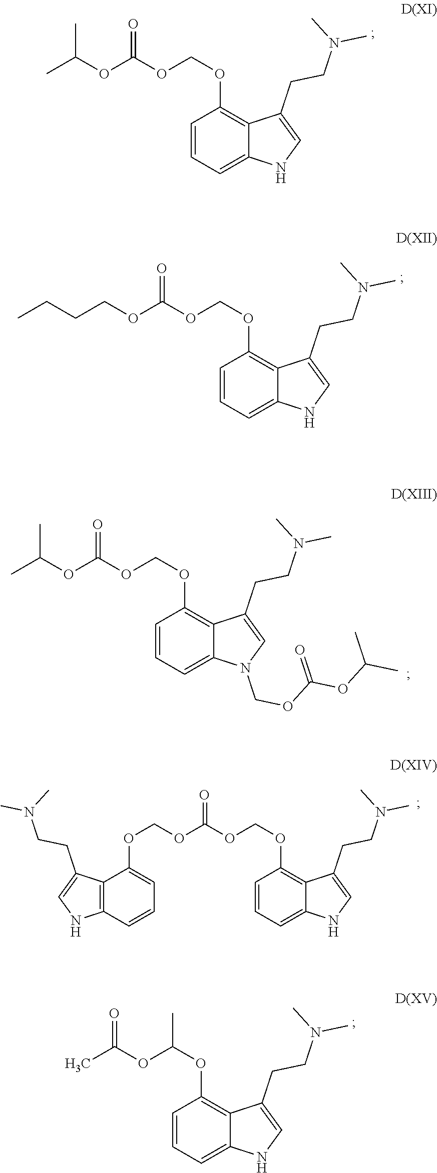

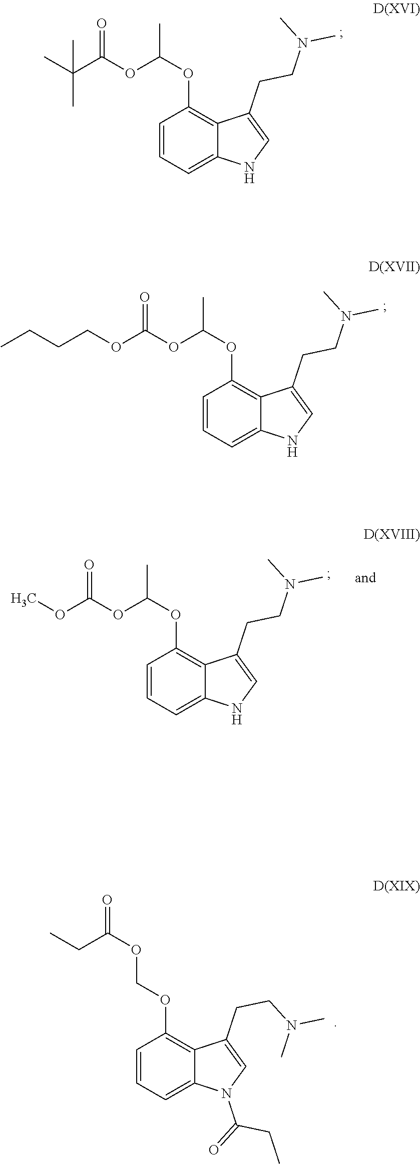

In at least one embodiment, in an aspect, in the compound having chemical formula (I) wherein R₄ is a phosphate moiety or derivative thereof, the compound having formula (I) can have chemical formula (XVI):

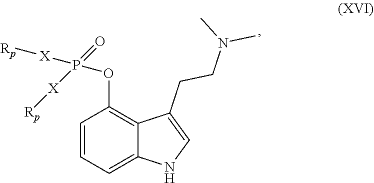

wherein,
X is O or NH;
each Rₚ is independently or simultaneously alkyl, aryl, or each Rₚ is joined together to form an optionally substituted 4-7-membered heterocyclic ring.

In at least one embodiment, in an aspect, Rₚ can be a phenyl group.

In at least one embodiment, in an aspect, each Rₚ can be joined together to form an optionally substituted 5-6-membered heterocyclic ring, in which the optional substituents are a phenyl group, or alkyl group, or two substituents are joined together to form an optionally substituted phenyl ring, substituted by an alkyl group or an alkoxy group.

In at least one embodiment, in an aspect, the alkyl group can be a methyl group.

In at least one embodiment, in an aspect, the alkoxy group can be a methoxy group.

In at least one embodiment, in an aspect, Rₚ can be an optionally substituted C₁-C₁₀-alkyl group, wherein one or more atoms in the C₁-C₁₀-alkyl group are replaced with an oxygen (O) atom or sulfur (S) atom, and the optional substituents are oxo, wherein two hydrogens on the same carbon are taken together to form an oxo group (═O), or alkyl.

In at least one embodiment, in an aspect, the alkyl group can be a methyl group or ethyl group.

In at least one embodiment, in an aspect, in the compound having chemical formula (I) wherein R₄ is a phosphate moiety or derivative thereof, the compound can be selected from the group consisting of F(I), F(II), F(III), F(IV), F(V), F(VI), F(VII), F(VIII), F(IX), F(X), F(XI), F(XII), F(XIII), F(XIV), and F(XV):

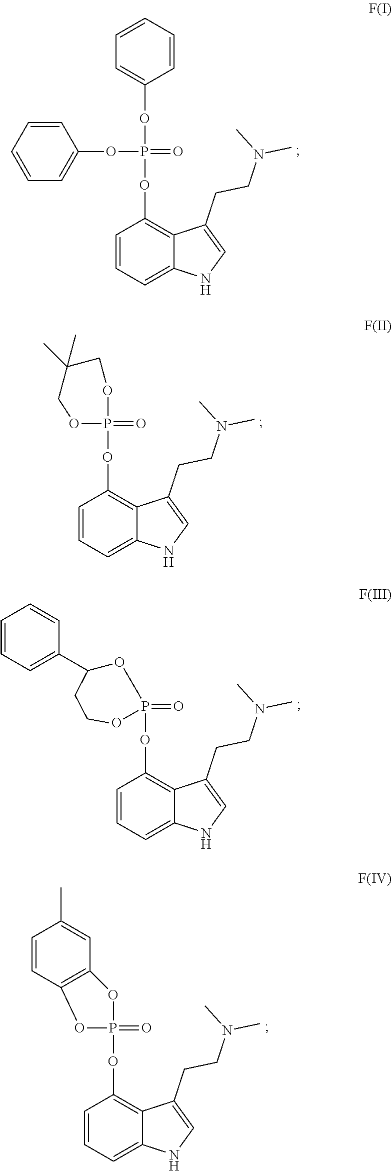

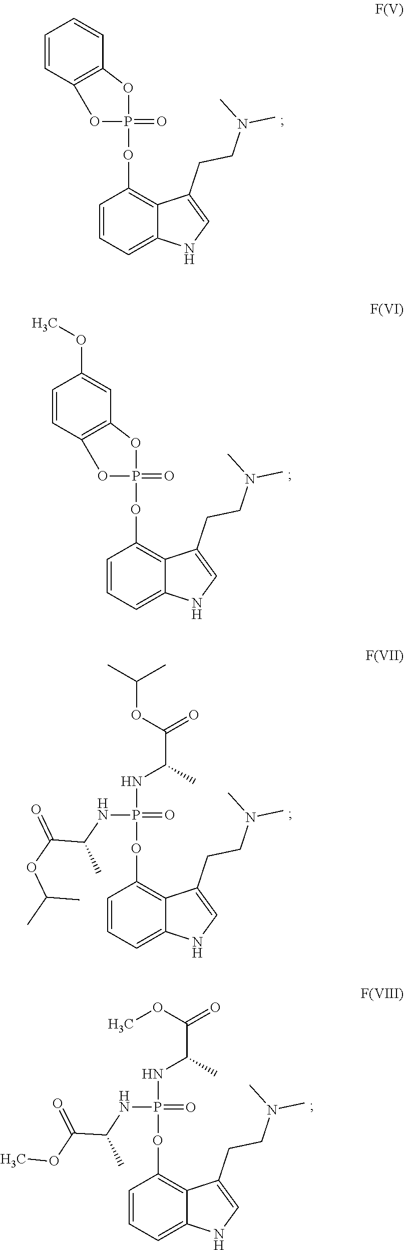

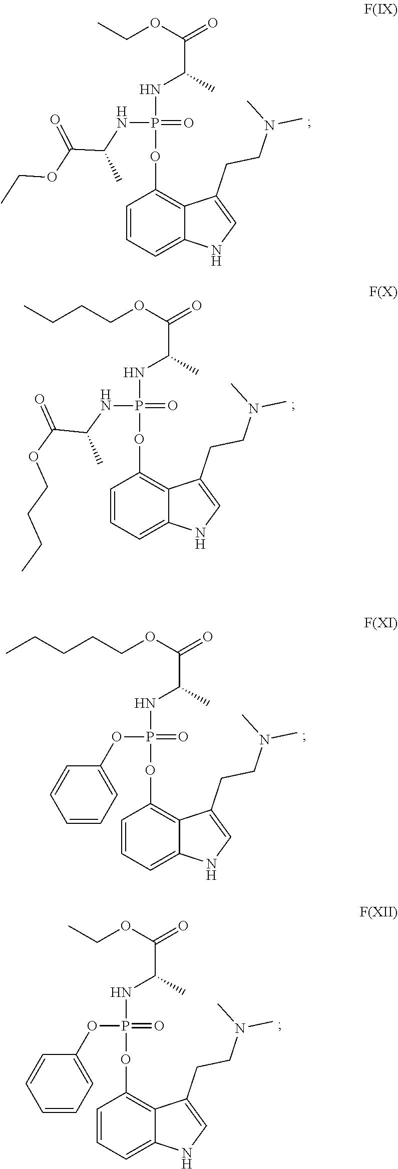

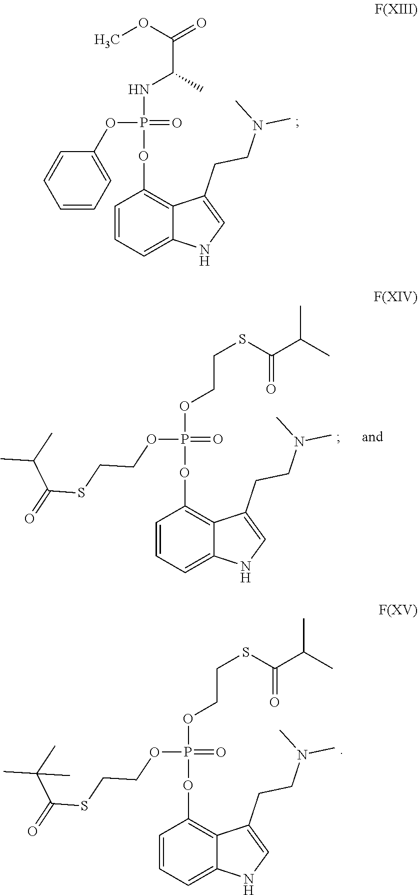

In at least one embodiment, in an aspect, R₁ can be a carbonic ester having chemical formula (V) or (XIII):

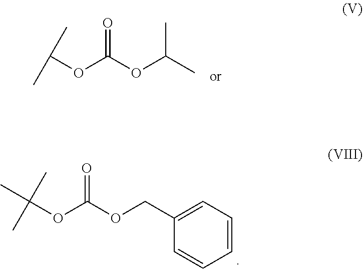

In at least one embodiment, in an aspect, R₁ can be a ketone or a ketone derivative having formula (XVII):

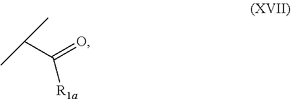

wherein R₁ₐ is a hydrogen atom or an alkyl group.

In at least one embodiment, in an aspect, R₁ can have formula (XVIII) or (XIX):

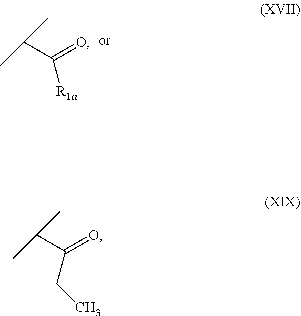

In at least one embodiment, in an aspect, R₄ can be a substituent selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof,
   wherein (NR₃ₐR_{3b}) has the formula (N⁺HR₃ₐR_{3b}Z), wherein Z is a counter-balancing anion, and wherein compound (I) is a salt.

In at least one embodiment, in an aspect, Z can be a mono-valent counter-balancing ion (Z⁻), a di-valent counter-balancing ion (Z²⁻), or a tri-valent counter-balancing ion (Z³⁻).

In at least one embodiment, in an aspect, Z can be a mono-valent counter-balancing anion (Z⁻) selected from a halide ion (Cl⁻, Br⁻, F⁻, I⁻), a nitrate ion (NO₃⁻), a benzoate ion (phenyl-COO⁻), a succinate ion (HOOC—(CH₂)₂—COO⁻), a fumarate ion (trans-HOOC—(CH═CH)—COO⁻), a tartarate ion (HOOC—(CHOH)₂—COO⁻), a malate ion (HOOC—CH₂—CHOH—COO⁻), a maleate ion (cis-HOOC—(CH═CH)—COO⁻), a dibenzoyl tartarate ion (HOOC—(CHOBz)₂-COO⁻), a ditoluoyl tartarate ion (HOOC—(CHOCOTol)₂-COO⁻), a malonate ion (HOOC—CH₂—COO⁻), a dihydrogen phosphate ion (H₂PO₄⁻), and an acetate ion (CH₃—COO⁻), wherein the salt compound has the formula (Iₐ):

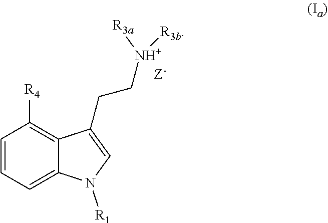

In at least one embodiment, in an aspect, Z can be a di-valent counter-balancing anion (Z²—) selected from a sulfate ion (SO₄²⁻), a hydrogen phosphate ion (HPO₄²⁻), a succinate dianion (⁻OOC—(CH₂)₂—COO⁻), a fumarate dianion (trans-⁻OOC—(CH═CH)—COO⁻), a tartarate dianion (⁻OOC—(CHOH)₂—COO⁻), a malate dianion (⁻OOC—CH₂—CHOH—COO⁻), a maleate dianion (cis-⁻OOC—(CH═CH)—COO⁻), a dibenzoyl tartarate dianion (⁻OOC—(CHOBz)₂-COO⁻), a ditoluoyl tartarate dianion (⁻OOC—(CHOCOTol)₂-COO⁻), and a malonate dianion (⁻OOC—CH₂—COO⁻), wherein the salt compound has the formula (I_{b}):

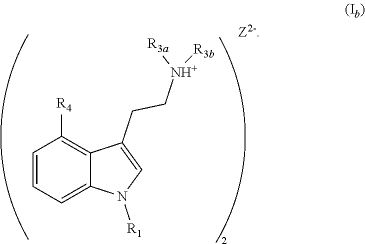

In at least one embodiment, in an aspect, Z can be a tri-valent counter-balancing anion (Z³⁻) selected from a phosphate ion (PO₄³⁻) and a citrate ion (⁻OOC—CH₂—C(OH)(COO⁻)—CH₂—COO⁻, and the salt compound has the formula (I_{c}):

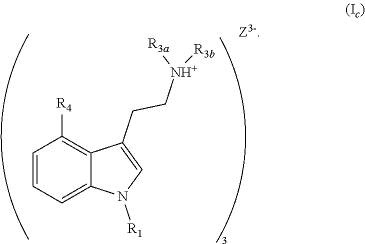

In another aspect, the present disclosure relates to pharmaceutical and recreational drug formulations comprising C₄-substituted derivative compounds. Accordingly, in one aspect, the present disclosure provides, in at least one embodiment, a pharmaceutical or recreational drug formulation comprising an effective amount of a chemical compound having a formula (I):

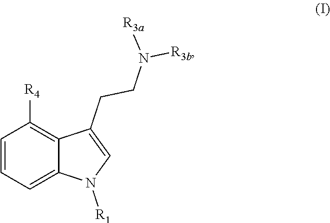

wherein R₄ is a substituent selected from:
(A) an ether moiety or derivative thereof;
(B) a carbonic ester moiety or derivative thereof;
(C) a carboxylic acid moiety or derivative thereof;
(D) a polyether moiety or derivative thereof;
(E) a carbonothioate moiety or derivative thereof; and
(F) a phosphate moiety or derivative thereof;
   wherein R₁ is a hydrogen atom or a moiety containing a carbonic ester or derivative thereof, or a ketone or ketone derivative; and
   wherein R₃ₐ and R_{3b} are each independently a hydrogen atom or an alkyl group, together with a pharmaceutically acceptable excipient, diluent, or carrier.

In at least one embodiment, in an aspect, R₄ can be a substituent selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof,
wherein (NR₃ₐR_{3b}) has the formula (N⁺HR₃ₐR_{3b}Z), wherein Z is a pharmaceutically acceptable counter-balancing anion, and wherein compound (I) is a salt.

In at least one embodiment, in an aspect, Z can be a pharmaceutically acceptable mono-valent counter-balancing ion (Z⁻), a pharmaceutically acceptable di-valent counter-balancing ion (Z²⁻), or a pharmaceutically acceptable tri-valent counter-balancing ion (Z³⁻).

In at least one embodiment, in an aspect, Z can be a mono-valent counter-balancing anion (Z⁻) selected from a halide ion (Cl⁻, Br⁻, F⁻, I⁻), a nitrate ion (NO₃⁻), a benzoate ion (phenyl-COO⁻), a succinate ion (HOOC—(CH₂)₂—COO⁻), a fumarate ion (trans-HOOC—(CH═CH)—COO⁻), a tartarate ion (HOOC—(CHOH)₂—COO⁻), a malate ion (HOOC—CH₂—CHOH—COO⁻), a maleate ion (cis-HOOC—(CH═CH)—COO⁻), a dibenzoyl tartarate ion (HOOC—(CHOBz)₂-COO⁻), a ditoluoyl tartarate ion (HOOC—(CHOCOTol)₂-COO⁻), a malonate ion (HOOC—CH₂—COO⁻), a dihydrogen phosphate ion (H₂PO₄⁻), and an acetate ion (CH₃—COO⁻), wherein the salt compound has the formula (Iₐ):

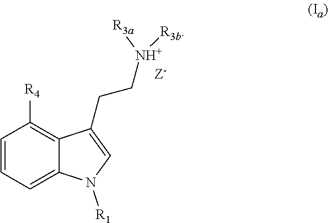

In at least one embodiment, in an aspect, Z can be a di-valent counter-balancing anion (Z²—) selected from a sulfate ion (SO₄²⁻), a hydrogen phosphate ion (HPO₄²⁻), a succinate dianion (⁻OOC—(CH₂)₂—COO⁻), a fumarate dianion (trans-⁻OOC—(CH═CH)—COO⁻), a tartarate dianion (⁻OOC—(CHOH)₂—COO⁻), a malate dianion (⁻OOC—CH₂—CHOH—COO⁻), a maleate dianion (cis-⁻OOC—(CH═CH)—COO⁻), a dibenzoyl tartarate dianion (⁻OOC—(CHOBz)₂-COO⁻), a ditoluoyl tartarate dianion (⁻OOC—(CHOCOTol)₂-COO⁻), and a malonate dianion (⁻OOC—CH₂—COO⁻), wherein the salt compound has the formula (I_{b}):

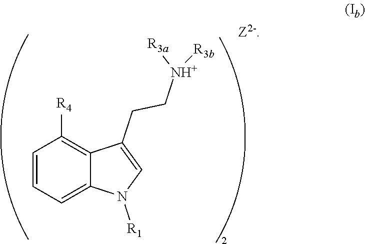

In at least one embodiment, in an aspect, Z can be a pharmaceutically acceptable tri-valent counter-balancing anion (Z³⁻) selected from a phosphate ion (PO₄³⁻) and a citrate ion (⁻OOC—CH₂—C(OH)(COO⁻)—CH₂—COO⁻, and the salt compound has the formula (I_{c}):

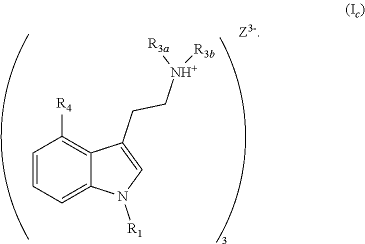

In at least one embodiment, in an aspect, the pharmaceutical formulation can be a pro-drug pharmaceutical formulation, wherein the compound having formula (I) is in vivo hydrolyzed to form a compound having chemical formula (VIₐ) or (VI_{b}):

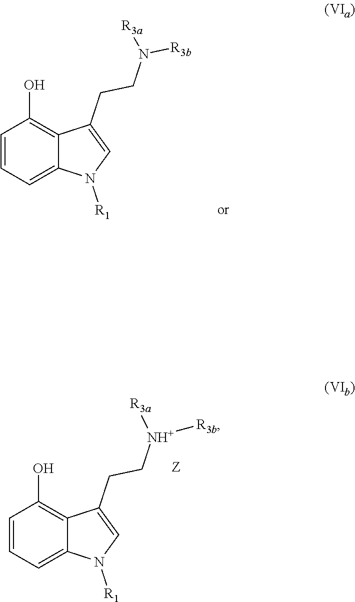

wherein R₁ is a hydrogen atom or a moiety containing a carbonic ester or derivative thereof, or a ketone or ketone derivative; and wherein R₃ₐ and R_{3b} are each independently a hydrogen atom or an alkyl group.

In another aspect, the present disclosure relates to methods of treatment of psychiatric disorders. Accordingly, the present disclosure further provides, in one embodiment a method for treating a psychiatric disorder, the method comprising administering to a subject in need thereof a pharmaceutical formulation comprising a chemical compound having a formula (I):

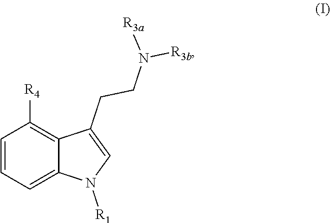

wherein R₄ is a substituent selected from:
(A) an ether moiety or derivative thereof;
(B) a carbonic ester moiety or derivative thereof;
(C) a carboxylic acid moiety or derivative thereof;
(D) a polyether moiety or derivative thereof;
(E) a carbonothioate moiety or derivative thereof; and
(F) a phosphate moiety or derivative thereof,
   wherein R₁ is a hydrogen atom or a moiety containing a carbonic ester or derivative thereof, or a ketone or ketone derivative; and
   wherein R₃ₐ and R_{3b} are each independently a hydrogen atom or an alkyl group,
   wherein the pharmaceutical formulation is administered in an effective amount to treat the psychiatric disorder in the subject.

In at least one embodiment, in an aspect, R₄ can be a substituent selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof.

In at least one embodiment, in an aspect, upon administration the compound having formula (I) interacts with a receptor in the subject to thereby modulate the receptor and exert a pharmacological effect.

In at least one embodiment, in an aspect, the receptor can be a 5-HT_{1A} receptor, a 5-HT_{2A} receptor, a 5-HT_{1B} receptor, a 5-HT_{2B} receptor, a 5-HT_{3A} receptor, an ADRA1A receptor, an ADRA2A receptor, a CHRM1 receptor, a CHRM2 receptor, a CNR1 receptor, a DRD1 receptor, a DRD2S receptor, or an OPRD1 receptor.

In at least one embodiment, in an aspect, upon administration the compound having formula (I) can interact with an enzyme or transmembrane transport protein in the subject to thereby modulate the enzyme or transmembrane transport protein and exert a pharmacological effect.

In at least one embodiment, in an aspect, the enzyme can be monoamine oxidase A (MOA-A), and the transmembrane transport protein can be a dopamine active transporter (DAT), a norephedrine transporter (NET), or a serotonin transporter (SERT) transmembrane transport protein.

In at least one embodiment, in an aspect, upon administration the compound having formula (I) can be in vivo hydrolyzed to form a compound having chemical formula (VIₐ) or (VI_{b}):

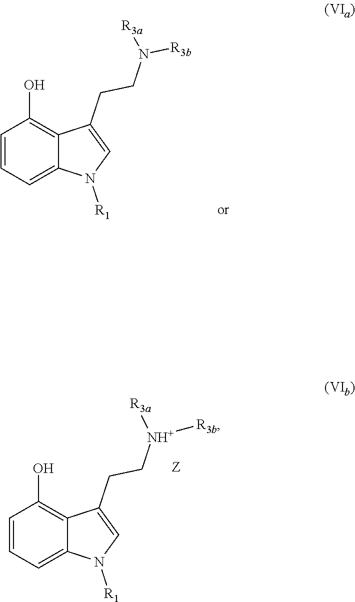

wherein R₁ is a hydrogen atom or a moiety containing a carbonic ester or derivative thereof, or a ketone or ketone derivative, and wherein Z is a counterbalancing anion; and
wherein R₃ₐ and R_{3b} are each independently a hydrogen atom or an alkyl group, and wherein the compound having chemical formula (VIₐ) or (VI_{b}) interacts with a receptor to thereby modulate the receptor in the subject and exert a pharmacological effect.

In at least one embodiment, in an aspect, the receptor can be a5-HT_{1A} receptor, a 5-HT_{2A} receptor, a 5-HT_{1B} receptor, a 5-HT_{2B} receptor, a 5-HT_{3A} receptor, an ADRA1A receptor, an ADRA2A receptor, a CHRM1 receptor, a CHRM2 receptor, a CNR1 receptor, a DRD1 receptor, a DRD2S receptor, or an OPRD1 receptor.

In at least one embodiment, in an aspect, the disorder can be a 5-HT_{1A} receptor mediated disorder, a 5-HT_{2A} receptor mediated disorder, a 5-HT_{1B} receptor mediated disorder, a 5-HT_{2B} receptor mediated disorder, a 5-HT_{3A} receptor mediated disorder, an ADRA1A receptor mediated disorder, an ADRA2A receptor mediated disorder, a CHRM1 receptor mediated disorder, a CHRM2 receptor mediated disorder, a CNR1 receptor mediated disorder, a DRD1 receptor mediated disorder, a DRD2S receptor r mediated disorder, or an OPRD1 receptor mediated disorder.

In at least one embodiment, in an aspect, a dose can be administered of about 0.001 mg to about 5,000 mg.

In another aspect, the present disclosure provides, in at least one embodiment, a method for modulating (i) a receptor selected from 5-HT_{1A} receptor, a 5-HT_{2A} receptor, a 5-HT_{1B} receptor, a 5-HT_{2B} receptor, a 5-HT_{3A} receptor, an ADRA1A receptor, an ADRA2A receptor, a CHRM1 receptor, a CHRM2 receptor, a CNR1 receptor, a DRD1 receptor, a DRD2S receptor, or an OPRD1 receptor; (ii) an enzyme, the enzyme being MOA-1; or (iii) a transmembrane transport protein selected from a dopamine active transporter (DAT), a norephedrine transporter (NET) or a serotonin transporter (SERT) transmembrane transport protein, the method comprising contacting (i) the 5-HT_{1A} receptor, the 5-HT_{2A} receptor, the 5-HT_{1B} receptor, the 5-HT_{2B} receptor, the 5-HT_{3A} receptor, the ADRA1A receptor, the ADRA2A receptor, the CHRM1 receptor, the CHRM2 receptor, the CNR1 receptor, the DRD1 receptor, the DRD2S receptor, or the OPRD1 receptor; (ii) MOA-1; or (iii) the dopamine active transporter (DAT), the norephedrine transporter (NET), or the serotonin transporter (SERT) transmembrane transport protein with a chemical compound having a formula (I):

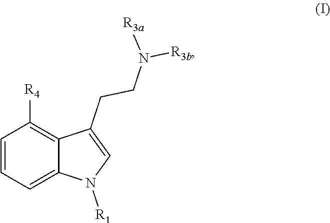

wherein R₄ is a substituent selected from:
(A) an ether moiety or derivative thereof;
(B) a carbonic ester moiety or derivative thereof;
(C) a carboxylic acid moiety or derivative thereof;
(D) a polyether moiety or derivative thereof;
(E) a carbonothioate moiety or derivative thereof; and
(F) a phosphate moiety or derivative thereof,
   wherein R₁ is a hydrogen atom or a moiety containing a carbonic ester or derivative thereof, or a ketone or ketone derivative; and
   wherein R₃ₐ and R_{3b} are each independently a hydrogen atom or an alkyl group, under reaction conditions sufficient to modulate (i) the 5-HT_{1A} receptor, the 5-HT_{2A} receptor, the 5-HT_{1B} receptor, the 5-HT_{2B} receptor, the 5-HT_{3A} receptor, the ADRA1A receptor, the ADRA2A receptor, the CHRM1 receptor, the CHRM2 receptor, the CNR1 receptor, the DRD1 receptor, the DRD2S receptor, or the OPRD1 receptor; (ii) MOA-1; or (iii) the dopamine active transporter (DAT), the norephedrine transporter (NET), or the serotonin transporter (SERT) transmembrane transport protein.

In at least one embodiment, in an aspect, R₄ can be a substituent selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof.

In at least one embodiment, in an aspect, the reaction conditions can be in vitro reaction conditions.

In at least one embodiment, in an aspect, the reaction conditions can be in vivo reaction conditions.

In another aspect, the present disclosure relates to methods of making a chemical compound having a formula (I):

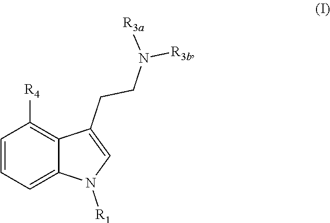

wherein R₄ is a substituent selected from:
(A) an ether moiety or derivative thereof;
(B) a carbonic ester moiety or derivative thereof;
(C) a carboxylic acid moiety or derivative thereof;
(D) a polyether moiety or derivative thereof;
(E) a carbonothioate moiety or derivative thereof; and
(F) a phosphate moiety or derivative thereof,
   wherein R₁ is a hydrogen atom or a moiety containing a carbonic ester or derivative thereof, or a ketone or ketone derivative; and
   wherein R₃ₐ and R_{3b} are each independently a hydrogen atom or an alkyl group, the method comprising the performance of at least one chemical synthesis reaction selected from the reactions depicted in FIGS. 3A, 4A, 5A, 6A, 7A, 8A, 9A (i), 9A (ii), 10A, 11A, 12A, 13A, 14A, and 15A.

In at least one embodiment, R₄ can be a substituent selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof.

In at least one embodiment, the chemical compound can have the formula F(I):

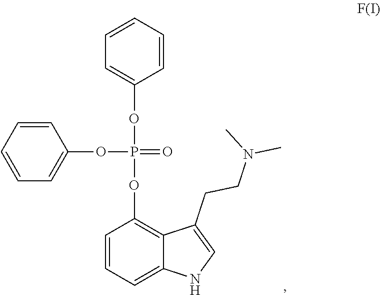 [SMILES: CN(C)CCc1c[nH]c2cccc(OP(=O)(Oc3ccccc3)Oc3ccccc3)c12]

and the at least one chemical synthesis reaction is the reaction depicted in FIG. 3A.

In at least one embodiment, the chemical compound can have the formula A(V):

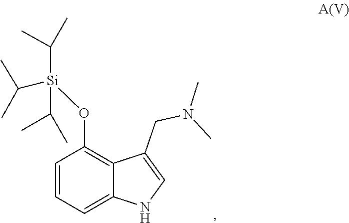 [SMILES: CC(C)[Si](Oc1cccc2[nH]cc(CN(C)C)c12)(C(C)C)C(C)C]

and the at least one chemical synthesis reaction is the reaction depicted in FIG. 4A.

In at least one embodiment, the chemical compound can have the formula A(III):

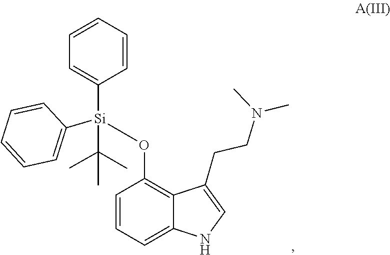 [SMILES: CN(C)CCc1c[nH]c2cccc(O[Si](c3ccccc3)(c3ccccc3)C(C)(C)C)c12]

and the at least one chemical synthesis reaction is the reaction depicted in FIG. 5A.

In at least one embodiment, the chemical compound can have the formula B(IV):

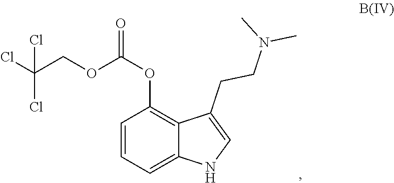 [SMILES: CN(C)CCc1c[nH]c2cccc(OC(=O)OCC(Cl)(Cl)Cl)c12]

and the at least one chemical synthesis reaction is the reaction depicted in FIG. 6A.

In at least one embodiment, the chemical compound can have the formula B(V):

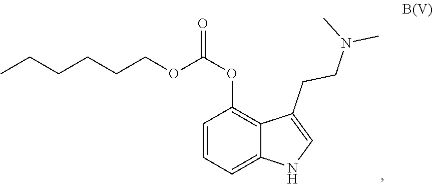 [SMILES: CCCCCCOC(=O)Oc1cccc2[nH]cc(CCN(C)C)c12]

and the at least one chemical synthesis reaction is the reaction depicted in FIG. 7A.

In at least one embodiment, the chemical compound can have the formula B(II):

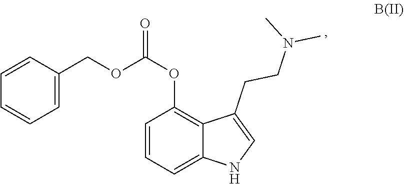 [SMILES: CN(C)CCc1c[nH]c2cccc(OC(=O)OCc3ccccc3)c12]

and the at least one chemical synthesis reaction is the reaction depicted in FIG. 8A.

In at least one embodiment, the chemical compound can have the formula B(III):

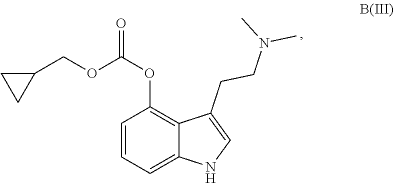 [SMILES: CN(C)CCc1c[nH]c2cccc(OC(=O)OCC3CC3)c12]

and the at least one chemical synthesis reaction is the chemical reaction (h); (g) and (h); (f), (g), and (h); (e), (f), (g), and (h); (d), (e), (f), (g), and (h); (c), (d), (e), (f), (g), and (h); (b), (c), (d), (e), (f), (g), and (h); and (a), (b), (c), (d), (e), (f), (g), and (h) depicted in FIGS. 9A (i) and 9A (ii).

In at least one embodiment, the chemical compound can have the formula D(III):

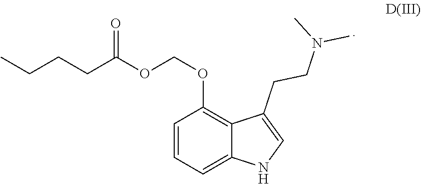 [SMILES: CCCCC(=O)OCOc1cccc2[nH]cc(CCN(C)C)c12]

and the at least one chemical synthesis reaction is the reaction depicted in FIG. 10A.

In at least one embodiment, the chemical compound can have the formula D(IV):

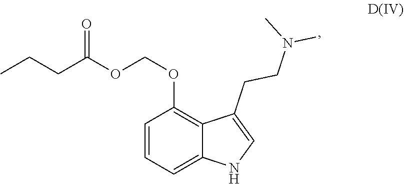

and the at least one chemical synthesis reaction is the reaction depicted in FIG. 11A.

In at least one embodiment, the chemical compound can have the formula D(XIII):

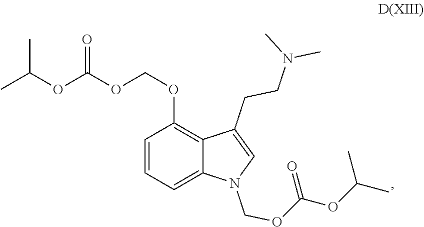 [SMILES: CC(C)OC(=O)OCOc1cccc2c1c(CCN(C)C)cn2COC(=O)OC(C)C]

and the at least one chemical synthesis reaction is the reaction depicted in FIG. 12A.

In at least one embodiment, the chemical compound can have the formula D(XIX):

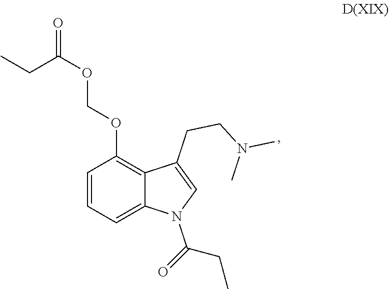 [SMILES: CCC(=O)OCOc1cccc2c1c(CCN(C)C)cn2C(=O)CC]

and the at least one chemical synthesis reaction is the reaction depicted in FIG. 13A.

In at least one embodiment, the chemical compound can have the formula F(II):

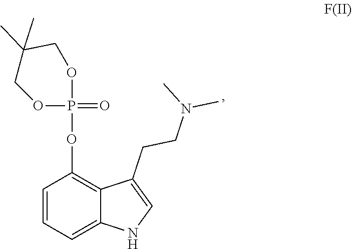

and the at least one chemical synthesis reaction is the reaction depicted in FIG. 14A.

In at least one embodiment, the chemical compound can have the formula A(IV):

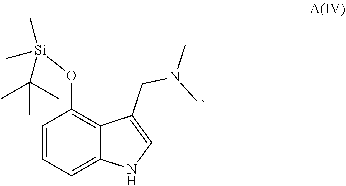 [SMILES: CN(C)Cc1c[nH]c2cccc(O[Si](C)(C)C(C)(C)C)c12]

and the at least one chemical synthesis reaction is the reaction depicted in FIG. 15A.

In another aspect, the present disclosure relates to a use of a chemical compound having a formula (I):

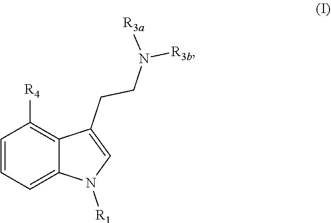

wherein R₄ is a substituent selected from:
(A) an ether moiety or derivative thereof;
(B) a carbonic ester moiety or derivative thereof;
(C) a carboxylic acid moiety or derivative thereof;
(D) a polyether moiety or derivative thereof;
(E) a carbonothioate moiety or derivative thereof; and
(F) a phosphate moiety or derivative thereof,
   wherein R₁ is a hydrogen atom or a moiety containing a carbonic ester or derivative thereof, or a ketone or ketone derivative; and
   wherein R₃ₐ and R_{3b} are each independently a hydrogen atom or an alkyl group, in the manufacture of a pharmaceutical or recreational drug formulation.

In at least one embodiment, in an aspect, R₄ can be a substituent selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof.

In at least one embodiment the manufacture can comprise formulating the chemical compound with an excipient, diluent, or carrier.

In another aspect the present disclosure provides, in at least one embodiment, a use of a chemical compound having a formula (I):

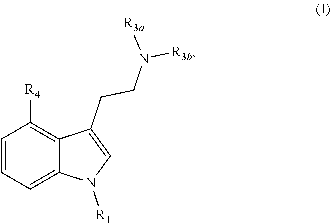

wherein R₄ is a substituent selected from:
(A) an ether moiety or derivative thereof;
(B) a carbonic ester moiety or derivative thereof;
(C) a carboxylic acid moiety or derivative thereof;
(D) a polyether moiety or derivative thereof;
(E) a carbonothioate moiety or derivative thereof; and
(F) a phosphate moiety or derivative thereof,
   wherein R₁ is a hydrogen atom or a moiety containing a carbonic ester or derivative thereof, or a ketone or a derivative thereof; and
   wherein R₃ₐ and R_{3b} are each independently a hydrogen atom or an alkyl group, together with a diluent, carrier, or excipient as a pharmaceutical or recreational drug formulation.

In at least one embodiment, in an aspect, R₄ can be a substituent selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof.

Other features and advantages will become apparent from the following detailed description. It should be understood, however, that the detailed description, while indicating preferred implementations of the disclosure, are given by way of illustration only, since various changes and modifications within the spirit and scope of the disclosure will become apparent to those of skill in the art from the detailed description.

### BRIEF DESCRIPTION OF THE DRAWINGS

The disclosure is in the hereinafter provided paragraphs described, by way of example, in relation to the attached figures. The figures provided herein are provided for a better understanding of the example embodiments and to show more clearly how the various embodiments may be carried into effect. The figures are not intended to limit the present disclosure.

FIG. 1 depicts the chemical structure of tryptamine.

FIG. 2 depicts a certain prototype structure of tryptamine and tryptamine derivative compounds, namely an indole. Certain carbon and nitrogen atoms may be referred to herein by reference to their position within the indole structure, i.e., N₁, C₂, C₃ etc. The pertinent atom numbering is shown.

The figures together with the following detailed description make apparent to those skilled in the art how the disclosure may be implemented in practice.

FIGS. 3A, 3B, 3C, 3D, 3E, 3F, 3G, 3H, 3I, 3J, 3K, 3L, 3M (i), 3M (ii), 3N (i), 3N (ii), 3O (i), 3O (ii), and 3P depict an example chemical reaction to make an example chemical compound provided by the present disclosure, namely a compound having chemical formula F(I), and various graphs representing certain experimental results (FIGS. 3B-3P), notably graphs obtained in the performance experimental assays to evaluate the efficacy of an example compound a compound having chemical formula F(I) (compound (F(I) designated as “F-I”), notably a cell viability assay (FIGS. 3B and 3C (compound (F(I) designated as “F-I”)); a saturation binding assay for [³H]ketanserin at the 5-HT_{2A} receptor (FIG. 3D); a competition assay for psilocin as a positive control (binding) (FIG. 3E); a competition assay for psilocybin and tryptophan as a control and negative control (no binding), respectively (FIG. 3F); a competition assay for a compound with formula F(I) (designated as “F-I”) (FIG. 3G); a cAMP assay in the presence of increasing forskolin concentrations in +5HT_{1A} cells and −5HT_{1A} cells (FIG. 3H); a cAMP assay in the presence of varying concentrations of tryptophan in +5HT_{1A} cells and −5HT_{1A} cells with 4 μM forskolin (FIG. 3I); a cAMP assay in the presence of varying concentrations of psilocin in +5HT_{1A} cells and −5HT_{1A} cells stimulated with 4 μM forskolin (FIG. 3J); a cAMP assay in the presence of varying concentrations of serotonin in +5HT_{1A} cells and −5HT_{1A} cells stimulated with 4 μM forskolin (FIG. 3K); a cAMP assay in the presence of varying concentrations of the compound having chemical formula F(I), designated “F-I” in +5HT_{1A} cells and −5HT_{1A} cells with 4 μM forskolin (FIG. 3L); psilocybin metabolic conversion assays (FIGS. 3M (i)-3M (ii)); assay controls for psilocin metabolic release assays (FIGS. 3N (i)-3N (ii)); metabolic stability assays for a compound with formula C(V) (FIGS. 3O (i)-3O (ii)); and Drug-induced Head Twitch Response (HTR) assays using the compound having formula F(I) (designated as “F-I”) (FIG. 3P).

FIGS. 4A, 4B, 4C, 4D, 4E, 4F (i), 4F (ii), and 4G depict an example chemical reaction to make an example chemical compound provided by the present disclosure, namely a compound having chemical formula A(V), (FIG. 4A), and various graphs representing certain experimental results (FIGS. 4B-4G), notably graphs obtained in the performance experimental assays to evaluate the efficacy of an example compound a compound having chemical formula A(V), notably a cell viability assay (FIGS. 4B and 4C (compound (A(V) designated as “A-V”)); a competition assay for a compound with formula A(V) (designated as “A-V”) (FIG. 4D); a cAMP assay in the presence of varying concentrations of the compound having chemical formula A(V) (designated as “A-V”), in +5HT_{1A} cells and −5HT_{1A} cells with 4 μM forskolin (FIG. 4E); metabolic stability assays and assays to evaluate the capacity of assayed molecules to release psilocin under various in vitro conditions (FIGS. 4F (i)-4F (ii)); and Drug-induced Head Twitch Response (HTR) assays using the compound having formula A(V) (designated as “A-V”), (FIG. 4G).

FIGS. 5A, 5B, 5C, 5D, 5E, 5F (i), 5F (ii), and 5G depict an example chemical reaction to make an example chemical compound provided by the present disclosure, namely a compound having chemical formula A(III), (FIG. 5A), and various graphs representing certain experimental results (FIGS. 5B-5G), notably graphs obtained in the performance experimental assays to evaluate the efficacy of an example compound a compound having chemical formula A(III), notably a cell viability assay (FIGS. 5B and 5C (compound (A(III) designated as “A-III”)); a competition assay for a compound with formula A(III) (designated as “A-III”) (FIG. 5D); a cAMP assay in the presence of varying concentrations of the compound having chemical formula A(III) (designated as “A-III”), in +5HT_{1A} cells and −5HT_{1A} cells with 4 μM forskolin (FIG. 5E); metabolic stability assays and assays to evaluate the capacity of assayed molecules to release psilocin under various in vitro conditions (FIGS. 5F (i)-5F (ii)); and Drug-induced Head Twitch Response (HTR) assays using the compound having formula A(III) (designated as “A-III”), (FIG. 5G).

FIGS. 6A, 6B, 6C, 6D, 6E, 6F (i), 6F (ii), and 6G depict an example chemical reaction to make an example chemical compound provided by the present disclosure, namely a compound having chemical formula B(IV), (FIG. 6A), and various graphs representing certain experimental results (FIGS. 6B-6G), notably graphs obtained in the performance experimental assays to evaluate the efficacy of an example compound a compound having chemical formula B(IV), notably a cell viability assay (FIGS. 6B and 6C (compound (B(IV) designated as “B-IV”)); a competition assay for a compound with formula B(IV) (designated as “B-IV”) (FIG. 6D); a cAMP assay in the presence of varying concentrations of the compound having chemical formula B(IV) (designated as “B-IV”), in +5HT_{1A} cells and −5HT_{1A} cells with 4 μM forskolin (FIG. 6E); metabolic stability assays and assays to evaluate the capacity of assayed molecules to release psilocin under various in vitro conditions (FIGS. 6F (i)-6F (ii)); and Drug-induced Head Twitch Response (HTR) assays using the compound having formula B(IV) (designated as “B-IV”), (FIG. 6G).

FIGS. 7A, 7B, 7C, 7D, 7E, and 7F depict an example chemical reaction to make an example chemical compound provided by the present disclosure, namely a compound having chemical formula B(V), (FIG. 7A), and various graphs representing certain experimental results (FIGS. 7B-7G), notably graphs obtained in the performance experimental assays to evaluate the efficacy of an example compound a compound having chemical formula B(V), notably a cell viability assay (FIGS. 7B and 7C (compound (B(V) designated as “B-V”)); a competition assay for a compound with formula B(V) (designated as “B-V”) (FIG. 7D); a cAMP assay in the presence of varying concentrations of the compound having chemical formula B(V) (designated as “B-V”), in +5HT_{1A} cells and −5HT_{1A} cells with 4 μM forskolin (FIG. 7E); and Drug-induced Head Twitch Response (HTR) assays using the compound having formula B(V) (designated as “B-V”), (FIG. 7F).

FIGS. 8A, 8B, 8C, 8D, 8E, 8F (i), 8F (ii), and 8G depict an example chemical reaction to make an example chemical compound provided by the present disclosure, namely a compound having chemical formula B(II), (FIG. 8A), and various graphs representing certain experimental results (FIGS. 8B-8G), notably graphs obtained in the performance experimental assays to evaluate the efficacy of an example compound a compound having chemical formula B(II), notably a cell viability assay (FIGS. 8B and 8C (compound (B(II) designated as “B-II”)); a competition assay for a compound with formula B(II) (designated as “B-II”) (FIG. 8D); a cAMP assay in the presence of varying concentrations of the compound having chemical formula B(II) (designated as “B-II”), in +5HT_{1A} cells and −5HT_{1A} cells with 4 μM forskolin (FIG. 8E); metabolic stability assays and assays to evaluate the capacity of assayed molecules to release psilocin under various in vitro conditions (FIGS. 8F (i)-8F (ii)); and Drug-induced Head Twitch Response (HTR) assays using the compound having formula B(II) (designated as “B-II”), (FIG. 8G).

FIGS. 9A (i) 9A (ii), 9B, 9C, 9D, 9E, 9F (i), 9F (ii), and 9G depict certain example chemical reactions to make an example chemical compound provided by the present disclosure, namely a compound having chemical formula B(III), (FIG. 9A (i), 9A (ii)), and various graphs representing certain experimental results (FIGS. 9B-9G), notably graphs obtained in the performance experimental assays to evaluate the efficacy of an example compound a compound having chemical formula B(III), notably a cell viability assay (FIGS. 9B and 9C (compound (B(III) designated as “B-III”)); a competition assay for a compound with formula B(III) (designated as “B-III”) (FIG. 9D); a cAMP assay in the presence of varying concentrations of the compound having chemical formula B(III) (designated as “B-III”), in +5HT_{1A} cells and −5HT_{1A} cells with 4 μM forskolin (FIG. 9E); metabolic stability assays and assays to evaluate the capacity of assayed molecules to release psilocin under various in vitro conditions (FIGS. 9F (i)-9F (ii)); and Drug-induced Head Twitch Response (HTR) assays using the compound having formula B(III) (designated as “B-III”), (FIG. 9G).

FIGS. 10A, 10B, 10C, 10D, 10E, 10F (i), 10F (ii), and 10G depict an example chemical reaction to make an example chemical compound provided by the present disclosure, namely a compound having chemical formula D(III), (FIG. 10A), and various graphs representing certain experimental results (FIGS. 10B-10G), notably graphs obtained in the performance experimental assays to evaluate the efficacy of an example compound a compound having chemical formula D(III), notably a cell viability assay (FIGS. 10B and 10C (compound (D(III) designated as “D-III”)); a competition assay for a compound with formula D(III) (designated as “D-III”) (FIG. 10D); a cAMP assay in the presence of varying concentrations of the compound having chemical formula D(III) (designated as “D-III”), in +5HT_{1A} cells and −5HT_{1A} cells with 4 μM forskolin (FIG. 10E); metabolic stability assays and assays to evaluate the capacity of assayed molecules to release psilocin under various in vitro conditions (FIGS. 10F (i)-10F (ii)); and Drug-induced Head Twitch Response (HTR) assays using the compound having formula D(III) (designated as “D-III”), (FIG. 10G).

FIGS. 11A, 11B, 11C, 11D, 11E, 11F (i), 11F (ii), and 11G depict an example chemical reaction to make an example chemical compound provided by the present disclosure, namely a compound having chemical formula D(IV), (FIG. 11A), and various graphs representing certain experimental results (FIGS. 11B-11G), notably graphs obtained in the performance experimental assays to evaluate the efficacy of an example compound a compound having chemical formula D(IV), notably a cell viability assay (FIGS. 11B and 11C (compound (D(IV) designated as “D-IV”)); a competition assay for a compound with formula D(IV) (designated as “D-IV”) (FIG. 11D); a cAMP assay in the presence of varying concentrations of the compound having chemical formula D(IV) (designated as “D-IV”), in +5HT_{1A} cells and −5HT_{1A} cells with 4 μM forskolin (FIG. 11E); metabolic stability assays and assays to evaluate the capacity of assayed molecules to release psilocin under various in vitro conditions (FIGS. 11F (i)-11F (ii)); and Drug-induced Head Twitch Response (HTR) assays using the compound having formula D(IV) (designated as “D-IV”), (FIG. 11G).

FIGS. 12A, 12B, 12C, 12D, 12E, 12F (i), 12F (ii), and 12G depict an example chemical reaction to make an example chemical compound provided by the present disclosure, namely a compound having chemical formula D(XIII), (FIG. 12A), and various graphs representing certain experimental results (FIGS. 12B-12G), notably graphs obtained in the performance experimental assays to evaluate the efficacy of an example compound a compound having chemical formula D(XIII), notably a cell viability assay (FIGS. 12B and 12C (compound (D(XIII) designated as “D-XIII”)); a competition assay for a compound with formula D(XIII) (designated as “D-XIII”) (FIG. 12D); a cAMP assay in the presence of varying concentrations of the compound having chemical formula D(XIII) (designated as “D-XIII”), in +5HT_{1A} cells and −5HT_{1A} cells with 4 μM forskolin (FIG. 12E); metabolic stability assays and assays to evaluate the capacity of assayed molecules to release psilocin under various in vitro conditions (FIGS. 12F (i)-12F (ii)); and Drug-induced Head Twitch Response (HTR) assays using the compound having formula D(XIII) (designated as “D-XIII”), (FIG. 12G).

FIGS. 13A, 13B, 13C, 13D, 13E, 13F (i), 13F (ii), and 13G depict an example chemical reaction to make an example chemical compound provided by the present disclosure, namely a compound having chemical formula D(XIX), (FIG. 13A), and various graphs representing certain experimental results (FIGS. 13B-13G), notably graphs obtained in the performance experimental assays to evaluate the efficacy of an example compound a compound having chemical formula D(XIX), notably a cell viability assay (FIGS. 13B and 13C (compound (D(XIX) designated as “D-XIX”)); a competition assay for a compound with formula D(XIX) (designated as “D-XIX”) (FIG. 13D); a cAMP assay in the presence of varying concentrations of the compound having chemical formula D(XIX) (designated as “D-XIX”), in +5HT_{1A} cells and −5HT_{1A} cells with 4 μM forskolin (FIG. 13E); metabolic stability assays and assays to evaluate the capacity of assayed molecules to release psilocin under various in vitro conditions (FIGS. 13F (i)-13F (ii)); and Drug-induced Head Twitch Response (HTR) assays using the compound having formula D(XIX) (designated as “D-XIX”), (FIG. 13G).

FIGS. 14A, 14B, 14C, 14D, 14E, 14F (i), 14F (ii), and 14G depict an example chemical reaction to make an example chemical compound provided by the present disclosure, namely a compound having chemical formula F(II), (FIG. 14A), and various graphs representing certain experimental results (FIGS. 14B-14G), notably graphs obtained in the performance experimental assays to evaluate the efficacy of an example compound a compound having chemical formula F(II), notably a cell viability assay (FIGS. 14B and 14C (compound (F(II) designated as “F-II”)); a competition assay for a compound with formula F(II) (designated as “F-II”) (FIG. 14D); a cAMP assay in the presence of varying concentrations of the compound having chemical formula F(II) (designated as “F-II”), in +5HT_{1A} cells and −5HT_{1A} cells with 4 μM forskolin (FIG. 14E); metabolic stability assays and assays to evaluate the capacity of assayed molecules to release psilocin under various in vitro conditions (FIGS. 14F (i)-14F (ii)); and Drug-induced Head Twitch Response (HTR) assays using the compound having formula F(II) (designated as “F-II”), (FIG. 14G).

FIGS. 15A, 15B, 15C, 15D, 15E, 15F (i), 15F (ii), and 15G depict an example chemical reaction to make an example chemical compound provided by the present disclosure, namely a compound having chemical formula A(IV), (FIG. 15A), and various graphs representing certain experimental results (FIGS. 15B-15G), notably graphs obtained in the performance experimental assays to evaluate the efficacy of an example compound a compound having chemical formula A(IV), notably a cell viability assay (FIGS. 15B and 15C (compound (A(IV) designated as “A-IV”)); a competition assay for a compound with formula A(IV) (designated as “A-IV”) (FIG. 15D); a cAMP assay in the presence of varying concentrations of the compound having chemical formula F(II) (designated as “A-IV”), in +5HT_{1A} cells and −5HT_{1A} cells with 4 μM forskolin (FIG. 15E); metabolic stability assays and assays to evaluate the capacity of assayed molecules to release psilocin under various in vitro conditions (FIGS. 15F (i)-15F (ii)); and Drug-induced Head Twitch Response (HTR) assays using the compound having formula A(IV) (designated as “A-IV”), (FIG. 15G).

### DETAILED DESCRIPTION

Various compositions, systems or processes will be described below to provide an example of an embodiment of each claimed subject matter. No embodiment described below limits any claimed subject matter and any claimed subject matter may cover processes, compositions or systems that differ from those described below. The claimed subject matter is not limited to compositions, processes or systems having all of the features of any one composition, system or process described below or to features common to multiple or all of the compositions, systems or processes described below. It is possible that a composition, system, or process described below is not an embodiment of any claimed subject matter. Any subject matter disclosed in a composition, system or process described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

As used herein and in the claims, the singular forms, such “a”, “an” and “the” include the plural reference and vice versa unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers.

Various compositions, systems or processes will be described below to provide an example of an embodiment of each claimed subject matter. No embodiment described below limits any claimed subject matter and any claimed subject matter may cover processes, compositions or systems that differ from those described below. The claimed subject matter is not limited to compositions, processes or systems having all of the features of any one composition, system or process described below or to features common to multiple or all of the compositions, systems or processes described below. It is possible that a composition, system, or process described below is not an embodiment of any claimed subject matter. Any subject matter disclosed in a composition, system or process described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

When ranges are used herein for physical properties, such as molecular weight, or chemical properties, such as chemical formulae, all combinations and sub-combinations of ranges and specific embodiments therein are intended to be included. Other than in the operating examples, or where otherwise indicated, all numbers expressing quantities of ingredients or reaction conditions used herein should be understood as modified in all instances by the term “about.” The term “about” when referring to a number or a numerical range means that the number or numerical range referred to is an approximation within experimental variability (or within statistical experimental error), and thus the number or numerical range may vary between 1% and 15% of the stated number or numerical range, as will be readily recognized by context. Furthermore, any range of values described herein is intended to specifically include the limiting values of the range, and any intermediate value or sub-range within the given range, and all such intermediate values and sub-ranges are individually and specifically disclosed (e.g., a range of 1 to 5 includes 1, 1.5, 2, 2.75, 3, 3.90, 4, and 5). Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

Unless otherwise defined, scientific and technical terms used in connection with the formulations described herein shall have the meanings that are commonly understood by those of ordinary skill in the art. The terminology used herein is for the purpose of describing particular embodiments only and is not intended to limit the scope of the present invention, which is defined solely by the claims.

All publications, patents and patent applications are herein incorporated by reference in their entirety to the same extent as if each individual publication, patent, or patent application was specifically and individually indicated to be incorporated by reference in its entirety.

### Terms and Definitions

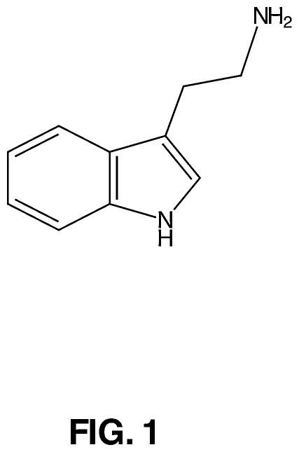

The term “tryptamine” refers to a chemical compound having the structure set forth in FIG. 1.

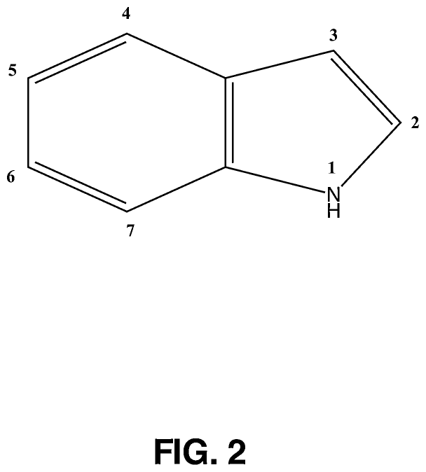

The term “indole prototype structure” refers to the chemical structure shown in FIG. 2. It is noted that specific carbon atoms and a nitrogen atom in the indole prototype structure are numbered. Reference may be made to these carbon and nitrogen numbers herein, for example C₂, C₄, N₁, and so forth. Furthermore, reference may be made to chemical groups attached to the indole prototype structure in accordance with the same numbering, for example, R₄ and R₆ reference chemical groups attached to the C₄ and C₆ atom, respectively. In addition, R₃ₐ and R_{3b}, in this respect, reference chemical groups extending from the ethyl-amino group extending in turn from the C₃ atom of the prototype indole structure.

The term “tryptamine derivative”, as used herein, refers to compounds that can be derivatized from tryptamine, wherein such compounds include an indole prototype structure and a C₃ ethylamine or ethylamine derivative group having the formula (VII):

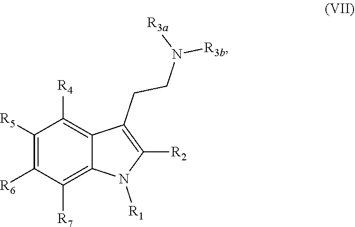

wherein at least one of R₁, R₂, R₄, R₅, R₆ and R₇ is a substituent (any atom or group other than a hydrogen atom), and wherein R₃ₐ and R_{3b} are each independently a hydrogen atom or an alkyl group. Thus, tryptamine derivative compounds include compounds containing one or more substituents at each of C₂, C₄, C₅, C₆ and C₇. Moreover, in this respect, tryptamine derivatives containing a substituent atom or group at e.g., C₄ may be referred to as C₄-substituted tryptamine derivatives. In chemical formula (VII), R₄, can, for example, be any of (A) an ether moiety or derivative thereof and the C₄-substituted tryptamine derivative may be referred to a C₄-ether-substituted tryptamine derivative; (B) a carbonic ester moiety or derivative thereof and the C₄-substituted tryptamine derivative may be referred to a C₄-carbonic ester-substituted tryptamine derivative; (C) a carboxylic acid moiety or derivative thereof and the C₄-substituted tryptamine derivative may be referred to a C₄-carboxylic acid-substituted tryptamine derivative; (D) a polyether moiety or derivative thereof and the C₄-substituted tryptamine derivative may be referred to a C₄-polyether-substituted tryptamine derivative; (E) a carbonothioate moiety or derivative thereof and the C₄-substituted tryptamine derivative may be referred to a C₄-carbonothioate-substituted tryptamine derivative; or (F) a phosphate moiety or derivative thereof and the C₄-substituted tryptamine derivative may be referred to a C₄-phosphate substituted tryptamine derivative.

The term “ether moiety or derivative thereof”, as used herein, refers to a derivative including a group having chemical formula (VIII):

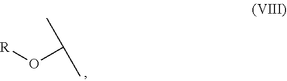

wherein R is a hydrocarbon group, for example, an alkyl group, an aryl group, or a silyl group. It is noted that the oxygen atom of the group having formula (VIII) can be bonded to another entity, including, for example, to the C₄ atom of tryptamine.

The term “carbonic ester moiety or derivative thereof”, as used herein, refers to a derivative including a group having chemical formula (IX):

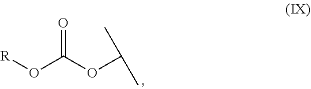

wherein R is a hydrocarbon group, for example, an alkyl group or an aryl group. It is noted that the partially bonded oxygen atom of the group having formula (IX) can be bonded to another entity, including, for example, to the C₄ atom of tryptamine.

The terms “carboxyl group”, “carboxyl”, “carboxylic acid” and “carboxy”, as used herein, refer to a molecule containing one atom of carbon bonded to an oxygen atom and a hydroxy group and having the formula —COOH. A carboxyl group includes a deprotonated carboxyl group, i.e., a carboxyl ion, having the formula —COO⁻. In its deprotonated form a carboxyl group may form a carboxyl salt, for example, a sodium or potassium carboxyl salt, or an organic carboxyl salt.

The term “carboxylic acid moiety or derivative thereof”, as used herein, refers to a modulated carboxyl group wherein the hydroxy group of the carboxyl group has been substituted by another atom or group. Thus, a carboxylic acid moiety or derivative thereof includes a group having chemical formula (X):

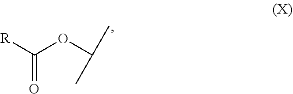

wherein, wherein R, for example, is an alkyl group, or an aryl group. It is noted that the partially bonded oxygen atom of the group having formula (IX) can be bonded to another entity, including, for example, to the C₄ atom of tryptamine. It is further noted that R can herein additionally include numerical subscripts, such as _{4a, 4b, 4c} etc., and be represented, for example, as R₄ₐ, R_{4b} or R_{4c}, respectively. Where such numerical values are included, they reference a chemical entity extending from the carboxyl group extending in turn from the thus numbered C atom of the prototype indole structure. Thus, for example, R₄ₐ is a chemical entity extending from a carboxyl group attached to the C₄ atom of the indole ring structure.

The term “polyether moiety or derivative thereof”, as used herein, refers to a derivative including a group having chemical formula (XI):

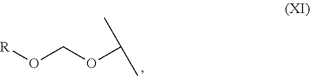

wherein R is a hydrocarbon group, for example, an alkyl group or an aryl group. It is noted that the partially bonded oxygen atom of the group having formula (XI) can be bonded to another entity, including, for example, to the C₄ atom of tryptamine.

The term “carbonothioate moiety or derivative thereof”, as used herein, refers to a derivative including a group having chemical formula (XII):

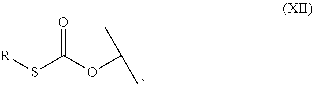

wherein R is a hydrocarbon group, for example, an alkyl group or an aryl group, and further including a sulfur atom containing hydrocarbon group, wherein the sulfur atom is bonded directly to the sulfur atom of the compound having formula (XII) to thereby form a disulfide group. It is noted that the partially bonded oxygen atom of the group having formula (XII) can be bonded to another entity, including, for example, to the C₄ atom of tryptamine. It is further noted that R can herein additionally include numerical subscripts, such as _{4a, 4b, 4c, 4d} etc., and be represented, for example, as R₄ₐ, R_{4b}, R_{4c} or R_{4d}, respectively. Where such numerical values are included, they reference a chemical entity extending from the carboxyl group extending in turn from the thus numbered C atom of the prototype indole structure. Thus, for example, R_{4c} is a chemical entity extending from a carboxyl group attached to the C₄ atom of the indole ring structure.

The terms “phosphate group” or “phospho group”, as used herein, is a molecule containing one atom of phosphorus, covalently bound to four oxygen atoms (three single bonds and one double bond). Of the four oxygen atoms one oxygen atom may be a hydroxy group, and one of the non-hydroxylated oxygen atoms may be chemically bonded to another entity.

The term “phosphate moiety or derivative thereof”, as used herein, is a phosphate group wherein at least one of the oxygen atoms is substituted, or extended, for example, by an alkyl group.

The terms “halogen”, “halogenated” and “halo-”, as used herein, refer to the class of chemical elements consisting of fluorine (F), chlorine (Cl), bromine (Br), and iodine (I). Accordingly, halogenated compounds can refer to “fluorinated”, “chlorinated”, “brominated”, or “iodinated” compounds.

The terms “hydroxy group”, and “hydroxy”, as used herein refers to a molecule containing one atom of oxygen bonded to one atom of hydrogen and having the formula —OH. A hydroxy group through its oxygen atom may be chemically bonded to another entity.

The term “alkyl group”, as used herein, refers to a straight and/or branched chain, saturated alkyl radical containing from one to “p” carbon atoms (“C₁-Cₚ-alkyl”) and includes, depending on the identity of “p”, methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl, and the like, where the variable p is an integer representing the largest number of carbon atoms in the alkyl radical. Alkyl groups further include hydrocarbon groups arranged in a chain having the chemical formula —CₙH₂ₙ₊₁, including, without limitation, methyl groups (—CH₃), ethyl groups (—C₂H₅), propyl groups (—C₃H₇), and butyl groups (—C₄H₉), further also includes cyclic alkyl groups, including cyclo-propane, cyclo-butane, cyclo-pentane, cyclo-hexane, and cyclo-heptane.

The term “cyclo-alkyl” refers to cyclic alkyl groups, including (C₃-C₂₀), (C₃-C₁₀), and (C₃-C₆) cyclo-alkyl groups, and further including cyclo-propane, cyclo-butane, cyclo-pentane, cyclo-hexane, and cyclo-heptane.

The term “O-alkyl group”, as used herein, refers to a hydrocarbon group arranged in a chain having the chemical formula —O—CₙH₂ₙ₊₁. O-alkyl groups include, without limitation, O-methyl groups (—O—CH₃), O-ethyl groups (—O—C₂H₅), O-propyl groups (—O—C₃H₇) and O-butyl groups (—O—C₄H₉).

The term “aryl group”, as used herein, refers to a hydrocarbon group arranged in an aromatic ring and can, for example, be a C₆-C₁₄-aryl, a C₆-C₁₀-aryl. Aryl groups further include phenyl, naphthyl, tetrahydronaphthyl, phenanthrenyl, biphenylenyl, indanyl, tolyl, xylyl, or indenyl groups, and the like.

The term “alcohol group” or “hydroxylalkyl”, as used herein, refers to a hydrocarbon group arranged in a chain having the chemical formula CₙHₙ₊₁OH. Depending on the carbon chain, length specific alcohol groups may be termed a methanol group (n=1) or hydroxymethyl, an ethanol group (n=2) or hydroxyethyl, a propanol group (n=3) or hydroxypropyl, a butanol group (n=4) or hydroxybutyl etc.

The term “alkyl-aryl”, as used herein, refers to an alkylene substituted with aryl, wherein the aryl is further optionally substituted.

The term “alkyl-heterocycle”, as used herein, refers to an alkylene substituted with heterocycle, wherein the heterocycle is further optionally substituted.

The term “alkyl-heteroaryl”, as used herein, refers to an alkylene substituted with heteroaryl, wherein the heteroaryl is further optionally substituted.

The term “oxo” as used herein refers to the group ═O and can be formed by replacing two hydrogens bonded to the same carbon atom with ═O.

The term “receptor”, as used herein, refers to a protein present on the surface of a cell, or in a cell not associated with a cellular surface (e.g., a soluble receptor) capable of mediating signaling to and/or from the cell, or within the cell and thereby affect cellular physiology. Example receptors include, 5-HT_{1A} receptors, 5-HT_{1B} receptors, 5-HT_{2A} receptors, and “5-HT_{2B} receptors”, and so on. In this respect, “signaling” refers to a response in the form of a series of chemical reactions which can occur when a molecule, including, for example, the C₄-substituted tryptamine derivatives disclosed herein, interacts with a receptor. Signaling generally proceeds across a cellular membrane and/or within a cell, to reach a target molecule or chemical reaction, and results in a modulation in cellular physiology. Thus, signaling can be thought of as a transduction process by which a molecule interacting with a receptor can modulate cellular physiology, and, furthermore, signaling can be a process by which molecules inside a cell can be modulated by molecules outside a cell. Signaling and interactions between molecules and receptors, including for example, affinity, binding efficiency, and kinetics, can be evaluated through a variety of assays, including, for example, assays known as receptor binding assays (for example, radioligand binding assays, such as e.g., [³H]ketanserin assays may be used to evaluate receptor 5-HT_{2A} receptor activity), competition assays, and saturation binding assays, and the like.

The term “5-HT_{1A} receptor”, as used herein, refers to a subclass of a family of receptors for the neurotransmitter and peripheral signal mediator serotonin. 5-HT_{1A} receptors can mediate a plurality of central and peripheral physiologic functions of serotonin. Ligand activity at 5-HT_{1A} is generally not associated with hallucination, although many hallucinogenic compounds are known to modulate 5-HT_{1A} receptors to impart physiological responses (Inserra et al., 2020, Pharmacol. Rev 73: 202). 5-HT_{1A} receptors are implicated in various brain neurological disorders, including depression and anxiety, schizophrenia, and Parkinson's disease (Behav. Pharm. 2015, 26:45-58).

The term “5-HT_{1B} receptor”, as used herein, refers to a subclass of a family of receptors for the neurotransmitter and peripheral signal mediator serotonin. 5-HT_{1B} receptors can mediate a plurality of central and peripheral physiologic functions of serotonin. Ligand activity at 5-HT_{1B} is generally not associated with hallucination, although many hallucinogenic compounds are known to modulate 5-HT_{1A} receptors to impart physiological responses (Inserra et al., 2020, Pharmacol. Rev. 73: 202). 5-HT_{1B} receptors are implicated in various brain neurological disorders, including depression (Curr. Pharm. Des. 2018, 24:2541-2548).

The term “5-HT_{2A} receptor”, as used herein, refers to a subclass of a family of receptors for the neurotransmitter and peripheral signal mediator serotonin. 5-HT_{2A} receptors can mediate a plurality of central and peripheral physiologic functions of serotonin. Central nervous system effects can include mediation of hallucinogenic effects of hallucinogenic compounds. 5-HT_{2A} receptors are implicated in various brain neurological disorders (Nat. Rev. Drug Discov. 2022, 21:463-473; Science 2022, 375:403-411).

The term “5-HT_{2B} receptor”, as used herein, refers to a subclass of a family of receptors for the neurotransmitter and peripheral signal mediator serotonin. 5-HT_{2B} receptors can mediate a plurality of central and peripheral physiologic functions of serotonin. Central nervous system effects can include mediation of hallucinogenic effects of hallucinogenic compounds. 5-HT_{bA} receptors are implicated in various brain neurological disorders, including schizophrenia (Pharmacol. Ther. 2018, 181:143-155) and migraine (Cephalalgia 2017, 37:365-371).

The term “5-HT_{3A} receptor”, as used herein, refers to a subclass of a family of receptors for the neurotransmitter and peripheral signal mediator serotonin. 5-HT_{3A} receptors can mediate a plurality of central and peripheral physiologic functions of serotonin. 5-HT_{3A} receptors are implicated in various brain neurological disorders, including depression (Expert Rev. Neurother. 2016, 16:483-95).

The term “ADRA1A receptor”, as used herein, refers to a subclass of a family of receptors, also known as a1-adrenergic receptors, which can be modulated by selective serotonin reuptake inhibitors (SSRIs) and tricyclic antidepressant (TCA) (Int. J. Mol Sci. 2021, 22: 4817; Brain Res. 1285 2009, 148-157). ADRA1A receptors are implicated in various brain neurological disorders, including depression.

The term “ADRA2A receptor”, as used herein, refers to a subclass of a family of receptors, also known as a2-adrenergic receptors. ADRA2A receptors are implicated in various brain neurological disorders, including Attention Deficit Hyperactivity Disorder (ADHD) (J. Am. Acad. Child. Adolesc. Psychiatry 2014, 53:153-73), mania, bipolar disorder, and schizophrenia.

The term “CHRM1 receptor”, as used herein, refers to a subclass of receptors also known as “cholinergic receptor muscarinic 1”, which can be modulated by selective serotonin reuptake inhibitors (SSRIs) (e.g., paroxetine) and tricyclic antidepressant (TCA). The class of CHRM receptors are implicated in various brain neurological disorders, including depression, major depression disorder (MDD), and bipolar disorder (Mol. Psychiatry 2019, 24: 694-709).

The term “CHRM2 receptor”, as used herein, refers to a subclass of receptors also known as “cholinergic receptor muscarinic 2”, which can be modulated by tricyclic antidepressant (TCA). The class of CHRM receptors are implicated in various brain neurological disorders, including depression, major depression disorder (MDD), and bipolar disorder (Mol. Psychiatry 2019, 24: 694-709).

The term “CNR1 receptor”, as used herein, refers to a subclass of receptors also known as “cannabinoid receptor CB₁”, which can be modulated by cannabinoid compounds. CNR receptors are implicated in various brain neurological disorders, including depression and schizophrenia (Pharmacol. Res. 2021, 170: 105729).

The term “DRD1 receptor”, as used herein, refers to a subclass of receptors also known as “dopamine receptor D₁”, which can be modulated by dopamine. Dopamine receptors are implicated in various brain neurological disorders, including schizophrenia, psychosis, and depression (Neurosci. Lett. 2019, 691:26-34).

The term “DRD2S receptor”, as used herein, refers to a subclass of receptors also known as “dopamine receptor D₂S”, which can be modulated by dopamine. Dopamine receptors are implicated in various brain neurological disorders, including schizophrenia, psychosis, and depression (Neurosci. Lett. 2019, 691:26-34).

The term “OPRD1 receptor”, as used herein, refers to a subclass of receptors also known as “opioid receptor D₁”, which can be modulated by opioid compounds. OPRD1 receptors are implicated in various brain neurological disorders, including psychopathy, and substance abused disorder (Mol. Psychiatry 2020, 25:3432-3441).

The term “MAO-A”, as used herein, refers to an enzyme involved in signaling also known as “Monoamine oxygenase A”, which can catalyze reactions which modulate signaling molecules, notably, for example, the deamination of the signaling molecules dopamine, norepinephrine, and serotonin. Compounds capable of modulating MOA, e.g., inhibitors of MOA, may be used to treat various brain neurological disorders, including panic disorders, depression, and Parkinson's disease (J. Clin. Psychiatry 2012, 73 Suppl. 1:37-41).

The term “DAT”, as used herein, refers to a transmembrane transport protein also known as “dopamine active transporter”, which is involved of transporting dopamine into the cytosol. DAT is implicated in various brain neurological disorders, notably dopamine related disorders such as attention deficit hyperactivity disorder (ADHD), bipolar disorder, and clinical depression, anxiety (Am. J. Med. Genet. B Neuropsychiatr. Genet. 2018, 177:211-231).

The term “NET”, as used herein, refers to a transmembrane transport protein also known as “norepinephrine transporter” or “noradrenaline transporter” or “NAT” which is involved in Na⁺/Cl⁻ dependent re-uptake of extracellular norepinephrine or noradrenaline. NET is implicated in various brain neurological disorders, including attention deficit hyperactivity disorder (ADHD) and clinical depression (Neurosci. Biobehav. Rev, 2013, 37:1786-800).

The term “SERT”, as used herein, refers to a transmembrane transport protein also known as “serotonin transporter” which is involved in neuronal serotonin transport, notably from the synaptic cleft back to the presynaptic neuron, thereby terminating the action of serotonin. SERT is implicated in various brain neurological disorders, including anxiety and depression (Pharmacol. Rep. 2018, 70:37-46).

The term “modulating receptors”, as used herein, refers to the ability of a compound disclosed herein to alter the function of receptors. A receptor modulator may activate the activity of a receptor or inhibit the activity of a receptor depending on the concentration of the compound exposed to the receptor. Such activation or inhibition may be contingent on the occurrence of a specific event, such as activation of a signal transduction pathway, and/or maybe manifest only in particular cell types. The term “modulating receptors,” also refers to altering the function of a receptor by increasing or decreasing the probability that a complex forms between a receptor and a natural binding partner to form a multimer. A receptor modulator may increase the probability that such a complex forms between the receptor and the natural binding partner, may increase or decrease the probability that a complex forms between the receptor and the natural binding partner depending on the concentration of the compound exposed to the receptor, and or may decrease the probability that a complex forms between the receptor and the natural binding partner. It is further noted that the C₄-carboxylic acid-substituted tryptamine derivatives or the C₄-carbonothioate-substituted tryptamine derivatives of the present disclosure may alter the function of a receptor by acting as an agonist or antagonist of the receptor, and that C₄-carboxylic acid-substituted tryptamine derivatives or the C₄-carbonothioate-substituted tryptamine derivatives according to the present disclosure may alter the function of a receptor by directly interacting therewith or binding thereto, or by indirectly interacting therewith through one or more other molecular entities. In general, the receptor may be any receptor, including any receptor set forth herein, such as any of a 5-HT_{1A}, 5-HT_{1B}, 5-HT_{2A}, a 5-HT_{2B}, 5-HT_{3A}, ADRA1A, ADRA2A, CHRM1, CHRM2, CNR1, DRD1, DRD2S, or OPRD1 receptor, for example. Accordingly, it will be clear, that in order to refer modulating specific receptors, terms such as “modulating 5-HT_{1A} receptors”, “modulating 5-HT_{1B} receptors”, “modulating 5-HT_{2A} receptors”, “modulating 5-HT_{2B} receptors”, and so forth, may be used herein.

The term “receptor-mediated disorder”, as used herein, refers to a disorder that is characterized by abnormal receptor activity. A receptor-mediated disorder may be completely or partially mediated by modulating a receptor. In particular, a receptor-mediated disorder is one in which modulation of the receptor results in some effect on an underlying disorder e.g., administration of a receptor modulator results in some improvement in at least some of the subjects being treated. In general, the receptor may be any receptor, including any receptor set forth herein, such as any of a 5-HT_{1A}, 5-HT_{1B}, 5-HT_{2A}, a 5-HT_{2B}, 5-HT_{3A}, ADRA1A, ADRA2A, CHRM1, CHRM2, CNR1, DRD1, DRD2S, or OPRD1 receptor, for example. Accordingly, it will be clear, that in order to refer specific receptor-mediated disorders, terms such as “5-HT_{1A} receptor-mediated disorder”, “5-HT_{1B} receptor-mediated disorder”, “5-HT_{2A} receptor-mediated disorder”, “5-HT_{2B} receptor-mediated disorder”, and so forth, may be used.

The term “pharmaceutical formulation”, as used herein, refers to a preparation in a form which allows an active ingredient, including a psychoactive ingredient, contained therein to provide effective treatment, and which does not contain any other ingredients which cause excessive toxicity, an allergic response, irritation, or other adverse response commensurate with a reasonable risk/benefit ratio. The pharmaceutical formulation may contain other pharmaceutical ingredients such as excipients, carriers, diluents, or auxiliary agents.

The term “recreational drug formulation”, as used herein, refers to a preparation in a form which allows a psychoactive ingredient contained therein to be effective for administration as a recreational drug, and which does not contain any other ingredients which cause excessive toxicity, an allergic response, irritation, or other adverse response commensurate with a reasonable risk/benefit ratio. The recreational drug formulation may contain other ingredients such as excipients, carriers, diluents, or auxiliary agents.

The term “effective for administration as a recreational drug”, as used herein, refers to a preparation in a form which allows a subject to voluntarily induce a psychoactive effect for non-medical purposes upon administration, generally in the form of self-administration. The effect may include an altered state of consciousness, satisfaction, pleasure, euphoria, perceptual distortion, or hallucination.

The term “effective amount”, as used herein, refers to an amount of an active agent, pharmaceutical formulation, or recreational drug formulation, sufficient to induce a desired biological or therapeutic effect, including a prophylactic effect, and further including a psychoactive effect. Such effect can include an effect with respect to the signs, symptoms or causes of a disorder, or disease or any other desired alteration of a biological system. The effective amount can vary depending, for example, on the health condition, injury stage, disorder stage, or disease stage, weight, or sex of a subject being treated, timing of the administration, manner of the administration, age of the subject, and the like, all of which can be determined by those of skill in the art.

The terms “treating” and “treatment”, and the like, as used herein, are intended to mean obtaining a desirable physiological, pharmacological, or biological effect, and includes prophylactic and therapeutic treatment. The effect may result in the inhibition, attenuation, amelioration, or reversal of a sign, symptom or cause of a disorder, or disease, attributable to the disorder, or disease, which includes mental and psychiatric diseases and disorders. Clinical evidence of the prevention or treatment may vary with the disorder, or disease, the subject, and the selected treatment.

The term “pharmaceutically acceptable”, as used herein, refers to materials, including excipients, carriers, diluents, or auxiliary agents, that are compatible with other materials in a pharmaceutical or recreational drug formulation and within the scope of reasonable medical judgement suitable for use in contact with a subject without excessive toxicity, allergic response, irritation, or other adverse response commensurate with a reasonable risk/benefit ratio.

The terms “substantially pure” and “isolated”, as may be used interchangeably herein describe a compound, e.g., a C₄-substituted tryptamine derivative, which has been separated from components that naturally accompany it. Typically, a compound is substantially pure when at least 60%, more preferably at least 75%, more preferably at least 90%, 95%, 96%, 97%, or 98%, and most preferably at least 99% of the total material (by volume, by wet or dry weight, or by mole percent or mole fraction) in a sample is the compound of interest. Purity can be measured by any appropriate method, e.g., by chromatography, gel electrophoresis or HPLC analysis.

### General Implementation

As hereinbefore mentioned, the present disclosure relates to tryptamine derivatives. In particular, the present disclosure provides novel C₄-substituted tryptamine derivatives. In general, the herein provided compositions exhibit functional properties which deviate from the functional properties o tryptamine. Thus, for example, the C₄-substituted tryptamine derivatives can exhibit pharmacological properties which deviate from tryptamine. Furthermore, the C₄-tryptamine derivatives may exhibit physico-chemical properties which differ from tryptamine. Thus, for example, C₄-substituted tryptamine derivatives may exhibit superior solubility in a solvent, for example, an aqueous solvent. The C₄-substituted tryptamine derivatives in this respect are useful in the formulation of pharmaceutical and recreational drug formulations.

In what follows selected embodiments are described with reference to the drawings.

Accordingly, in one aspect the present disclosure provides derivatives of a compound known as tryptamine of which the chemical structure is shown in FIG. 1. The derivatives herein provided are, in particular, C₄-substituted tryptamine derivatives, i.e., derivatives, wherein the C₄ atom is bonded to a substituent group.

Thus, in one aspect, the present disclosure provides, in accordance with the teachings herein, in at least one embodiment, a compound having chemical formula (I):

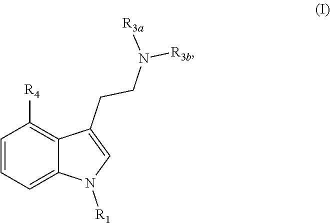

wherein R₄ is a substituent selected from:
(A) an ether moiety or derivative thereof;
(B) a carbonic ester moiety or derivative thereof;
(C) a carboxylic acid moiety or derivative thereof;
(D) a polyether moiety or derivative thereof;
(E) a carbonothioate moiety or derivative thereof; and
(F) a phosphate moiety or derivative thereof;
   wherein R₁ is a hydrogen atom or a moiety containing a carbonic ester or derivative thereof, or a ketone or a derivative thereof; and
   wherein R₃ₐ and R_{3b} are each independently a hydrogen atom or an alkyl group.

Thus, referring to the chemical compound having the formula (I), initially it is noted that, in an aspect hereof, R₄, is a substituent entity selected from (A) an ether moiety or derivative thereof; (B) a carbonic ester moiety or derivative thereof; (C) a carboxylic acid moiety or derivative thereof; (D) a polyether moiety or derivative thereof; (E) a carbonothioate moiety or derivative thereof; and (F) a phosphate moiety or derivative thereof.

In a preferred embodiment, R₄, is a substituent entity selected from (A) an ether moiety or derivative thereof; (B) a carbonic ester moiety or derivative thereof; (D) a polyether moiety or derivative thereof; and (F) a phosphate moiety or derivative thereof.

Thus, referring to the chemical compound having the formula (I), in an aspect hereof, in some embodiments, R₄ can be an ether moiety or derivative thereof.

In some embodiments, in an aspect, the ether moiety or derivative thereof can be an aryl ether group, a heteroaryl ether group, an alkyl-arylether group, an alkyl heterocycle group, or an alkyl heteroaryl group. The aryl group, heteroaryl group, alkyl-aryl group, alkyl-heterocycle group or alkyl-heteroaryl groups can be substituted or, in other embodiments, be unsubstituted. Where these groups are substituted, substituents, can, for example, be are a halo group (fluoro, iodo, chloro, or bromo), an amide group, an oxo group, or an alkyl group, wherein two or more alkyl groups optionally form an additional ring.

In some embodiments, the aryl group can be a phenyl group or a naphthyl group, for example.

In some embodiments, the additional ring can be a cyclopentyl or cyclohexyl group, for example.

In some embodiments, the alkaryl ether or alkheteroaryl ether can be a C₁-C₆-alkylene-aryl group or a C₁-C₆-alkylene-heteroaryl group, for example, a C₁-C₆-alkylene-phenyl or C₁-C₆-alkylene-naphthyl, respectively.

In some embodiments, the ether moiety or derivative can be a cycloalkyl ether, in which at least one carbon of the ring is optionally a heteroatom, for example, an oxygen (O) atom or nitrogen (N) atom. The cycloalkyl can be a C₃-C₆-cycloalkyl group, for example.

In some embodiments, the ether moiety or derivative thereof can be a silyl ether wherein the silicon atom is substituted with one or more alkyl groups or aryl groups.

Thus, in some embodiments, the alkyl group can be a C₁-C₆-alkyl group and the aryl group can be a phenyl group. The C₁-C₆-alkyl group can, in one embodiment, be a methyl, ethyl, propyl, n-butyl, s-butyl, or t-butyl.

In an aspect, the present disclosure provides a compound having chemical formula (I) wherein R₄ is an ether moiety or derivative thereof, the compound having the chemical formula A(I):

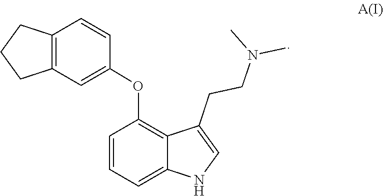 [SMILES: CN(C)CCc1c[nH]c2cccc(Oc3ccc4c(c3)CCC4)c12]

In an aspect, the present disclosure provides a compound having chemical formula (I) wherein R₄ is an ether moiety or derivative thereof, the compound having the chemical formula A(II):

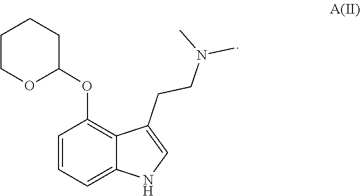 [SMILES: CN(C)CCc1c[nH]c2cccc(OC3CCCCO3)c12]

In an aspect, the present disclosure provides a compound having chemical formula (I) wherein R₄ is an ether moiety or derivative thereof, the compound having the chemical formula A(III):

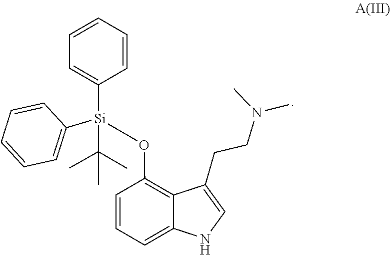 [SMILES: CN(C)CCc1c[nH]c2cccc(O[Si](c3ccccc3)(c3ccccc3)C(C)(C)C)c12]

In an aspect, the present disclosure provides a compound having chemical formula (I) wherein R₄ is an ether moiety or derivative thereof, the compound having the chemical formula A(IV):

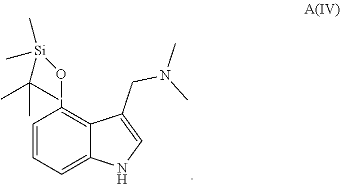 [SMILES: CN(C)Cc1c[nH]c2cccc(O[Si](C)(C)C(C)(C)C)c12]

In an aspect, the present disclosure provides a compound having chemical formula (I) wherein R₄ is an ether moiety or derivative thereof, the compound having the chemical formula A(V):

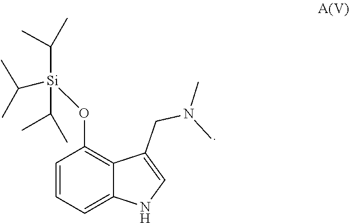 [SMILES: CC(C)[Si](Oc1cccc2[nH]cc(CN(C)C)c12)(C(C)C)C(C)C]

In some embodiments, in an aspect, in the compound having chemical formula (I) wherein R₄ is an ether moiety or derivative thereof, the compound having the chemical formula A(VI):

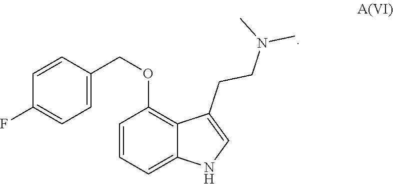 [SMILES: CN(C)CCc1c[nH]c2cccc(OCc3ccc(F)cc3)c12]

In an aspect, the present disclosure provides a compound having chemical formula (I) wherein R₄ is an ether moiety or derivative thereof, the compound having the chemical formula A(VII):

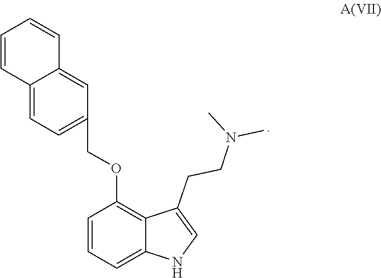 [SMILES: CN(C)CCc1c[nH]c2cccc(OCc3ccc4ccccc4c3)c12]

In an aspect, the present disclosure provides a compound having chemical formula (I) wherein R₄ is an ether moiety or derivative thereof, the compound having the chemical formula A(VIII):

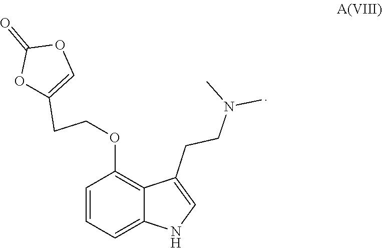 [SMILES: CN(C)CCc1c[nH]c2cccc(OCCc3coc(=O)o3)c12]

In an aspect, the present disclosure provides a compound having chemical formula (I) wherein R₄ is an ether moiety or derivative thereof, the compound having the chemical formula A(IX):

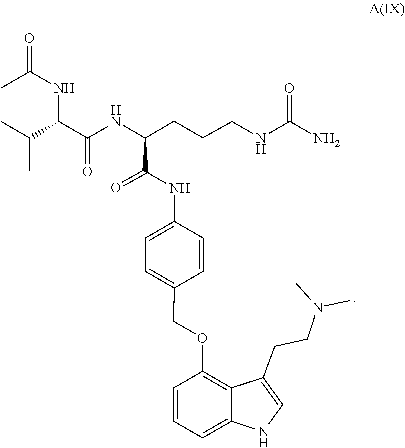 [SMILES: CC(=O)N[C@H](C(=O)N[C@@H](CCCNC(N)=O)C(=O)Nc1ccc(COc2cccc3[nH]cc(CCN(C)C)c23)cc1)C(C)C]

In an aspect, the present disclosure provides a compound having chemical formula (I) wherein R₄ is an ether moiety or derivative thereof, the compound having the chemical formula A(X):

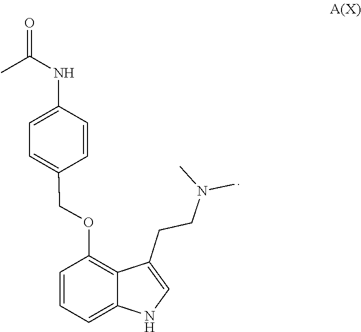 [SMILES: CC(=O)Nc1ccc(COc2cccc3[nH]cc(CCN(C)C)c23)cc1]

Referring next to the chemical compound having the formula (I), in an aspect hereof, in some embodiments, R₄ can be a carbonic ester moiety or derivative thereof.

In some embodiments, the compound having chemical formula (I) wherein R₄ is a carbonic ester moiety or derivative thereof, the compound can have the chemical formula (XIV):

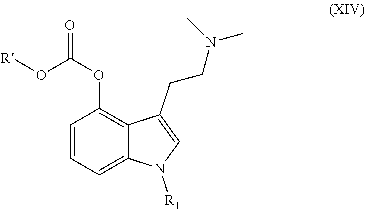

wherein,
R₁ is a hydrogen atom or a carbonic ester moiety —O—C(═O)—O—(C₁-C₆)-alkyl, wherein the alkyl is optionally substituted with halo or C₆-C₁₀-aryl;
R′ is C₁-C₁₀-alkyl, wherein the alkyl group is optionally substituted with one or more of halo, C₆-C₁₀-aryl, C₃-C₆-cycloalkyl, or a polycyclic hydrocarbon.

In at least one embodiment, R₁ can be O—C(═O)—O—CH₂-phenyl.

In some embodiments, the optional substituent can be fluorenyl, phenyl, cyclopropyl, or chloro.

In some embodiments, the compound having chemical formula (I) wherein R₄ is a carbonic ester moiety or derivative thereof, the compound having the chemical formula B(I):

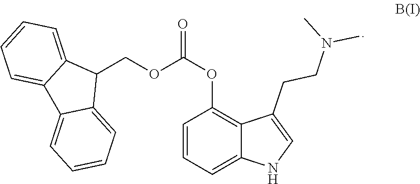 [SMILES: CN(C)CCc1c[nH]c2cccc(OC(=O)OCC3c4ccccc4-c4ccccc43)c12]

In an aspect, the present disclosure provides a compound having chemical formula (I) wherein R₄ is a carbonic ester moiety or derivative thereof, the compound having the chemical formula B(II):

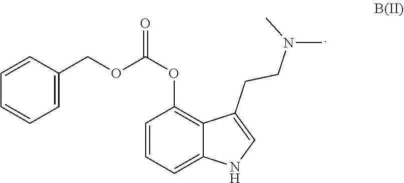 [SMILES: CN(C)CCc1c[nH]c2cccc(OC(=O)OCc3ccccc3)c12]

In an aspect, the present disclosure provides a compound having chemical formula (I) wherein R₄ is a carbonic ester moiety or derivative thereof, the compound having the chemical formula B(III):

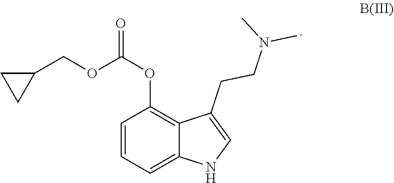 [SMILES: CN(C)CCc1c[nH]c2cccc(OC(=O)OCC3CC3)c12]

In an aspect, the present disclosure provides a compound having chemical formula (I) wherein R₄ is a carbonic ester moiety or derivative thereof, the compound having the chemical formula B(IV):

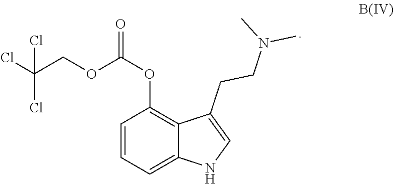 [SMILES: CN(C)CCc1c[nH]c2cccc(OC(=O)OCC(Cl)(Cl)Cl)c12]

In an aspect, the present disclosure provides a compound having chemical formula (I) wherein R₄ is a carbonic ester moiety or derivative thereof, the compound having the chemical formula B(V):

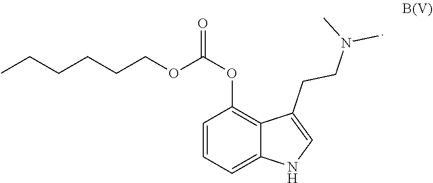 [SMILES: CCCCCCOC(=O)Oc1cccc2[nH]cc(CCN(C)C)c12]

In an aspect, the present disclosure provides a compound having chemical formula (I) wherein R₄ is a carbonic ester moiety or derivative thereof, the compound having the chemical formula B(VI):

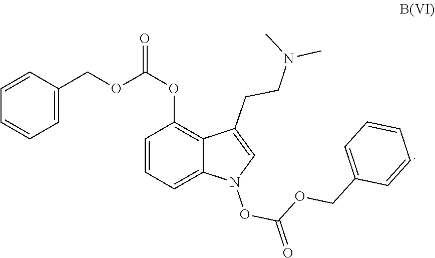 [SMILES: CN(C)CCc1cn(OC(=O)OCc2ccccc2)c2cccc(OC(=O)OCc3ccccc3)c12]

Referring next to the chemical compound having the formula (I), in an aspect hereof, in some embodiments, R₄ can be a carboxylic acid moiety or derivative thereof.

In some embodiments, in an aspect, the carboxylic acid moiety or derivative thereof can have the chemical formula (II):

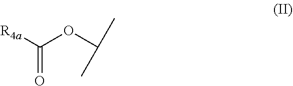

wherein R₄ₐ is an aryl group, a substituted aryl group, an alkyl group, a substituted alkyl group, an amide group, or a substituted amide group.

In some embodiments, in an aspect, the aryl group and substituted aryl group can be a phenyl group and a substituted phenyl group, respectively.

In some embodiments, the substituted aryl group can be a halo-substituted phenyl group, for example, a bromo-phenyl group.

In some embodiments, the substituted alkyl group can be a C₁-C₁₀ alkyl group, in which optionally, at least one carbon atom in the alkyl chain is replaced with an oxygen (O) atom, and wherein the optional substituents are at least one of halo, C₃-C₆alkyl, or amino (NH₂).

In some embodiments, the aryl group can be a phenyl group in which two substituents on the phenyl group are joined together to form an additional 5-7-membered ring.

In some embodiments, the 5-7-membered ring can be a methylene-dioxy ring, an ethylene-dioxy ring or a dihydrofuryl ring.

In some embodiments, the substituted aryl group can be an optionally substituted phenyl group which is substituted with an acetamidyl group or an alkoxycarbonyl group, such as methoxycarbonyl (CH₃OC(═O)—).

In some embodiments, in an aspect, the substituted phenyl group is an O-alkylated phenyl group.

In some embodiments, the substituted phenyl group can be an O-alkylated phenyl group, in which the phenyl group can be substituted with one or more O-alkyl groups.

In some embodiments, the O-alkyl group can be a methoxy group, an ethoxy group, a propoxy group, an iso-propoxy group, or a butoxy group (n-but, s-but or t-but).

In some embodiments, in an aspect, the O-alkyl group can be a methoxy group, for example, 1, 2, or 3 methoxy groups.

In some embodiments, in an aspect, the substituted phenyl group can be a halogenated phenyl group.

In some embodiments, the substituted phenyl group can be a per-halogenated phenyl group, such as a fluorinated phenyl group.

In some embodiments, in an aspect, the substituted phenyl group can be a trifluoromethylated phenyl group.

In some embodiments, the substituted aryl group can be a substituted phenyl group having one or more substituents which are halo, alkoxy, alkyl, or halo-substituted alkyl.

In some embodiments, the phenyl group can be substituted with one or more of a trifluoromethoxy group, a methoxy group or a halogen atom (fluoro, chloro, bromo, iodo).

In some embodiments, in an aspect, R₄ₐ can be a substituted pyridine group.

In some embodiments, in an aspect, the substituted pyridine group can be an O-alkylated pyridine group, an O-arylated pyridine group or a halogenated pyridine group (chloro, fluoro, bromo, or iodo).

In some embodiments, in an aspect, the O-alkyl group can be a one or more methoxy groups, for example one or two groups.

In some embodiments, the substituted pyridine group can be an O-alkylated pyridine group, an O-arylated pyridine group, or a halogenated pyridine group.

In some embodiments, the O-alkylated pyridine group can be O-alkylated by one or more methoxy groups.

In some embodiments, the O-alkylated pyridine group can be O-alkylated by one or more methoxy groups and one or more halogen atoms (chloro, fluoro, bromo or iodo).

In some embodiments, the pyridine group can be substituted with a O-aryl group.

In some embodiments, the O-aryl group can be an O-phenyl group.

In some embodiments, the substituted aryl group can be a substituted phenyl group which is substituted by a carboxylate moiety.

In an aspect, the present disclosure provides a compound having chemical formula (I) wherein R₄ is a carboxylic acid moiety or derivative thereof, the compound having the chemical formula C(I):

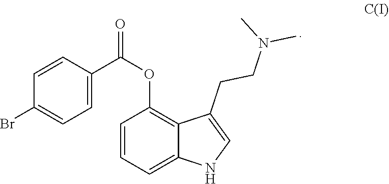 [SMILES: CN(C)CCc1c[nH]c2cccc(OC(=O)c3ccc(Br)cc3)c12]

In an aspect, the present disclosure provides a compound having chemical formula (I) wherein R₄ is a carboxylic acid moiety or derivative thereof, the compound having the chemical formula C(II):

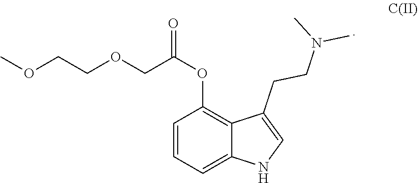

In an aspect, the present disclosure provides a compound having chemical formula (I) wherein R₄ is a carboxylic acid moiety or derivative thereof, the compound having the chemical formula C(III):

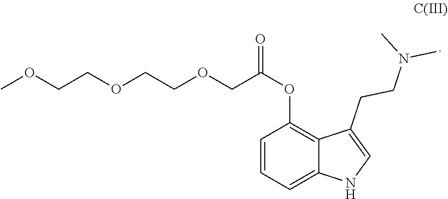

In an aspect, the present disclosure provides a compound having chemical formula (I) wherein R₄ is a carboxylic acid moiety or derivative thereof, the compound having the chemical formula C(IV):

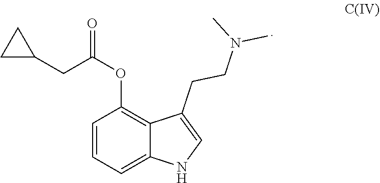 [SMILES: CN(C)CCc1c[nH]c2cccc(OC(=O)CC3CC3)c12]

In an aspect, the present disclosure provides a compound having chemical formula (I) wherein R₄ is a carboxylic acid moiety or derivative thereof, the compound having the chemical formula C(V):

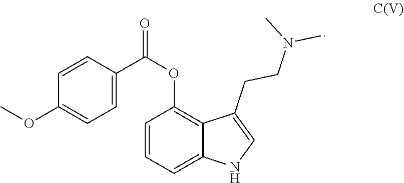

In some embodiments, in an aspect, in the compound having chemical formula (I) wherein R₄ is a carboxylic acid moiety or derivative thereof, the compound having the chemical formula C(VI):

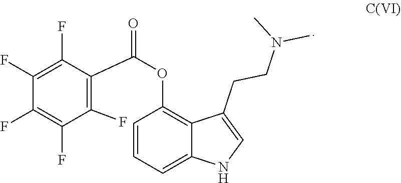 [SMILES: CN(C)CCc1c[nH]c2cccc(OC(=O)c3c(F)c(F)c(F)c(F)c3F)c12]

In an aspect, the present disclosure provides a compound having chemical formula (I) wherein R₄ is a carboxylic acid moiety or derivative thereof, the compound having the chemical formula C(VII):

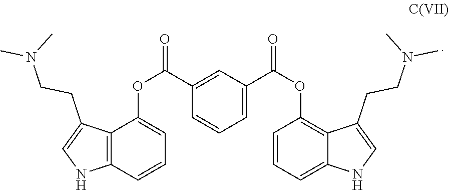

In an aspect, the present disclosure provides a compound having chemical formula (I) wherein R₄ is a carboxylic acid moiety or derivative thereof, the compound having the chemical formula C(VIII):

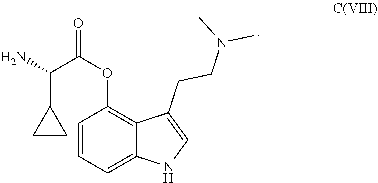 [SMILES: CN(C)CCc1c[nH]c2cccc(OC(=O)[C@@H](N)C3CC3)c12]

In an aspect, the present disclosure provides a compound having chemical formula (I) wherein R₄ is a carboxylic acid moiety or derivative thereof, the compound having the chemical formula C(IX):

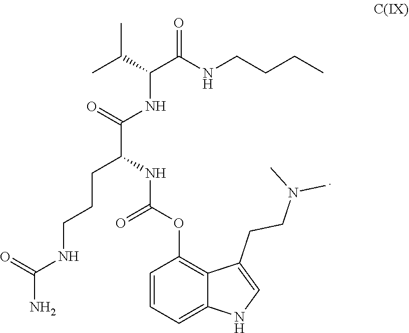 [SMILES: CCCCNC(=O)[C@H](NC(=O)[C@@H](CCCNC(N)=O)NC(=O)Oc1cccc2[nH]cc(CCN(C)C)c12)C(C)C]

In an aspect, the present disclosure provides a compound having chemical formula (I) wherein R₄ is a carboxylic acid moiety or derivative thereof, the compound having the chemical formula C(X):

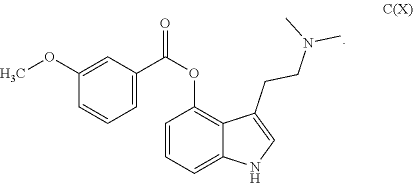

In an aspect, the present disclosure provides a compound having chemical formula (I) wherein R₄ is a carboxylic acid moiety or derivative thereof, the compound having the chemical formula C(XI):

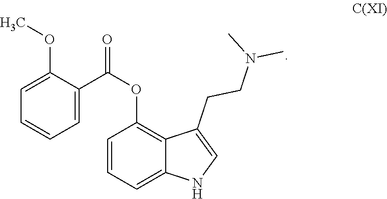 [SMILES: COc1ccccc1C(=O)Oc1cccc2[nH]cc(CCN(C)C)c12]

In an aspect, the present disclosure provides a compound having chemical formula (I) wherein R₄ is a carboxylic acid moiety or derivative thereof, the compound having the chemical formula C(XII):

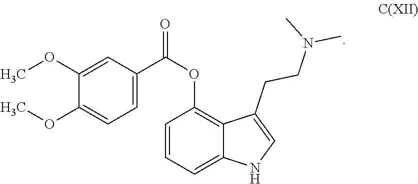

In an aspect, the present disclosure provides a compound having chemical formula (I) wherein R₄ is a carboxylic acid moiety or derivative thereof, the compound having the chemical formula C(XIII):

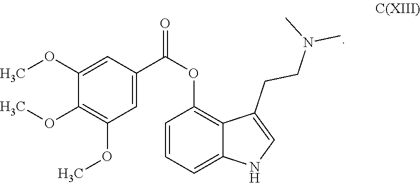

In an aspect, the present disclosure provides a compound having chemical formula (I) wherein R₄ is a carboxylic acid moiety or derivative thereof, the compound having the chemical formula C(XIV):

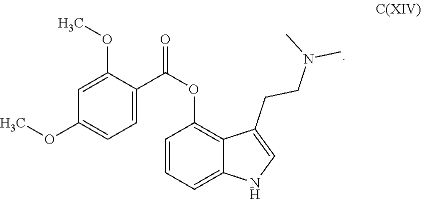

In an aspect, the present disclosure provides a compound having chemical formula (I) wherein R₄ is a carboxylic acid moiety or derivative thereof, the compound having the chemical formula C(XV):

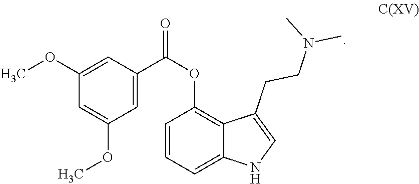

In some embodiments, in an aspect, in the compound having chemical formula (I) wherein R₄ is a carboxylic acid moiety or derivative thereof, the compound having the chemical formula C(XVI):

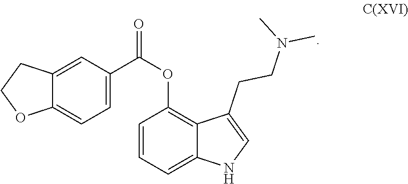 [SMILES: CN(C)CCc1c[nH]c2cccc(OC(=O)c3ccc4c(c3)CCO4)c12]

In an aspect, the present disclosure provides a compound having chemical formula (I) wherein R₄ is a carboxylic acid moiety or derivative thereof, the compound having the chemical formula C(XVII):

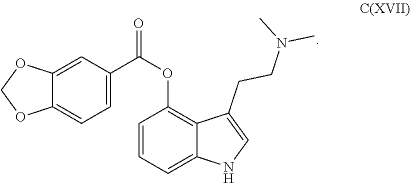 [SMILES: CN(C)CCc1c[nH]c2cccc(OC(=O)c3ccc4c(c3)OCO4)c12]

In an aspect, the present disclosure provides a compound having chemical formula (I) wherein R₄ is a carboxylic acid moiety or derivative thereof, the compound having the chemical formula C(XVIII):

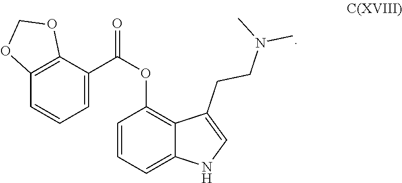 [SMILES: CN(C)CCc1c[nH]c2cccc(OC(=O)c3cccc4c3OCO4)c12]

In an aspect, the present disclosure provides a compound having chemical formula (I) wherein R₄ is a carboxylic acid moiety or derivative thereof, the compound having the chemical formula C(XIX):

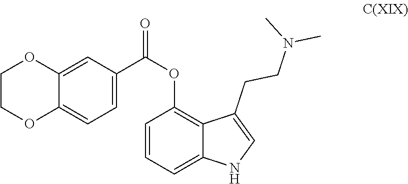 [SMILES: CN(C)CCc1c[nH]c2cccc(OC(=O)c3ccc4c(c3)OCCO4)c12]

In an aspect, the present disclosure provides a compound having chemical formula (I) wherein R₄ is a carboxylic acid moiety or derivative thereof, the compound having the chemical formula C(XX):

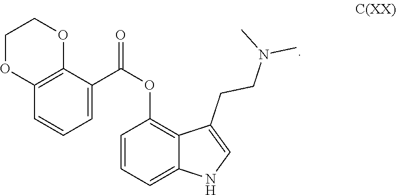 [SMILES: CN(C)CCc1c[nH]c2cccc(OC(=O)c3cccc4c3OCCO4)c12]

In an aspect, the present disclosure provides a compound having chemical formula (I) wherein R₄ is a carboxylic acid moiety or derivative thereof, the compound having the chemical formula C(XXI):

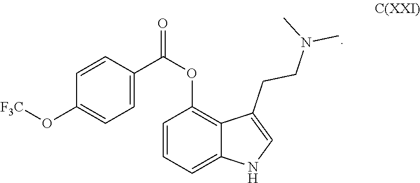 [SMILES: CN(C)CCc1c[nH]c2cccc(OC(=O)c3ccc(OC(F)(F)F)cc3)c12]

In an aspect, the present disclosure provides a compound having chemical formula (I) wherein R₄ is a carboxylic acid moiety or derivative thereof, the compound having the chemical formula C(XXII):

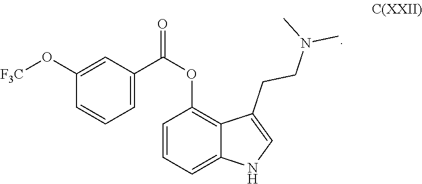 [SMILES: CN(C)CCc1c[nH]c2cccc(OC(=O)c3cccc(OC(F)(F)F)c3)c12]

In an aspect, the present disclosure provides a compound having chemical formula (I) wherein R₄ is a carboxylic acid moiety or derivative thereof, the compound having the chemical formula C(XXIII):

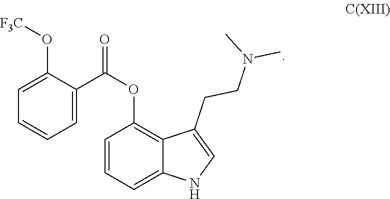 [SMILES: CN(C)CCc1c[nH]c2cccc(OC(=O)c3ccccc3OC(F)(F)F)c12]

In an aspect, the present disclosure provides a compound having chemical formula (I) wherein R₄ is a carboxylic acid moiety or derivative thereof, the compound having the chemical formula C(XXIV):

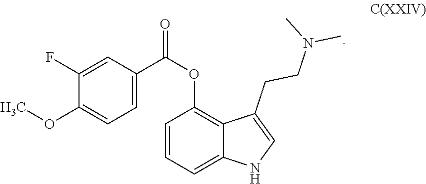

In an aspect, the present disclosure provides a compound having chemical formula (I) wherein R₄ is a carboxylic acid moiety or derivative thereof, the compound having the chemical formula C(XXV):

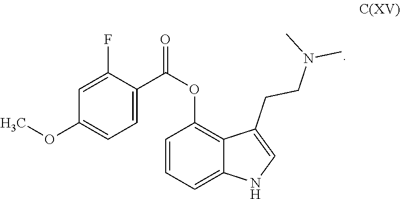

In some embodiments, in an aspect, in the compound having chemical formula (I) wherein R₄ is a carboxylic acid moiety or derivative thereof, the compound having the chemical formula C(XXVI):

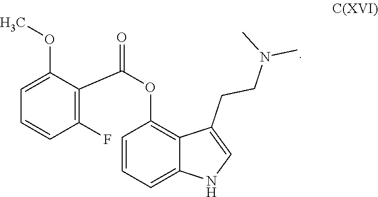 [SMILES: COc1cccc(F)c1C(=O)Oc1cccc2[nH]cc(CCN(C)C)c12]

In an aspect, the present disclosure provides a compound having chemical formula (I) wherein R₄ is a carboxylic acid moiety or derivative thereof, the compound having the chemical formula C(XXVII):

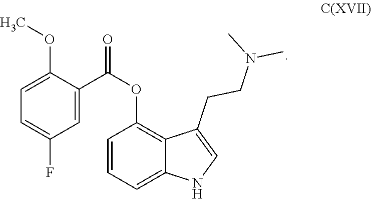 [SMILES: COc1ccc(F)cc1C(=O)Oc1cccc2[nH]cc(CCN(C)C)c12]

In an aspect, the present disclosure provides a compound having chemical formula (I) wherein R₄ is a carboxylic acid moiety or derivative thereof, the compound having the chemical formula C(XXVIII):

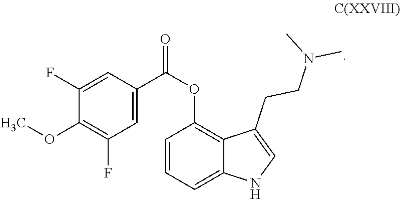

In an aspect, the present disclosure provides a compound having chemical formula (I) wherein R₄ is a carboxylic acid moiety or derivative thereof, the compound having the chemical formula C(XXIX):

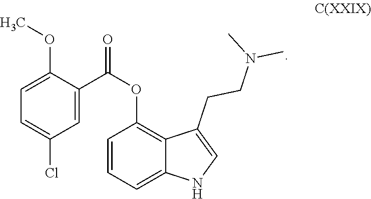 [SMILES: COc1ccc(Cl)cc1C(=O)Oc1cccc2[nH]cc(CCN(C)C)c12]

In an aspect, the present disclosure provides a compound having chemical formula (I) wherein R₄ is a carboxylic acid moiety or derivative thereof, the compound having the chemical formula C(XXX):

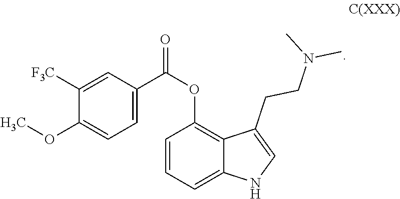

In an aspect, the present disclosure provides a compound having chemical formula (I) wherein R₄ is a carboxylic acid moiety or derivative thereof, the compound having the chemical formula C(XXXI):

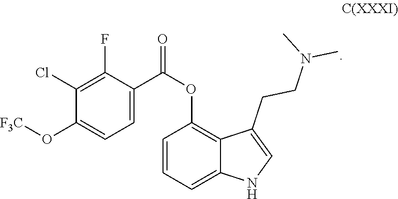 [SMILES: CN(C)CCc1c[nH]c2cccc(OC(=O)c3ccc(OC(F)(F)F)c(Cl)c3F)c12]

In an aspect, the present disclosure provides a compound having chemical formula (I) wherein R₄ is a carboxylic acid moiety or derivative thereof, the compound having the chemical formula C(XXXII):

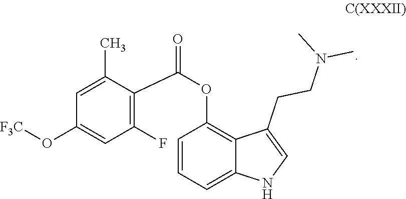 [SMILES: Cc1cc(OC(F)(F)F)cc(F)c1C(=O)Oc1cccc2[nH]cc(CCN(C)C)c12]

In an aspect, the present disclosure provides a compound having chemical formula (I) wherein R₄ is a carboxylic acid moiety or derivative thereof, the compound having the chemical formula C(XXXIII):

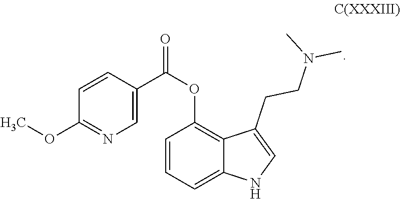

In an aspect, the present disclosure provides a compound having chemical formula (I) wherein R₄ is a carboxylic acid moiety or derivative thereof, the compound having the chemical formula C(XXXIV):

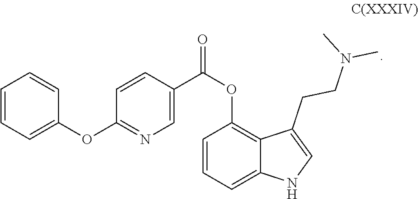 [SMILES: CN(C)CCc1c[nH]c2cccc(OC(=O)c3ccc(Oc4ccccc4)nc3)c12]

In an aspect, the present disclosure provides a compound having chemical formula (I) wherein R₄ is a carboxylic acid moiety or derivative thereof, the compound having the chemical formula C(XXXV):

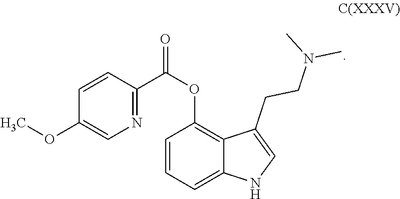

In some embodiments, in an aspect, in the compound having chemical formula (I) wherein R₄ is a carboxylic acid moiety or derivative thereof, the compound having the chemical formula C(XXXVI):

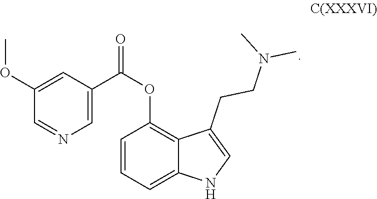 [SMILES: COc1cncc(C(=O)Oc2cccc3[nH]cc(CCN(C)C)c23)c1]

In an aspect, the present disclosure provides a compound having chemical formula (I) wherein R₄ is a carboxylic acid moiety or derivative thereof, the compound having the chemical formula C(XXXVII):

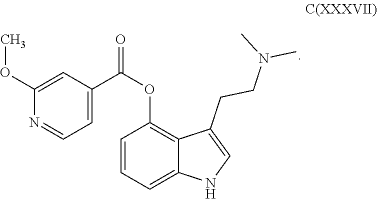 [SMILES: COc1cc(C(=O)Oc2cccc3[nH]cc(CCN(C)C)c23)ccn1]

In an aspect, the present disclosure provides a compound having chemical formula (I) wherein R₄ is a carboxylic acid moiety or derivative thereof, the compound having the chemical formula C(XXXVIII):

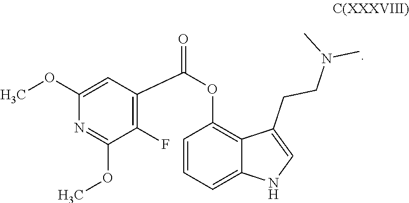

In an aspect, the present disclosure provides a compound having chemical formula (I) wherein R₄ is a carboxylic acid moiety or derivative thereof, the compound having the chemical formula C(XXXIX):

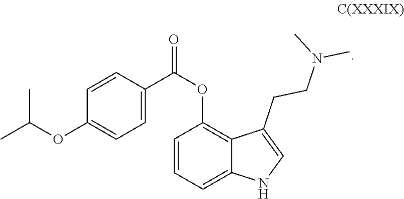 [SMILES: CC(C)Oc1ccc(C(=O)Oc2cccc3[nH]cc(CCN(C)C)c23)cc1]

In an aspect, the present disclosure provides a compound having chemical formula (I) wherein R₄ is a carboxylic acid moiety or derivative thereof, the compound having the chemical formula C(XL):

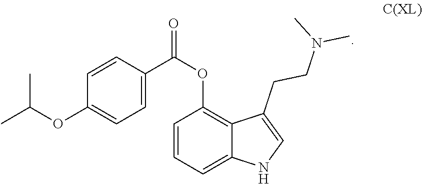 [SMILES: CC(C)Oc1ccc(C(=O)Oc2cccc3[nH]cc(CCN(C)C)c23)cc1]

In an aspect, the present disclosure provides a compound having chemical formula (I) wherein R₄ is a carboxylic acid moiety or derivative thereof, the compound having the chemical formula C(XLI):

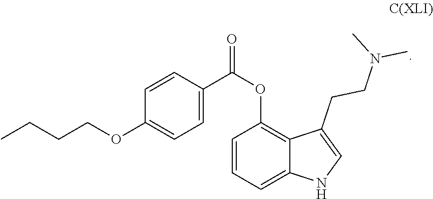 [SMILES: CCCCOc1ccc(C(=O)Oc2cccc3[nH]cc(CCN(C)C)c23)cc1]

In an aspect, the present disclosure provides a compound having chemical formula (I) wherein R₄ is a carboxylic acid moiety or derivative thereof, the compound having the chemical formula C(XLII):

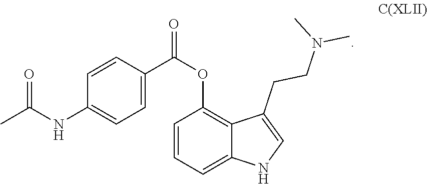 [SMILES: CC(=O)Nc1ccc(C(=O)Oc2cccc3[nH]cc(CCN(C)C)c23)cc1]

In an aspect, the present disclosure provides a compound having chemical formula (I) wherein R₄ is a carboxylic acid moiety or derivative thereof, the compound having the chemical formula C(XLII):

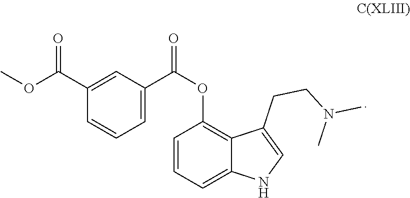

Referring next to the chemical compound having the formula (I), in an aspect hereof, in some embodiments, R₄ can be a polyether moiety or derivative thereof.

In some embodiments, in an aspect, the polyether moiety or derivative thereof can include a carboxylic acid moiety or derivative thereof.

In some embodiments, in an aspect, the polyether moiety or derivative thereof can include an alkyl group, a branched alkyl group, or an O-alkyl group.

In some embodiments, the polyether derivative can be a compound having the formula (XV):

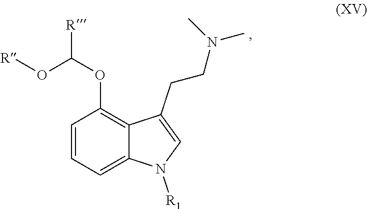

wherein
R₁ is a hydrogen atom; a (C₁-C₆)-alkyl group, in which two hydrogens are optionally replaced with an oxo group; a carbonic ester moiety —C—O—C(═O)—O—(C₁-C₆)-alkyl; or an acyl moiety —C(═O)—(C₁-C₆)-alkyl,
R″ is a polyether moiety or derivative thereof, and R′″ is H or C₁-C₆-alkyl.

In at least one embodiment, in an aspect, R₁ is —C(═O)CH₂CH₃.

In some embodiments, R′″ can be a methyl group.

In some embodiments, R″ can be optionally substituted C₁-C₁₀ alkyl, in which the optional substituents can be alkyl, cycloalkyl, aryl, oxo, (two hydrogens on the alkyl group form an oxo group (═O) group, and in which one or more carbon atoms in the alkyl group are optionally replaced with an oxygen (O) atom.

In some embodiments, R″ can be alkyl substituted by phenyl (—CH₂— phenyl).

In some embodiments, R″ can be a methyl group

In some embodiments, R″ can be a C₁-C₆-alkyl group in which two geminal hydrogens on the alkyl group are joined together to form an oxo group (═O).

In some embodiments, the C₁-C₆-alkyl group substituted by an oxo group, is further substituted by a C₃-C₆-cycloalkyl group (such as a cyclopropyl group) or additional alkyl groups, such as methyl.

In some embodiments, the C₃-C₆ cycloalkyl group can be a cyclopropyl group.

In some embodiments, R″ can be an optionally substituted C₁-C₁₀ alkyl group in which at least one of the carbon atoms in the alkyl group are optionally replaced with an oxygen (O) atom.

In some embodiments, R″ can be an optionally substituted C₁-C₆ alkyl group in which at least one of the carbon atoms in the alkyl group are optionally replaced with an oxygen (O) atom, and which is further substituted by an oxo group or an alkyl group.

In an aspect, the present disclosure provides a compound having chemical formula (I) wherein R₄ is a polyether moiety or derivative thereof, the compound having the chemical formula D(I):

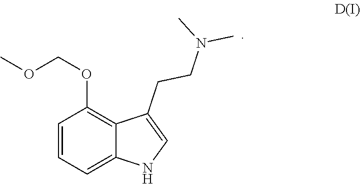

In an aspect, the present disclosure provides a compound having chemical formula (I) wherein R₄ is a polyether moiety or derivative thereof, the compound having the chemical formula D(II):

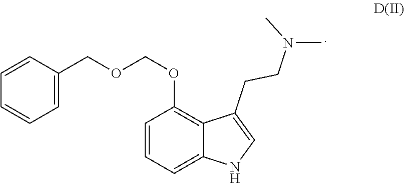 [SMILES: CN(C)CCc1c[nH]c2cccc(OCOCc3ccccc3)c12]

In an aspect, the present disclosure provides a compound having chemical formula (I) wherein R₄ is a polyether moiety or derivative thereof, the compound having the chemical formula D(III):

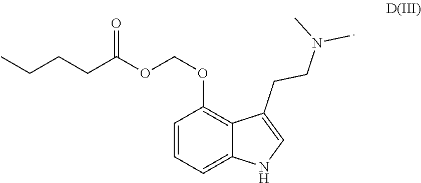 [SMILES: CCCCC(=O)OCOc1cccc2[nH]cc(CCN(C)C)c12]

In an aspect, the present disclosure provides a compound having chemical formula (I) wherein R₄ is a polyether moiety or derivative thereof, the compound having the chemical formula D(IV):

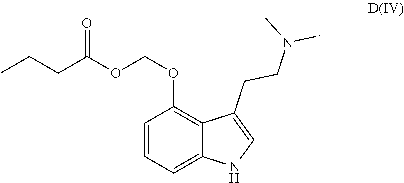

In an aspect, the present disclosure provides a compound having chemical formula (I) wherein R₄ is a polyether moiety or derivative thereof, the compound having the chemical formula D(V):

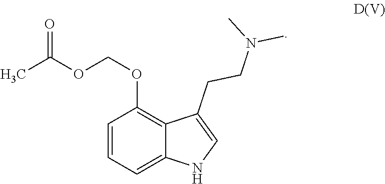 [SMILES: CC(=O)OCOc1cccc2[nH]cc(CCN(C)C)c12]

In some embodiments, in an aspect, in the compound having chemical formula (I) wherein R₄ is a polyether moiety or derivative thereof, the compound having the chemical formula D(VI):

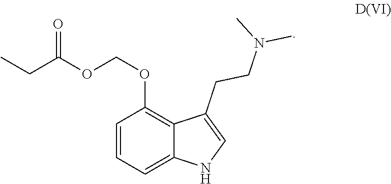 [SMILES: CCC(=O)OCOc1cccc2[nH]cc(CCN(C)C)c12]

In an aspect, the present disclosure provides a compound having chemical formula (I) wherein R₄ is a polyether moiety or derivative thereof, the compound having the chemical formula D(VII):

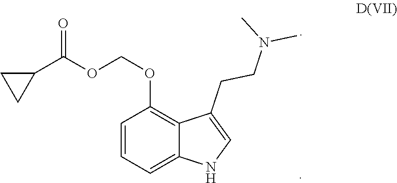 [SMILES: CN(C)CCc1c[nH]c2cccc(OCOC(=O)C3CC3)c12]

In an aspect, the present disclosure provides a compound having chemical formula (I) wherein R₄ is a polyether moiety or derivative thereof, the compound having the chemical formula D(VIII):

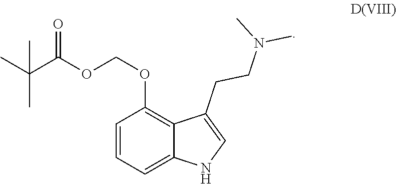 [SMILES: CN(C)CCc1c[nH]c2cccc(OCOC(=O)C(C)(C)C)c12]

In an aspect, the present disclosure provides a compound having chemical formula (I) wherein R₄ is a polyether moiety or derivative thereof, the compound having the chemical formula D(IX):

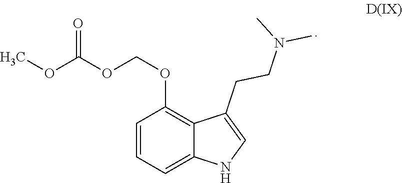

In an aspect, the present disclosure provides a compound having chemical formula (I) wherein R₄ is a polyether moiety or derivative thereof, the compound having the chemical formula D(X):

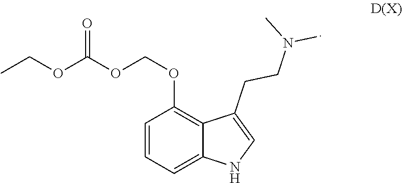

In an aspect, the present disclosure provides a compound having chemical formula (I) wherein R₄ is a polyether moiety or derivative thereof, the compound having the chemical formula D(XI):

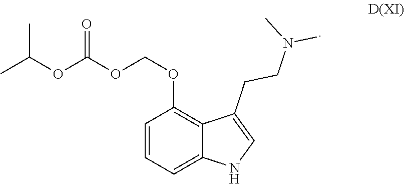

In an aspect, the present disclosure provides a compound having chemical formula (I) wherein R₄ is a polyether moiety or derivative thereof, the compound having the chemical formula D(XII):

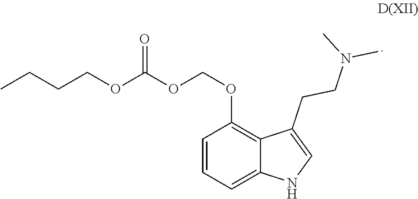 [SMILES: CCCCOC(=O)OCOc1cccc2[nH]cc(CCN(C)C)c12]

In an aspect, the present disclosure provides a compound having chemical formula (I) wherein R₄ is a polyether moiety or derivative thereof, the compound having the chemical formula D(XIII):

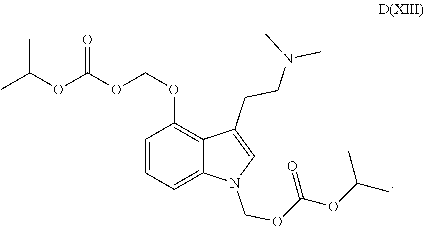 [SMILES: CC(C)OC(=O)OCOc1cccc2c1c(CCN(C)C)cn2COC(=O)OC(C)C]

In an aspect, the present disclosure provides a compound having chemical formula (I) wherein R₄ is a polyether moiety or derivative thereof, the compound having the chemical formula D(XIV):

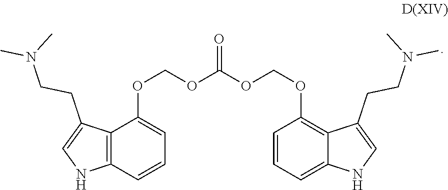

In an aspect, the present disclosure provides a compound having chemical formula (I) wherein R₄ is a polyether moiety or derivative thereof, the compound having the chemical formula D(XV):

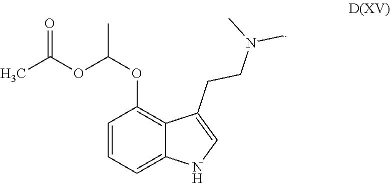 [SMILES: CC(=O)OC(C)Oc1cccc2[nH]cc(CCN(C)C)c12]

In some embodiments, in an aspect, in the compound having chemical formula (I) wherein R₄ is a polyether moiety or derivative thereof, the compound having the chemical formula D(XVI):

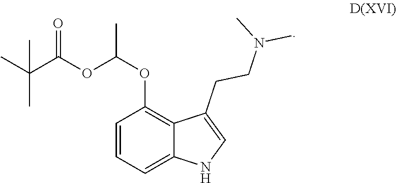 [SMILES: CC(OC(=O)C(C)(C)C)Oc1cccc2[nH]cc(CCN(C)C)c12]

In an aspect, the present disclosure provides a compound having chemical formula (I) wherein R₄ is a polyether moiety or derivative thereof, the compound having the chemical formula D(XVII):

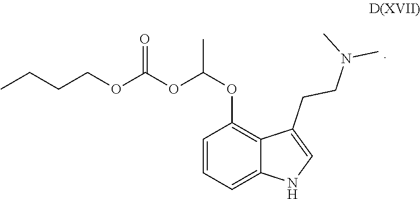 [SMILES: CCCCOC(=O)OC(C)Oc1cccc2[nH]cc(CCN(C)C)c12]

In an aspect, the present disclosure provides a compound having chemical formula (I) wherein R₄ is a polyether moiety or derivative thereof, the compound having the chemical formula D(XVIII):

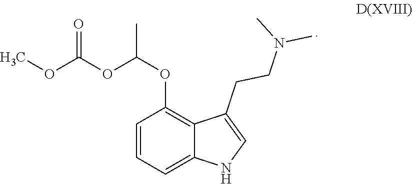

In an aspect, the present disclosure provides a compound having chemical formula (I) wherein R₄ is a polyether moiety or derivative thereof, the compound having the chemical formula D(XIX):

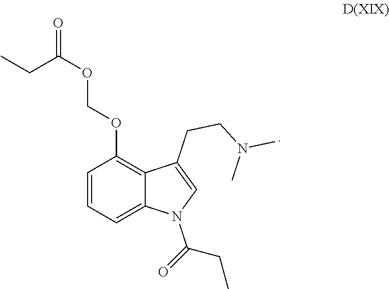 [SMILES: CCC(=O)OCOc1cccc2c1c(CCN(C)C)cn2C(=O)CC]

Referring next to the chemical compound having the formula (I), in an aspect hereof, in some embodiments, R₄ can be a carbonothioate moiety or derivative thereof.

In some embodiments, in an aspect, the carbonothioate moiety or derivative thereof can have the chemical formula (III):

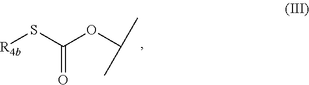

wherein R_{4b} is an alkyl group, a cyclo-alkyl group, or an aryl group, each of which are optionally substituted.

In some embodiments, the carbonothioate moiety or derivative thereof can have the chemical formula (IV):

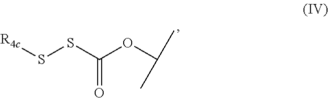

Wherein R_{4c} is an alkyl group, a cyclo-alkyl group, or an aryl group, each of which are optionally substituted.

In some embodiments, in the compound having chemical formula (III), R_{4b} can be C₁-C₆ alkyl optionally substituted with a halogen atom (chloro, fluoro, bromo iodo), alkyl group, cycloalkyl group, or an aryl group, a phenyl group, for example.

In some embodiments, in the compound having chemical formula (III) R_{4b} can be methyl, ethyl, isopropyl, butyl, —CH₂-cyclopropyl, —CH(CH₃)-cyclopropyl, —C(CH₃)₂-cyclopropyl or —CH₂-phenyl.

In some embodiments, R_{4b} can be an aryl group, a phenyl group, for example.

In some embodiments, in the compound having chemical formula (III), R_{4b} can be C₁-C₆ alkyl optionally substituted with a halogen atom (chloro, fluoro, bromo iodo), alkyl group, cycloalkyl group, or aryl group, and wherein one or more of the carbon atoms in the C₁-C₆ alkyl group are replaced with oxygen (O) atoms.

In some embodiments, in the compound having chemical formula (IV), R_{4c} can be C₁-C₆ alkyl optionally substituted with a halogen atom (chloro, fluoro, bromo iodo), alkyl group, cycloalkyl group, or aryl group, a phenyl group for example.

In some embodiments, in the compound having chemical formula (IV) R_{4c} can be methyl, ethyl, isopropyl, butyl, —CH₂-cyclopropyl, —CH(CH₃)-cyclopropyl, —C(CH₃)₂-cyclopropyl or —CH₂-phenyl.

In some embodiments, in the compound having chemical formula (IV) R_{4c} can be an aryl group, a phenyl group for example.

In some embodiments, in the compound having chemical formula (IV, R_{4c} can be C₁-C₆ alkyl optionally substituted with a halogen atom (chloro, fluoro, bromo iodo), alkyl group, cycloalkyl group, or aryl group, and wherein one or more of the carbon atoms in the C₁-C₆ alkyl group are replaced with oxygen (O) atoms.

In an aspect, the present disclosure provides a compound having chemical formula (I) wherein R₄ is a carbonothioate moiety or derivative thereof, the compound having the chemical formula E(I):

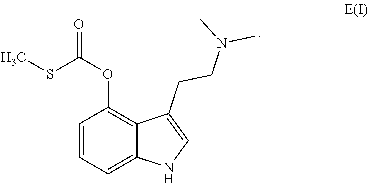

In an aspect, the present disclosure provides a compound having chemical formula (I) wherein R₄ is a carbonothioate moiety or derivative thereof, the compound having the chemical formula E(II):

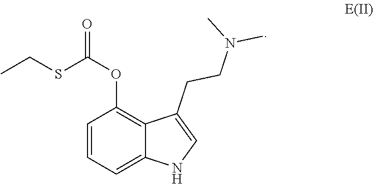 [SMILES: CCSC(=O)Oc1cccc2[nH]cc(CCN(C)C)c12]

In an aspect, the present disclosure provides a compound having chemical formula (I) wherein R₄ is a carbonothioate moiety or derivative thereof, the compound having the chemical formula E(III):

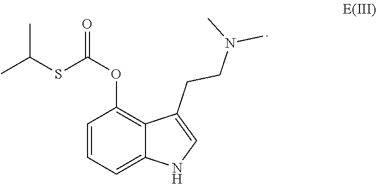 [SMILES: CC(C)SC(=O)Oc1cccc2[nH]cc(CCN(C)C)c12]

In an aspect, the present disclosure provides a compound having chemical formula (I) wherein R₄ is a carbonothioate moiety or derivative thereof, the compound having the chemical formula E(IV):

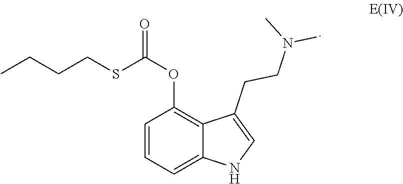

In an aspect, the present disclosure provides a compound having chemical formula (I) wherein R₄ is a carbonothioate moiety or derivative thereof, the compound having the chemical formula E(V):

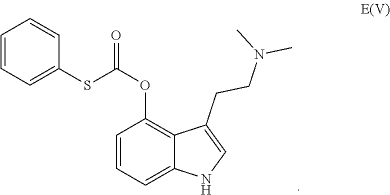 [SMILES: CN(C)CCc1c[nH]c2cccc(OC(=O)Sc3ccccc3)c12]

In some embodiments, in an aspect, in the compound having chemical formula (I) wherein R₄ is a carbonothioate moiety or derivative thereof, the compound having the chemical formula E(VI):

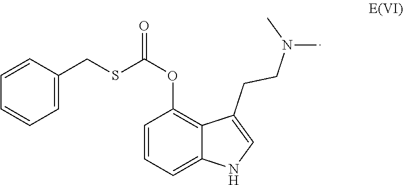

In an aspect, the present disclosure provides a compound having chemical formula (I) wherein R₄ is a carbonothioate moiety or derivative thereof, the compound having the chemical formula E(VII):

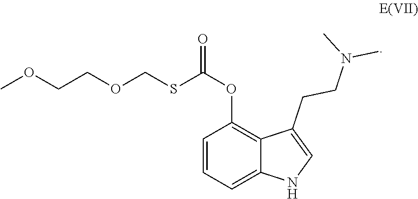

In an aspect, the present disclosure provides a compound having chemical formula (I) wherein R₄ is a carbonothioate moiety or derivative thereof, the compound having the chemical formula E(VIII):

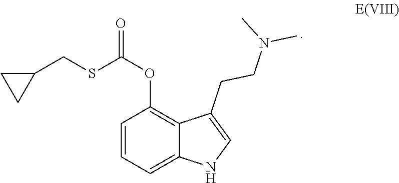 [SMILES: CN(C)CCc1c[nH]c2cccc(OC(=O)SCC3CC3)c12]

In an aspect, the present disclosure provides a compound having chemical formula (I) wherein R₄ is a carbonothioate moiety or derivative thereof, the compound having the chemical formula E(IX):

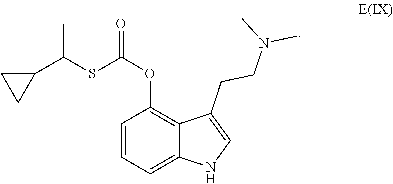 [SMILES: CC(SC(=O)Oc1cccc2[nH]cc(CCN(C)C)c12)C1CC1]

In an aspect, the present disclosure provides a compound having chemical formula (I) wherein R₄ is a carbonothioate moiety or derivative thereof, the compound having the chemical formula E(X):

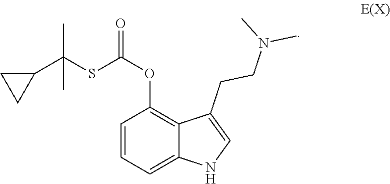 [SMILES: CN(C)CCc1c[nH]c2cccc(OC(=O)SC(C)(C)C3CC3)c12]

In an aspect, the present disclosure provides a compound having chemical formula (I) wherein R₄ is a carbonothioate moiety or derivative thereof, the compound having the chemical formula E(XI):

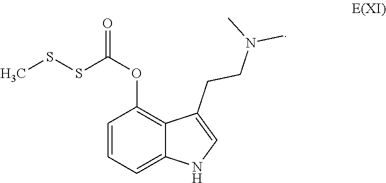

In an aspect, the present disclosure provides a compound having chemical formula (I) wherein R₄ is a carbonothioate moiety or derivative thereof, the compound having the chemical formula E(XII):

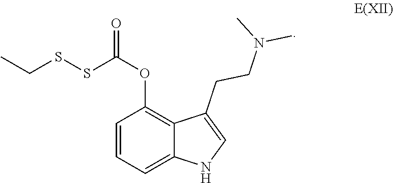 [SMILES: CCSSC(=O)Oc1cccc2[nH]cc(CCN(C)C)c12]

In an aspect, the present disclosure provides a compound having chemical formula (I) wherein R₄ is a carbonothioate moiety or derivative thereof, the compound having the chemical formula E(XIII):

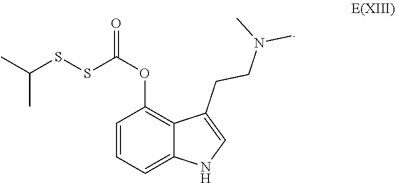 [SMILES: CC(C)SSC(=O)Oc1cccc2[nH]cc(CCN(C)C)c12]

In an aspect, the present disclosure provides a compound having chemical formula (I) wherein R₄ is a carbonothioate moiety or derivative thereof, the compound having the chemical formula E(XIV):

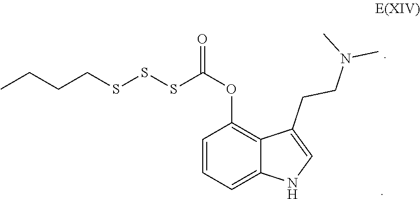 [SMILES: CCCCSSSC(=O)Oc1cccc2[nH]cc(CCN(C)C)c12]

In an aspect, the present disclosure provides a compound having chemical formula (I) wherein R₄ is a carbonothioate moiety or derivative thereof, the compound having the chemical formula E(XV):

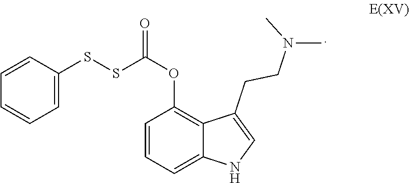 [SMILES: CN(C)CCc1c[nH]c2cccc(OC(=O)SSc3ccccc3)c12]

In some embodiments, in an aspect, in the compound having chemical formula (I) wherein R₄ is a carbonothioate moiety or derivative thereof, the compound having the chemical formula E(XVI):

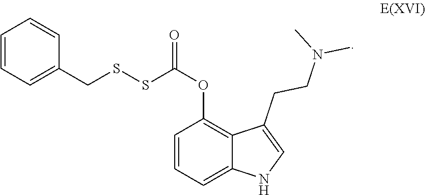 [SMILES: CN(C)CCc1c[nH]c2cccc(OC(=O)SSCc3ccccc3)c12]

In an aspect, the present disclosure provides a compound having chemical formula (I) wherein R₄ is a carbonothioate moiety or derivative thereof, the compound having the chemical formula E(XVII):

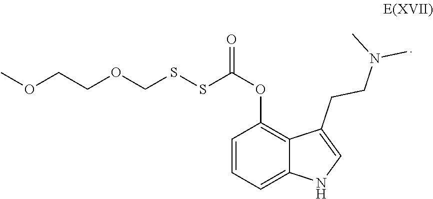

In an aspect, the present disclosure provides a compound having chemical formula (I) wherein R₄ is a carbonothioate moiety or derivative thereof, the compound having the chemical formula E(XVIII):

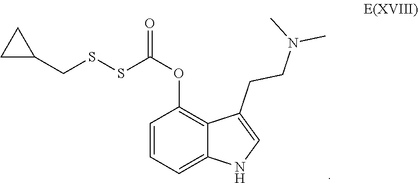 [SMILES: CN(C)CCc1c[nH]c2cccc(OC(=O)SSCC3CC3)c12]

In an aspect, the present disclosure provides a compound having chemical formula (I) wherein R₄ is a carbonothioate moiety or derivative thereof, the compound having the chemical formula E(XIX):

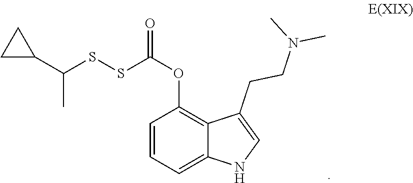 [SMILES: CC(SSC(=O)Oc1cccc2[nH]cc(CCN(C)C)c12)C1CC1]

In an aspect, the present disclosure provides a compound having chemical formula (I) wherein R₄ is a carbonothioate moiety or derivative thereof, the compound having the chemical formula E(XX):

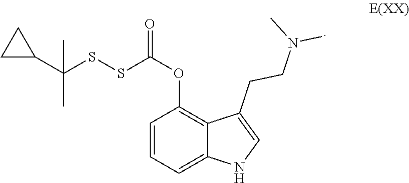

Referring next to the chemical compound having the formula (I), in an aspect hereof, in some embodiments, R₄ can be a phosphate moiety or derivative thereof.

In some embodiments, in the compound having chemical formula (I) wherein R₄ is a phosphate moiety or derivative thereof, the compound can have chemical formula (XVI):

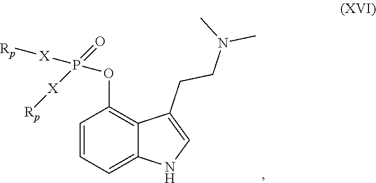

wherein,
X is O or NH;
each Rₚ is independently or simultaneously alkyl, aryl, or each Rₚ is joined together to form an optionally substituted 4-7-membered heterocyclic ring.

In some embodiments, Rₚ can be a phenyl group.

In some embodiments, in an aspect, each Rₚ can be joined together to form an optionally substituted 5-6-membered heterocyclic ring, in which the optional substituents are a phenyl group, or alkyl group (a methyl group, for example), or two substituents are joined together to form an optionally substituted phenyl ring, substituted by an alkyl group (a methyl group, for example) or an alkoxy group (a methoxy group, for example).

In some embodiments, Rₚ can be an optionally substituted C₁-C₁₀-alkyl group, wherein one or more atoms in the C₁-C₁₀ alkyl group are replaced with an oxygen (O) atom or sulfur (S) atom, and the optional substituents are oxo, wherein two hydrogens on the same carbon are taken together to form ═O, or alkyl, a methyl group, or ethyl group, for example.

In an aspect, the present disclosure provides a compound having chemical formula (I) wherein R₄ is a phosphate moiety or derivative thereof, the compound having the chemical formula F(I):

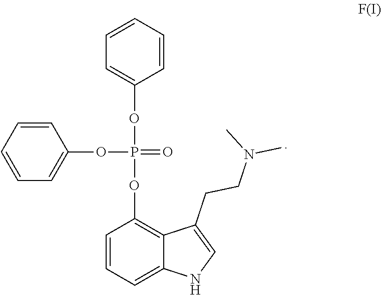 [SMILES: CN(C)CCc1c[nH]c2cccc(OP(=O)(Oc3ccccc3)Oc3ccccc3)c12]

In an aspect, the present disclosure provides a compound having chemical formula (I) wherein R₄ is a phosphate moiety or derivative thereof, the compound having the chemical formula F(II):

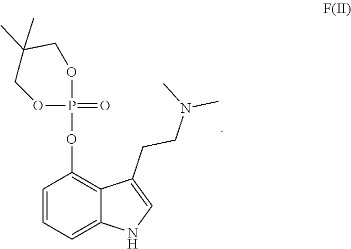

In an aspect, the present disclosure provides a compound having chemical formula (I) wherein R₄ is a phosphate moiety or derivative thereof, the compound having the chemical formula F(III):

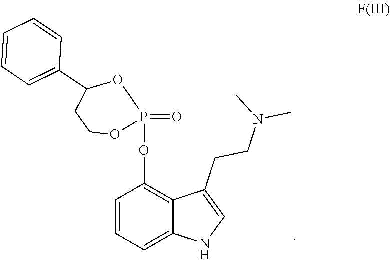 [SMILES: CN(C)CCc1c[nH]c2cccc(OP3(=O)OCCC(c4ccccc4)O3)c12]

In an aspect, the present disclosure provides a compound having chemical formula (I) wherein R₄ is a phosphate moiety or derivative thereof, the compound having the chemical formula F(IV):

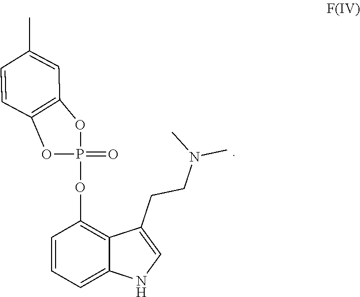 [SMILES: Cc1ccc2c(c1)OP(=O)(Oc1cccc3[nH]cc(CCN(C)C)c13)O2]

In an aspect, the present disclosure provides a compound having chemical formula (I) wherein R₄ is a phosphate moiety or derivative thereof, the compound having the chemical formula F(V):

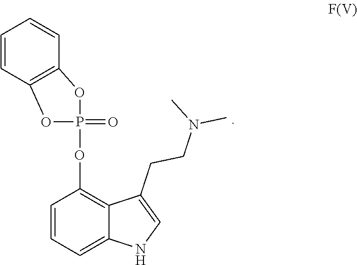 [SMILES: CN(C)CCc1c[nH]c2cccc(OP3(=O)Oc4ccccc4O3)c12]

In some embodiments, in an aspect, in the compound having chemical formula (I) wherein R₄ is a phosphate moiety or derivative thereof, the compound having the chemical formula F(VI):

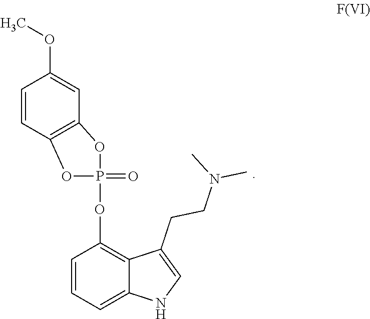 [SMILES: COc1ccc2c(c1)OP(=O)(Oc1cccc3[nH]cc(CCN(C)C)c13)O2]

In an aspect, the present disclosure provides a compound having chemical formula (I) wherein R₄ is a phosphate moiety or derivative thereof, the compound having the chemical formula F(VII):

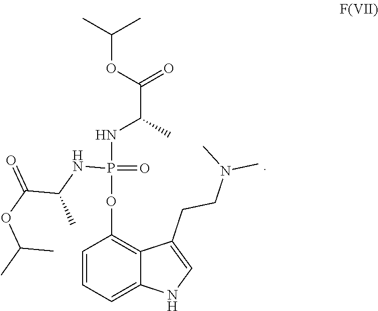 [SMILES: CC(C)OC(=O)[C@H](C)NP(=O)(N[C@H](C)C(=O)OC(C)C)Oc1cccc2[nH]cc(CCN(C)C)c12]

In an aspect, the present disclosure provides a compound having chemical formula (I) wherein R₄ is a phosphate moiety or derivative thereof, the compound having the chemical formula F(VIII):

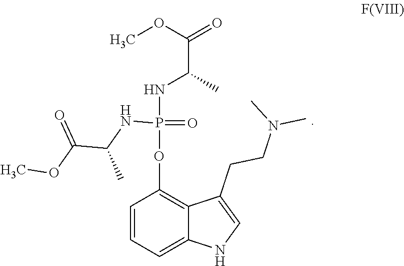

In an aspect, the present disclosure provides a compound having chemical formula (I) wherein R₄ is a phosphate moiety or derivative thereof, the compound having the chemical formula F(IX):

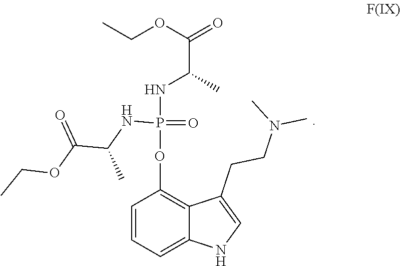 [SMILES: CCOC(=O)[C@H](C)NP(=O)(N[C@H](C)C(=O)OCC)Oc1cccc2[nH]cc(CCN(C)C)c12]

In an aspect, the present disclosure provides a compound having chemical formula (I) wherein R₄ is a phosphate moiety or derivative thereof, the compound having the chemical formula F(X):

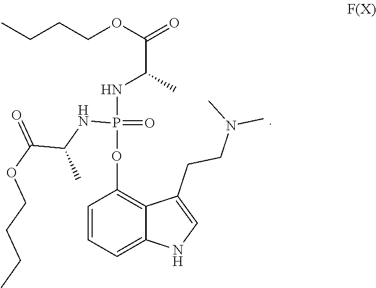 [SMILES: CCCCOC(=O)[C@H](C)NP(=O)(N[C@H](C)C(=O)OCCCC)Oc1cccc2[nH]cc(CCN(C)C)c12]

In an aspect, the present disclosure provides a compound having chemical formula (I) wherein R₄ is a phosphate moiety or derivative thereof, the compound having the chemical formula F(XI):

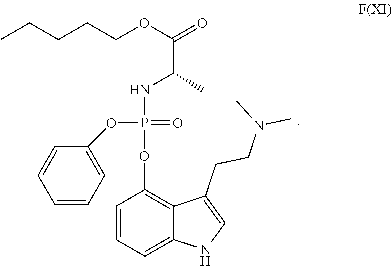 [SMILES: CCCCCOC(=O)[C@H](C)NP(=O)(Oc1ccccc1)Oc1cccc2[nH]cc(CCN(C)C)c12]

In an aspect, the present disclosure provides a compound having chemical formula (I) wherein R₄ is a phosphate moiety or derivative thereof, the compound having the chemical formula F(XII):

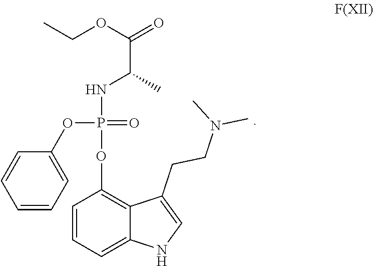 [SMILES: CCOC(=O)[C@H](C)NP(=O)(Oc1ccccc1)Oc1cccc2[nH]cc(CCN(C)C)c12]

In an aspect, the present disclosure provides a compound having chemical formula (I) wherein R₄ is a phosphate moiety or derivative thereof, the compound having the chemical formula F(XIII):

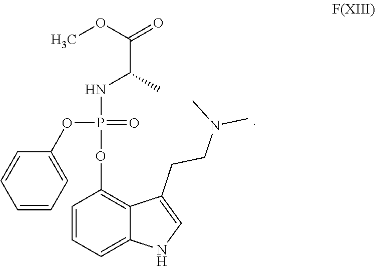 [SMILES: COC(=O)[C@H](C)NP(=O)(Oc1ccccc1)Oc1cccc2[nH]cc(CCN(C)C)c12]

In an aspect, the present disclosure provides a compound having chemical formula (I) wherein R₄ is a phosphate moiety or derivative thereof, the compound having the chemical formula F(XIV):

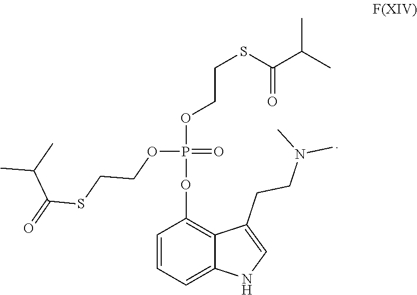 [SMILES: CC(C)C(=O)SCCOP(=O)(OCCSC(=O)C(C)C)Oc1cccc2[nH]cc(CCN(C)C)c12]

In an aspect, the present disclosure provides a compound having chemical formula (I) wherein R₄ is a phosphate moiety or derivative thereof, the compound having the chemical formula F(XV):

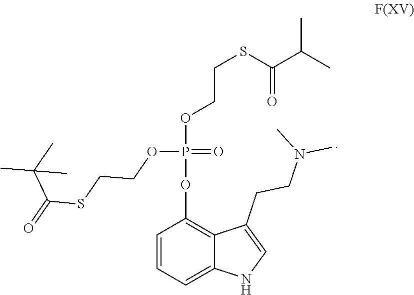 [SMILES: CC(C)C(=O)SCCOP(=O)(OCCSC(=O)C(C)(C)C)Oc1cccc2[nH]cc(CCN(C)C)c12]

Referring now again to the compound having chemical formula (I), it is noted that in various embodiments, R₃ₐ and R_{3b} may each independently selected from a hydrogen atom and an alkyl group.

Referring further to the compound having chemical formula (I), it is noted that in various embodiments, R₁ may be a carbonic ester moiety or derivative thereof. Suitable examples in this respect are a carbonic ester moiety or derivative thereof having chemical formula (V):

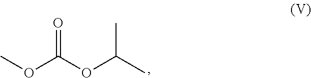

and a carbonic ester moiety or derivative thereof having chemical formula (XIII):

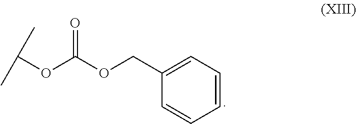

Referring further to the compound having chemical formula (I), it is noted that in various embodiments, R₁ may be a ketone or derivative thereof. Suitable examples in this respect are a carbonic ester moiety or derivative thereof having chemical formula (XVII):

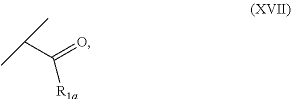

wherein R₁ₐ is a hydrogen atom or an alkyl group, including for example a C₁-C₁₀ alkyl group, a C₁-C₆ alkyl group, and a C₁-C₃ alkyl group. Thus, in suitable examples R₁, can be (XVIII) or (XIX):

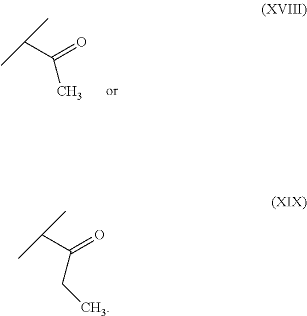

Referring again to the compound having formula (I) in some embodiments, R₄ can be a substituent selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof,
   wherein (NR₃ₐR_{3b}) has the formula (N+HR₃ₐR_{3b}Z), wherein Z is a counter-balancing anion, and wherein compound (I) is a salt.

In one embodiment, Z can be a mono-valent counter-balancing ion (Z⁻), a di-valent counter-balancing ion (Z²⁻), or a tri-valent counter-balancing ion (Z³⁻).

In one embodiment, Z can be a mono-valent counter-balancing anion (Z⁻) selected from a halide ion (Cl⁻, Br⁻, F⁻, I⁻), a nitrate ion (NO₃⁻), a benzoate ion (phenyl-COO⁻), a succinate ion (HOOC—(CH₂)₂—COO⁻), a fumarate ion (trans-HOOC—(CH═CH)—COO⁻), a tartarate ion (HOOC—(CHOH)₂—COO⁻), a malate ion (HOOC—CH₂—CHOH—COO⁻), a maleate ion (cis-HOOC—(CH═CH)—COO⁻), a dibenzoyl tartarate ion (HOOC—(CHOBz)₂-COO⁻;

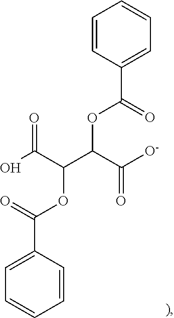 [SMILES: O=C(OC(C(=O)[O-])C(OC(=O)c1ccccc1)C(=O)O)c1ccccc1]

a ditoluoyl tartarate ion (HOOC—(CHOCOTol)₂-COO⁻;

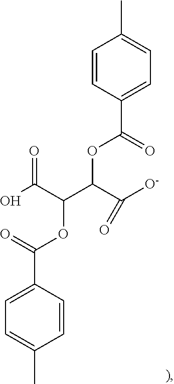 [SMILES: Cc1ccc(C(=O)OC(C(=O)[O-])C(OC(=O)c2ccc(C)cc2)C(=O)O)cc1]

a malonate ion (HOOC—CH₂—COO⁻), a dihydrogen phosphate ion (H₂PO₄⁻), and an acetate ion (CH₃—COO⁻), wherein the salt compound has the formula (Iₐ):

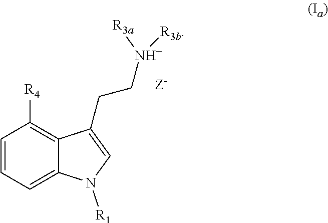

In one embodiment, Z can be a di-valent counter-balancing anion (Z²⁻) selected from a sulfate ion (SO₄²⁻), a hydrogen phosphate ion (HPO₄²⁻), a succinate dianion (⁻OOC—(CH₂)₂—COO⁻), a fumarate dianion (trans-⁻OCC—(CH═CH)—COO⁻), a tartarate dianion (⁻OOC—(CHOH)₂—COO⁻), a malate dianion (⁻OOC—CH₂—CHOH—COO⁻), a maleate dianion (cis-⁻OOC—(CH═CH)—COO⁻), a dibenzoyl tartarate dianion (⁻OOC—(CHOBz)₂-COO⁻;

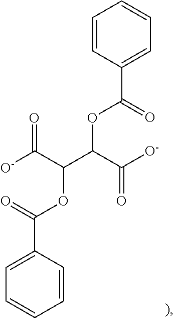 [SMILES: O=C(OC(C(=O)[O-])C(OC(=O)c1ccccc1)C(=O)[O-])c1ccccc1]

a ditoluoyl tartarate dianion (⁻OOC—(CHOCOTol)₂-COO⁻;

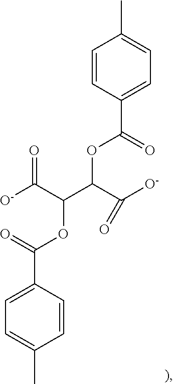 [SMILES: Cc1ccc(C(=O)OC(C(=O)[O-])C(OC(=O)c2ccc(C)cc2)C(=O)[O-])cc1]

and a malonate dianion (⁻OOC—CH₂—COO⁻), wherein the salt compound has the formula (I_{b}):

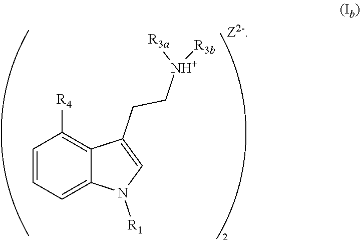

In one embodiment, Z can be a tri-valent counter-balancing anion (Z³⁻) selected from a phosphate ion (PO₄³⁻) and a citrate ion (⁻OOC—CH₂—C(OH)(COO⁻)—CH₂—COO⁻, and the salt compound has the formula (I_{c}):

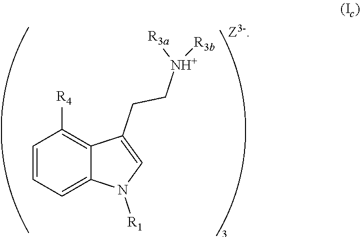

Thus, to briefly recap, the present disclosure provides C₄-substituted tryptamine derivatives. The disclosure provides, in particular, a chemical compound having a formula (I):

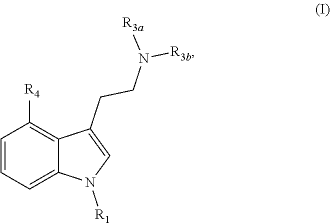

wherein R₄ is a substituent selected from:
(A) an ether moiety or derivative thereof;
(B) a carbonic ester moiety or derivative thereof;
(C) a carboxylic acid moiety or derivative thereof;
(D) a polyether moiety or derivative thereof;
(E) a carbonothioate moiety or derivative thereof; and
(F) a phosphate moiety or derivative thereof;
   wherein R₁ is a hydrogen atom or a moiety containing a carbonic ester or derivative thereof, or a ketone or ketone derivative; and
   wherein R₃ₐ and R_{3b} are each independently a hydrogen atom or an alkyl group.

In some embodiments, the C₄-substituted tryptamine derivatives can comprise a substituent R₄, wherein R₄ is selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof.

The foregoing derivatives (A), (B), (C), (D), (E) and (F) may include alkyl groups, including O-alkyl groups. In one embodiment, the alkyl groups (including O-alkyl) is C₁-C₂₀-alkyl. In another embodiment, the alkyl group is C₁-C₁₀-alkyl. In another embodiment, the alkyl group is C₁-C₆-alkyl. In another embodiment, the alkyl group is methyl, ethyl, propyl, butyl or pentyl.

The foregoing derivatives may include aryl groups. In one embodiment, the aryl groups are optionally substituted C₆-C₁₄-aryl. In another embodiment, the aryl group is optionally substituted C₆-C₁₀-aryl, or phenyl. In another embodiment, the aryl group is phenyl, naphthyl, tetrahydronaphthyl, phenanthrenyl, biphenylenyl, indanyl, or indenyl, and the like.

In another embodiment, R₃ₐ and R_{3b} are a hydrogen atom or a (C₁-C₂₀)-alkyl group. In another embodiment, R₃ₐ and R_{3b} are a hydrogen atom or a (C₁-C₁₀)-alkyl group. In another embodiment, R₃ₐ and R_{3b} are a hydrogen atom or a (C₁-C₆)-alkyl group. In another embodiment, R₃ₐ and R_{3b} are a hydrogen atom, a methyl group, an ethyl group, or a propyl group.

The C₄-substitutent tryptamine derivatives of the present disclosure may be used to prepare a pharmaceutical or recreational drug formulation. Thus, in one embodiment, the present disclosure further provides in another aspect, pharmaceutical and recreational drug formulations comprising C₄-substituted tryptamine derivatives. Accordingly, in one aspect, the present disclosure provides in a further embodiment a pharmaceutical or recreational drug formulation comprising a chemical compound having a formula (I):

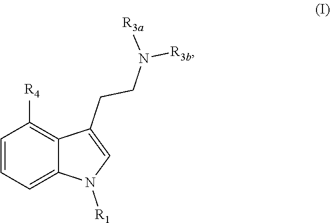

wherein R₄ is a substituent selected from:
(A) an ether moiety or derivative thereof;
(B) a carbonic ester moiety or derivative thereof;
(C) a carboxylic acid moiety or derivative thereof;
(D) a polyether moiety or derivative thereof;
(E) a carbonothioate moiety or derivative thereof; and
(F) a phosphate moiety or derivative thereof;
   wherein R₁ is a hydrogen atom or a moiety containing a carbonic ester or derivative thereof, or a ketone or ketone derivative; and
   wherein R₃ₐ and R_{3b} are each independently a hydrogen atom or an alkyl group.

In some, embodiments, the pharmaceutical or recreational drug formulation comprising a compound having formula (I) can be formulated as a salt, and R₄ can be a substituent selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof,
   wherein (NR₃ₐR_{3b}) has the formula (N⁺HR₃ₐR_{3b}Z), wherein is Z is a pharmaceutically acceptable counter-balancing anion.

In one embodiment, Z can be a pharmaceutically acceptable mono-valent counter-balancing ion (Z⁻), a pharmaceutically acceptable di-valent counter-balancing ion (Z²⁻), or a pharmaceutically acceptable tri-valent counter-balancing ion (Z³⁻).

In one embodiment, Z can be a pharmaceutically acceptable mono-valent counter-balancing anion (Z⁻) selected from a chloride ion (Cl⁻), a benzoate ion (phenyl-COO⁻), a succinate ion (HOOC—(CH₂)₂—COO⁻), a fumarate ion (cis-HOOC—(CH═CH)—COO⁻), a tartarate ion (HOOC—(CHOH)₂—COO⁻), a malate ion (HOOC—CH₂—CHOH—COO⁻), a maleate ion (trans-HOOC—(CH═CH)—COO⁻), a bromide ion (Br⁻), a dihydrogen phosphate ion (H₂PO₄⁻), and an acetate ion (CH₃—COO⁻), wherein the salt compound has the formula (Iₐ):

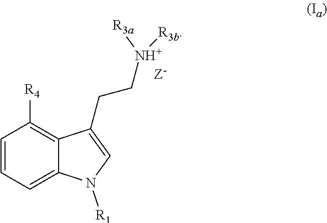

In one embodiment, Z can be a pharmaceutically acceptable di-valent counter-balancing anion (Z²⁻) selected from a sulfate ion (SO₄²⁻), a hydrogen phosphate ion (HPO₄²⁻), a hemi-succinate ion (—OOC—(CH₂)₂—COO⁻), a hemi-fumarate ion (cis-⁻OOC—(CH═CH)—COO⁻), a hemi-tartarate ion (⁻COO—(CHOH)₂—COO⁻), a hemi-malate ion (⁻OOC—CH₂—CHOH—COO⁻), and a hemimaleate ion (trans-⁻OOC—(CH═CH)—COO⁻), wherein the salt compound has the formula (I_{b}):

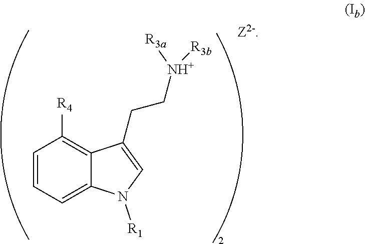

In one embodiment, in an aspect, Z can be a pharmaceutically acceptable tri-valent counter-balancing anion (Z³⁻) selected from a phosphate ion (PO₄³⁻) and a citrate ion (⁻OOC—CH₂—C(OH)(COO⁻)—CH₂—COO⁻, and the salt compound has the formula (I_{c}):

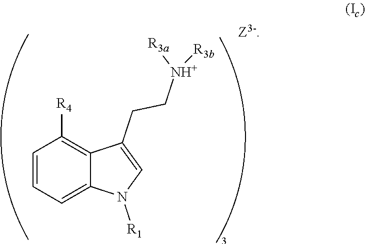

The pharmaceutical or recreational drug formulations may be prepared as liquids, tablets, capsules, microcapsules, nanocapsules, trans-dermal patches, gels, foams, oils, aerosols, nanoparticulates, powders, creams, emulsions, micellar systems, films, sprays, ovules, infusions, teas, decoctions, suppositories, etc. and include a pharmaceutically acceptable salt or solvate of the C₄-substituted tryptamine derivative compound together with an excipient. The term “excipient” as used herein means any ingredient other than the chemical compound of the disclosure. As will readily be appreciated by those of skill in art, the selection of excipient may depend on factors such as the particular mode of administration, the effect of the excipient on solubility of the chemical compounds of the present disclosure and methods for their preparation will be readily apparent to those skilled in the art. Such compositions and methods for their preparation may be found, for example, in “Remington's Pharmaceutical Sciences”, 22^{nd} Edition (Pharmaceutical Press and Philadelphia College of Pharmacy at the University of the Sciences, 2012).

The dose when using the compounds of the present disclosure can vary within wide limits, and as is customary and is known to those of skill in the art, the dose can be tailored to the individual conditions in each individual case. The dose depends, for example, on the nature and severity of the illness to be treated, on the condition of the patient, on the compound employed or on whether an acute or chronic disease state is treated, or prophylaxis is conducted, on the mode of delivery of the compound, or on whether further active compounds are administered in addition to the compounds of the present disclosure. Representative doses of the present invention include, but are not limited to, about 0.001 mg to about 5000 mg, about 0.001 mg to about 2500 mg, about 0.001 mg to about 1000 mg, about 0.001 mg to about 500 mg, about 0.001 mg to about 250 mg, about 0.001 mg to about 100 mg, about 0.001 mg to about 50 mg, and about 0.001 mg to about 25 mg. Representative doses of the present disclosure include, but are not limited to, about 0.0001 to about 1,000 mg, about 10 to about 160 mg, about 10 mg, about 20 mg, about 40 mg, about 80 mg or about 160 mg. Multiple doses may be administered during the day, especially when relatively large amounts are deemed to be needed, for example 2, 3 or 4, doses. Depending on the subject and as deemed appropriate from the patient's physician or care giver it may be necessary to deviate upward or downward from the doses described herein.

The pharmaceutical and drug formulations comprising the C₄-substituted tryptamine derivative compounds of the present disclosure may be administered orally. Oral administration may involve swallowing, so that the compound enters the gastrointestinal tract, or buccal or sublingual administration may be employed by which the compound enters the blood stream directly from the mouth. Formulations suitable for oral administration include both solid and liquid formulations.

Solid formulations include tablets, capsules (containing particulates, liquids, microcapsules, or powders), lozenges (including liquid-filled lozenges), chews, multi- and nano-particulates, gels, solid solutions, liposomal preparations, microencapsulated preparations, creams, films, ovules, suppositories, and sprays.

Liquid formulations include suspensions, solutions, syrups, and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose.

Tablets may also contain diluents, such as lactose (monohydrate, spray-dried monohydrate, anhydrous and the like), mannitol, xylitol, dextrose, sucrose, sorbitol, microcrystalline cellulose, starch, and dibasic calcium phosphate dihydrate.

Tablets may also optionally comprise surface active agents, such as sodium lauryl sulfate and polysorbate 80. When present, surface active agents may comprise from 0.2% (w/w) to 5% (w/w) of the tablet.

Tablets may further contain lubricants such as magnesium stearate, calcium stearate, zinc stearate, sodium stearyl fumarate, and mixtures of magnesium stearate with sodium lauryl sulphate. Lubricants generally comprise from 0.25% (w/w) to 10% (w/w), from 0.5% (w/w) to 3% (w/w) of the tablet.

In addition to the C₄-substituted tryptamine derivative compounds, tablets may contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1% (w/w) to 25% (w/w) or from 5% (w/w) to 20% (w/w) of the dosage form.

Other possible auxiliary ingredients include anti-oxidants, colourants, flavouring agents, preservatives, and taste-masking agents.

For tablet dosage forms, depending on the desired effective amount of the chemical compound, the chemical compound of the present disclosure may make up from 1% (w/w) to 80% (w/w) of the dosage form, more typically from 5% (w/w) to 60% (w/w) of the dosage form.

Exemplary tablets contain up to about 80% (w/w) of the chemical compound, from about 10% (w/w) to about 90% (w/w) binder, from about 0% (w/w) to about 85% (w/w) diluent, from about 2% (w/w) to about 10% (w/w) disintegrant, and from about 0.25% (w/w) to about 10% (w/w) lubricant.

The formulation of tablets is discussed in “Pharmaceutical Dosage Forms: Tablets”, Vol. 1-Vol. 3, by CRC Press (2008).

The pharmaceutical and recreational drug formulations comprising the C₄-substituted tryptamine derivative compound of the present disclosure may also be administered directly into the blood stream, into muscle, or into an internal organ. Thus, the pharmaceutical and recreational drug formulations can be administered parenterally (for example, by subcutaneous, intravenous, intraarterial, intrathecal, intraventricular, intracranial, intramuscular, or intraperitoneal injection). Parenteral formulations are typically aqueous solutions which may contain excipients such as salts, carbohydrates, and buffering agents (in one embodiment, to a pH of from 3 to 9), but, for some applications, they may be more suitably formulated as a sterile non-aqueous solution or as a dried form to be used in conjunction with a suitable vehicle such as sterile water.

Formulations comprising the C₄-substituted tryptamine derivative compound of the present disclosure for parenteral administration may be formulated to be immediate and/or modified release. Modified release formulations include delayed-, sustained-, pulsed-, controlled-, targeted and programmed release. Thus, the chemical compounds of the disclosure may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The pharmaceutical or recreational drug formulations of the present disclosure also may be administered topically to the skin or mucosa, i.e., dermally, or transdermally. Example pharmaceutical and recreational drug formulations for this purpose include gels, hydrogels, lotions, solutions, creams, ointments, dusting powders, cosmetics, oils, eye drops, dressings, foams, films, skin patches, wafers, implants, sponges, fibres, bandages and microemulsions. Liposomes may also be used. Example carriers include alcohol, water, mineral oil, liquid petrolatum, white petrolatum, glycerin, polyethylene glycol and propylene glycol. Penetration enhancers may be incorporate (see: for example, Finnin, B. and Morgan, T. M., 1999 J. Pharm. Sci, 88 (10), 955-958).

Other means of topical administration include delivery by electroporation, iontophoresis, phonophoresis, sonophoresis and microneedle or needle-free (e.g., Powderject™, Bioject™, etc.) injection.

Pharmaceutical and recreational drug formulations for inhalation or insufflation include solutions and suspensions in pharmaceutically acceptable, aqueous, or organic solvents, or mixtures thereof, and powders. The liquid or solid pharmaceutical compositions can contain suitable pharmaceutically acceptable excipients. In some embodiments, the pharmaceutical compositions are administered by the oral or nasal respiratory route for local or systemic effect. Pharmaceutical compositions in pharmaceutically acceptable solvents can be nebulized by use of inert gases. Nebulized solutions can be inhaled directly from the nebulizing device, or the nebulizing device can be attached to a face mask tent, or intermittent positive pressure breathing machine. Solution, suspension, or powder pharmaceutical compositions can be administered, e.g., orally, or nasally, from devices that deliver the formulation in an appropriate manner.

It is noted that in some embodiments, the chemical compounds in the pharmaceutical formulation may act as pro-drugs. Pro-drugs represent a modality to control drug bioavailability, control timing of drug release, and/or reduce negative side-effects. Similarly, formulation and delivery considerations can achieve these outcomes. Thus, optimization of all three variables together (prodrug moiety, formulation, delivery system) can be an effective strategy in drug development. Examples of ‘targeting systems’ designed to specifically reach cells within the brain, obtained by simultaneously leveraging pro-drug, nanoparticle. And nasal administration strategies are described, for example by Botti et al., 2021 Pharmaceutics 13:1114).

In further embodiments, in which the C₄-substituted tryptamine derivative compounds of present disclosure are used as a recreational drug, the compounds may be included in compositions such as a food or food product, a beverage, a food seasoning, a personal care product, such as a cosmetic, perfume or bath oil, or oils (both for topical administration as massage oil, or to be burned or aerosolized). The chemical compounds of the present disclosure may also be included in a “vape” product, which may also include other drugs, such as nicotine, and flavorings.

Thus, it will be clear that the C₄-substituted tryptamine derivative compounds may be used as a pharmaceutical or recreational drug. Accordingly, in another aspect the present disclosure provides, in at least one embodiment, a use of a chemical compound having a formula (I):

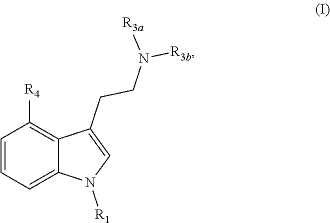

wherein R₄ is a substituent selected from:
(A) an ether moiety or derivative thereof;
(B) a carbonic ester moiety or derivative thereof;
(C) a carboxylic acid moiety or derivative thereof;
(D) a polyether moiety or derivative thereof;
(E) a carbonothioate moiety or derivative thereof; and
(F) a phosphate moiety or derivative thereof;
   wherein R₁ is a hydrogen atom or a moiety containing a carbonic ester or derivative thereof, or a ketone or ketone derivative; and
   wherein R₃ₐ and R_{3b} are each independently a hydrogen atom or an alkyl group, as a pharmaceutical or recreational drug.

The pharmaceutical formulations comprising the chemical compounds of the present disclosure may be used to treat a subject, and to treat a psychiatric disorder in a subject. Accordingly, the present disclosure includes in a further embodiment, a method for treating a psychiatric disorder, the method comprising administering to a subject in need thereof a pharmaceutical formulation comprising a chemical compound having a formula (I):

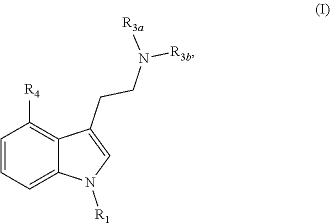

wherein R₄ is a substituent selected from:
(A) an ether moiety or derivative thereof;
(B) a carbonic ester moiety or derivative thereof;
(C) a carboxylic acid moiety or derivative thereof;
(D) a polyether moiety or derivative thereof;
(E) a carbonothioate moiety or derivative thereof; and
(F) a phosphate moiety or derivative thereof,
   wherein R₁ is a hydrogen atom or a moiety containing a carbonic ester or derivative thereof, or a ketone or ketone derivative; and
   wherein R₃ₐ and R_{3b} are each independently a hydrogen atom or an alkyl group.

Psychiatric disorders that may be treated include, for example, neurodevelopmental disorders such as intellectual disability, global development delay, communication disorders, autism spectrum disorder, and attention-deficit hyperactivity disorder (ADHD); bipolar and related disorders, such as mania, and depressive episodes; anxiety disorder, such as generalized anxiety disorder (GAD), agoraphobia, social anxiety disorder, specific phobias (natural events, medical, animal, situational, for example), panic disorder, and separation anxiety disorder; stress disorders, such as acute stress disorder, adjustment disorders, post-traumatic stress disorder (PTSD), and reactive attachment disorder; dissociative disorders, such as dissociative amnesia, dissociative identity disorder, and depersonalization/derealization disorder; somatoform disorders, such as somatic symptom disorders, illness anxiety disorder, conversion disorder, and factitious disorder; eating disorders, such as anorexia nervosa, bulimia nervosa, rumination disorder, pica, and binge-eating disorder; sleep disorders, such as narcolepsy, insomnia disorder, hypersomnolence, breathing-related sleep disorders, parasomnias, and restless legs syndrome; disruptive disorders, such as kleptomania, pyromania, intermittent explosive disorder, conduct disorder, and oppositional defiant disorder; depressive disorders, such as disruptive mood dysregulation disorder, major depressive disorder, persistent depressive disorder (dysthymia), premenstrual dysphoric disorder, substance/medication-induced depressive disorder, postpartum depression, and depressive disorder caused by another medical condition, for example, psychiatric and existential distress within life-threatening cancer situations (ACS Pharmacol. Transl. Sci. 4: 553-562; J Psychiatr. Res. 137: 273-282); substance-related disorders, such as alcohol-related disorders, cannabis related disorders, inhalant-use related disorders, stimulant use disorders, and tobacco use disorders; neurocognitive disorders, such as delirium; schizophrenia; compulsive disorders, such as obsessive compulsive disorders (OCD), body dysmorphic disorder, hoarding disorder, trichotillomania disorder, excoriation disorder, substance/medication induced obsessive-compulsive disorder, and obsessive-compulsive disorder related to another medical condition; and personality disorders, such as antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder.

In an aspect, the compounds of the present disclosure may be used to be contacted with a receptor to thereby modulate the receptor. Such contacting includes bringing a compound of the present disclosure and receptor together under in vitro conditions, for example, by introducing the compounds in a sample containing a receptor, for example, a sample containing purified receptors, or a sample containing cells comprising receptors. In vitro conditions further include the conditions described in Example 1 hereof. Contacting further includes bringing a compound of the present disclosure and receptor together under in vivo conditions. Such in vivo conditions include the administration to an animal or human subject, for example, of a pharmaceutically effective amount of the compound of the present disclosure, when the compound is formulated together with a pharmaceutically active carrier, diluent, or excipient, as hereinbefore described, to thereby treat the subject. Upon having contacted the receptor, the compound may activate the receptor or inhibit the receptor.

In an aspect, receptors with which the compounds of the present disclosure may be contacted include, for example, the 5-HT_{1A} receptor, the 5-HT_{2A} receptor, the 5-HT_{1B} receptor, the 5-HT_{2B} receptor, the 5-HT_{3A} receptor, the ADRA1A receptor, the ADRA2A receptor, the CHRM1 receptor, the CHRM2 receptor, the CNR1 receptor, the DRD1 receptor, the DRD2S receptor, or the OPRD1 receptor.

Thus, in a further aspect, the condition that may be treated in accordance herewith can be any receptor mediated disorder, including, for example, a 5-HT_{1A} receptor-mediated disorder, a 5-HT_{2A} receptor-mediated disorder, a 5-HT_{1B} receptor-mediated disorder, a 5-HT_{2B} receptor-mediated disorder, a 5-HT_{3A} receptor-mediated disorder, a ADRA1A receptor-mediated disorder, a ADRA2A receptor-mediated disorder, a CHRM1 receptor-mediated disorder, a CHRM2 receptor-mediated disorder, a CNR1 receptor-mediated disorder, a DRD1 receptor-mediated disorder, a DRD2S receptor-mediated disorder, or a OPRD1 receptor-mediated disorder. Such disorders include, but are not limited to schizophrenia, psychotic disorder, attention deficit hyperactivity disorder, autism, and bipolar disorder.

In some embodiments, upon having contacted a receptor and a receptor, the compound may modulate the receptor. However, at the same time other receptors may not be modulated. E.g., a compound may activate or inhibit a first receptor, e.g., a 5-HT_{1A} receptor, however the compound may at the same time not modulate a second receptor, e.g., a 5-HT_{2A} receptor, or upon having contacted a first 5-HT_{2A} receptor and a second 5-HT_{1A} receptor, the compound may modulate the first 5-HT_{2A} receptor, e.g., activate or inhibit the 5-HT_{2A} receptor, however the compound may at the same time not modulate the second 5-HT_{1A} receptor.

In one embodiment, in an aspect, upon administration the compounds of the present disclosure can interact with an enzyme or transmembrane transport protein in the subject to thereby modulate the enzyme or transmembrane transport protein and exert a pharmacological effect. Such contacting includes bringing a compound of the present disclosure and enzyme or transmembrane transport protein together under in vitro conditions, for example, by introducing the compounds in a sample containing an enzyme or transmembrane transport protein, for example, a sample containing a purified enzyme or transmembrane transport protein, or a sample containing cells comprising an enzyme or transmembrane transport protein. Contacting further includes bringing a compound of the present disclosure and an enzyme or transmembrane transport protein together under in vivo conditions. Such in vivo conditions include the administration to an animal or human subject, for example, of a pharmaceutically effective amount of the compound of the present disclosure, when the compound is formulated together with a pharmaceutically active carrier, diluent, or excipient, as hereinbefore described, to thereby treat the subject.

In one embodiment, in an aspect, the enzyme can be monoamine oxidase A (MOA-A),

In one embodiment, in an aspect, the transmembrane transport protein can be a dopamine active transporter (DAT), a norephedrine transporter (NET), or a serotonin transporter (SERT) transmembrane transport protein.

It is noted that in one embodiment, in an aspect, upon administration the compound having formula (I) may be in vivo hydrolyzed to form a compound having chemical formula (VIₐ) or (VI_{b}):

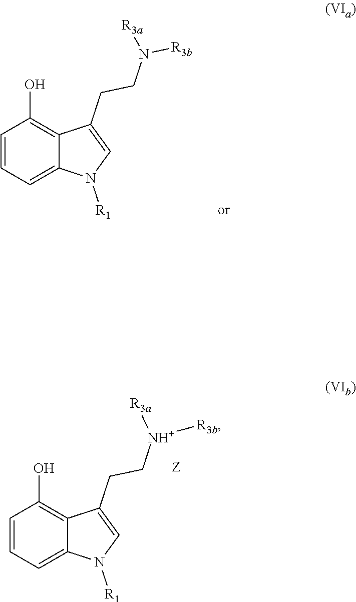

wherein R₃ₐ and R_{3b} are each independently a hydrogen atom, an alkyl group, or an aryl group,
wherein R₁ is a hydrogen atom or a moiety containing a carbonic ester or derivative thereof, or a ketone or ketone derivative
wherein Z is a counterbalancing anion,
and wherein the compound having chemical formula (VIₐ) or (VI_{b}) interacts with a receptor to thereby modulate the receptor in the subject and exert a pharmacological effect. In this respect, the compounds of the present disclosure may be formulated as a pro-drug pharmaceutical formulation, i.e., a formulation wherein it is not the formulated compound itself that mediates a pharmacological effect, but rather a compound that is obtained following in vivo hydrolyzation of the formulated compound by the subject. Hydrolyzation may occur, for example, in the gastro-intestinal tract of a person upon oral delivery of a pro-drug pharmaceutical formulation.

Turning now to methods of making the C₄-substituted tryptamine derivative compounds of the present disclosure, it is initially noted, by way of general comment that the C₄-substituted tryptamine derivative compounds of the present disclosure may be prepared in any suitable manner, including by any organic chemical synthesis methods, biosynthetic methods, or a combination thereof.

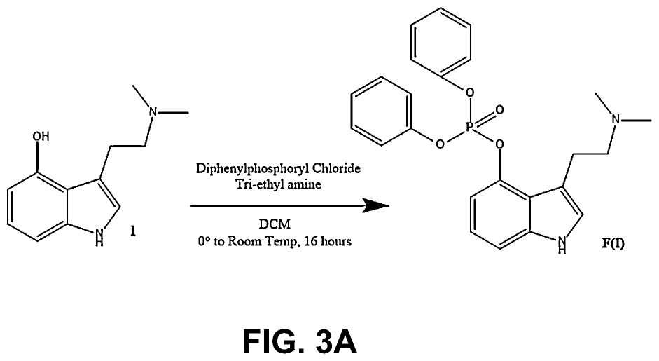

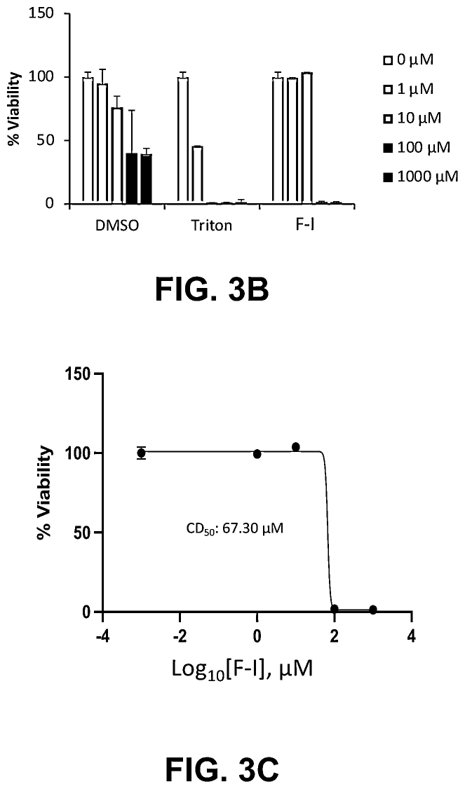

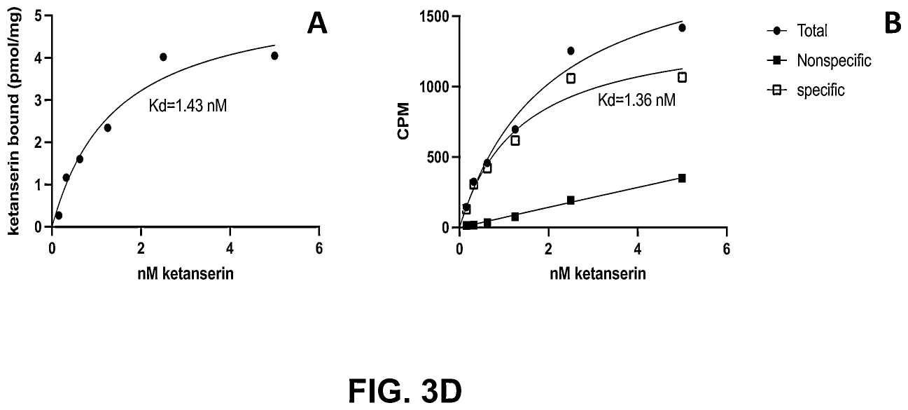

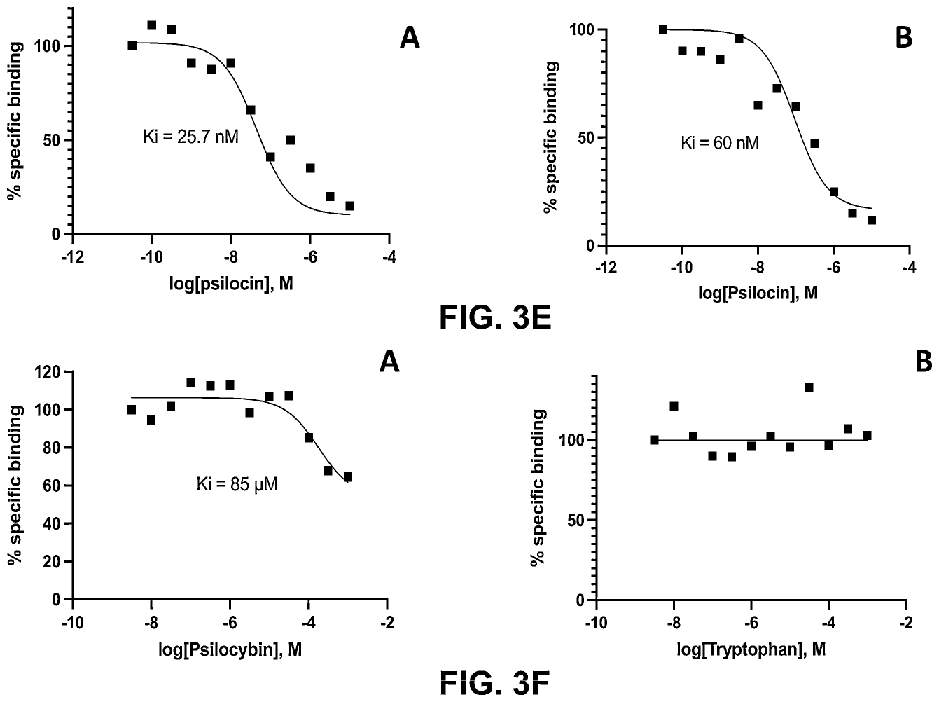

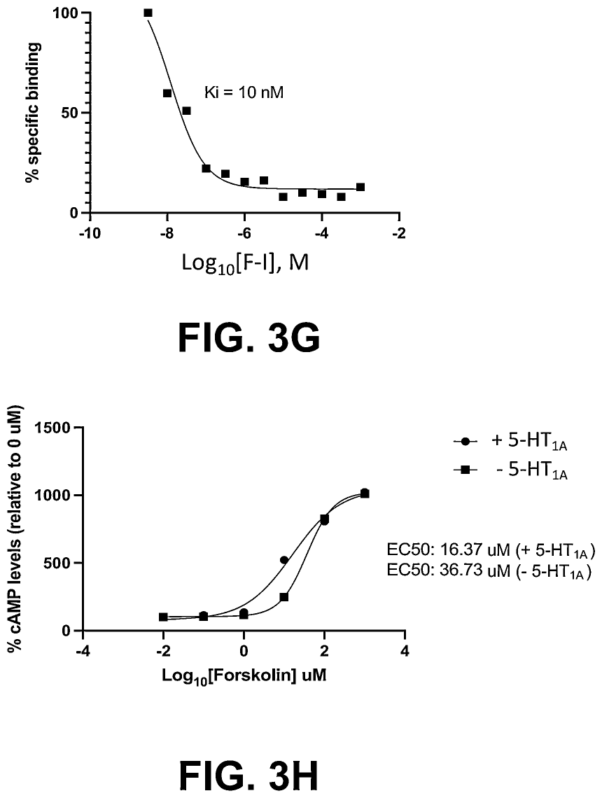

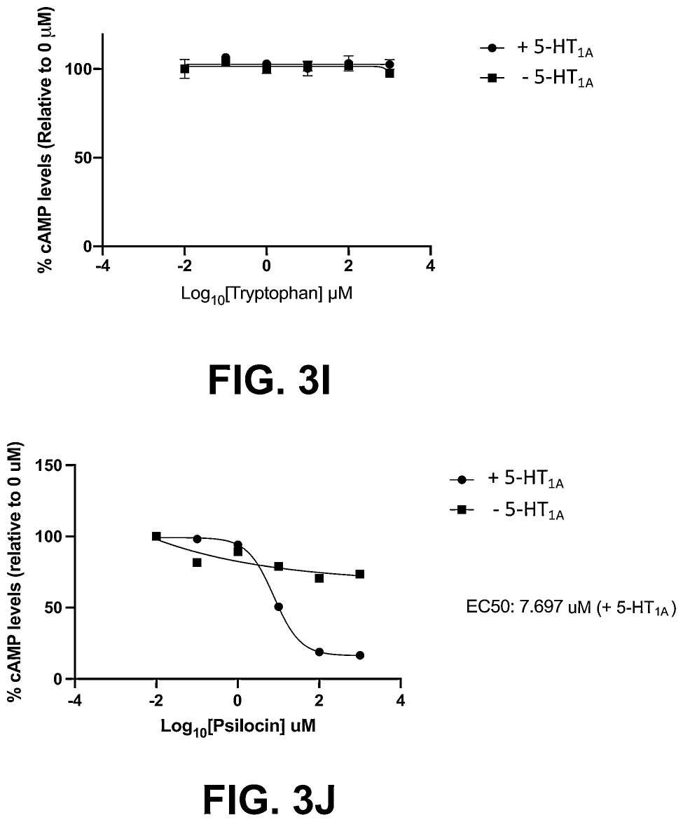

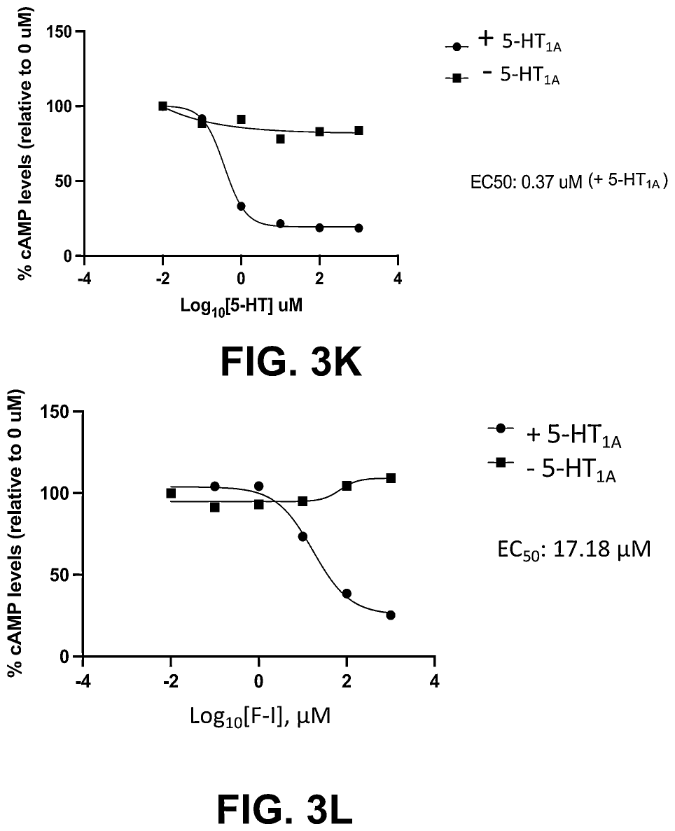

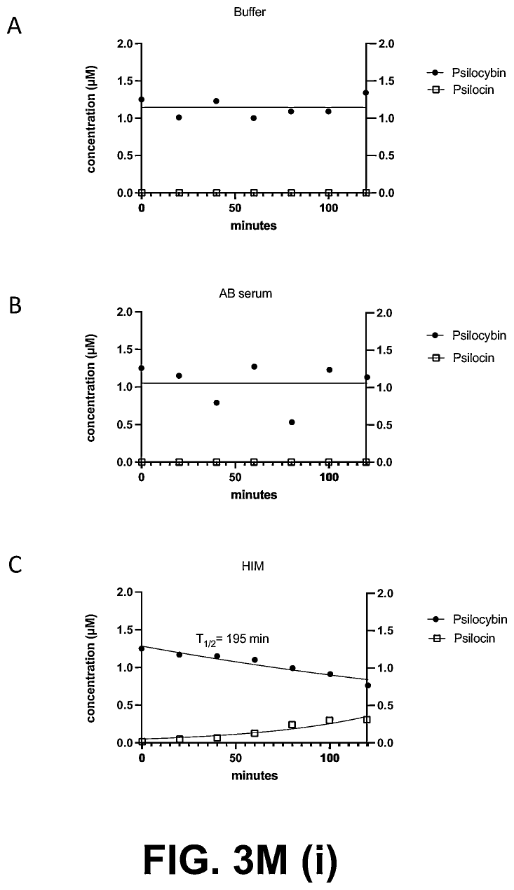

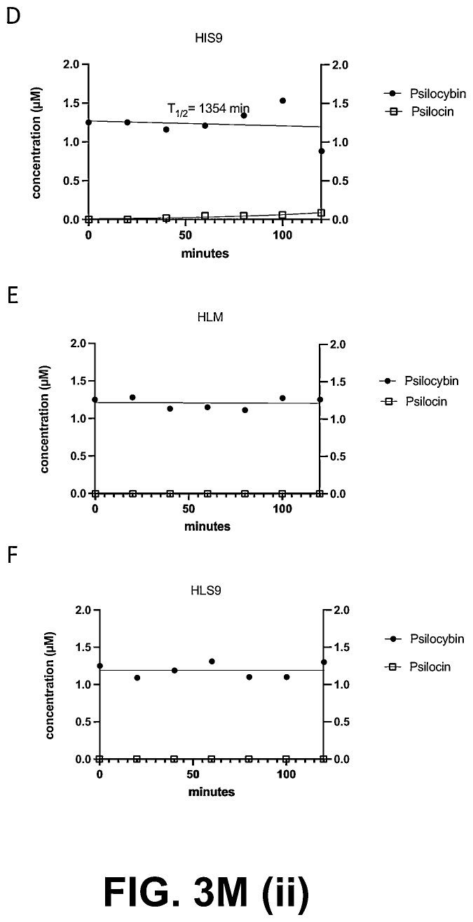

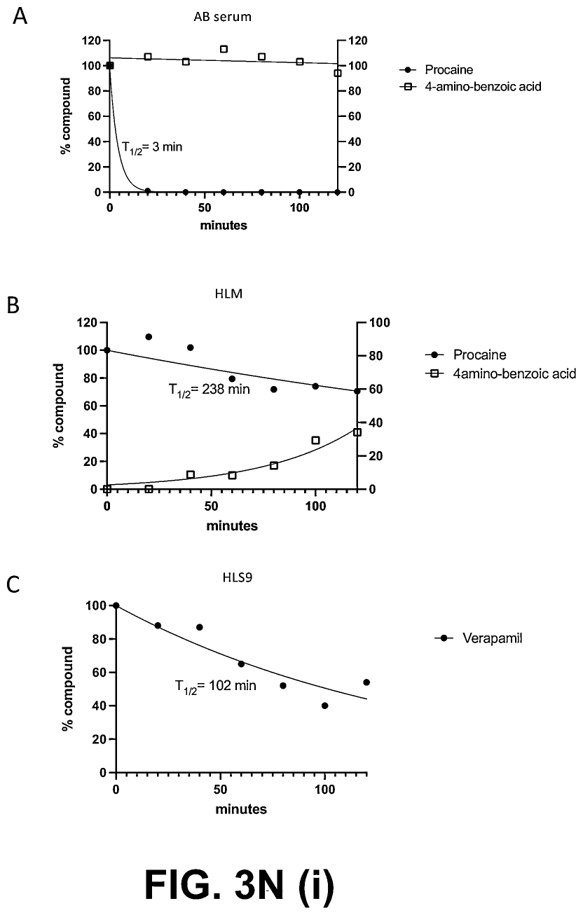

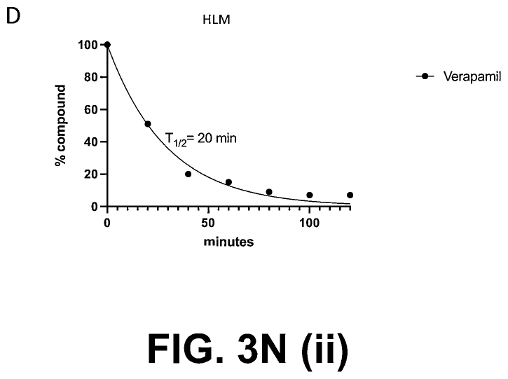

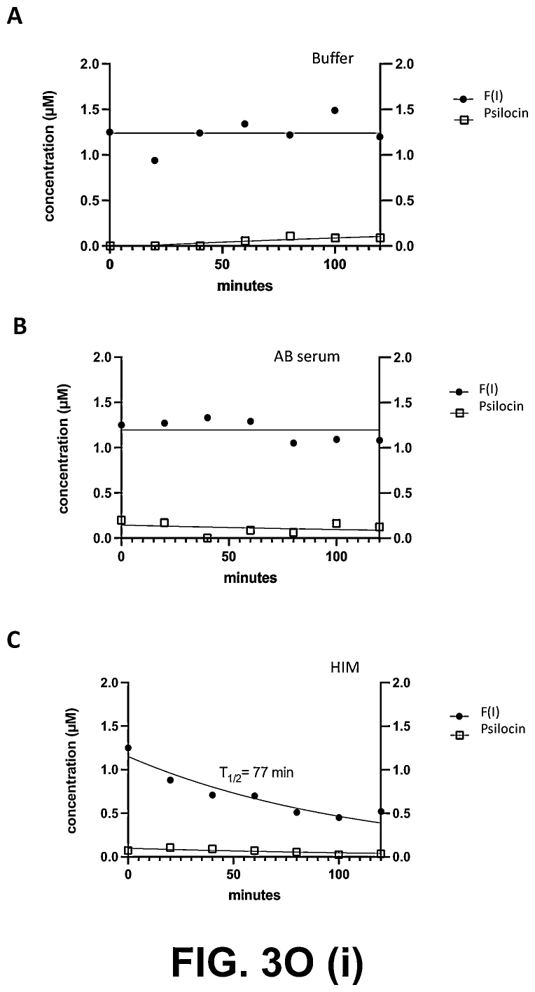

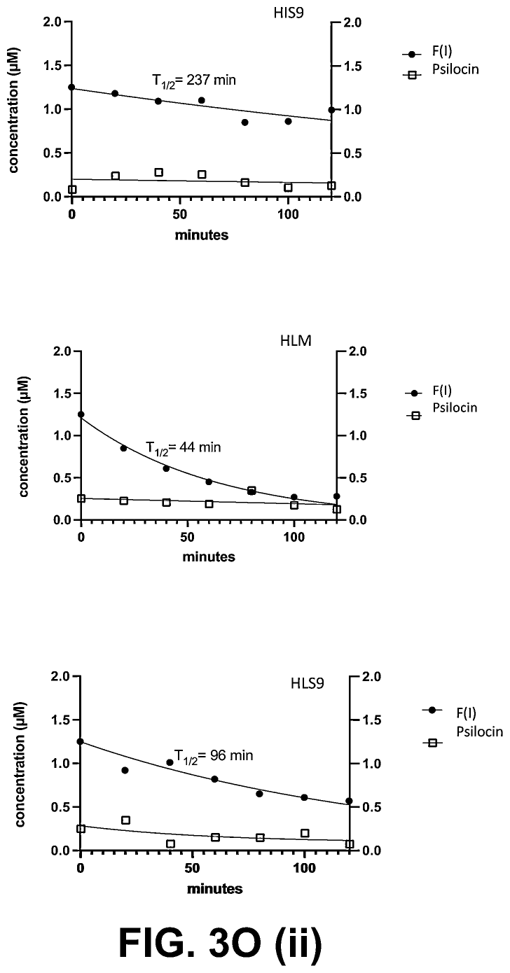

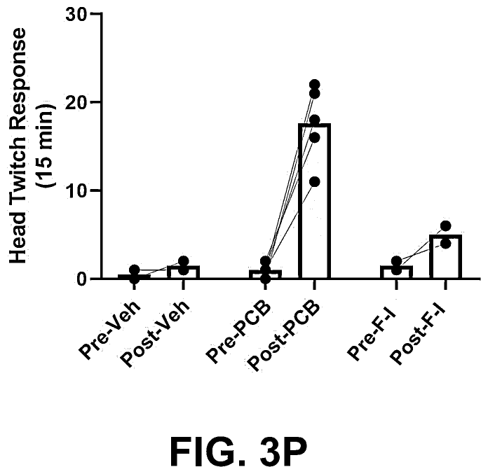

Examples of suitable chemical reactions that may be performed in accordance herewith are depicted in FIGS. 3A, 4A, 5A, 6A, 7A, 8A, 9A (i), 9A (ii), 10A, 11A, 12A, 13A, 14A and 15A and are further additionally detailed hereinafter in the Example section.

In general, as is known to those of skill in the art, in order to perform chemical synthetic reactions selected reactants are reacted under reaction conditions which permit the reactants to chemically react with each other and form a product, i.e., the C₄-carboxylic acid-substituted tryptamine derivative compounds or C₄-carbonothioate-substituted tryptamine derivative compounds of the present disclosure. Such reactions conditions may be selected, adjusted, and optimized as known by those of skill in the art. The reactions may be conducted in any suitable reaction vessel (e.g., a tube, bottle). Suitable solvents that may be used are polar solvents such as, for example, dichloromethane, dichloroethane, toluene, and so-called participating solvents such as acetonitrile and diethyl ether. Suitable temperatures may range from, for example, e.g., from about −78° C. to about 60° C. Furthermore, catalysts, also known as promoters, may be included in the reaction such as iodonium dicollidine perchlorate (IDCP), any silver or mercury salts, trimethylsilyl trifluoromethanesulfonate (TMS-triflate, TMSOTf), or trifluoronmethanesulfonic acid (triflic acid, TfOH), N-iodosuccinimide, methyl triflate. Furthermore, reaction times may be varied. As will readily be appreciated by those of skill in the art, the reaction conditions may be optimized, for example, by preparing several reactant preparations and reacting these in separate reaction vessels under different reaction conditions, for example, different temperatures, using different solvents etc., evaluating the obtained C₄-substituted tryptamine derivative product compounds, adjusting reaction conditions, and selecting a desired reaction condition.

In accordance with the foregoing, in an aspect, disclosed herein are methods of making a chemical compound having a formula (I):

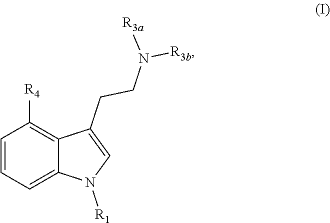

wherein R₄ is a substituent selected from:
(G) an ether moiety or derivative thereof;
(H) a carbonic ester moiety or derivative thereof;
(I) a carboxylic acid moiety or derivative thereof;
(J) a polyether moiety or derivative thereof;
(K) a carbonothioate moiety or derivative thereof; and
(L) a phosphate moiety or derivative thereof,
   wherein R₁ is a hydrogen atom or a moiety containing a carbonic ester or derivative thereof, or a ketone or ketone derivative; and
   wherein R₃ₐ and R_{3b} are each independently a hydrogen atom or an alkyl group, the method comprising the performance of at least one chemical synthesis reaction selected from the reactions depicted in FIGS. 3A, 4A, 5A, 6A, 7A, 8A, 9A (i), 9A (ii), 10A, 11A, 12A, 13A, 14A, and 15A.

In one embodiment, R₄ can be a substituent selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof.

Referring to FIG. 3A, in one embodiment, the chemical compound can have the formula F(I):

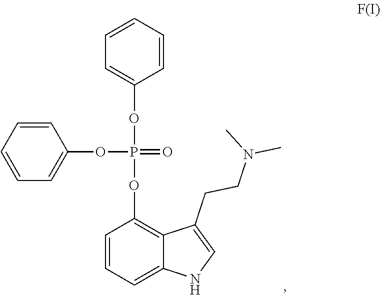 [SMILES: CN(C)CCc1c[nH]c2cccc(OP(=O)(Oc3ccccc3)Oc3ccccc3)c12]

and the at least one chemical synthesis reaction is the reaction depicted in FIG. 3A.

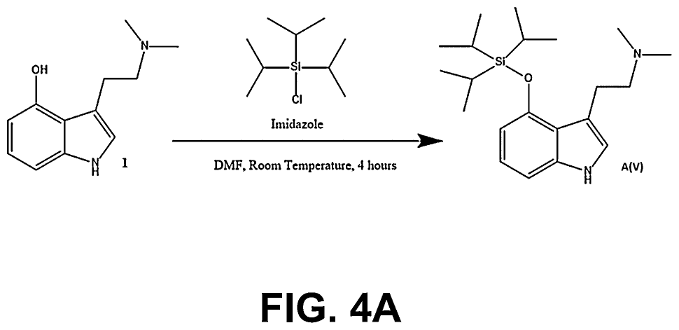

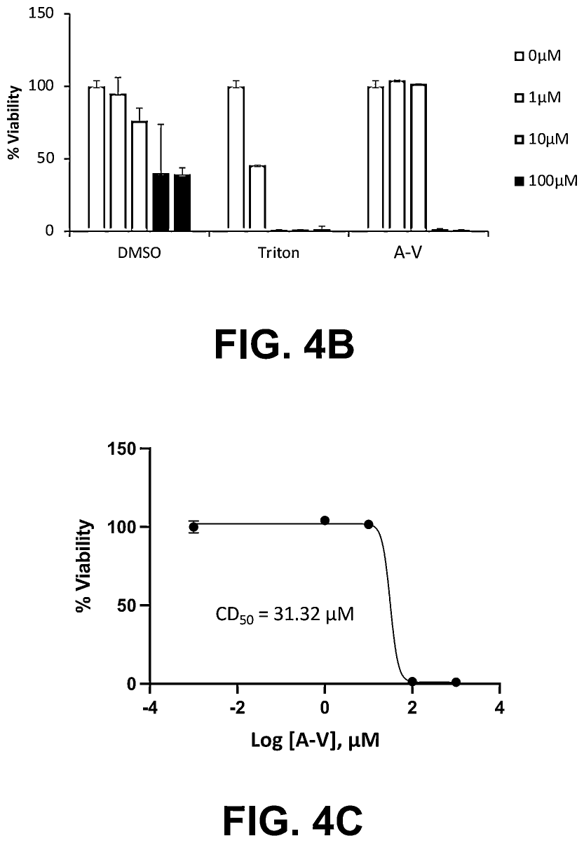

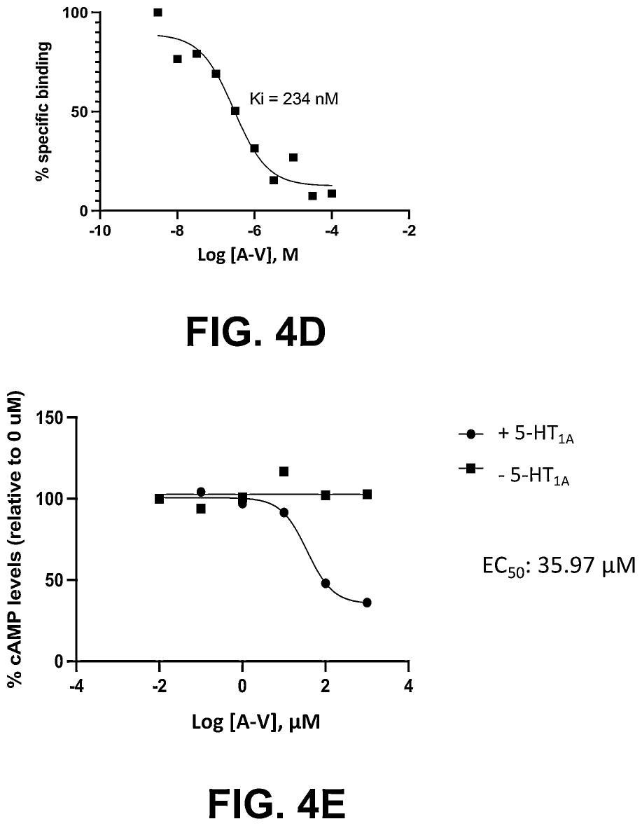

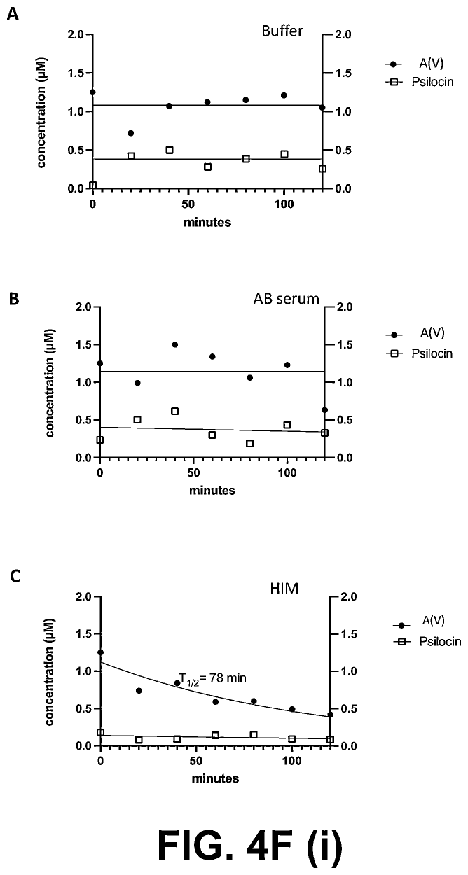

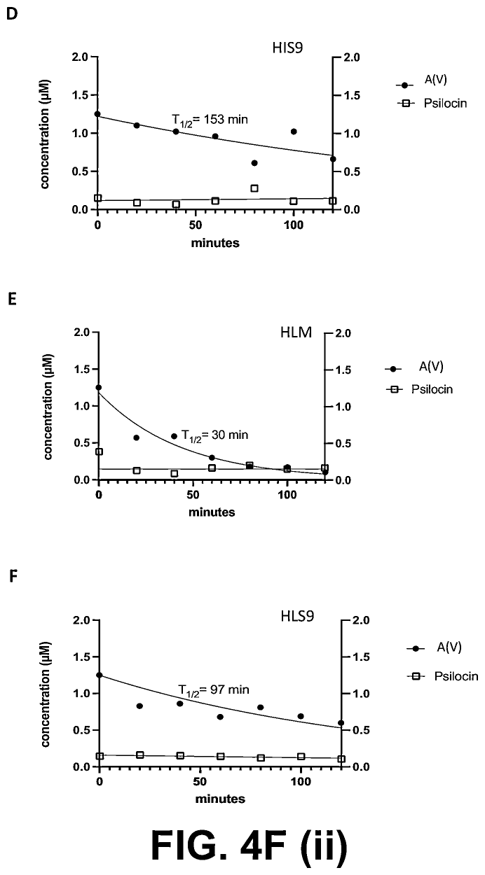

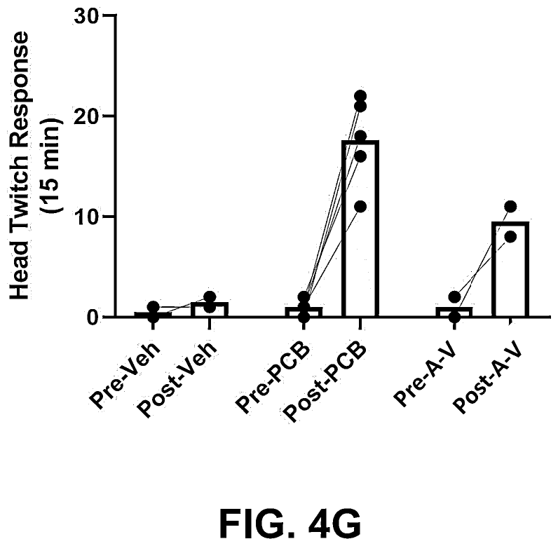

Referring to FIG. 4A, in one embodiment, the chemical compound can have the formula A(V):

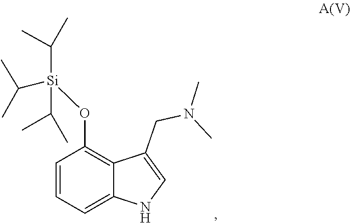 [SMILES: CC(C)[Si](Oc1cccc2[nH]cc(CN(C)C)c12)(C(C)C)C(C)C]

and the at least one chemical synthesis reaction is the reaction depicted in FIG. 4A.

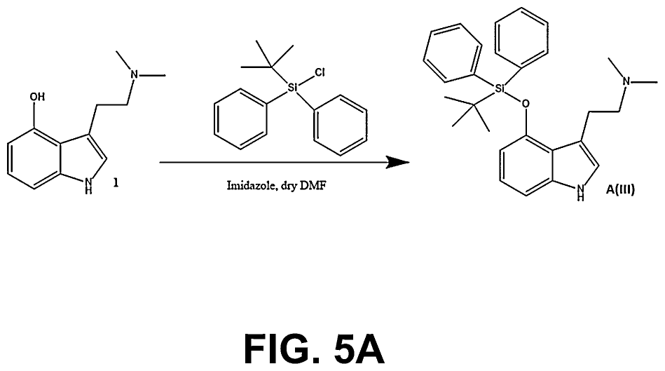

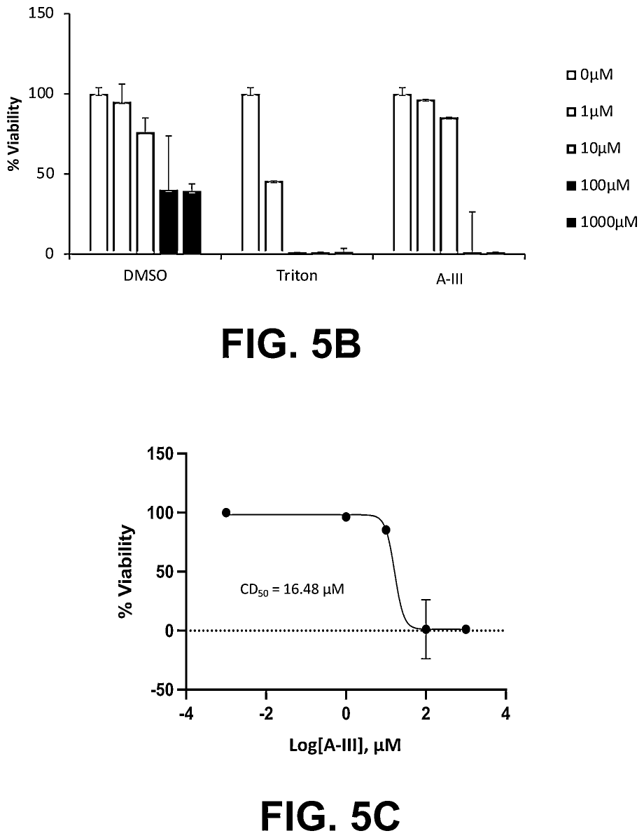

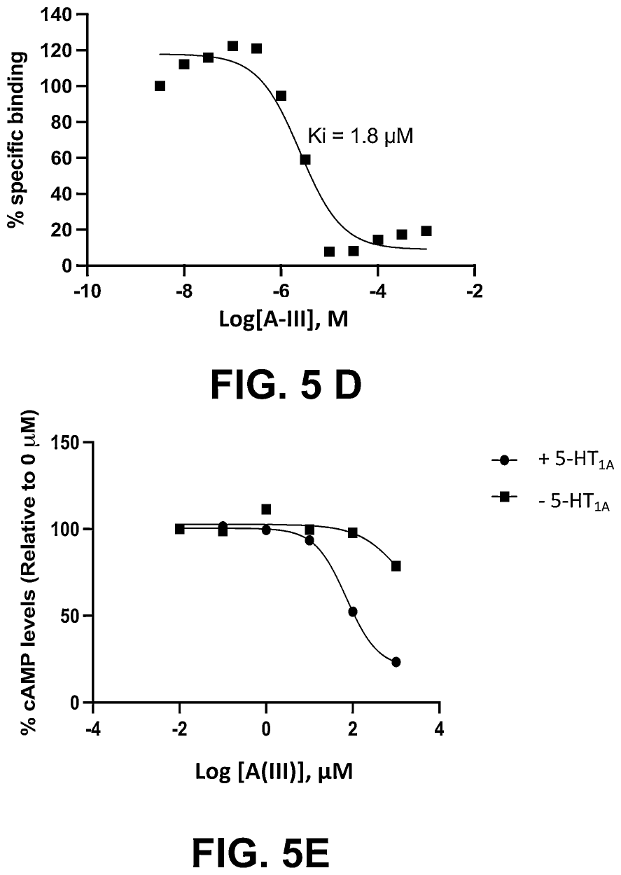

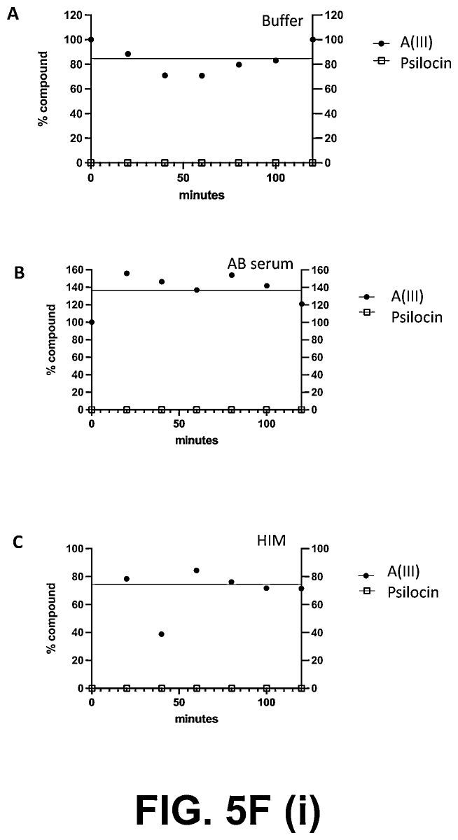

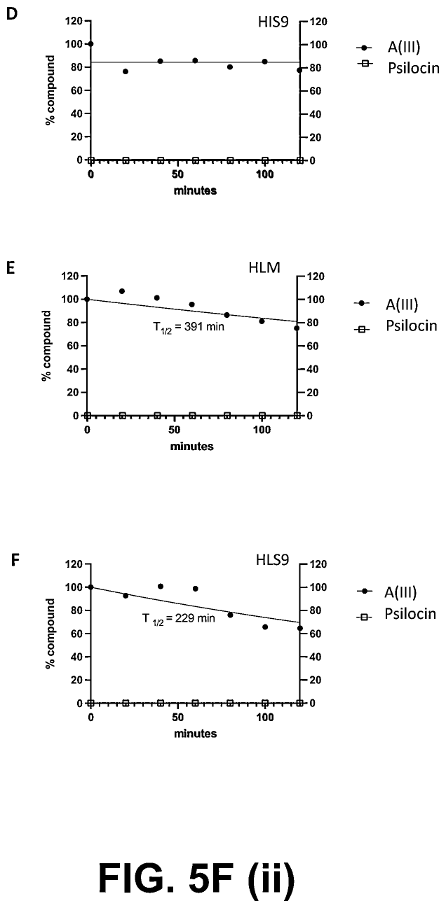

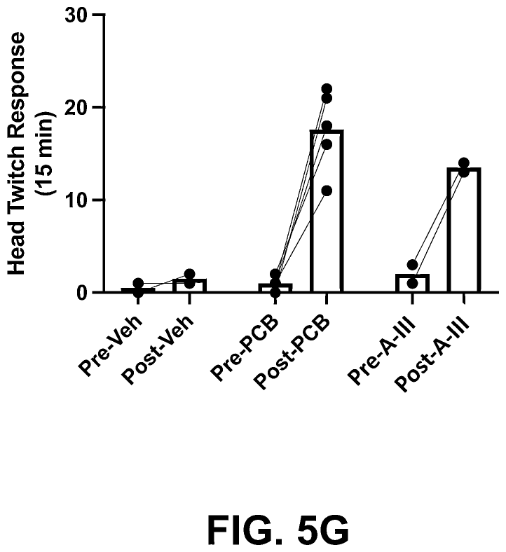

Referring to FIG. 5A, in one embodiment, the chemical compound can have the formula A(III):

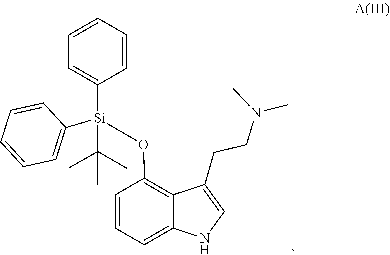 [SMILES: CN(C)CCc1c[nH]c2cccc(O[Si](c3ccccc3)(c3ccccc3)C(C)(C)C)c12]

and the at least one chemical synthesis reaction is the reaction depicted in FIG. 5A.

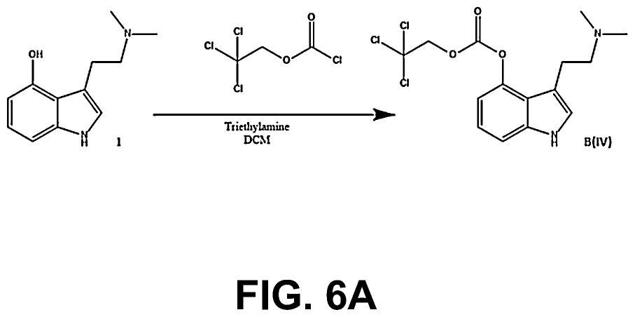

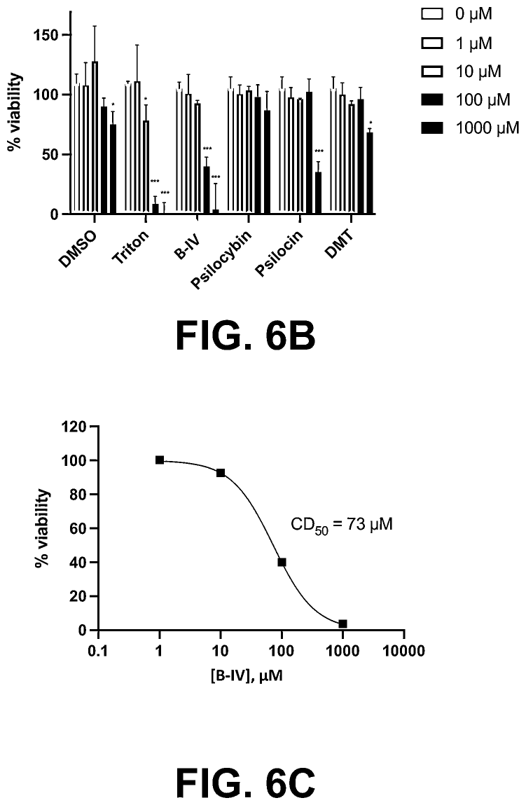

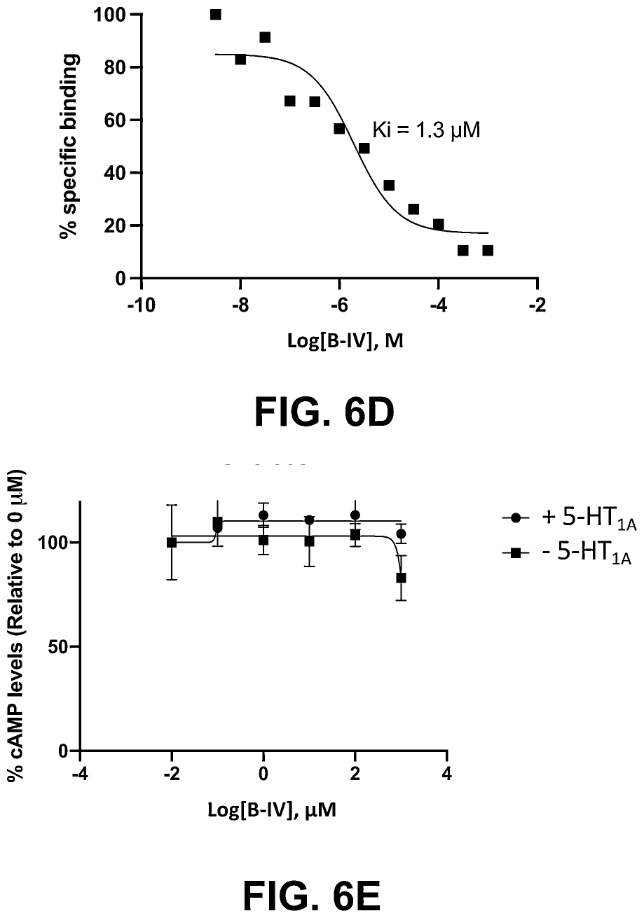

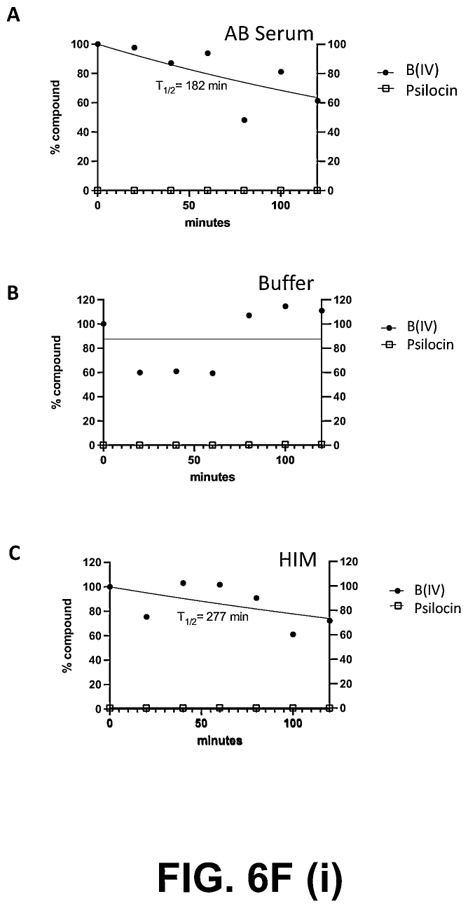

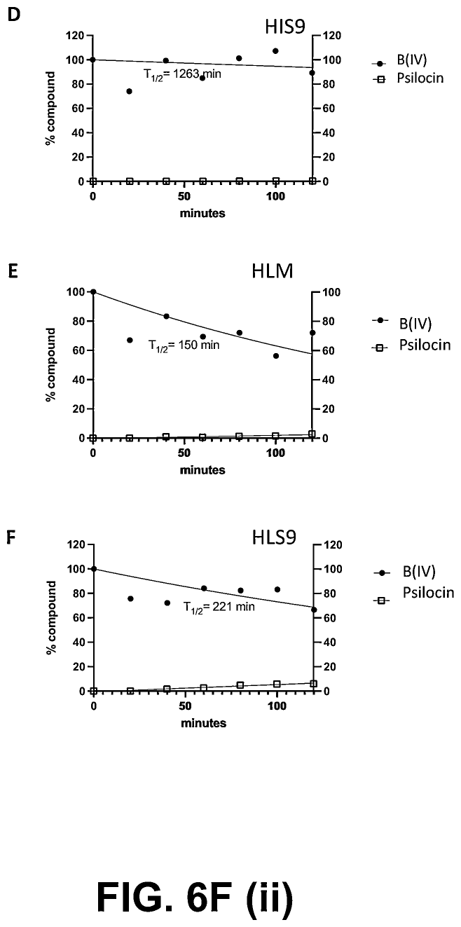

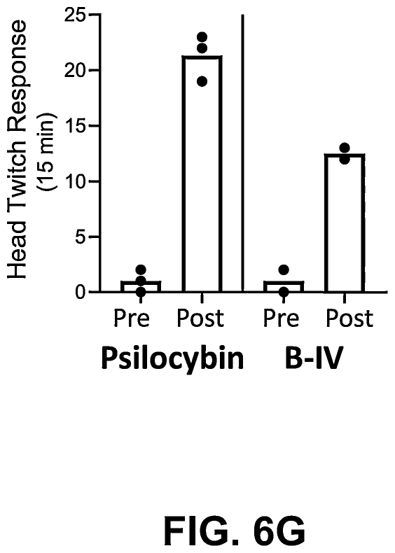

Referring to FIG. 6A, in one embodiment, the chemical compound can have the formula B(IV):

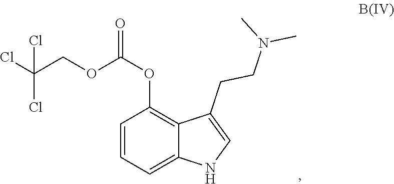 [SMILES: CN(C)CCc1c[nH]c2cccc(OC(=O)OCC(Cl)(Cl)Cl)c12]

and the at least one chemical synthesis reaction is the reaction depicted in FIG. 6A.

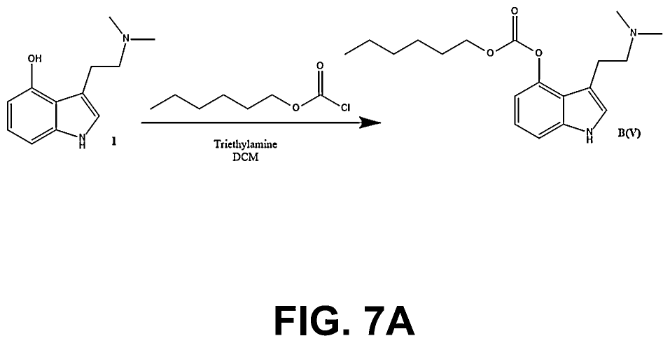

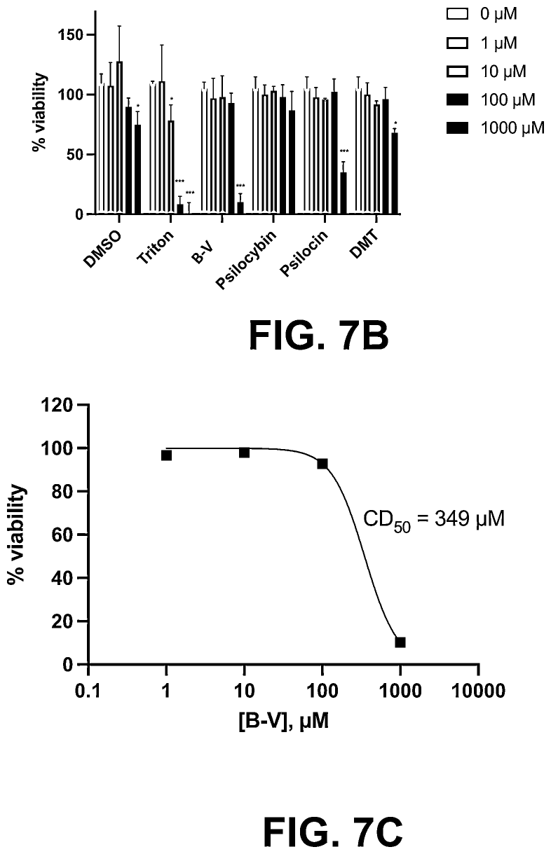

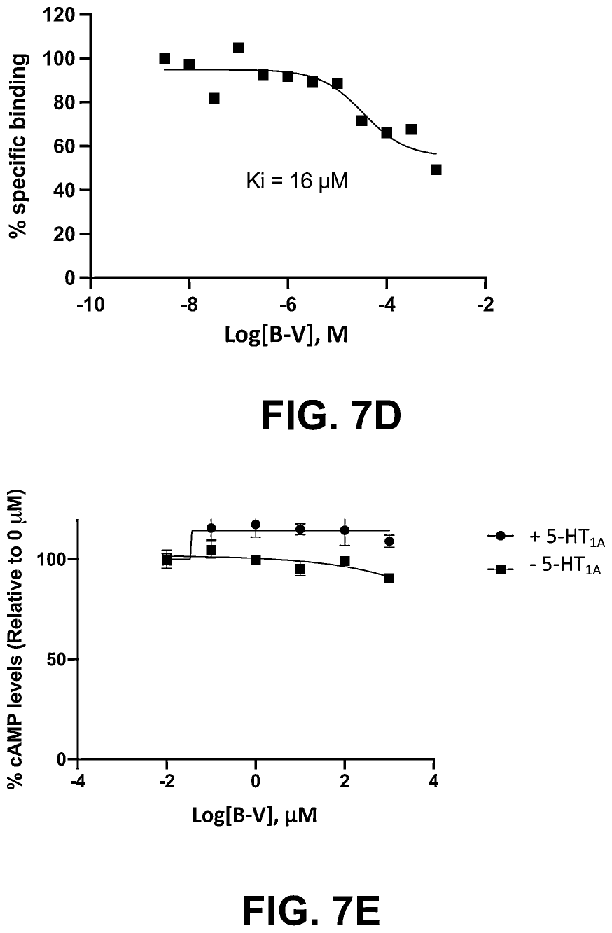

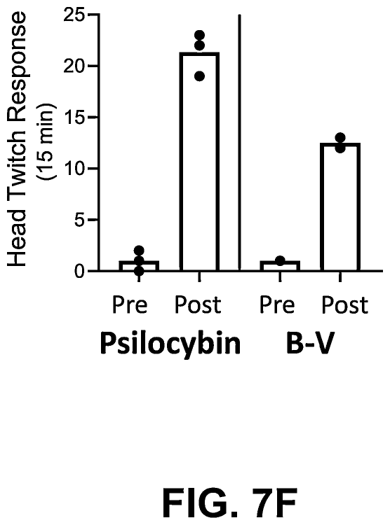

Referring to FIG. 7A, in one embodiment, the chemical compound can have the formula B(V):

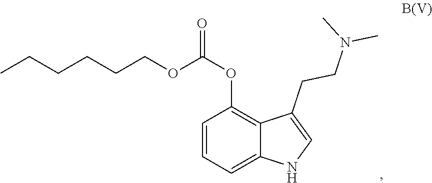 [SMILES: CCCCCCOC(=O)Oc1cccc2[nH]cc(CCN(C)C)c12]

and the at least one chemical synthesis reaction is the reaction depicted in FIG. 7A.

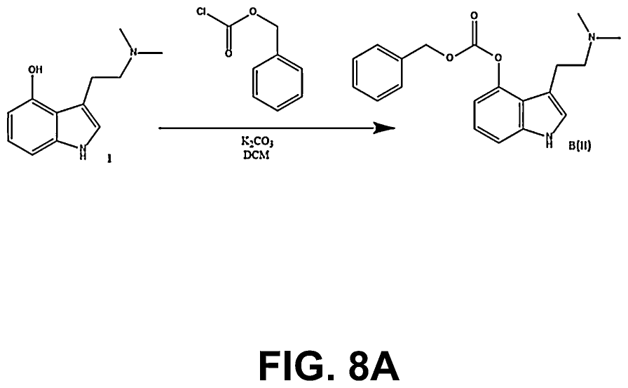

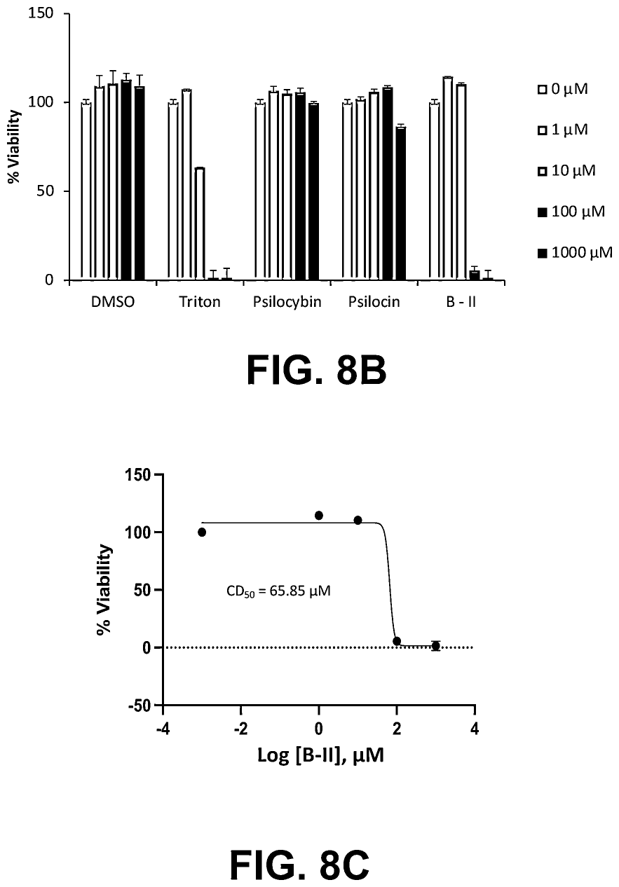

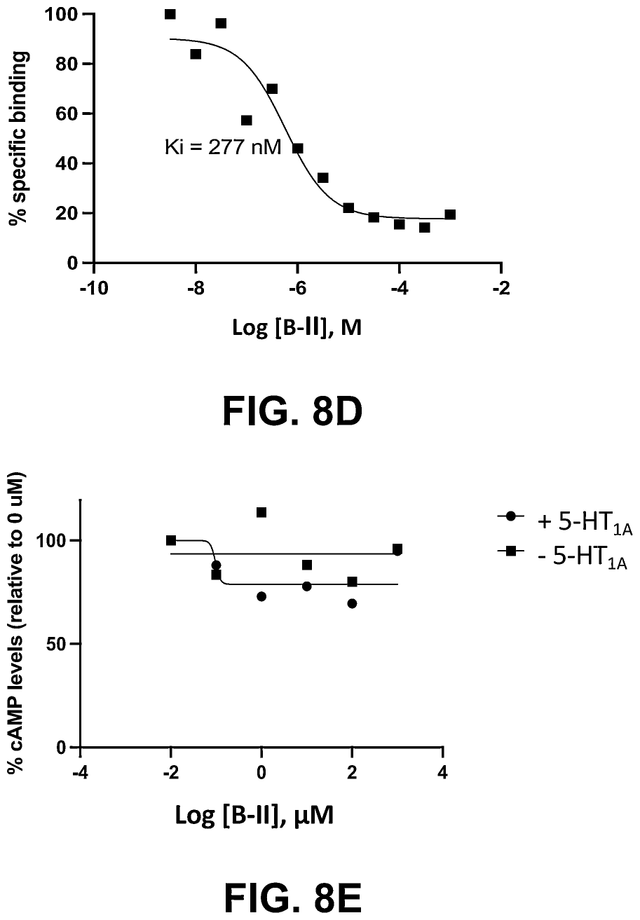

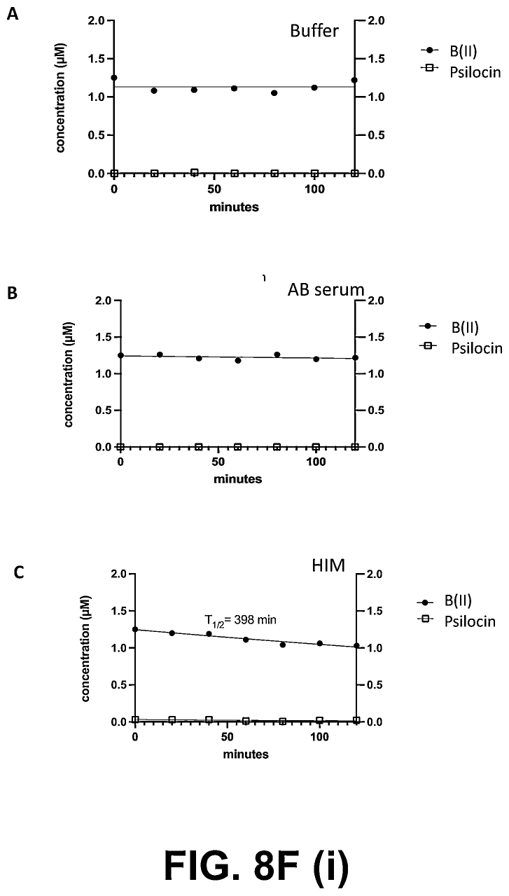

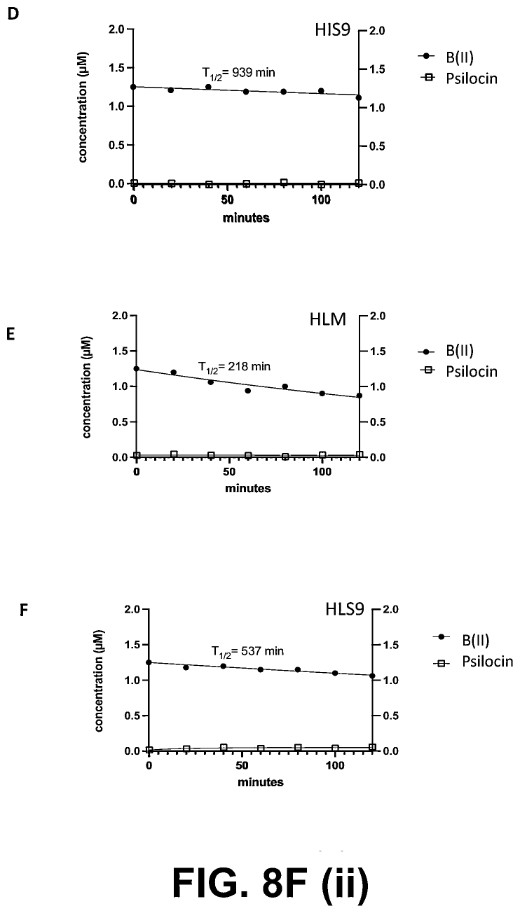

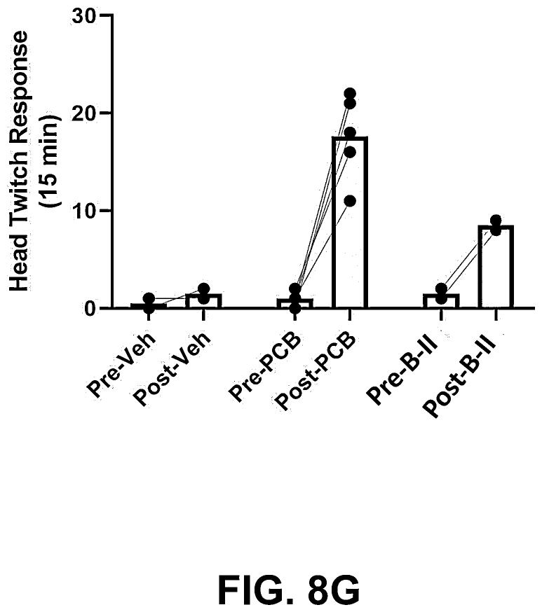

Referring to FIG. 8A, in one embodiment, the chemical compound can have the formula B(II):

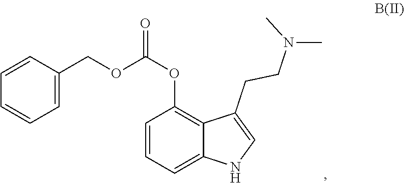 [SMILES: CN(C)CCc1c[nH]c2cccc(OC(=O)OCc3ccccc3)c12]

and the at least one chemical synthesis reaction is the reaction depicted in FIG. 8A.

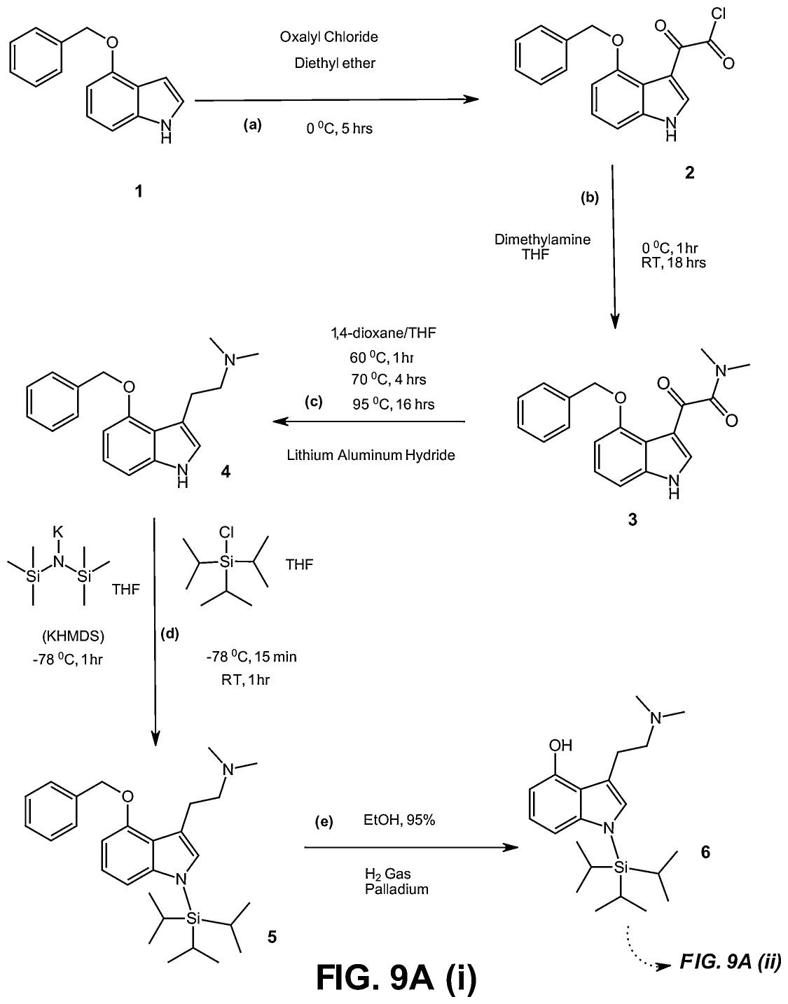

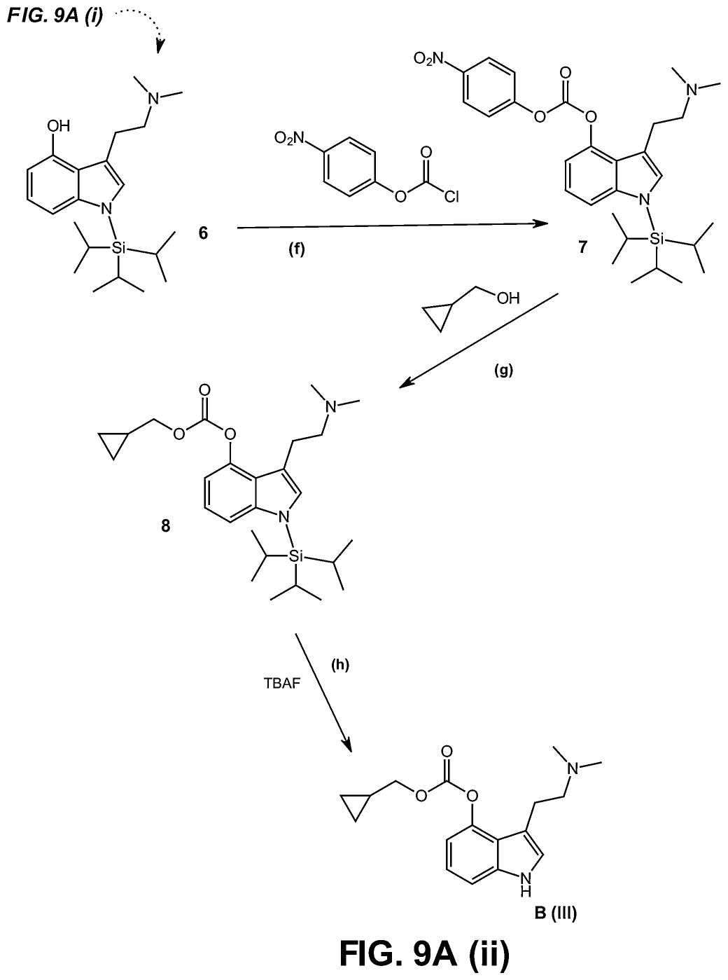

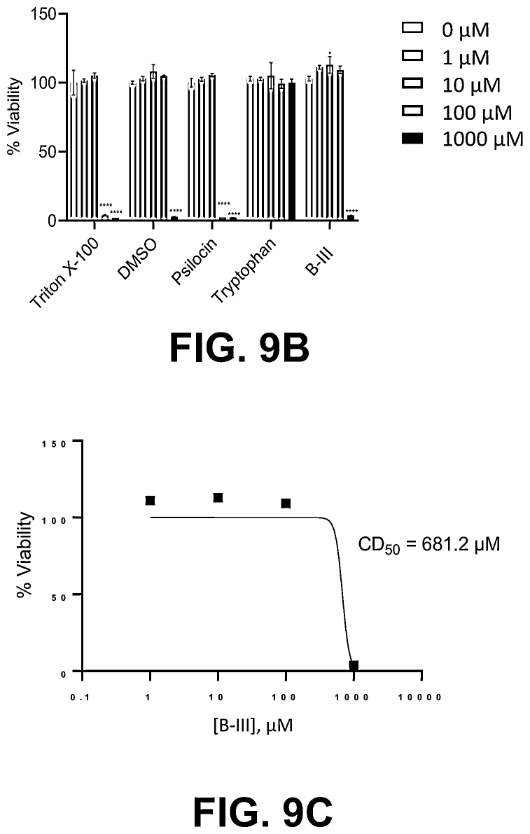

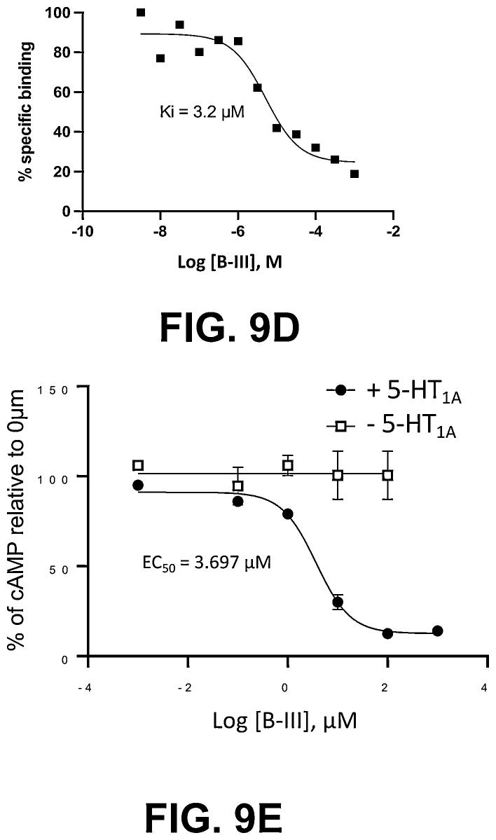

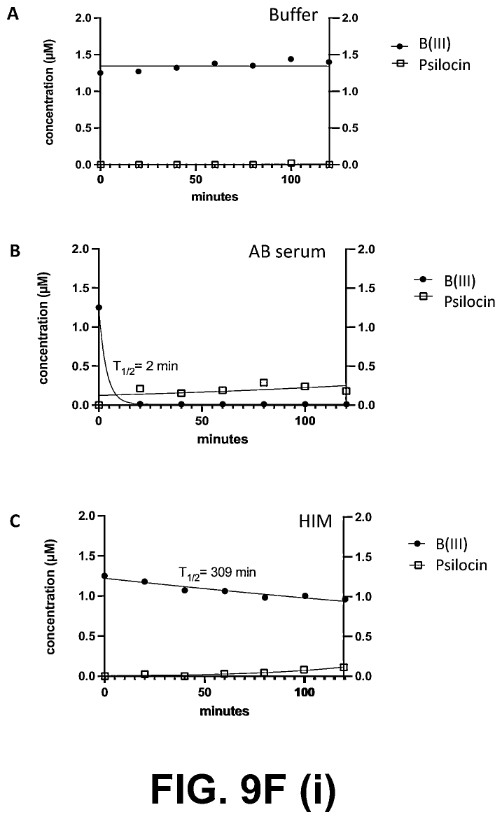

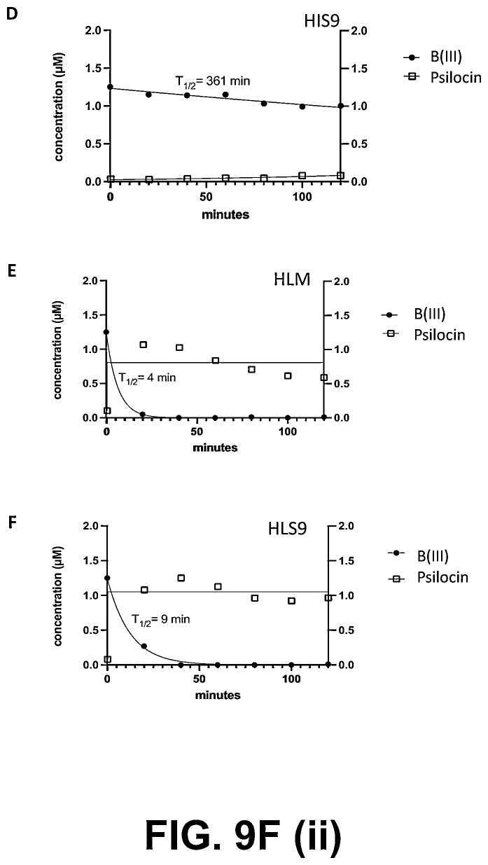

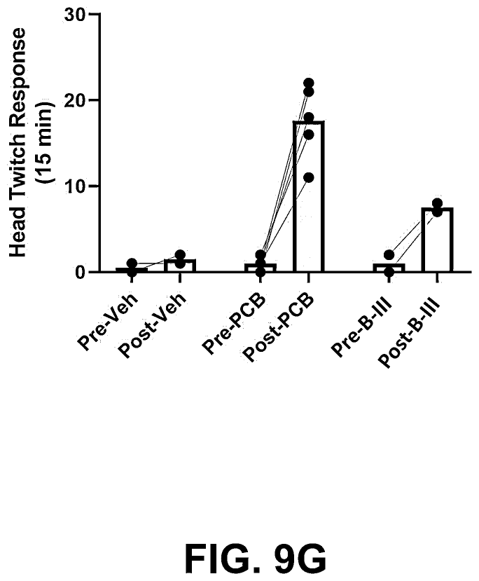

Referring to FIGS. 9A (i) and 9A (ii), in one embodiment, the chemical compound can have the formula B(III):

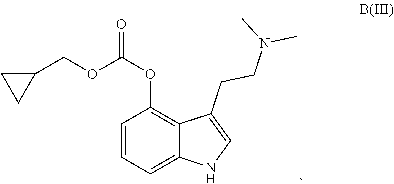 [SMILES: CN(C)CCc1c[nH]c2cccc(OC(=O)OCC3CC3)c12]

and the at least one chemical synthesis reaction is the chemical reaction (h); (g) and (h); (f), (g), and (h); (e), (f), (g), and (h); (d), (e), (f), (g), and (h); (c), (d), (e), (f), (g), and (h); (b), (c), (d), (e), (f), (g), and (h); and (a), (b), (c), (d), (e), (f), (g), and (h) depicted in FIGS. 9A (i) and 9A (ii).

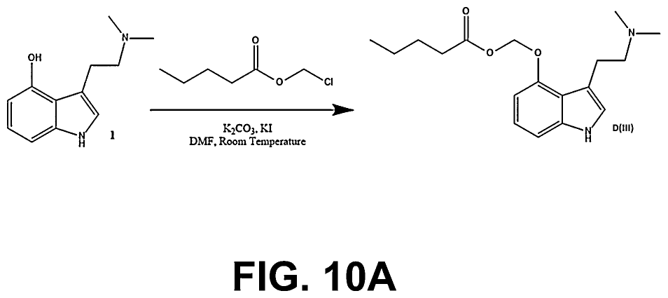

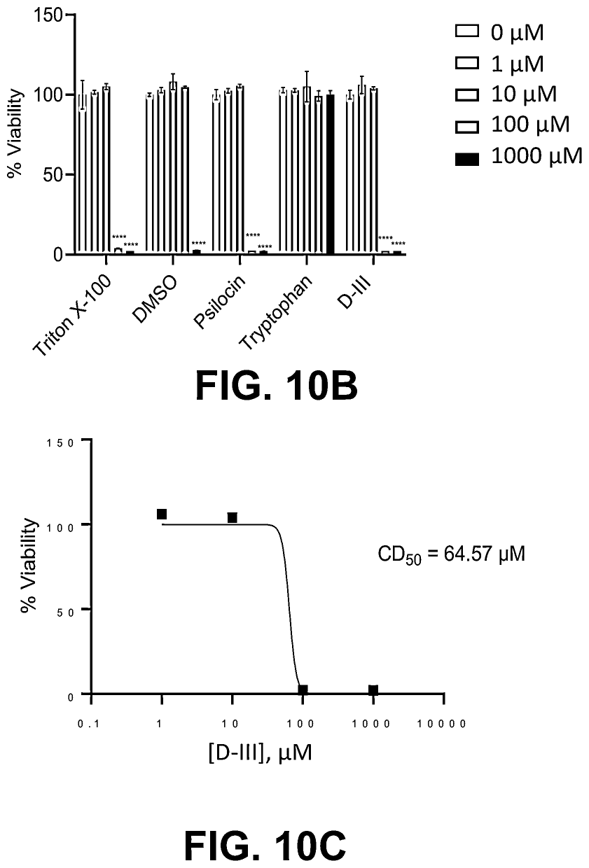

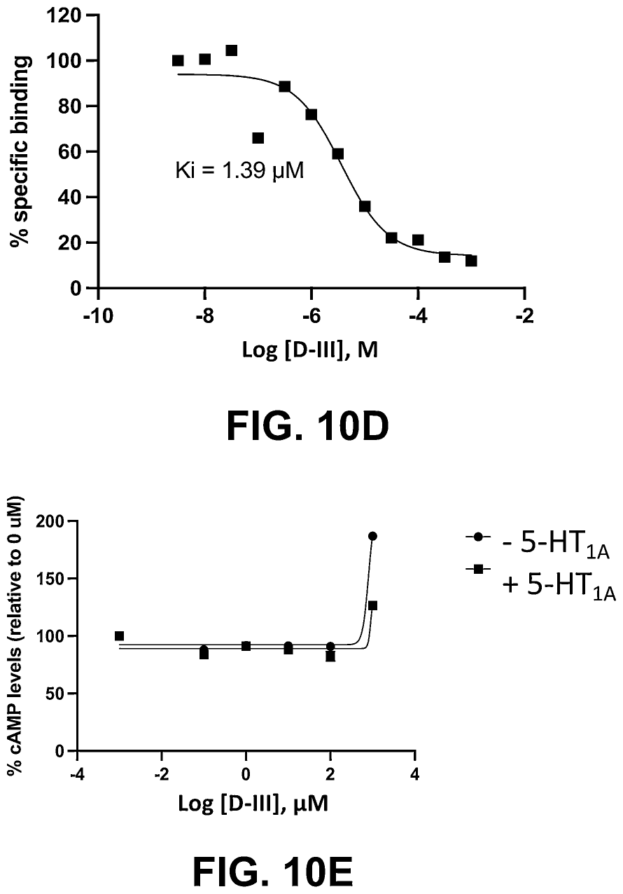

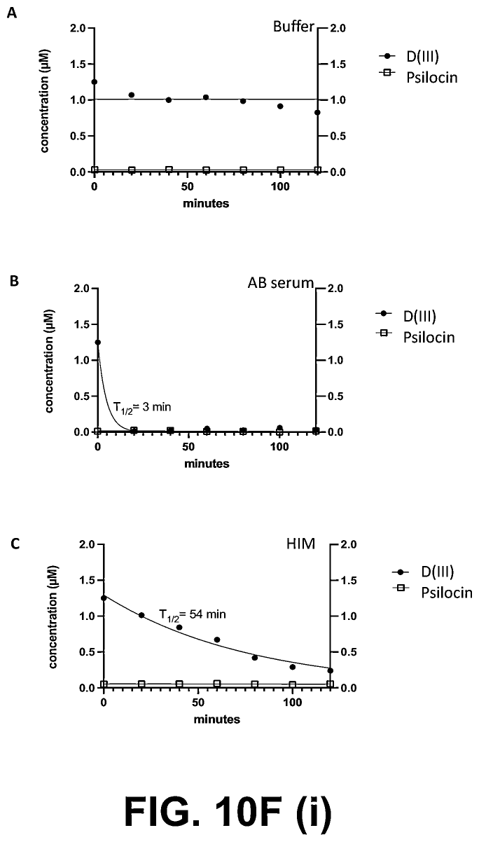

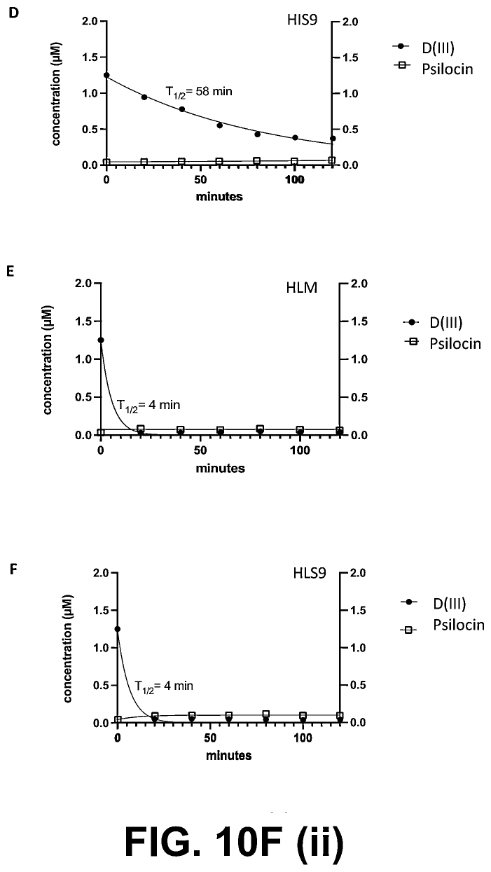

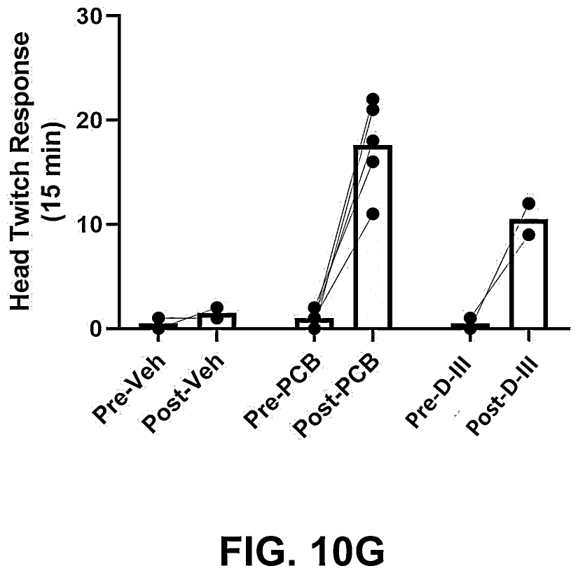

Referring to FIG. 10A, in one embodiment, the chemical compound can have the formula D(III):

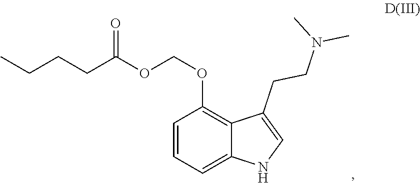 [SMILES: CCCCC(=O)OCOc1cccc2[nH]cc(CCN(C)C)c12]

and the at least one chemical synthesis reaction is the reaction depicted in FIG. 10A.

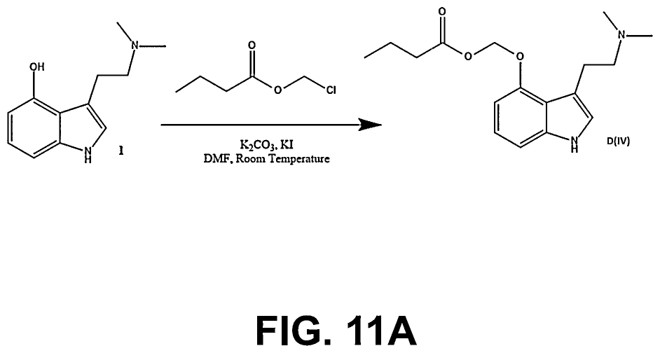

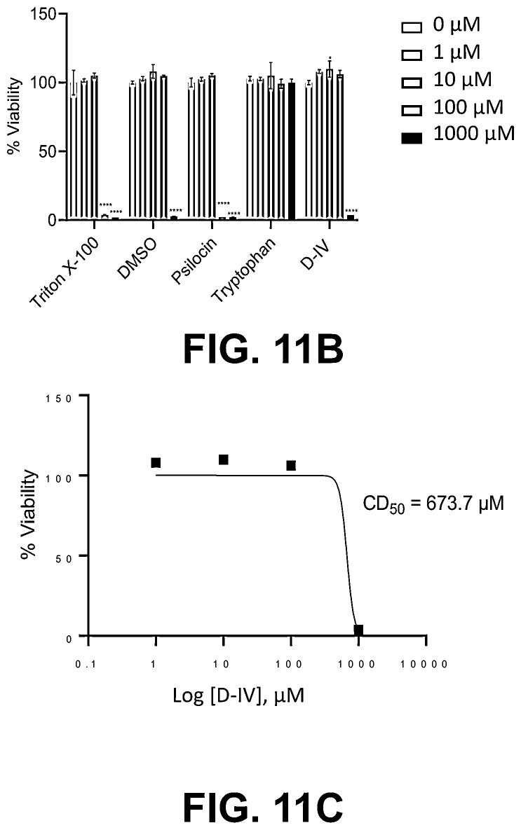

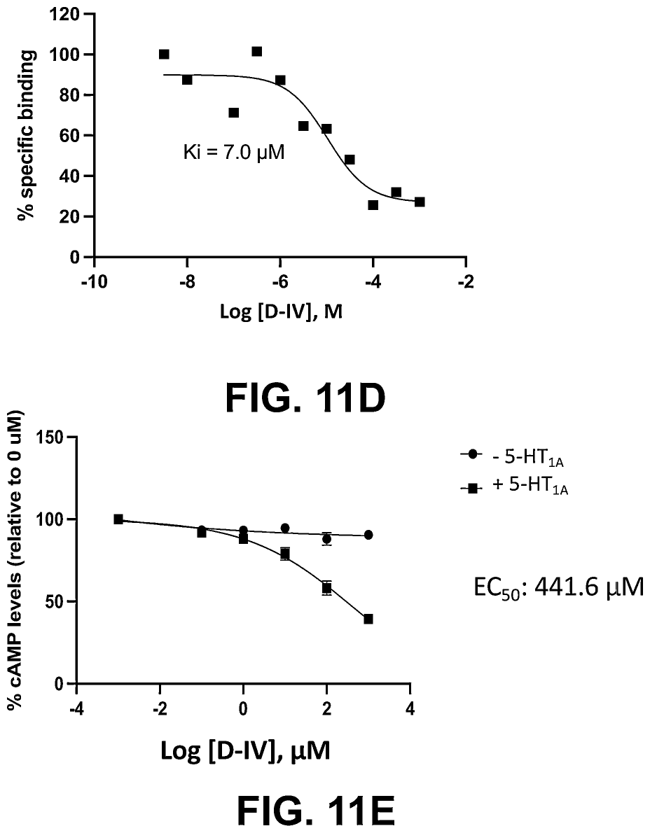

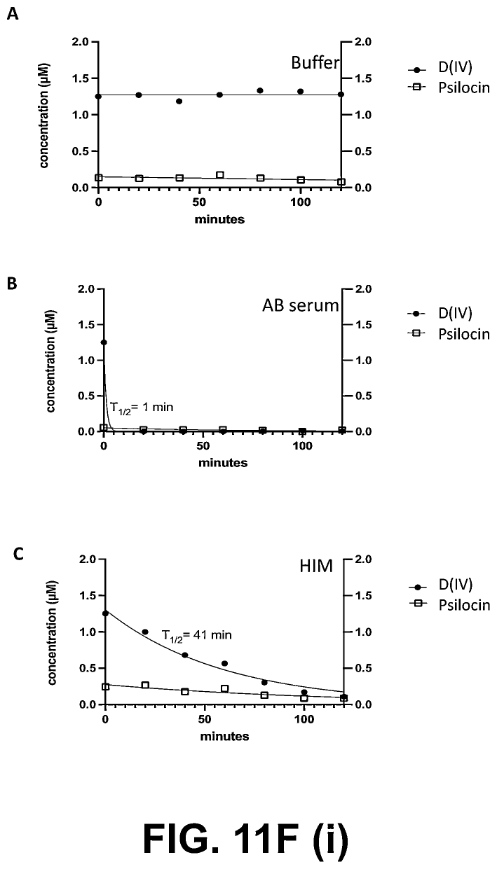

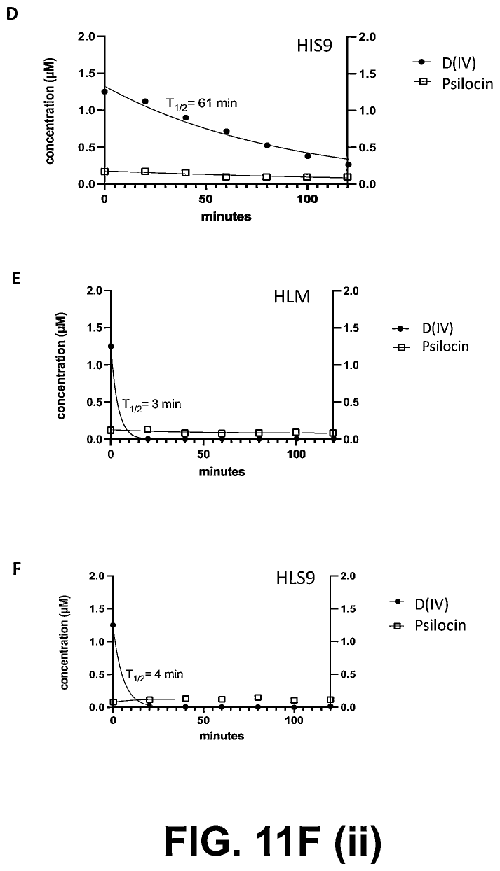

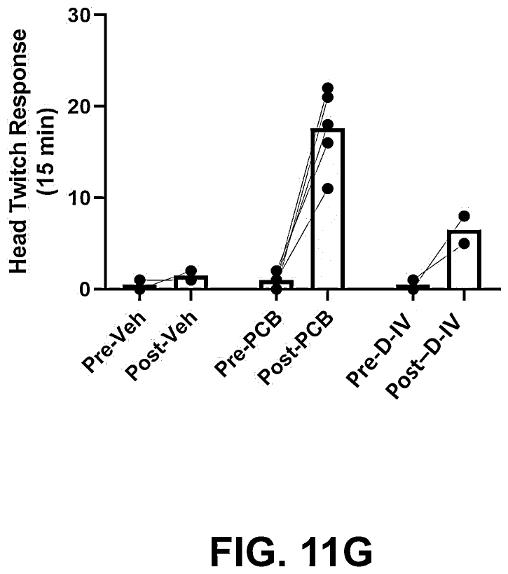

Referring to FIG. 11A, in one embodiment, the chemical compound can have the formula D(IV):

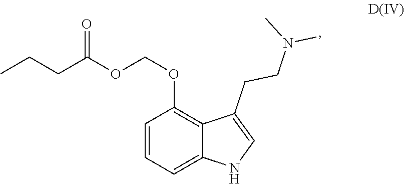

and the at least one chemical synthesis reaction is the reaction depicted in FIG. 11A.

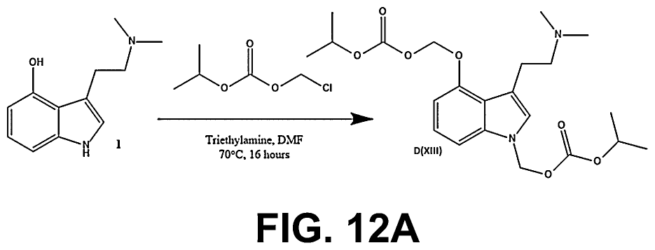

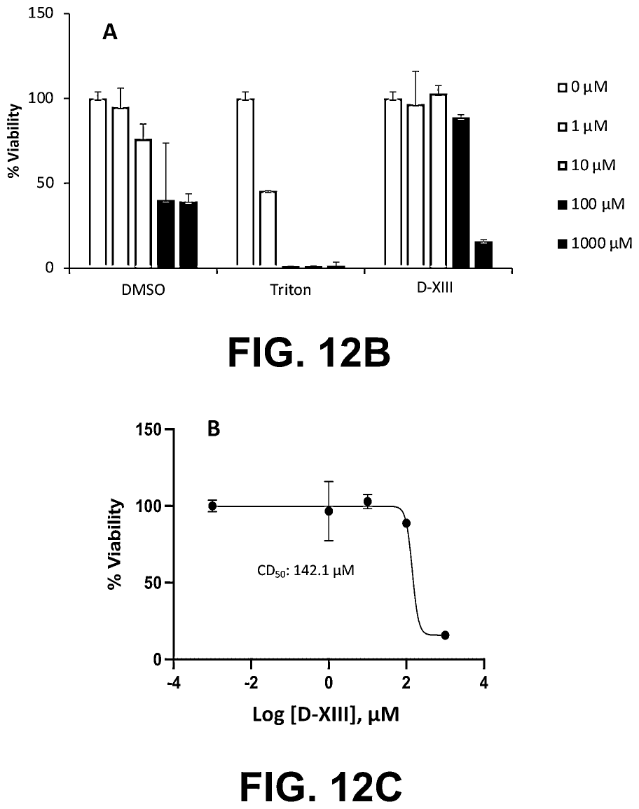

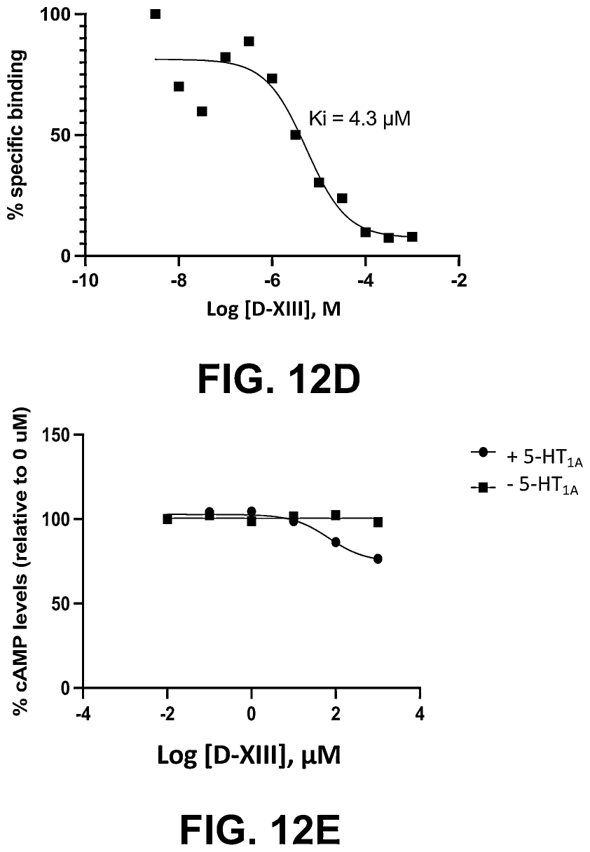

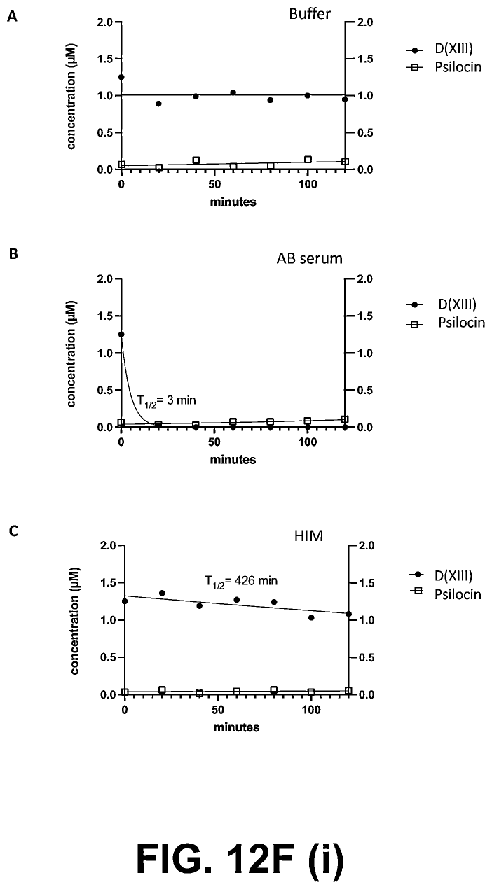

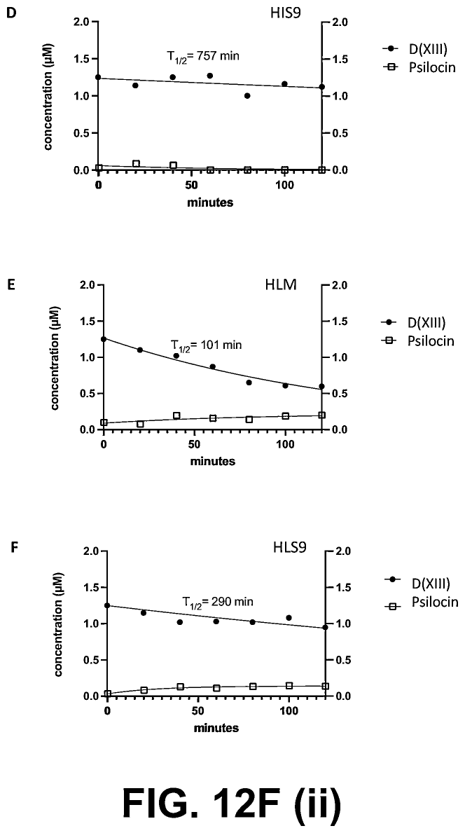

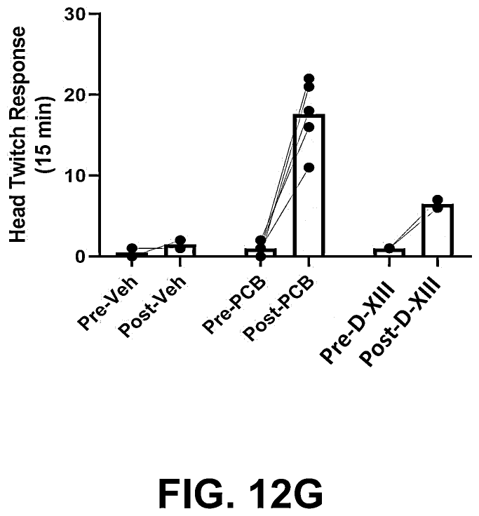

Referring to FIG. 12A, in one embodiment, the chemical compound can have the formula D(XIII):

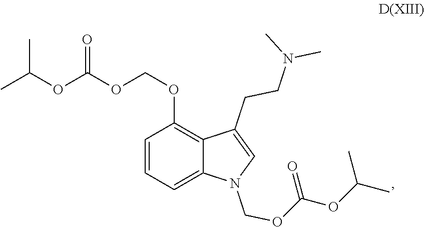 [SMILES: CC(C)OC(=O)OCOc1cccc2c1c(CCN(C)C)cn2COC(=O)OC(C)C]

and the at least one chemical synthesis reaction is the reaction depicted in FIG. 12A.

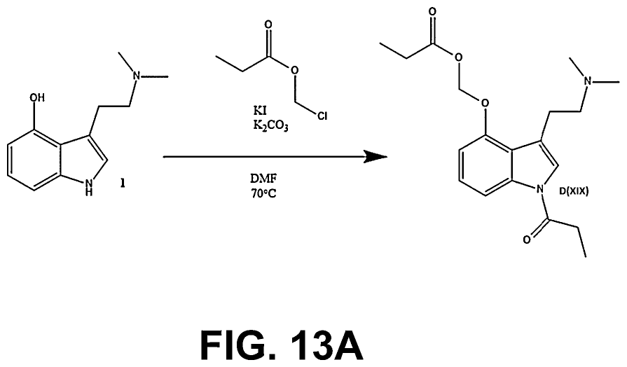

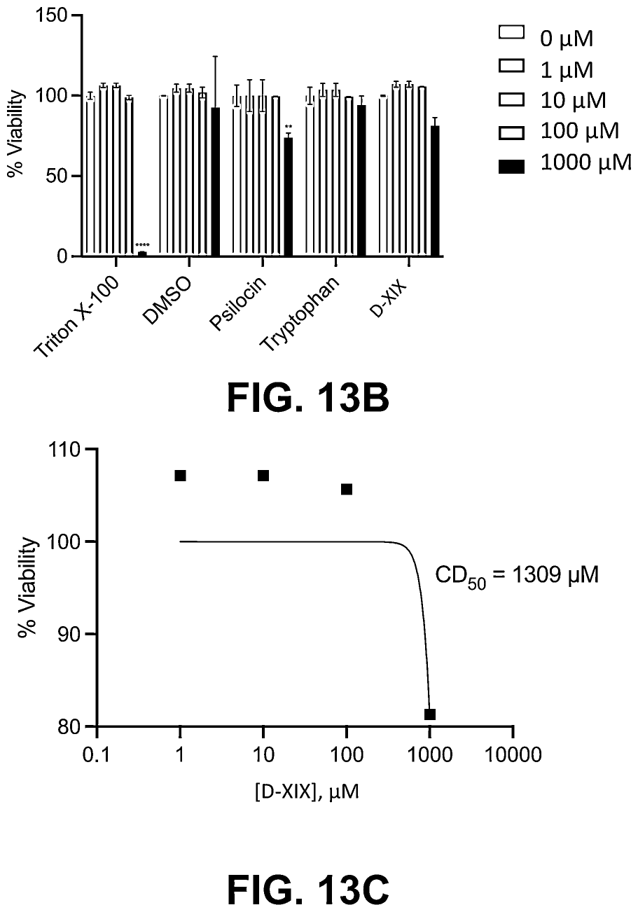

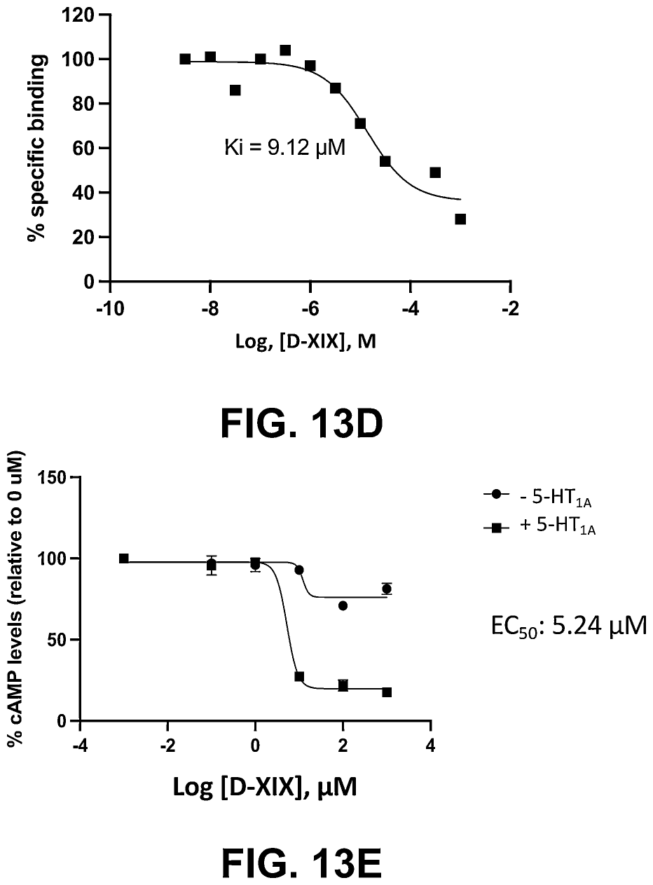

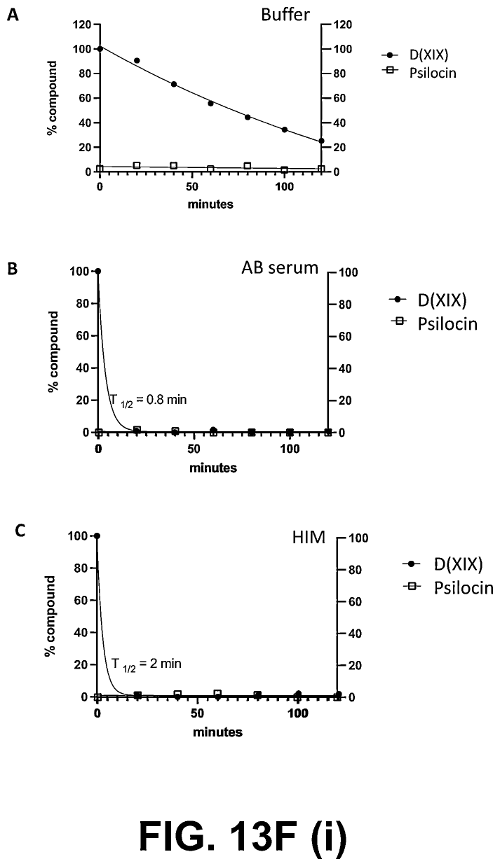

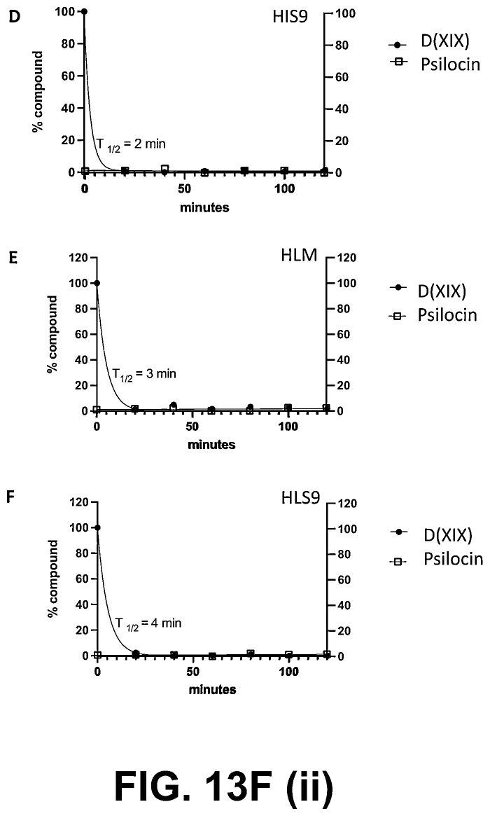

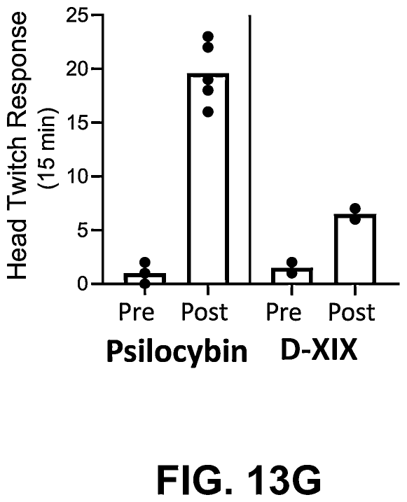

Referring to FIG. 13A, in one embodiment, the chemical compound can have the formula D(XIX):

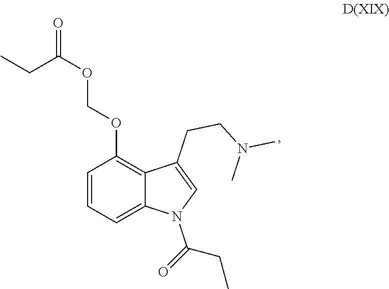 [SMILES: CCC(=O)OCOc1cccc2c1c(CCN(C)C)cn2C(=O)CC]

and the at least one chemical synthesis reaction is the reaction depicted in FIG. 13A.

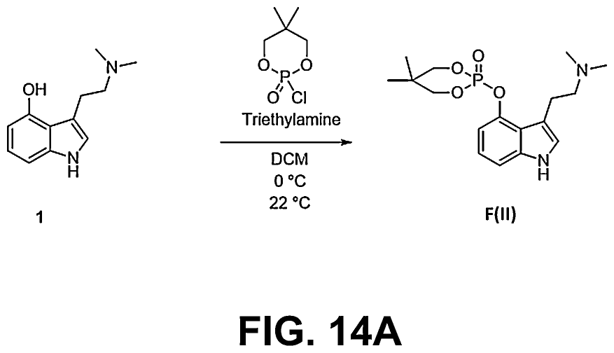

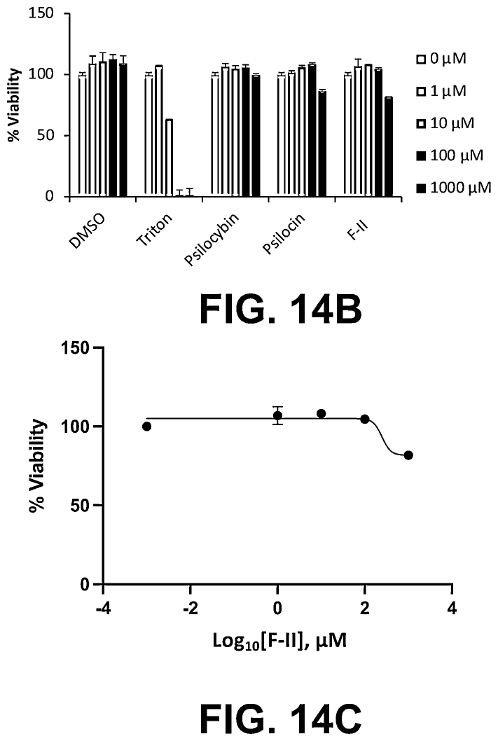

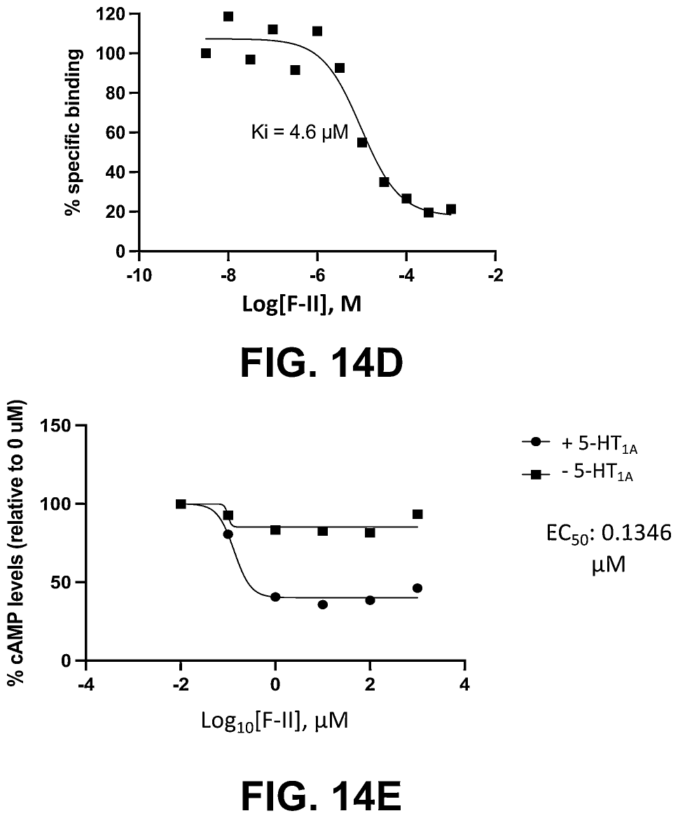

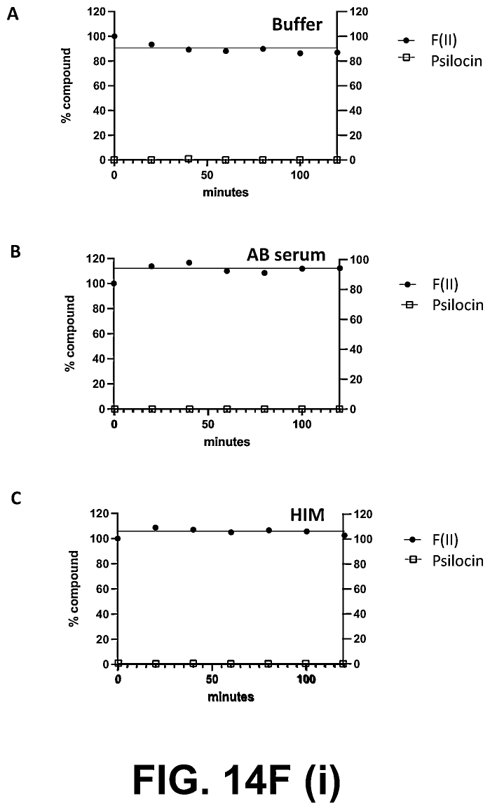

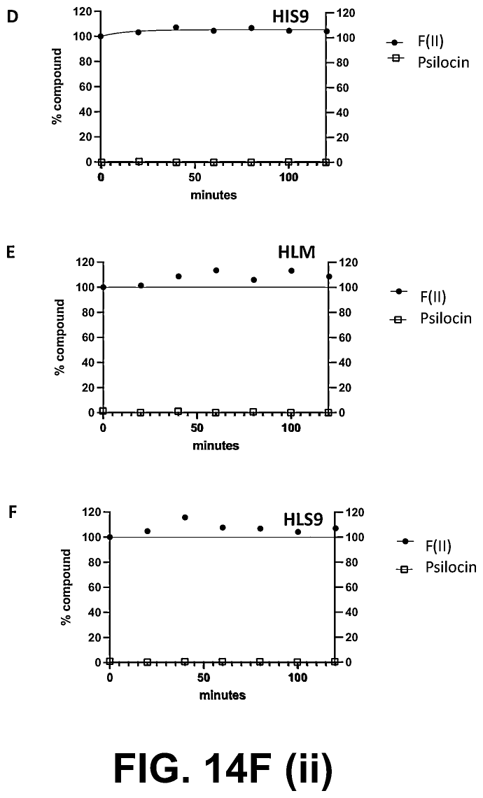

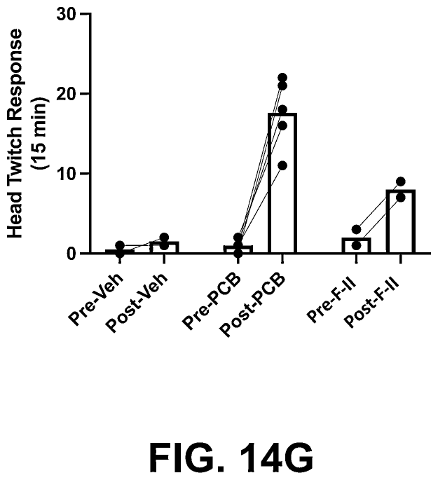

Referring to FIG. 14A, in one embodiment, the chemical compound can have the formula F(II):

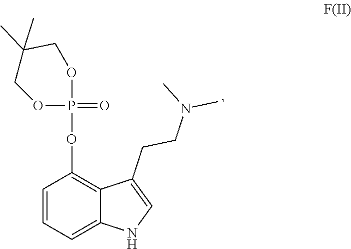

and the at least one chemical synthesis reaction is the reaction depicted in FIG. 14A.

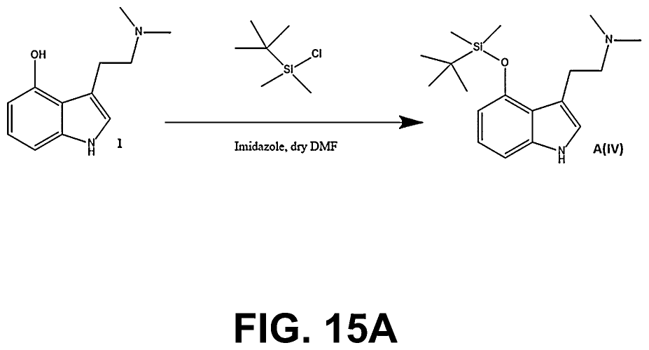

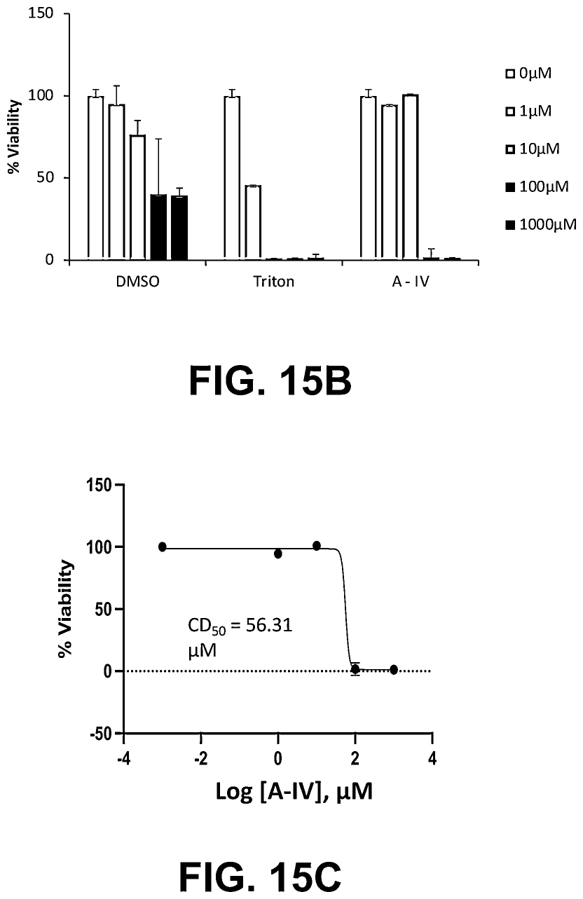

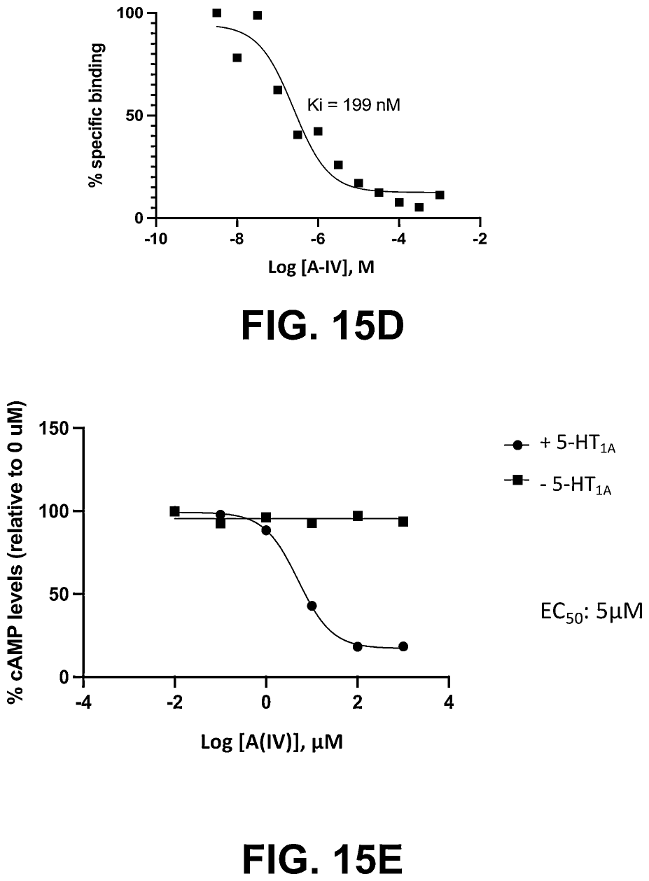

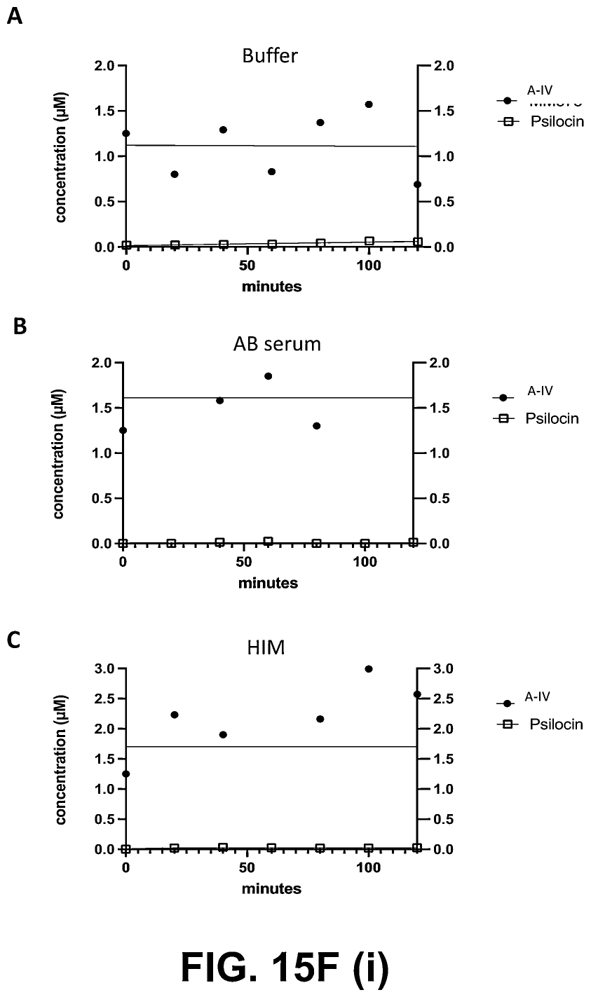

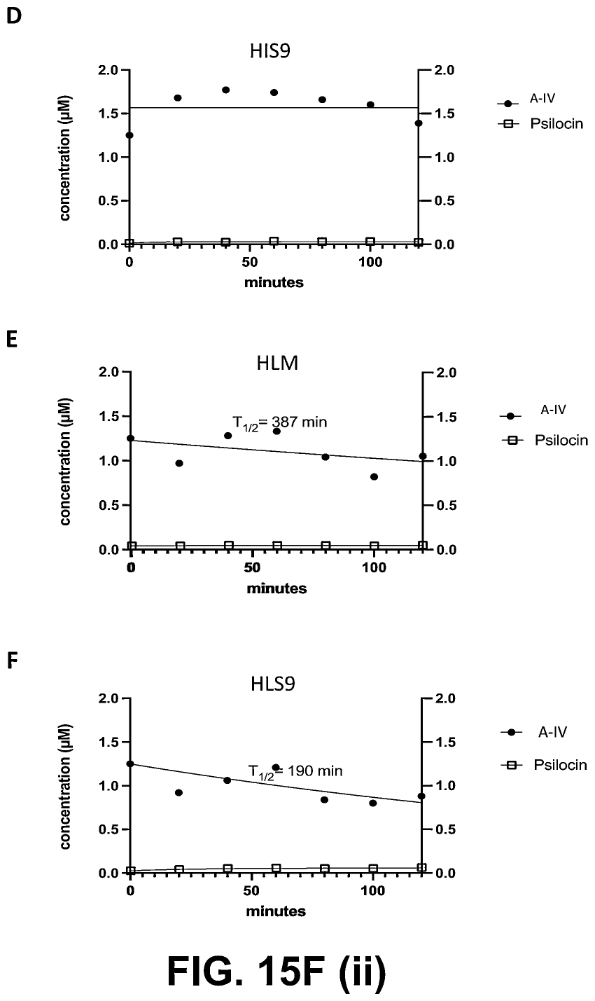

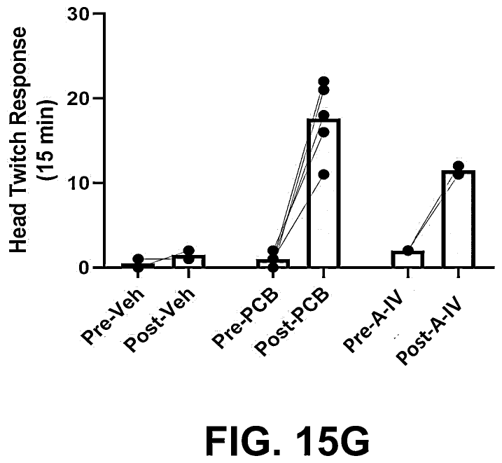

Referring to FIG. 15A, in one embodiment, the chemical compound can have the formula A(IV):

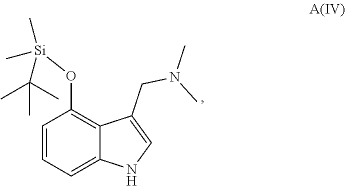 [SMILES: CN(C)Cc1c[nH]c2cccc(O[Si](C)(C)C(C)(C)C)c12]

and the at least one chemical synthesis reaction is the reaction depicted in FIG. 15A.

In some embodiments, the chemical compounds may be isolated in pure or substantially pure form. Thus, the compounds may be, for example, at least 90%, 95%, 96%, 97%, or 98%, or at least 99% pure.

It will now be clear from the foregoing that novel C₄-substituted tryptamine derivatives are disclosed herein. The C₄-substituted tryptamine derivatives may be formulated for use as a pharmaceutical drug or recreational drug.

### EXAMPLES

### Example 1—Synthesis and Analysis of a First C₄-Phosphate-Substituted Tryptamine Derivative

Referring to FIG. 3A, a dry round-bottom flask was charged with psilocin 1 (137 mg, 0.67 mmol, 1.0 eq) and dry DCM (1 mL) under argon. Notably, the synthesis of psilocin (1) has been described previously (Shirota et al., J. Nat. Prod. 2003, 66:885-887; Kargbo et al., ACS Omega 2020, 5:16959-16966). To this suspension, Et₃N (188 μL, 2.0 eq) was added and the resulting mixture was cooled down to 0° C. To the reaction mixture, a solution of diphenylphosphoryl chloride (278 μL, 2.0 eq) in DCM (0.5 mL) was added, and the resulting mixture was stirred at room temperature for 16 h. Upon completion (TLC), the mixture was diluted with DCM (10 mL) and washed with saturated aq. NaHCO₃ (2×25 mL) and brine. The organic layer was dried over anhydrous MgSO₄, filtered and concentrated. The crude material was purified by flash chromatography using silica gel (MeOH/DCM, 0:100 to 10:90, gradient) to yield the compound F(I) as a light-yellow oil (109 mg, 37%). Purity was determined to be 95%. MS and NMR characterization is as follows: MS-ESI: calculated: 437.1625; observed: 437.1620 m/z [M+H]⁺. ¹H NMR (400 MHz, Methanol-d₄): δ 7.37-7.10 (m, 14H), 3.11-2.97 (m, 2H), 2.81-2.69 (m, 2H), 2.35 (s, 6H).

### Assessment of Cell Viability Upon Treatment of a Psilocin Derivative.

To establish suitable ligand concentrations for competitive binding assays, PrestoBlue assays were first performed. The PrestoBlue assay measures cell viable activity based on the metabolic reduction of the redox indicator resazurin and is a preferred method for routine cell viability assays (Terrasso et al., 2017, J Pharmacol. Toxicol. Methods 83: 72). Results of these assays were conducted using novel derivatives, in part as a pre-screen for any remarkable toxic effects on cell cultures up to concentrations of 1 mM. A known cellular toxin (Triton X-100, Pyrgiotakis G. et al., 2009, Ann. Biomed. Eng. 37: 1464-1473) was included as a general marker of toxicity. Drug-induced changes in cell health within simple in vitro systems such as the HepG2 cell line are commonly adopted as first-line screening approaches in the pharmaceutical industry (Weaver et al., 2017, Expert Opin. Drug Metab. Toxicol. 13: 767). HepG2 is a human hepatoma that is most commonly used in drug metabolism and hepatotoxicity studies (Donato et al., 2015, Methods Mol Biol 1250: 77). Herein, HepG2 cells were cultured using standard procedures using the manufacture's protocols (ATCC, HB-8065). Briefly, cells were cultured in Eagle's minimum essential medium supplemented with 10% fetal bovine serum and grown at 37° C. in the presence of 5% CO₂. To test the various compounds with the cell line, cells were seeded in a clear 96-well culture plate at 20,000 cells per well. After allowing cells to attach and grow for 24 hours, compounds were added at 1 mM, 10 mM, 100 mM, and 1 mM. Methanol or DMSO were used as vehicles, at concentrations 0, 0.001, 0.01, 0.1, and 1% (methanol) or 0, 0.001, 0.01, 0.1, and 1% (DMSO), respectively. As a positive control for toxicity, TritonX concentrations used were 0.0001, 0.001, 0.01 and 0.1%. Cells were incubated with compounds for 48 hours before assessing cell viability with the PrestoBlue assay following the manufacture's protocol (ThermoFisher Scientific, P50200). PrestoBlue reagent was added to cells and allowed to incubate for 1 hour before reading. Absorbance readings were performed at 570 nm with the reference at 600 nm on a SpectraMax iD3 plate reader. Non-treated cells were assigned 100% viability. Bar graphs show the mean+/−SD, n=3. Significance was determined by 2-way ANOVA followed by Dunnett's multiple comparison test. Data acquired for the derivative having chemical formula F(I) is displayed as “F-I” on the x-axes in FIG. 3B and FIG. 3C.

### Radioligand Receptor Binding Assays.

Evaluation of drug binding is an essential step to characterization of all drug-target interactions (Fang 2012, Exp Opin Drug Discov 7:969). The binding affinity of a drug to a target is traditionally viewed as an acceptable surrogate of its in vivo efficacy (Nún\tilde{e}z et al., 2012, Drug Disc Today 17: 10). Competition assays, also called displacement or modulation binding assays, are a common approach to measure activity of a ligand at a target receptor (Flanagan 2016, Methods Cell Biol 132: 191). In these assays, standard radioligands acting either as agonists or antagonists are ascribed to specific receptors. In the case of G protein-coupled receptor 5-HT_{2A}, [³H]ketanserin is a well-established antagonist used routinely in competition assays to evaluate competitive activity of novel drug candidates at the 5-HT_{2A} receptor (Maguire et al., 2012, Methods Mol Biol 897: 31). Thus, to evaluate activity of novel C₄-substituted tryptamine derivatives at the 5-HT_{2A} receptor, competition assays using [³H]ketanserin were employed as follows. SPA beads (RPNQ0010), [³H]ketanserin (NET1233025UC), membranes containing 5-HT_{2A} (ES-313-M400UA), and isoplate-96 microplate (6005040) were all purchased from PerkinElmer. Radioactive binding assays were carried out using Scintillation Proximity Assay (SPA). For saturation binding assays, mixtures of 10 μg of membrane containing 5-HT_{2A} receptor was pre-coupled to 1 mg of SPA beads at room temperature in a tube rotator for 1 hour in binding buffer (50 mM Tris-HCl pH7.4, 4 mM CaCl₂, 1 mM ascorbic acid, 10 mM pargyline HCl). After pre-coupling, the beads and membrane were aliquoted in an isoplate-96 microplate with increasing amounts of [³H]ketanserin (0.1525 nM to 5 nM) and incubated for two hours at room temperature in the dark with shaking. After incubation, the samples were read on a MicroBeta 2 Microplate Counter (Perkin Elmer). Determination of non-specific binding was carried out in the presence of 20 mM of spiperone (S7395-250MG, Sigma). Equilibrium binding constants for ketanserin (K_{d}) were determined from saturation binding curves using the ‘one-site saturation binding analysis’ method of GraphPad PRISM software (Version 9.2.0). Competition binding assays were performed using fixed (1 nM) [³H]ketanserin and different concentrations of tryptophan (3 nM to 1 mM), psilocin (30 μM to 10 mM) or unlabeled test compound (3 nM to 1 mM) similar to the saturation binding assay. Kᵢ values were calculated from the competition displacement data using the competitive binding analysis from GraphPad PRISM software. Tryptophan was included as a negative control as it has no activity at the 5-HT_{2A} receptor. In contrast, psilocin was used as a positive control since it has established binding activity at the 5-HT_{2A} receptor (Kim et al., 2020, Cell 182: 1574). FIG. 3D depicts the saturation binding curves for [³H]ketanserin at the 5-HT_{2A} receptor. Panel A shows the specific saturation ligand binding of [³H]ketanserin (from 0.1525 nM to 5 nM) to membranes containing 5-HT_{2A} receptor, which was obtained after subtracting non-specific binding values (shown in Panel B). Specific binding in counts per minute (cpm) was calculated by subtracting non-specific binding from total binding. Specific binding (pmol/mg) was calculated from pmol of [³H]ketanserin bound per mg of protein in the assay. The K_{d} was calculated by fitting the data with the one-site binding model of PRISM software (version 9.2.0). FIG. 3E shows the competition binding curves for psilocin as a positive control (binding). This assay was conducted twice, yielding data shown in Panels A and B, respectively. FIG. 3F shows the competition binding curves for psilocybin (Panel A) and tryptophan (Panel B). Psilocybin is known to release the 5-HT_{2A}-binding metabolite psilocin in vivo; however, the intact psilocybin molecule itself displays very weak (McKenna and Peroutka 1989, J Neurosci 9: 3482) or arguably negligible (PDSP Certified Data; https://pdsp.unc.edu/databases/pdsp.php) binding at 5-HT_{2A}. Tryptophan is included as a negative control (no binding). The competition binding curve for compound with formula F(I), designated “F-I”, is presented in FIG. 3G.

### Cell Lines and Control Ligands Used to Assess Activity at 5-HT_{1A}

CHO-K1/Ga₁₅ (GenScript, M00257) (−5-HT_{1A}) and CHO-K1/5-HT_{1A}/Ga₁₅ (GenScript, M00330) (+5-HT_{1A}) cells lines were used. Briefly, CHO-K1/Ga₁₅ is a control cell line that constitutively expresses Ga₁₅ which is a promiscuous G_{q} protein. This control cell line lacks any transgene encoding 5-HT_{1A} receptors, but still responds to forskolin; thus, cAMP response to forskolin should be the same regardless of whether or not 5-HT_{1A} agonists are present. Conversely, CHO-K1/5-HT_{1A}/Ga₁₅ cells stably express 5-HT_{1A} receptor in the CHO-K1 host background. Notably, Ga₁₅ is a promiscuous G protein known to induce calcium flux response, present in both control and 5-HT_{1A} cell lines. In +5-HT_{1A} cells, Ga₁₅ may be recruited in place of G_{ai/io}, which could theoretically dampen cAMP response (Rojas and Fiedler 2016, Front Cell Neurosci 10: 272). Thus, we included two known 5-HT_{1A} agonists, psilocin (Ray 2010, PLoS ONE 5(2): e9019) and serotonin (Rojas and Fiedler 2016, Front Cell Neurosci 10: 272) as positive controls to ensure sufficient cAMP response was observed, thereby indicating measurable recruitment of G_{ai/io} protein to activated 5-HT_{1A} receptors. In contrast, tryptophan is not known to activate 5-HT_{1A} receptors and was thus used as a negative control. Cells were maintained in complete growth media as recommended by supplier (GenScript) which is constituted as follows: Ham's F12 Nutrient mix (HAM's F12, GIBCO #11765-047) with 10% fetal bovine serum (FBS) (Thermo Scientific #12483020), 200 mg/ml zeocin (Thermo Scientific #R25005) and/or 100 mg/ml hygromycin (Thermo Scientific #10687010). The cells were cultured in a humidified incubator with 37° C. and 5% CO₂. Cell maintenance was carried out as recommended by the cell supplier. Briefly, vials with cells were removed from the liquid nitrogen and thawed quickly in 37° C. water bath. Just before the cells were completely thawed the vial's outside was decontaminated by 70% ethanol spray. The cell suspension was then retrieved from the vial and added to warm (37° C.) complete growth media and centrifuged at 1,000 rpm for 5 minutes. The supernatant was discarded, and the cell pellet was then resuspended in another 10 ml of complete growth media and added to the 10 cm cell culture dish (Greiner Bio-One #664160). The media was changed every third day until the cells were about 90% confluent. The ˜90% confluent cells were then split 10:1 for maintenance or used for experiment.

### Evaluation of 5-HT_{1A} Receptor Modulation

As 5-HT_{1A} activation inhibits cAMP formation, the agonist activity of test molecules on 5-HT_{1A} was measured via the reduction in the levels of cAMP produced due to application of 4 mM forskolin. The change in intracellular cAMP levels due to the treatment of novel molecules was measured using cAMP-Glo Assay kit (Promega #V1501). Briefly, +5-HT_{1A} cells were seeded on 1-6 columns and base −5-HT_{1A} cells were seeded on columns 7-12 of the white walled clear bottom 96-well plate (Corning, #3903). Both cells were seeded at the density of 30,000 cells/well in 100 ml complete growth media and cultured 24 hrs in humidified incubator at 37° C. and 5% CO₂. On the experiment day, the media of cells was replaced with serum/antibiotic free culture media. Then the cells were treated for 20 minutes with test molecules dissolved in induction medium (serum/antibiotic free culture media containing 4 mM forskolin, 500 mM IBMX (isobutyl-1-methylxanthine, Sigma-Aldrich, Cat. #17018) and 100 mM (RO 20-1724, Sigma-Aldrich, Cat. #B8279)). Forskolin induced cAMP formation whereas IBMX and RO 20-1724 inhibited the degradation of cAMP. The level of luminescence in cells incubated with induction medium (containing 4 mM forskolin) without test molecules was normalized to represent 100% cAMP in this assay. PKA was added to the lysate, mixed, and subsequently the substrate of the PKA was added. PKA was activated by cAMP, and the amount of ATP consumed due to PKA phosphorylation directly corresponded to cAMP levels in the lysate. Reduced ATP caused reduced conversion of luciferin to oxyluciferin, conferring diminished luminescence as the result of 5-HT_{1A} activation. FIG. 3H shows increasing levels of cAMP in cultured cells incubated with increasing concentrations of forskolin independent of 5-HT_{1A} expression. FIG. 3I illustrates no reduction in cellular cAMP levels in either cell culture (+5-HT_{1A} and −5-HT_{1A}) stimulated with induction medium and treated with increasing doses of tryptophan, indicating a lack of 5-HT_{1A} activity by this molecule in +5-HT_{1A} cells. FIG. 3J illustrates reduction in cAMP levels in 5-HT_{1A} receptor expressing cells (+5-HT_{1A}) stimulated with 4 mM forskolin as levels of psilocin increase, indicating 5-HT_{1A} receptor binding by psilocin in these cells. Conversely, this trend of decreasing % cAMP levels with increasing psilocin is not observed in cells lacking expression of 5-HT_{1A} receptor. FIG. 3K illustrates reduction in cAMP levels in 5-HT_{1A} receptor expressing cells stimulated with 4 mM forskolin as levels of serotonin (5-HT) increase, indicating 5-HT_{1A} receptor binding by serotonin (5-HT) in these cells. Conversely, this trend of decreasing % cAMP levels with increasing serotonin (5-HT) is not observed in cells lacking expression of 5-HT_{1A} receptor. 5-HT_{1A} receptor binding evaluation for compound with formula F(I) (designated simply “F-I” along the x-axis) is shown in FIG. 3L. Comparison of data acquired in +5-HT_{1A} cultures with those acquired in −5-HT_{1A} cultures reveals receptor modulation by compound with formula F(I).

### In Vitro Metabolic Stability Assays Using Intestinal Fractions, Liver Fractions, Serum Fractions, and Control Buffer.

A fundamental evaluation in drug development is the assessment of absorption, distribution, metabolism, excretion, and pharmacokinetics (ADME/PK) (Eddershaw et al., 2000, Drug Discovery Today 5(9): 409-414). The first ADME screen that a novel chemical entity is subjected to is an in vitro metabolic stability screen (Ackley et al., 2004, Methods in Pharmacology and Toxicology Optimization in Drug Discovery (in vitro methods), Yan Z, Caldwell G. W. Eds; Humana Press Inc, New Jersey, pp. 151-164). Drug stability upon exposure to human liver microsomes and liver S9 cellular fractions is a common in vitro assay to approximate in vivo, liver-based drug metabolism (Richardson et al., 2016 Drug Metabolism Letters 10:83-90). First-pass metabolism is also often approximated in vitro using intestinal microsome and cellular S9 fractions (Hatley et al., 2017, Biopharmaceuticals & Drug Disposition, 38(2):155-160). Further, it is well known that human serum, and particularly circulating serum esterases can contribute to systemic drug metabolism (Williams, F M 1987, Pharmacology and Therapeutics, 34:99-109). Many pharmacological agents are classified as prodrugs, as they undergo metabolic transformation in vivo upon administration to release the active drug compound into the systemic compartment (Zawilska J B, et al. 2013, Pharmacological Reports, 65:1-14). Psilocybin, a serotonergic psychedelic agent, is well known prodrug that is metabolized into the psychoactive product, psilocin (Dinis-Oliveira, R J 2017, Drug Metabolism Reviews, 49(1):84-91). To evaluate the capacity of test molecules to similarly serve as prodrugs of psilocin, time-dependent, metabolic stability assays using human AB serum, human intestinal microsomes (HIM), human intestinal S9 fractions (HIS9), human liver microsomes (HLM), and human liver S9 fractions (HLS9) were performed. Assays in enzyme-free buffer were also performed for control purposes, and for general assessment of compound stability. Liquid chromatography coupled mass spectrometry (LC-MS) was employed to track the conversion of the test molecules into psilocin. All intestinal and liver fractions and NADPH RapidStart reagent was purchased from Sekisui/XenoTech. Human AB serum was purchased from Sigma. For intestine and liver metabolism assays, 2.5 μM candidate compounds were incubated in 400 μg/ml of each cellular fraction (HLM, HLS9, HIM, or HIS9) in 50 mM potassium phosphate buffer (pH 7.4) containing 3 mM MgCl2 and 1 mM EDTA supplemented with NADPH RapidStart at 37° C. Samples were taken at the start of the assay, and at every 20 minutes for 2 hours. Time-point samples were precipitated with 1:1 volume of acetonitrile to quench the reaction before centrifugation at 4000×g for 20 minutes. Supernatants were analyzed for the presence of candidate prodrugs (parent molecule) and psilocin (the predicted metabolite) using Orbitrap LC-MS (Thermo Scientific) using previously described methods (Menendez-Perdomo et al., 2021, J. Mass Spectrom., 56: e4683). The serum assays were carried out in 10% human AB serum in 50 mM potassium phosphate buffer (pH 7.4) containing 3 mM MgCl₂ and 1 mM EDTA. Assay concentrations (μM) of both parent ‘prodrug’ molecule and psilocin metabolite, as quantified through LC-MS using routine standard curve procedures, were plotted as functions of assay time (minutes). The metabolism rate (TI/2) was determined from the metabolism curve plot using the one phase decay feature of GraphPad PRISM software (Version 9.2.0). The quantity of parent prodrug at time zero was set as 100%.

Positive controls were first tested to ensure that assays were functioning properly. Psilocybin is known to be metabolized to psilocin in the intestine and through alkaline phosphatase (Dinis-Oliveira, 2017 Drug Metab. Rev. 49: 84-91) and thus served as a positive control for HIM and HIS9. Procaine is known to be metabolized to 4-amino benzoic acid in serum, liver, and through esterase (Henrikus and Kampffmeyer, 1992, Xenobiotica 22: 1357-1366) and thus served as a positive control for AB serum and HLM. Verapamil is known to be metabolized into a variety of metabolites in liver (Hanada et al., 2008, Drug Metab. Dispos. 36: 2037-2042) (catabolites not examined in this study) and thus served as an additional control for HLS9 and HLM assays.

FIGS. 3M (i)-3M (ii) illustrate results of ‘psilocin-release’ metabolic conversion assays using psilocybin as the parent prodrug control for HIM (Panel C), HIS9 (Panel D) and HLM (Panel E) assays. For context, psilocybin was further submitted to negative control buffer assay (Panel A), AB serum (Panel B), and HLS9 (Panel F) assays. Notably, these plots demonstrate psilocybin is stable in liver fractions with no conversion to psilocin. Further, the stability of psilocybin was confirmed in assay buffer, confirming that transformation of this molecule is due to enzymes within the cellular fractions rather than due to buffer components. Finally, these results demonstrate psilocybin is stable in serum with no conversion to psilocin. FIGS. 3N (i)-3N (ii) illustrate results of additional controls for assay verification: procaine and AB serum (Panel A); procaine and HLM (Panel B); verapamil and HLS9 (Panel C); verapamil and HLM (Panel D). FIGS. 3O (i)-3O (iii) show the metabolic stability curves for compound with formula F(I), designated “F(I),” in control buffer (Panel A), AB serum (Panel B), HIM (Panel C), HIS9 (Panel D), HLM (Panel E), and HLS9 (Panel F).

### In Vivo Evaluation of 5-HT_{2A} Receptor Agonism in Mice.

Drug-induced Head Twitch Response (HTR), a rapid, involuntary movement of the mouse's head with little or no involvement of the trunk, is an established in vivo model behavior used to measure neuronal 5-HT_{2A} receptor (5-HT2AR) activation by established and novel hallucinogenic compounds (Canal and Morgan 2012, Drug Testing Analysis, 4:556-576). Indeed, HTR is widely utilized as a behavioral proxy in mice and rats to predict human hallucinogenic potential and can reliably differentiate between hallucinogenic and non-hallucinogenic 5-HT2AR agonists (Halberstadt and Geyer 2013, Psychopharmacology 227: 727-739; Gonzalez-Maeso et al., 2007, Neuron 53:439-452). To evaluate 5-HT2AR agonisms in vivo, HTR was measured in mice treated with a control and test compounds over a fixed window of time post-administration. All experiments were approved by the University of Calgary Animal Care and Use Committee in accordance with Canadian Council on Animal Care guidelines. Briefly, 8-week-old C57BL/6-Elite male and female mice were obtained from Charles River. Prior to compound administration, all mice were group-housed, then single-housed on a 12:12 h light/dark schedule (lights on at 07:00 hours) with ad libitum access to food and water. Before any behavioral screening, mice were handled and exposed to the testing chamber for at least 5 min each day for three successive days and habituated to the experimental room 1 h before testing. The testing chamber was cleaned with a 70% ethanol solution between experiments. Control and test compounds, which were prepared at stock concentrations of 100 mM in DMSO, were diluted in sterile saline solution (0.9% NaCl). The sterile saline solution without control or test compounds (i.e., 0.9% NaCl) was dosed with 100 mM DMSO to create equivalent ‘vehicle’ solution. Prior to drug administration, mice were video monitored for 30 minutes in a plexiglass testing chamber (25.5×12.5×12.5 cm [L×W×H]) to allow for acclimation to the testing environment and to examine pre-drug spontaneous HTRs. After 30 minutes, compounds were administered via intraperitoneal (i.p.) injection at 1 mg/kg and mice were video monitored for 30 minutes then returned to their home cage. HTR analysis was conducted by an individual blinded to the subject treatment group using Behavioral Observation Research Interactive Software (BORIS, version 7, DOI: 10.1111/2041-210X.12584). Pre-drug behavior was examined during the 15-to-30-minute window prior to drug administration. Post-drug behavior was analyzed during the 15-to-30-minute window following drug administration. HTR associated with i.p. administration of psilocybin or vehicle were included as positive or negative control measures, respectively. Elevated incidences of HTR within the defined period of monitoring was observed in (1) psilocybin-treated mice, and (2) those treated with compound with formula F(I) is designated “F-I”, relative to control mice treated with i.p. injected vehicle. These results are illustrated in FIG. 3P, wherein vehicle is designated “veh,” psilocybin is designated “PCB,” compound with formula F(I) is designated “F-I,” pre-drug data is designated “pre-”, and post-drug data is designated “pro-.” Each replicate mouse is shown as a black dot along the corresponding vertical bars (N=2-6 per compound).

### In Vitro Survey of Pharmacological Interaction Profiles at Receptors, Transporters and Enzymes Linked to Targeted Health Conditions.

To expand pharmacological profiling to include a broader range of targets with known involvement in, or connection to, brain neurological disorders, compound F(I) was evaluated with respect to receptor interaction (https://www.eurofinsdiscoveryservices.com/). Specifically, the cell-based screening assay panel known as “SAFETYscan E/IC150 ELECT” was used to generate data regarding interaction of derivative molecules with 20 different proteins, including 12 GPCR receptors (ADRA1A, ADRA2A, AVPR1A, CHRM1, CHRM2, CNR1, DRD1, DRD2S, HTR1A (5-HT_{1A}), HTR1B (5-HTR_{1B}), HTR2B (5-HT_{2B}), OPRD1), 3 ion channels (GABAA, HTR3A (5-HT_{3A}), NMDAR), one enzyme (MAO-A), and 3 transporters (DAT, NET, SERT).

i. EFC-Based cAMP Secondary Messenger Assay.

Of the 12 GPCR proteins, 8 were assayed via a cAMP secondary messenger assay: ADRA2A, CHRM2, CNR1, DRD1, DRD2S, HTR1A, HTR1B, OPRD1. Briefly, employed a panel of cell lines stably expressing non-tagged GPCR proteins that endogenously signal through cAMP. These assays monitored the activation of a GPCR through Gᵢ or Gₛ secondary messenger signaling in a homogenous, non-imaging assay format using a technology termed EnzymeFragment Complementation (EFC). EFC uses β-galactosidase (β-gal) as the functional endpoint. The β-gal enzyme is split into two complementary portions: Enzyme Acceptor (EA) and Enzyme Donor (ED). In the assay, exogenously introduced ED fused to cAMP (ED-cAMP) competes with endogenously generated cAMP for binding to an anti-cAMP-specific antibody. Active β-gal is formed by complementation of exogenous EA to any unbound ED-cAMP. Active enzyme can then convert a chemiluminescent substrate, generating an output signal detectable on a standard microplate reader.

These 8 cAMP-based assays were conducted in both agonist and antagonist modes, either in Gₛ format (no forskolin) or in Gᵢ format (in the presence of EC₈₀ forskolin). For Gₛ and Gᵢ agonist assays: cell media was aspirated from GPCR-containing cultures and replaced with 15 μl 2:1HBSS/1-mM HEPES:cAMP XS+Ab reagent. Five microlitres of derivative compound, prepared as a stock solution (also containing EC₈₀ forskolin in the case of Gᵢ format) were added to the cells at final target concentrations and pre-incubated for 30 minutes. Final assay vehicle concentration was 1%. After pre-incubation, assay signal was generated through the addition of (1) 20 μL cAMP XS+ED/CL lysis cocktail, and (2) 20 μLcAMP XS+EA reagent, allowing incubation periods of one and three hours, respectively. Antagonist assays were performed in the same manner as agonist assays, except pre-incubation entailed exposure to the test derivative (30 minutes) followed by exposure to an established agonist at EC₈₀ (“agonist challenge”, 30 minutes). In the case of antagonist assays of Gᵢ-coupled GPCRs, EC₈₀ forskolin was included in assay buffers.

In all 8 cAMP assays (agonist or antagonist mode), the resulting chemiluminescent signal was measured using a PerkinElmer Envision™ instrument. Compound activity was analyzed using CBIS data analysis suite (ChemInnovation, CA). Percent activity (%) was calculated according to standard procedures. For example: in Gₛ agonist mode assays, percentage activity was calculated using the following formula: % activity=100%×[mean RLU of test derivative−mean RLU of vehicle control]/[mean RLU of control ligand−mean RLU of vehicle control]. For Gₛ antagonist mode assays, percentage inhibition was calculated using the following formula: % inhibition=100%×[1−[mean RLU of test derivative−mean RLU of vehicle control]/[mean RLU of EC₈₀ control ligand−mean RLU of vehicle control]]. For Gᵢ agonist mode assays, percentage activity was calculated using the following formula: % activity=100%×[1−[mean RLU of test derivative−mean RLU of control ligand]/[mean RLU of vehicle control−mean RLU of control ligand]]. For Gᵢ antagonist or negative allosteric mode assays, percentage inhibition was calculated using the following formula: % inhibition=100%×[mean RLU of test compound−mean RLU of EC₈₀ control ligand]/[mean RLU of forskolin positive control−mean RLU of EC₈₀ control]. For primary screens, percent response was capped at 0% or 100% where calculated percent response returned a negative value or a value greater than 100, respectively. To assess assay performance and establish positive control benchmarks, ligands listed in Table 1A were evaluated alongside test derivatives. Results for EFC-based cAMP secondary messenger assays on GPCRs using compound F(I) ligand or positive controls are shown in Table 1B.

ii. Calcium Secondary Messenger Assay.

Of the 12 GPCR proteins, 4 were assayed via a calcium secondary messenger assay: ADRA1A, AVPR1A, CHRM1, HTR2B. Briefly, the Calcium No WashPLUS assay monitors GPCR activity via G_{q} secondary messenger signaling in a live cell, non-imaging assay format. Eurofins DiscoverX employed proprietary cell lines stably expressing G_{q}-coupled GPCR proteins. Calcium mobilization was monitored using a calcium-sensitive dye loaded into cells. GPCR activation by a test or control compound resulted in the release of calcium from intracellular stores and an increase in dye fluorescence that is measured in real-time.

The four GPCR proteins assayed via calcium secondary messenger assay were surveyed in both agonist and antagonist modes. Cell lines were expanded from freezer stocks according to standard procedures, seeded into microplates and incubated at 37° C. prior to testing. Assays were performed in 1× dye loading buffer consisting of 1× dye (DiscoverX, Calcium No WashPLUS kit, Catalog No. 90-0091), 1× Additive A and 2.5 mM probenecid in HBSS/20 mM Hepes. Cells were loaded with dye prior to testing. Media was aspirated from cells and replaced with 25 μL dye loading buffer, incubated for 45 minutes at 37° C. and then 20 minutes at room temperature. For agonist determination, cells were incubated with sample compound to induce response. After dye loading, cells were removed from the incubator and 25 μL of 2× compound in HBSS/20 mM Hepes was added using a FLIPR Tetra (MDS). Compound agonist activity was measured on a FLIPR Tetra. Calcium mobilization was monitored for 2 minutes with a 5 second baseline read. For antagonist determination, cells were pre-incubated with sample compound followed by agonist challenge at the EC₈₀ concentration. After dye loading, cells were removed from the incubator and 25 μL 2× sample compound was added. Cells were incubated for 30 minutes at room temperature in the dark to equilibrate plate temperature. After incubation, antagonist determination was initiated with addition of 25 μL 1× derivative compound with 3× EC₈₀ agonist using FLIPR. Compound antagonist activity was measured on a FLIPR Tetra (MDS). Calcium mobilization was monitored for 2 minutes with a 5 second baseline read. In both agonist and antagonist modes, data analysis was initiated using FLIPR, where area under the curve was calculated for the entire two-minute read. Compound activity was analyzed using CBIS data analysis suite (ChemInnovation, CA). For agonist mode assays, percentage activity was calculated using the following formula: % activity=100%×[mean RFU of test compound−mean RFU of vehicle control]/[mean RFU control ligand−mean RFU of vehicle control]. For antagonist mode assays, percentage inhibition was calculated using the following formula: % inhibition=100%×[1−[mean RFU of test compound−mean RFU of vehicle control]/[mean RFU of EC₈₀ control−mean RFU of vehicle control]]. For primary screens, percent response was capped at 0% or 100%, where calculated percent response returned a negative value or a value greater than 100, respectively. To assess assay performance and establish positive control benchmarks, ligands listed in Table 1A were evaluated alongside test derivatives. Results for EFC-based cAMP secondary messenger assays on GPCRs using compound F(I) ligand or positive controls are shown in Table 1B.

iii. Ion Channel Assays.

Both ‘blocker’ and ‘opener’ activities of putative ligands on three distinct ion channels (GABAA, HTR3A, NMDAR) were surveyed. Briefly, Eurofins DiscoverX was employed in conjunction with the FLIPR Membrane Potential Assay Kit (Molecular Devices) which utilizes a proprietary fluorescent indicator dye in combination with a quencher to reflect real-time membrane potential changes associated with ionchannel activation and ion transporter proteins. Unlike traditional dyes such as DiBAC, the FLIPR Membrane Potential Assay Kit detects bidirectional ion fluxes so both variable and control conditions can be monitored within a single experiment. Cell lines were expanded from freezer stocks according to standard procedures, seeded onto microplates, and incubated at 37° C. Assays were performed in 1× Dye Loading Buffer consisting of 1× Dye and 2.5 mM probenecid when applicable. Cells were loaded with dye prior to testing and incubated for 30-60 minutes at 37° C. For agonist (‘Opener’) assays, cells were incubated with sample (i.e., containing derivative or control compound; Table 1) to induce response as follows. Dilution of sample stocks was performed to generate 2-5× sample (i.e., containing derivative or control compound) in assay buffer. Next, 10-25 μL of 2-5× sample was added to cells and incubated at 37° C. or room temperature for 30 minutes. Antagonist (‘Blocker’) assays were performed using the same procedure except that after dye loading, cells were removed from the incubator and 10-25 μL 2-5× sample (i.e., containing derivative or control compound) was added to cells in the presence of EC₈₀ agonist. Cells were incubated for 30 minutes at room temperature in the dark to equilibrate plate temperature. Compound activity was measured on a FLIPR Tetra (Molecular Devices). Compound activity was analyzed using CBIS data analysis suite (ChemInnovation, CA). For agonist mode assays, percentage activity was calculated using the following formula: % activity=100%×[mean RLU of test derivative−mean RLU of vehicle control]/[mean control ligand−mean RLU of vehicle control]. For antagonist mode, percentage inhibition was calculated using the following formula: % inhibition=100%×[1−[mean RLU of test derivative−mean RLU of vehicle control]/[mean RLU of EC₈₀ control−mean RLU of vehicle control]]. For primary screens, percent response was capped at 0% or 100% where calculated percent response returned a negative value or a value greater than 100, respectively. To assess assay performance and establish positive control benchmarks, ligands listed in Table 1A were evaluated alongside test derivatives. Results for EFC-based cAMP secondary messenger assays on GPCRs using compound F(I) ligand or positive controls are shown in Table 1B.

iii. Neurotransmitter Transporter Uptake Assays.

The Neurotransmitter Transporter Uptake Assay Kit from Molecular Devices was used to examine impact of test compounds on 3 distinct transporters (DAT, NET, SERT). This kit provided a homogeneous fluorescence-based assay for the detection of dopamine, norepinephrine or serotonin transporter activity in cells expressing these transporters. The kit employed a fluorescent substrate that mimics the biogenic amine neurotransmitters that are taken into the cell through the specific transporters, resulting in increased intracellular fluorescence intensity. Cell lines were expanded from freezer stocks according to standard procedures, seeded into microplates and incubated at 37° C. prior to testing. Assays were performed in 1× Dye Loading Buffer consisting of 1× Dye, and 2.5 mM probenecid as applicable. Next, cells were loaded with dye and incubated for 30-60 minutes at 37° C. “Blocker” or antagonist format assays were performed, where cells were pre-incubated with sample (i.e., containing sample derivative or positive control compound) as follows. Dilution of sample stocks (i.e., containing sample derivative or positive control compound; Table 1) was conducted to generate 2-5× sample in assay buffer. After dye loading, cells were removed from the incubator and 10-25 μL 2-5× sample (i.e., containing sample derivative or positive control compound) was added to cells in the presence of EC₈₀ agonist as appropriate. Cells were incubated for 30 minutes at room temperature in the dark to equilibrate plate temperature. Compound activity was measured on a FLIPR Tetra (Molecular Devices), and activity was analyzed using CBIS data analysis suite (ChemInnovation, CA). For antagonist (‘Blocker’) mode, percentage inhibition was calculated using the following formula: % inhibition=100%×[1−[mean RLU of test sample−mean RLU of vehicle control]/[mean RLU of EC₈₀ control−mean RLU of vehicle control]]. For primary screens, percent response was capped at 0% or 100% where calculated percent response returned a negative value or a value greater than 100, respectively. To assess assay performance and establish positive control benchmarks, ligands listed in Table 1A were evaluated alongside test derivative. Results for EFC-based cAMP secondary messenger assays on GPCRs using compound F(I) ligand or positive controls are shown in Table 1B.

v. MAO-A Enzyme Assay.

For the MAO-A assay, all chemicals and enzyme preparations were sourced from Sigma. Briefly, enzyme and test compound (i.e., derivative or control compound; see Table 1A) were preincubated for 15 minutes at 37° C. before substrate addition. The reaction was initiated by addition of kynuramine and incubated at 37° C. for 30 minutes. The reaction was terminated by addition of NaOH. The amount of 4-hydroquinoline formed was determined through spectrofluorimetric readout with the emission detection at 380 nm and excitation wavelength 310 nm. For each assay, microplates were transferred to a PerkinElmer Envision™ instrument for readouts as per standard procedures. Compound activity was analyzed using CBIS data analysis suite (ChemInnovation, CA). Percentage inhibition was calculated using the following formula: % inhibition=100%×[1−[mean RLU of test sample−mean RLU of vehicle control]/[mean RLU of positive control−mean RLU of vehicle control]]. For primary screens, percent response was capped at 0% or 100% where calculated percent response returned a negative value or a value greater than 100, respectively. To assess assay performance and establish positive control benchmarks, ligands listed in Table 1A were evaluated alongside test derivative. Results for EFC-based cAMP secondary messenger assays on GPCRs using compound F(I) ligand or positive controls are shown in Table 1B.

TABLE 1A
Control ligands used for target assays (GPCR, G-protein coupled
receptor; IC, ion channel; EN, enzyme; TR, transporter).

| Target | Assay | Type | Control ligand/modulator |
|---|---|---|---|
| ADRA1A | Agonist | GPCR | A 61603 Hydrobromide |
| ADRA1A | Antagonist | GPCR | Tamsulosin |
| ADRA2A | Agonist | GPCR | UK 14304 |
| ADRA2A | Antagonist | GPCR | Yohimbine |
| AVPR1A | Agonist | GPCR | [Arg8]-Vasopressin |
| AVPR1A | Antagonist | GPCR | SR 49059 |
| CHRM1 | Agonist | GPCR | Acetylcholine chloride |
| CHRM1 | Antagonist | GPCR | Atropine |
| CHRM2 | Agonist | GPCR | Acetylcholine chloride |
| CHRM2 | Antagonist | GPCR | Atropine |
| CNR1 | Agonist | GPCR | CP 55940 |
| CNR1 | Antagonist | GPCR | AM 251 |
| DRD1 | Agonist | GPCR | Dopamine |
| DRD1 | Antagonist | GPCR | SCH 39166 |
| DRD2S | Agonist | GPCR | Dopamine |
| DRD2S | Antagonist | GPCR | Risperidone |
| HTR1A | Agonist | GPCR | Serotonin hydrochloride |
| HTR1A | Antagonist | GPCR | Spiperone |
| HTR1B | Agonist | GPCR | Serotonin hydrochloride |
| HTR1B | Antagonist | GPCR | SB 224289 |
| HTR2B | Agonist | GPCR | Serotonin hydrochloride |
| HTR2B | Antagonist | GPCR | LY 272015 |
| OPRD1 | Agonist | GPCR | DADLE |
| OPRD1 | Antagonist | GPCR | Naltriben |
| GABAA | Opener | IC | GABA |
| GABAA | Blocker | IC | Picrotoxin |
| HTR3A | Opener | IC | Serotonin hydrochloride |
| HTR3A | Blocker | IC | Bemesetron |
| MAO-A | Inhibitor | EN | Clorgyline |
| DAT | Blocker | TR | GBR 12909 |
| NET | Blocker | TR | Desipramine |
| SERT | Blocker | TR | Clomipramine |
| NMDAR | Blocker | IC | (+)-MK 801 |
| NMDAR | Opener | IC | L-Glutamic acid |

TABLE 1B
Data summary table of target assays for compound F(I) (designated “F-
I”) and control ligands. Potency (EC₅₀ or IC₅₀) is provided in units of μM.

| Target | Target | Assay | EC₅₀ | IC₅₀ | EC₅₀ | IC₅₀ |
|---|---|---|---|---|---|---|
| name | type | type | (control) | (control) | (F-I) | (F-I) |
| ADRA1A | GPCR | AGN | 5.00E−05 | — | >100 | — |
| ADRA1A | GPCR | ANT | — | 9.60E−04 | — | 14.75 |
| ADRA2A | GPCR | AGN | 4.00E−05 | — | >100 | — |
| ADRA2A | GPCR | ANT | — | 3.10E−03 | — | 40.31 |
| AVPR1A | GPCR | AGN | 4.20E−04 | — | >100 | — |
| AVPR1A | GPCR | ANT | — | 1.60E−03 | — | >100 |
| CHRM1 | GPCR | AGN | 9.70E−03 | — | >100 | — |
| CHRM1 | GPCR | ANT | — | 6.10E−03 | — | 39.9 |
| CHRM2 | GPCR | AGN | 2.70E−02 | — | >100 | — |
| CHRM2 | GPCR | ANT | — | 3.20E−03 | — | 42.89 |
| CNR1 | GPCR | AGN | 1.00E−05 | — | >100 | — |
| CNR1 | GPCR | ANT | — | 6.20E−04 | — | 17.24 |
| DRD1 | GPCR | AGN | 9.10E−02 | — | >100 | — |
| DRD1 | GPCR | ANT | — | 7.10E−04 | — | 6.98 |
| DRD2S | GPCR | AGN | 5.10E−04 | — | 3.06 | — |
| DRD2S | GPCR | ANT | — | 9.60E−04 | — | >100 |
| HTR1A | GPCR | AGN | 1.70E−03 | — | 6.36 | — |
| HTR1A | GPCR | ANT | — | 4.60E−02 | — | >100 |
| HTR1B | GPCR | AGN | 9.00E−05 | — | 5.49E−02 | — |
| HTR1B | GPCR | ANT | — | 5.80E−03 | — | >100 |
| HTR2A | GPCR | AGN | 4.50E−03 | — | >100 | — |
| HTR2A | GPCR | ANT | — | 2.20E−03 | — | 1.68E−01 |
| HTR2B | GPCR | AGN | 6.30E−04 | — | >100 | — |
| HTR2B | GPCR | ANT | — | 4.00E−04 | — | 1.27E−01 |
| OPRD1 | GPCR | AGN | 5.00E−05 | — | >100 | — |
| OPRD1 | GPCR | ANT | — | 5.80E−04 | — | 57.05 |
| GABAA | Ion | OP | 6.2 | — | >100 | — |
| | channel | | | | | |
| GABAA | Ion | BL | — | 4.6 | — | 68.77 |
| | channel | | | | | |
| HTR3A | Ion | OP | 3.00E−01 | — | >100 | — |
| | channel | | | | | |
| HTR3A | Ion | BL | — | 1.90E−03 | — | 2.52E+00 |
| | channel | | | | | |
| MAO-A | Enzyme | IN | — | 2.90E−03 | — | 50.86 |
| DAT | transporter | BL | — | 1.40E−03 | — | >100 |
| NET | transporter | BL | — | 6.70E−03 | | 41.78 |
| SERT | transporter | BL | — | 1.80E−03 | — | 39.19 |
| NMDAR | Ion | BL | — | 8.00E−02 | — | >100 |
| | channel | | | | | |
| NMDAR | Ion | OP | 4.40E−01 | — | >100 | — |
| | channel | | | | | |

AGN, agonist; ANT, antagonist; OP, opener; BL, blocker; IN, inhibitor.

### Example 2—Synthesis and Analysis of a First C₄-Ether-Substituted Tryptamine Derivative

Referring to FIG. 4A, a dry round-bottom flask was charged with psilocin 1 (100 mg, 0.49 mmol, 1.0 eq) and imidazole (100 mg, 1.47 mmol, 3.0 eq) under Argon. Notably, the synthesis of psilocin (1) has been described previously (Shirota et al., J. Nat. Prod. 2003, 66:885-887; Kargbo et al., ACS Omega 2020, 5:16959-16966). Anhydrous DMF (1.50 mL) followed by triisopropylsilyl chloride (TIPSCl) (126 μL, 0.59 mmol, 1.2 eq) were added to the flask, and the resulting solution was stirred at room temperature for 4 h (or until completion, TLC). The volatiles were removed under reduced pressure, and the obtained residue was re-dissolved in EtOAc/MeOH 9:1 (10 mL). The resulting solution was washed with saturated aq. NaHCO₃, followed by brine. The organic layer was dried over anhydrous MgSO₄, filtered and concentrated. The crude residue was purified by flash chromatography using silica gel (MeOH/DCM, 0:100 to 10:90, gradient) to yield the compound A(V) as an off-white solid (22 mg, 22%). Purity was determined to be 95%. MS and NMR characterization is as follows: MS-HESI: calculated: 361.2670; observed: 361.2663 m/z [M+H]⁺. ¹H NMR (400 MHz, Methanol-d4): δ 6.96-6.86 (m, 3H), 6.44-6.40 (m, 1H), 3.20-3.14 (m, 2H), 2.80-2.73 (m, 2H), 2.33 (s, 6H), 1.50-1.39 (m, 3H), 1.19 (d, J=7.5 Hz, 18H).

### Assessment of Cell Viability Upon Treatment of a Psilocin Derivative.

Cell viability was assessed as described for Example 1, except the compound with formula A(V) was evaluated in place of the compound with formula F(I). FIGS. 4B and 4C show cell viability assay results for compound with formula A(V), depicted on the x-axis as “A-V”.

### Radioligand Receptor Binding Assays.

Activity at 5-HT_{2A} receptor was assessed as described for Example 1, except the compound with formula A(V) was evaluated in place of the compound with formula F(I). FIG. 4D shows radioligand competition assay results for compound with formula A(V), depicted on the x-axis simply as “A-V”.

### Cell Lines and Control Ligands Used to Assess Activity at 5-HT_{1A}.

Cell lines, cell line maintenance, and experimental procedures assessing modulation of 5-HT_{1A} were performed as described in Example 1, except that compound A(V) was evaluated in place of compound F(I). 5-HT_{1A} receptor binding evaluation for compound with formula A(V) (designated simply “A-V” along the x-axis) is shown in FIG. 4E. Comparison of data acquired in +5-HT_{1A} cultures with those acquired in −5-HT_{1A} cultures reveals receptor modulation by compound A(V).

### Evaluation of Metabolic Stability in Human Intestine, Liver, and Serum Fractions In Vitro.

Evaluations of metabolic stability and capacity of novel molecules to release psilocin under various in vitro conditions were performed as described in Example 1, except that compound with formula A(V) was used in place of compound with formula F(I) for all experiments. FIGS. 4F(i) and 4F(ii) show the metabolic stability curves for compound A(V) in assays containing Buffer (Panel A), AB serum (Panel B), HIM (Panel C), HIS9 (Panel D), HLM (Panel E), and HLS9 (Panel F).

### In Vivo Evaluation of 5-HT_{2A} Receptor Agonism in Mice.

Evaluation of in vivo HTR was conducted as described in Example 1, except that the compound with formula A(V) was used in place of the compound with formula F(I). Elevated incidences of HTR within the defined period of monitoring was observed in (1) psilocybin-treated mice, and (2) those treated with compound “A-V,” relative to control mice treated with i.p. injected vehicle (0.9% NaCl). These results are illustrated in FIG. 4G, wherein compound with formula A(V) is designated simply “A-V”.

### In Vitro Survey of Pharmacological Interaction Profiles at Receptors, Transporters and Enzymes Linked to Targeted Health Conditions.

All assays were performed as described in Example 1, except compound A(V) was used in place of F(I). To assess assay performance and establish positive control benchmarks, ligands listed in Table 1A were evaluated alongside test derivative. Results for all assays using compound A(V) (designated “A-V”) or positive controls are shown in Table 2.

TABLE 2
Data summary table of target assays for compound A(V) (designated “A-
V”) and control ligands. Potency (EC₅₀ or IC₅₀) is provided in units of μM.

| Target | Target | Assay | EC₅₀ | IC₅₀ | EC₅₀ | IC₅₀ |
|---|---|---|---|---|---|---|
| name | type | type | (control) | (control) | (A-V) | (A-V) |
| ADRA1A | GPCR | AGN | 5.00E−05 | — | >100 | — |
| ADRA1A | GPCR | ANT | — | 9.60E−04 | — | 19.63 |
| ADRA2A | GPCR | AGN | 4.00E−05 | — | >100 | — |
| ADRA2A | GPCR | ANT | — | 3.10E−03 | — | >100 |
| AVPR1A | GPCR | AGN | 4.20E−04 | — | >100 | — |
| AVPR1A | GPCR | ANT | — | 1.60E−03 | — | 17.05 |
| CHRM1 | GPCR | AGN | 9.70E−03 | — | >100 | — |
| CHRM1 | GPCR | ANT | — | 6.10E−03 | — | >100 |
| CHRM2 | GPCR | AGN | 2.70E−02 | — | >100 | — |
| CHRM2 | GPCR | ANT | — | 3.20E−03 | — | >100 |
| CNR1 | GPCR | AGN | 1.00E−05 | — | >100 | — |
| CNR1 | GPCR | ANT | — | 6.20E−04 | — | >100 |
| DRD1 | GPCR | AGN | 9.10E−02 | — | >100 | — |
| DRD1 | GPCR | ANT | — | 7.10E−04 | — | 9.67 |
| DRD2S | GPCR | AGN | 5.10E−04 | — | >100 | — |
| DRD2S | GPCR | ANT | — | 9.60E−04 | — | >100 |
| HTR1A | GPCR | AGN | 1.70E−03 | — | 10.72 | — |
| HTR1A | GPCR | ANT | — | 4.60E−02 | — | >100 |
| HTR1B | GPCR | AGN | 9.00E−05 | — | 1.19 | — |
| HTR1B | GPCR | ANT | — | 5.80E−03 | — | >100 |
| HTR2A | GPCR | AGN | 4.50E−03 | — | >100 | — |
| HTR2A | GPCR | ANT | — | 2.20E−03 | — | 6.95 |
| HTR2B | GPCR | AGN | 6.30E−04 | — | >100 | — |
| HTR2B | GPCR | ANT | — | 4.00E−04 | — | 3.14 |
| OPRD1 | GPCR | AGN | 5.00E−05 | — | 33.27 | — |
| OPRD1 | GPCR | ANT | — | 5.80E−04 | — | >100 |
| GABAA | Ion | OP | 6.2 | — | >100 | — |
| | channel | | | | | |
| GABAA | Ion | BL | — | 4.6 | — | 27.73 |
| | channel | | | | | |
| HTR3A | Ion | OP | 3.00E−01 | — | >100 | — |
| | channel | | | | | |
| HTR3A | Ion | BL | — | 1.90E−03 | — | 1.43 |
| | channel | | | | | |
| MAO-A | Enzyme | IN | — | 2.90E−03 | — | 48.79 |
| DAT | transporter | BL | — | 1.40E−03 | — | >100 |
| NET | transporter | BL | — | 6.70E−03 | — | 18.9 |
| SERT | transporter | BL | — | 1.80E−03 | — | >100 |
| NMDAR | Ion | BL | — | 8.00E−02 | — | 10.15 |
| | channel | | | | | |
| NMDAR | Ion | OP | 4.40E−01 | — | 87.46 | — |
| | channel | | | | | |

AGN, agonist; ANT, antagonist; OP, opener; BL, blocker; IN, inhibitor.

### Example 3—Synthesis and Analysis of a Second C₄-Ether-Substituted Tryptamine Derivative

Referring to FIG. 5A, compound 1 (100 mg, 0.49 mmol, 1.0 eq) and imidazole (100 mg, 1.5 mmol, 3.0 eq) were dissolved in anhydrous dimethylformamide (1.5 mL) under argon atmosphere, to which tert-butyl(chloro)diphenylsilane (153 μL, 0.59 mmol, 1.2 eq) was added. Notably, the synthesis of psilocin (1) has been described previously (Shirota et al., J. Nat. Prod. 2003, 66:885-887; Kargbo et al., ACS Omega 2020, 5:16959-16966). The solution was allowed to stir for 27 hours until complete as determined by TLC (20% methanol-dichloromethane). The solvent was removed under reduced pressure at 80° C. to dryness. The crude product was purified by flash column chromatography on 12 g normal-phase silica using a 0 to 10% methanol-dichloromethane gradient as eluent. The resulting material was dissolved in ethyl acetate (15 mL) and washed with water (3×10 mL) to remove residual imidazole. The organic phase was dried with magnesium sulphate and removed under reduced pressure to yield the compound A(III) (72 mg, 33%) as a white solid. Purity was determined to be 95%. MS and NMR characterization is as follows: MS-HESI: calculated: 443.2513; observed: 443.2509 m/z [M+H]⁺. ⁴H NMR (400 MHz, Methanol-d4): δ 7.85-7.78 (m, 4H), 7.49-7.34 (m, 6H), 6.99 (d, J=1.3 Hz, 1H), 6.86 (dt, J=8.1, 0.6 Hz, 1H), 6.57-6.48 (m, 1H), 6.02-5.96 (m, 1H), 3.38 (d, J=7.6 Hz, 2H), 2.91-2.84 (m, 2H), 2.37-2.30 (m, 6H), 1.15 (s, 9H).

### Assessment of Cell Viability Upon Treatment of a Psilocin Derivative.

Cell viability was assessed as described for Example 1, except the compound with formula A(III) was evaluated in place of the compound with formula F(I). Data acquired for the derivative having chemical formula A(III) is displayed as “A-III” on the x-axis in FIGS. 5B and 5C.

### Radioligand Receptor Binding Assays.

Activity at 5-HT_{2A} receptor was assessed as described for Example 1, except the compound with formula A(III) was evaluated in place of the compound with formula F(I). FIG. 5D shows radioligand competition assay results for compound with formula A(III), depicted on the x-axis simply as “A-III”.

### Cell Lines and Control Ligands Used to Assess Activity at 5-HT_{1A}.

Cell lines, cell line maintenance, and experimental procedures assessing modulation of 5-HT_{1A} were performed as described in Example 1, except that the compound with formula A(III) was evaluated in place of the compound with formula F(I). 5-HT_{1A} receptor binding evaluation for compound with formula A(III) (designated simply “A-III” along the x-axis) is shown in FIG. 5E. Comparison of data acquired in +5-HT_{1A} cultures with those acquired in −5-HT_{1A} cultures suggests receptor modulation at higher ligand concentrations.

### Evaluation of Metabolic Stability in Human Intestine, Liver, and Serum Fractions In Vitro.

Evaluations of metabolic stability and capacity of novel molecules to release psilocin under various in vitro conditions were performed as described in Example 1, except that the compound with formula A(III) was evaluated in place of the compound with formula F(I) for all experiments. FIGS. 5F (i) and 5F(ii) shows the metabolic stability curves for compound A(III) in assays containing Buffer (Panel A), AB serum (Panel B), HIM (Panel C), HIS9 (Panel D), HLM (Panel E) and HLS9 (Panel F).

### In Vivo Evaluation of 5-HT_{2A} Receptor Agonism in Mice.

Evaluation of in vivo HTR was conducted as described in Example 1, except that the compound with formula A(III) was used in place of the compound with formula F(I). Elevated incidences of HTR within the defined period of monitoring was observed in (1) psilocybin-treated mice, and (2) those treated with compound A(III) relative to control mice treated with i.p. injected vehicle (0.9% NaCl). These results are illustrated in FIG. 5G, wherein compound with formula A(III) is designated simply “A-III”.

### Example 4—Synthesis and Analysis of a First C₄-Carbonic Ester-Substituted Tryptamine Derivative

Referring to FIG. 6A, compound 1 (100 mg, 0.49 mmol, 1.0 eq) was dissolved in anhydrous dichloromethane (2 mL) under argon. Notably, the synthesis of psilocin (1) has been described previously (Shirota et al., J. Nat. Prod. 2003, 66:885-887; Kargbo et al., ACS Omega 2020, 5:16959-16966). To this solution, triethylamine (137 μL, 0.98 mmol, 2.0 eq) followed by 2,2,2-trichloroethyl chloroformate (135 μL, 0.98 mmol, 2.0 eq) were added. The mixture was stirred at room temperature for 4 h until complete as determined via TLC (20% methanol-dichloromethane). After dilution with dichloromethane (10 mL), the reaction mixture was washed with brine (15 mL), dried with magnesium sulphate, and concentrated under reduced pressure. The crude product was purified by flash chromatography on 12 g normal-phase silica using a 2.5 to 10% methanol-dichloromethane gradient as eluent to yield the compound B(IV) (10 mg, 5.5%) as a colourless oil. Purity was determined to be 95%. MS and NMR characterization is as follows: MS-HESI: calculated: 379.0378; observed: 379.0378 m/z [M+H]⁺. ¹H NMR (400 MHz, Methanol-d4) δ 7.69 (dd, J=8.3, 0.8 Hz, 1H), 7.36 (d, J=1.0 Hz, 1H), 7.14 (t, J=8.1 Hz, 1H), 6.65 (dd, J=7.9, 0.8 Hz, 1H), 5.15 (s, 2H), 3.08-3.01 (m, 2H), 2.80 (dd, J=7.5, 6.7 Hz, 2H), 2.39 (s, 6H).

### Assessment of Cell Viability Upon Treatment of a Psilocin Derivative.

Cell viability was assessed as described for Example 1, except the compound with formula B(IV) was evaluated in place of the compound with formula F(I) and additional assays were conducted using ligands psilocybin, psilocin, and DMT for comparative purposes. Data acquired for the derivative having chemical formula B(IV) is displayed as “B-IV” on the x-axes of FIG. 6B and FIG. 6C.

### Radioligand Receptor Binding Assays.

Activity at 5-HT_{2A} receptor was assessed as described for Example 1, except the compound with B(IV) was evaluated in place of the compound with formula F(I). FIG. 6D shows radioligand competition assay results for compound with formula B(IV), depicted on the x-axis simply as “B-IV”.

### Cell Lines and Control Ligands Used to Assess Activity at 5-HT_{1A}.

Cell lines, cell line maintenance, and experimental procedures assessing modulation of 5-HT_{1A} were performed as described in Example 1, except that B(IV) was evaluated in place of the compound with formula F(I). 5-HT_{1A} receptor binding evaluation for compound with formula B(IV) (designated simply “B-IV” along the x-axis) is shown in FIG. 6E. Comparison of data acquired in +5-HT_{1A} cultures with those acquired in −5-HT_{1A} cultures suggests no receptor modulation.

### Evaluation of Metabolic Stability in Human Intestine, Liver, and Serum Fractions In Vitro.

Evaluations of metabolic stability and capacity of novel molecules to release psilocin under various in vitro conditions were performed as described in Example 1, except that compound with formula B(IV) was used in place of the compound with formula F(I) for all experiments. FIGS. 6F (i)-6F (ii) show the metabolic stability curves for compound B(IV) in assays containing Buffer (Panel A), AB serum (Panel B), HIM (Panel C), HIS9 (Panel D), HLM (Panel E) and HLS9 (Panel F).

### In Vivo Evaluation of 5-HT_{2A} Receptor Agonism in Mice.

Evaluation of in vivo HTR was conducted as described in Example 1, except that compound with formula B(IV) was used in place of the compound with formula F(I). Elevated incidences of HTR within the defined period of monitoring was observed in (1) psilocybin-treated mice, and (2) those treated with compound B(IV), relative to control mice treated with i.p. injected vehicle (0.9% NaCl). These results are illustrated in FIG. 6G, wherein compound with formula B(IV) is designated simply “B-IV”. Results for control mice injected with vehicle are not shown in FIG. 6G but are the same as those in Example 1 (FIG. 3P) since HTR experiments were run with the same control cohorts.

### Example 5—Synthesis and Analysis of a Second C₄-Carbonic Ester-Substituted Tryptamine Derivative

Referring to FIG. 7A, to a suspension of psilocin 1 (50 mg, 0.25 mmol, 1.0 eq) in dry DCM under argon atmosphere, triethylamine (68 μL, 0.49 mmol, 2.0 eq) and hexyl chloroformate (0.16 mL, 0.98 mmol, 4.0 eq) were added. Notably, the synthesis of psilocin (1) has been described previously (Shirota et al., J. Nat. Prod. 2003, 66:885-887; Kargbo et al., ACS Omega 2020, 5:16959-16966). The reaction mixture was stirred at room temperature for 4 h until no starting material was present, as monitored by TLC (20% methanol-DCM). The reaction mixture was diluted with DCM (10 mL), washed with brine (2×15 mL), dried over anhydrous magnesium sulphate, and concentrated under reduced pressure. Purification by column chromatography on 4 g normal-phase silica using a 1 to 2% methanol-DCM eluent gradient yielded the compound B(V) as a white solid (10 mg, 0.030 mmol, 12%). MS characterization is as follows: MS-HESI: calculated: 333.2173; observed: 333.2164 m/z [M+H]⁺.

### Assessment of Cell Viability Upon Treatment of a Psilocin Derivative.

Cell viability was assessed as described for Example 1, except that compound with formula B(V) was evaluated in place of the compound with formula F(I) and additional assays were conducted using ligands psilocybin, psilocin, and DMT for comparative purposes. Data acquired for the derivative having chemical formula B(V) is displayed as “B-V” on the x-axes of FIG. 7B and FIG. 7C.

### Radioligand Receptor Binding Assays.

Activity at 5-HT_{2A} receptor was assessed as described for Example 1, except the compound with formula B(V) was evaluated in place of the compound with formula F(I). FIG. 7D shows radioligand competition assay results for compound with formula B(V), depicted on the x-axis simply as “B-V”.

### Cell Lines and Control Ligands Used to Assess Activity at 5-HT_{1A}.

Cell lines, cell line maintenance, and experimental procedures assessing modulation of 5-HT_{1A} were performed as described in Example 1, except that the compound with formula B(V) was evaluated in place of the compound with formula F(I). 5-HT_{1A} receptor binding evaluation, including EC₅₀ for compound with formula B(V) (designated simply “B-V” along the x-axis) is shown in FIG. 7E. Comparison of data acquired in +5-HT_{1A} cultures with those acquired in −5-HT_{1A} cultures suggests no receptor modulation.

### In Vivo Evaluation of 5-HT_{2A} Receptor Agonism in Mice.

Evaluation of in vivo HTR was conducted as described in Example 1, except that compound with formula B(V) was used in place of the compound with formula F(I). Elevated incidences of HTR within the defined period of monitoring was observed in (1) psilocybin-treated mice, and (2) those treated with compound B(IV), relative to control mice treated with i.p. injected vehicle (0.9% NaCl). These results are illustrated in FIG. 7F, wherein compound with formula B(V) is designated simply “B-V”. Results for control mice injected with vehicle are not shown in FIG. 7F but are the same as those in Example 1 (FIG. 3P) since HTR experiments were run with the same control cohorts.

### Example 6—Synthesis and Analysis of a Third C₄-Carbonic Ester-Substituted Tryptamine Derivative

Referring to FIG. 8A, to a suspension of 1 (100 mg, 0.49 mmol, 1.0 eq) and potassium carbonate (68 mg, 0.49 mmol, 1.0 eq) in dry DMF (1.2 mL) under argon atmosphere, benzyl chloroformate (70 μL, 0.49 mmol, 1.0 eq) was added. Notably, the synthesis of psilocin (1) has been described previously (Shirota et al., J. Nat. Prod. 2003, 66:885-887; Kargbo et al., ACS Omega 2020, 5:16959-16966). The reaction was allowed to stir at room temperature for 4 hours until completion as determined by TLC (20% methanol-DCM). Following dilution with water (10 mL), the reaction mixture was extracted with DCM (3×10 mL). The combined organic extracts were washed with brine (10 mL), dried over anhydrous magnesium sulphate, and concentrated under reduced pressure. Purification via flash column chromatography using 12 g normal-phase silica using a 0 to 10% methanol-dichloromethane gradient yielded the compound B(II) (38 mg, 23%) as a light-yellow oil. Purity was determined to be 95%. MS and NMR characterization are as follows: MS-HESI: calculated: 339.1703; observed: 437.2275 m/z [M+H]⁺. ¹H NMR (400 MHz, MeOD): δ 7.63 (d, J=8.3 Hz, 1H), 7.55-7.49 (m, 2H), 7.46-7.36 (m, 3H), 7.33 (s, 1H), 7.09 (t, J=8.1 Hz, 1H), 6.60 (dd, J=7.9, 0.8 Hz, 1H), 5.44 (s, 2H), 3.02 (td, J=7.3, 1.0 Hz, 2H), 2.78 (t, J=7.1 Hz, 2H), 2.38 (s, 6H).

### Assessment of Cell Viability Upon Treatment of a Psilocin Derivative.

Cell viability was assessed as described for Example 1, except that the compound with formula B(II) was evaluated in place of the compound with formula F(I) and additional assays were conducted using ligands psilocybin and psilocin for comparative purposes. Data acquired for the derivative having chemical formula (B-II) is displayed as “B-II” on the x-axes of FIG. 8B and FIG. 8C.

### Radioligand Receptor Binding Assays.

Activity at 5-HT_{2A} receptor was assessed as described for Example 1, except the compound with formula B(II) was evaluated in place of the compound with formula F(I). FIG. 8D shows radioligand competition assay results for compound with formula B(II), depicted on the x-axis simply as “B-II”.

### Cell Lines and Control Ligands Used to Assess Activity at 5-HT_{1A}.

Cell lines, cell line maintenance, and experimental procedures assessing modulation of 5-HT_{1A} were performed as described in Example 1, except that the compound with formula B(II) was evaluated in place of the compound with formula F(I). 5-HT_{1A} receptor binding evaluation for compound with formula B(II) (designated simply “B-II” along the x-axis) is shown in FIG. 8E. Comparison of data acquired in +5-HT_{1A} cultures with those acquired in −5-HT_{1A} cultures suggests no receptor modulation.

### Evaluation of Metabolic Stability in Human Intestine, Liver, and Serum Fractions In Vitro.

Evaluations of metabolic stability and capacity of novel molecules to release psilocin under various in vitro conditions were performed as described in Example 1, except that the compound with formula B(II) was used in place of the compound with formula F(I) for all experiments. FIGS. 8F (i)-8F(ii) show the metabolic stability curves for compound B(II) in assays containing Buffer (Panel A), AB serum (Panel B), HIM (Panel C), HIS9 (Panel D), HLM (Panel E) and HLS9 (Panel F).

In vivo evaluation of 5-HT_{2A} receptor agonism in mice.

Evaluation of in vivo HTR was conducted as described in Example 1, except that the compound with formula B(II) was used in place of the compound with formula F(I). Elevated incidences of HTR within the defined period of monitoring was observed in (1) psilocybin-treated mice, and (2) those treated with compound B(II), relative to control mice treated with i.p. injected vehicle (0.9% NaCl). These results are illustrated in FIG. 8G, wherein compound with formula B(II) is designated “B-II”.

### In Vitro Survey of Pharmacological Interaction Profiles at Receptors, Transporters and Enzymes Linked to Targeted Health Conditions.

All assays were performed as described in Example 1, except compound B(II) was used in place of F(I). To assess assay performance and establish positive control benchmarks, ligands listed in Table 1A were evaluated alongside test derivative. Results for all assays using compound B(II) (designated “B-II”) or positive controls are shown in Table 3.

TABLE 3
Data summary table of target assays for compound B(II) (designated “B-
II”) and control ligands. Potency (EC₅₀ or IC₅₀) is provided in units of μM.

| Target | Target | Assay | EC₅₀ | IC₅₀ | EC₅₀ | IC₅₀ |
|---|---|---|---|---|---|---|
| name | type | type | (control) | (control) | (B-II) | (B-II) |
| ADRA1A | GPCR | AGN | 5.00E−05 | — | >100 | — |
| ADRA1A | GPCR | ANT | — | 9.60E−04 | — | 3.66 |
| ADRA2A | GPCR | AGN | 4.00E−05 | — | >100 | — |
| ADRA2A | GPCR | ANT | — | 3.10E−03 | — | 9.08 |
| AVPR1A | GPCR | AGN | 4.20E−04 | — | >100 | — |
| AVPR1A | GPCR | ANT | — | 1.60E−03 | — | 42.64 |
| CHRM1 | GPCR | AGN | 9.70E−03 | — | >100 | — |
| CHRM1 | GPCR | ANT | — | 6.10E−03 | — | 24.54 |
| CHRM2 | GPCR | AGN | 2.70E−02 | — | >100 | — |
| CHRM2 | GPCR | ANT | — | 3.20E−03 | — | >100 |
| CNR1 | GPCR | AGN | 1.00E−05 | — | >100 | — |
| CNR1 | GPCR | ANT | — | 6.20E−04 | — | 68.63 |
| DRD1 | GPCR | AGN | 9.10E−02 | — | >100 | — |
| DRD1 | GPCR | ANT | — | 7.10E−04 | — | 5.39 |
| DRD2S | GPCR | AGN | 5.10E−04 | — | >100 | — |
| DRD2S | GPCR | ANT | — | 9.60E−04 | — | 12.39 |
| HTR1A | GPCR | AGN | 1.70E−03 | — | 13.6 | — |
| HTR1A | GPCR | ANT | — | 4.60E−02 | — | >100 |
| HTR1B | GPCR | AGN | 9.00E−05 | — | 4.46E−01 | — |
| HTR1B | GPCR | ANT | — | 5.80E−03 | — | >100 |
| HTR2A | GPCR | AGN | 4.50E−03 | — | >100 | — |
| HTR2A | GPCR | ANT | — | 2.20E−03 | — | 9.08E−01 |
| HTR2B | GPCR | AGN | 6.30E−04 | — | >100 | — |
| HTR2B | GPCR | ANT | — | 4.00E−04 | — | 2.81E−01 |
| OPRD1 | GPCR | AGN | 5.00E−05 | — | >100 | — |
| OPRD1 | GPCR | ANT | — | 5.80E−04 | — | >100 |
| GABAA | Ion | OP | 6.2 | — | >100 | — |
| | channel | | | | | |
| GABAA | Ion | BL | — | 4.6 | — | 21 |
| | channel | | | | | |
| HTR3A | Ion | OP | 3.00E−01 | — | >100 | — |
| | channel | | | | | |
| HTR3A | Ion | BL | — | 1.90E−03 | — | 2.34 |
| | channel | | | | | |
| MAO-A | Enzyme | IN | — | 2.90E−03 | — | 21.86 |
| DAT | transporter | BL | — | 1.40E−03 | — | 4.69 |
| NET | transporter | BL | — | 6.70E−03 | — | 26.53 |
| SERT | transporter | BL | — | 1.80E−03 | — | 10.36 |
| NMDAR | Ion | BL | — | 8.00E−02 | — | 16.02 |
| | channel | | | | | |
| NMDAR | Ion | OP | 4.40E−01 | — | >100 | — |
| | channel | | | | | |

AGN, agonist; ANT, antagonist; OP, opener; BL, blocker; IN, inhibitor.

### Example 7—Synthesis and Analysis of a Fourth C₄-Carbonic Ester-Substituted Tryptamine Derivative

Referring to FIGS. 9A (i) and 9A (ii), a dry, 3-neck RBF was charged with 4-benzyloxyindole 1 (14.0 g, 62.7 mmol) and Di-ethyl ether (327 mL) under Ar. The mixture was cooled down to 0° C. in an ice bath. An Argon sparge was placed on the RBF and into the reaction mixture to purge out the HCl gas released from the reaction. Oxalyl chloride (10.9 mL, 129 mmol) was added dropwise over 40 min, while maintaining the cold temperature. The mixture was stirred for 4 h at 0° C. to yield compound 2. The Argon sparge was removed, and dimethylamine (157 mL, 314 mmol) (2 M in THF) was added dropwise at 0° C. over 1 h using an addition funnel. The mixture was allowed to warm up to RT and stir overnight. Diethyl ether (200 mL) was added, and the mixture was cooled down to 0° C. The resulting precipitate (crude 3) was filtered and transferred to an erlenmeyer flask. The solid was suspended in water (300 mL) and stirred for 30 min. Then, it was filtered and washed with more H₂O to remove residual salts. The crude solid was further dried in vacuo and used in the next step without further purification.

Continuing to refer to FIG. 9A (i), lithium aluminum hydride (60.2 mL, 120 mmol) (2 M in THF) was added to a dry 3-neck flask under argon. The flask was fitted with a reflux condenser and an addition funnel. Dry 1,4-dioxane (100 mL) was added, and the mixture was heated to 60° C. in an oil bath. In a separate flask, compound 3 (7.46 g, 23.1 mmol) was dissolved in a mixture of THF (60 mL) and 1,4-dioxane (120 mL). With rapid stirring, this solution was added dropwise to the reaction flask over 1 h using an addition funnel. The oil bath temperature was held at 70° C. for 4 h, followed by vigorous reflux overnight (16 h) in an oil bath temperature of 95° C.

Continuing to refer to FIG. 9A (i), the reaction was placed in an ice bath, and a solution of distilled H₂O (25 mL) in THF (65 mL) was added dropwise to quench lithium aluminum hydroxide, resulting in a gray flocculent precipitate. Di-ethyl ether (160 mL) was added to assist breakup of the complex and improve filtration. This slurry was stirred for 1 h and the mixture was then filtered using a Buchner funnel. The filter cake was washed on the filter with warm Et₂O (2×200 mL) and was broken up, transferred back into the reaction flask, and vigorously stirred with additional warm diethyl ether (300 mL). This slurry was filtered, and the cake was washed on the filter with diethyl ether (120 mL) and hexane (2×120 mL). All the organic filtrates were combined and dried (MgSO₄). After the drying agent was removed by filtration, the filtrate was concentrated under vacuum and dried under high vacuum. The crude residue was triturated with EtOAc/hexane (1:9, 25 mL) to afford the crude product (4) which was used in the next step without further purification.

Continuing to refer to FIG. 9A (i), to a solution of 4 (5.00 g, 17.0 mmol) in dry THE (100 mL) cooled to −78° C. under argon, 1 M solution of KHMDS (18.7 mL, 18.7 mmol) in THE was added dropwise. After stirring at −78° C. for 1 h, a solution of TIPSCl (3.82 mL, 17.8 mmol) in THE (19.0 mL) was added dropwise over 15 minutes, and the reaction mixture was allowed to warm up to RT. After stirring at RT for 1 h, the reaction was quenched with H₂O (40 mL), THE was evaporated under reduced pressure, and the aqueous solution was further diluted with H₂O (75 mL) and extracted with DCM (3×100 mL). The organic layers were combined and washed with brine, dried over Na₂SO₄, filtered, and concentrated under reduced pressure. The crude product was purified by flash column chromatography (MeOH/DCM 5:95 to 10:90) to yield the compound (5) as a light brown oil (6.99 g, 91%). Purity was determined to be 95%. NMR characterization is as follows: ¹H NMR (400 MHz, CDCl₃): δ 7.58-7.51 (m, 2H), 7.44-7.39 (m, 2H), 7.38-7.33 (m, 1H), 7.12 (dd, J=8.4, 0.8 Hz, 1H), 7.08-6.99 (m, 1H), 6.94 (s, 1H), 6.60 (dd, J=7.7, 0.7 Hz, 1H), 5.20 (s, 2H), 3.12-3.04 (m, 2H), 2.67-2.58 (m, 2H), 2.16 (s, 6H), 1.69 (h, J=7.5 Hz, 3H), 1.16 (d, J=7.5 Hz, 18H).

Continuing to refer to FIG. 9A (i), to a stirring solution of 5 (6.99 g, 15.5 mmol) dissolved in EtOH, 95% (310 mL), 10% Palladium on carbon (1.65 g, 1.55 mmol) was added. This mixture was put under vacuum for five minutes, then alternately purged with H₂ gas until pressurized hydrogen atmosphere was established, then allowed to stir for 75 minutes at room temperature. The palladium on carbon was removed by filtration through Celite, the filtrate dried with anhydrous magnesium sulphate, and concentrated under reduced pressure to yield the compound 6 (4.67 g, 84%) as an off-white solid. Purity was determined to be 95%. MS and NMR characterization is as follows: MS-ESI: calculated: 361.2670; observed: 361. 2668 m/z [M+H]⁺. ¹H NMR (400 MHz, MeOD): δ 6.98 (d, J=8.6 Hz, 2H), 6.91 (dd, J=8.4, 7.5 Hz, 1H), 6.42 (dd, J=7.5, 0.8 Hz, 1H), 3.06 (t, J=6.9 Hz, 2H), 2.77 (t, J=6.9 Hz, 2H), 2.39 (s, 6H), 1.72 (p, J=7.5 Hz, 3H), 1.16 (d, J=7.5 Hz, 18H).

Referring next to FIG. 9A (ii), a solution of 6 (250 mg, 693 μmol) and 4-Nitrophenyl chloroformate (154 mg, 763 μmol) in DCM (3.47 mL) was cooled down to 0° C., and it was added dropwise to N,N-diisopropylethylamine (242 μL, 1.39 mmol). The reaction was warmed up to RT and stirred for 2 h. After 2 h, TLC (MeOH/DCM 12:88) showed almost complete conversion to the desired product.

Continuing to refer to FIG. 9A (ii), to the reaction mixture (7), cyclopropanemethanol (168 μL, 2.08 mmol) and N,N-diisopropylethylamine (362 μL, 2.08 mmol) were added with vigorous stirring at RT. After 2 h, volatiles were removed in vacuo and the crude mixture was purified on silica gel (MeOH/DCM 1:9) to afford the semi-pure product that contained p-nitrophenol. This material was dissolved in DCM (10 mL) and washed with saturated aq. NaHCO₃ (6×20 mL) to remove the p-nitrophenol. The compound 8 was obtained as a light-yellow oil (185 mg, 58%). Purity was determined to be 95%. MS and NMR characterization is as follows: MS-HESI: calculated: 459.3037; observed: 459.3031 m/z [M+H]⁺. ¹H NMR (400 MHz, CDCl₃) δ 7.34 (d, J=8.3 Hz, 1H), 7.09 (t, J=8.1 Hz, 1H), 7.01 (s, 1H), 6.92 (d, J=7.7 Hz, 1H), 4.12 (d, J=7.4 Hz, 2H), 3.02-2.94 (m, 2H), 2.66 (t, J=8.2 Hz, 2H), 2.38 (s, 6H), 1.66 (h, J=7.5 Hz, 3H), 1.31-1.22 (m, 1H), 1.13 (d, J=7.5 Hz, 18H), 0.69-0.58 (m, 2H), 0.43-0.34 (m, 2H).

Continuing to refer to FIG. 9A (ii), to a solution of crude 8 (165 mg, 360 μmol) in dry THE (1.80 mL), tetrabutylammonium fluoride solution (1 M in THF, 540 μL, 540 μmol) was added dropwise at 0° C. After 1 h, water (2 mL) was added, the aq. layer was separated and extracted with DCM (3×15 mL). The combined organic layers were washed with brine, dried over anhydrous Na₂SO₄, filtered and concentrated. The crude product was purified by flash chromatography using silica gel (MeOH/DCM 8:92) to yield the compound B(III) as a colorless oil (32 mg, 29%). Purity was determined to be 95%. MS and NMR characterization is as follows: MS-ESI: calculated: 303.1703; observed: 303.1702 m/z [M+H]⁺. ¹H NMR (400 MHz, CDCl₃) δ 8.35 (s, 1H), 7.22 (d, J=8.1 Hz, 1H), 7.13 (t, J=7.9 Hz, 1H), 6.97 (dt, J=2.1, 1.0 Hz, 1H), 6.91 (d, J=7.7 Hz, 1H), 4.12 (d, J=7.4 Hz, 2H), 3.03-2.93 (m, 2H), 2.72-2.65 (m, 2H), 2.38 (s, 6H), 1.35-1.21 (m, 1H), 1.17 (t, J=7.2 Hz, 1H), 0.71-0.59 (m, 2H), 0.43-0.34 (m, 2H).

### Assessment of Cell Viability Upon Treatment of a Psilocin Derivative.

Cell viability was assessed as described for Example 1, except that the compound with formula B(III) was evaluated in place of the compound with formula F(I) and additional assays were conducted using ligands psilocin and tryptophan for comparative purposes. Data acquired for the derivative having chemical formula (B-III) is displayed as “B-III” on the x-axes of FIG. 9B and FIG. 9C.

### Radioligand Receptor Binding Assays.

Activity at 5-HT_{2A} receptor was assessed as described for Example 1, except the compound with formula B(III) was evaluated in place of the compound with formula F(I). FIG. 9D shows radioligand competition assay results for compound with formula B(III), depicted on the x-axis simply as “B-III”.

### Cell Lines and Control Ligands Used to Assess Activity at 5-HT_{1A}.

Cell lines, cell line maintenance, and experimental procedures assessing modulation of 5-HT_{1A} were performed as described in Example 1, except that the compound with formula B(III) was evaluated in place of the compound with formula F(I). 5-HT_{1A} receptor binding evaluation for compound with formula B(III) (designated simply “B-III” along the x-axis) is shown in FIG. 9E. Comparison of data acquired in +5-HT_{1A} cultures with those acquired in −5-HT_{1A} cultures indicates receptor modulation.

### Evaluation of Metabolic Stability in Human Intestine, Liver, and Serum Fractions In Vitro.

Evaluations of metabolic stability and capacity of novel molecules to release psilocin under various in vitro conditions were performed as described in Example 1, except that the compound with formula B(III) was used in place of the compound with formula F(I) for all experiments. FIGS. 9F (i)-9F(ii) show the metabolic stability curves for compound B(III) in assays containing Buffer (Panel A), AB serum (Panel B), HIM (Panel C), HIS9 (Panel D), HLM (Panel E) and HLS9 (Panel F).

### In Vivo Evaluation of 5-HT_{2A} Receptor Agonism in Mice.

Evaluation of in vivo HTR was conducted as described in Example 1, except that the compound with formula B(III) was used in place of the compound with formula F(I). Elevated incidences of HTR within the defined period of monitoring was observed in (1) psilocybin-treated mice, and (2) those treated with compound B(III), relative to control mice treated with i.p. injected vehicle (0.9% NaCl). These results are illustrated in FIG. 9G, wherein compound with formula B(III) is designated “B-III”.

### In Vitro Survey of Pharmacological Interaction Profiles at Receptors, Transporters and Enzymes Linked to Targeted Health Conditions.

All assays were performed as described in Example 1, except compound B(III) was used in place of F(I). To assess assay performance and establish positive control benchmarks, ligands listed in Table 1A were evaluated alongside test derivative. Results for all assays using compound B(III) (designated “B-III”) or positive controls are shown in Table 4.

TABLE 4
Data summary table of target assays for compound B(III) (designated “B-
III”) and control ligands. Potency (EC₅₀ or IC₅₀) is provided in units of μM.

| Target | Target | Assay | EC₅₀ | IC₅₀ | EC₅₀ | IC₅₀ |
|---|---|---|---|---|---|---|
| name | type | type | (control) | (control) | (B-III) | (B-III) |
| ADRA1A | GPCR | AGN | 5.00E−05 | — | >100 | — |
| ADRA1A | GPCR | ANT | — | 9.60E−04 | — | 12.61 |
| ADRA2A | GPCR | AGN | 4.00E−05 | — | >100 | — |
| ADRA2A | GPCR | ANT | — | 3.10E−03 | — | >100 |
| AVPR1A | GPCR | AGN | 4.20E−04 | — | >100 | — |
| AVPR1A | GPCR | ANT | — | 1.60E−03 | — | >100 |
| CHRM1 | GPCR | AGN | 9.70E−03 | — | >100 | — |
| CHRM1 | GPCR | ANT | — | 6.10E−03 | — | 52.97 |
| CHRM2 | GPCR | AGN | 2.70E−02 | — | >100 | — |
| CHRM2 | GPCR | ANT | — | 3.20E−03 | — | >100 |
| CNR1 | GPCR | AGN | 1.00E−05 | — | >100 | — |
| CNR1 | GPCR | ANT | — | 6.20E−04 | — | >100 |
| DRD1 | GPCR | AGN | 9.10E−02 | — | >100 | — |
| DRD1 | GPCR | ANT | — | 7.10E−04 | — | 13.35 |
| DRD2S | GPCR | AGN | 5.10E−04 | — | 3.81 | — |
| DRD2S | GPCR | ANT | — | 9.60E−04 | — | >100 |
| HTR1A | GPCR | AGN | 1.70E−03 | — | 7.45 | — |
| HTR1A | GPCR | ANT | — | 4.60E−02 | — | >100 |
| HTR1B | GPCR | AGN | 9.00E−05 | — | 6.18E−02 | — |
| HTR1B | GPCR | ANT | — | 5.80E−03 | — | >100 |
| HTR2A | GPCR | AGN | 4.50E−03 | — | >100 | — |
| HTR2A | GPCR | ANT | — | 2.20E−03 | — | 2.96E−01 |
| HTR2B | GPCR | AGN | 6.30E−04 | — | >100 | — |
| HTR2B | GPCR | ANT | — | 4.00E−04 | — | 2.15E−02 |
| OPRD1 | GPCR | AGN | 5.00E−05 | — | >100 | — |
| OPRD1 | GPCR | ANT | — | 5.80E−04 | — | >100 |
| GABAA | Ion | OP | 6.2 | — | >100 | — |
| | channel | | | | | |
| GABAA | Ion | BL | — | 4.6 | — | >100 |
| | channel | | | | | |
| HTR3A | Ion | OP | 3.00E−01 | — | >100 | — |
| | channel | | | | | |
| HTR3A | Ion | BL | — | 1.90E−03 | — | 47.37 |
| | channel | | | | | |
| MAO-A | Enzyme | IN | — | 2.90E−03 | — | >100 |
| DAT | transporter | BL | — | 1.40E−03 | — | >100 |
| NET | transporter | BL | — | 6.70E−03 | — | >100 |
| SERT | transporter | BL | — | 1.80E−03 | — | >100 |
| NMDAR | Ion | BL | — | 8.00E−02 | — | >100 |
| | channel | | | | | |
| NMDAR | Ion | OP | 4.40E−01 | — | >100 | — |
| | channel | | | | | |

AGN, agonist; ANT, antagonist; OP, opener; BL, blocker; IN, inhibitor.

### Example 8—Synthesis and Analysis of a First C₄-Polyether-Substituted Tryptamine Derivative

Referring to FIG. 10A, to a solution of psilocin (1) (102 mg, 500 μmol) in dry DMF (2 ml) under argon was added potassium carbonate (69 mg, 500 μmol) and potassium iodide (83 mg, 500 μmol). Notably, the synthesis of psilocin (1) has been described previously (Shirota et al., J. Nat. Prod. 2003, 66:885-887; Kargbo et al., ACS Omega 2020, 5:16959-16966). After stirring at room temperature for 10 minutes, Chloromethyl pentanoate (75.2 mg, 500 μmol) was added, and the resulting mixture was stirred for 3-4 days at room temperature. The reaction was monitored by TLC. TLC showed a large new spot with higher Rf value. The reaction was quenched by addition of water. The mixture was then extracted with dichloromethane and the organic phase was washed with brine and dried over anhydrous MgSO₄. After concentration by rotavapor, the resulting residue was purified by silica chromatography (7.5%-10% methanol in DCM) on the Combiflash system to provide the compound D(III) as a white solid (40.7 mg, yield 25.6%). Purity was determined to be 95%. NMR characterization is as follows: 1H NMR (400 MHz, MeOD): δ 7.47 (dd, J=8.3, 0.8 Hz, 1H), 7.39 (d, J=0.9 Hz, 1H), 7.26 (t, J=8.0 Hz, 1H), 6.95-6.84 (m, 1H), 6.17 (s, 2H), 3.53-3.38 (m, 2H), 3.21-3.10 (m, 2H), 2.91 (s, 6H), 2.78 (q, J=7.5 Hz, 2H), 2.34 (q, J=7.5 Hz, 2H), 1.30 (td, J=7.5, 1.9 Hz, 3H), 1.09 (t, J=7.5 Hz, 3H).

### Assessment of Cell Viability Upon Treatment of a Psilocin Derivative.

Cell viability was assessed as described for Example 1, except that the compound with formula D(III) was evaluated in place of the compound with formula F(I) and additional assays were conducted using ligands psilocin and tryptophan for comparative purposes. Data acquired for the derivative having chemical formula D(III) is displayed as “D-III” on the x-axes of FIG. 10B and FIG. 10C.

### Radioligand Receptor Binding Assays.

Activity at 5-HT_{2A} receptor was assessed as described for Example 1, except the compound with formula D(III) was evaluated in place of the compound with formula F(I). FIG. 10D shows radioligand competition assay results for compound with formula D(III), depicted on the x-axis simply as “D-III”.

### Cell Lines and Control Ligands Used to Assess Activity at 5-HT_{1A}.

Cell lines, cell line maintenance, and experimental procedures assessing modulation of 5-HT_{1A} were performed as described in Example 1, except that the compound with formula D(III) was evaluated in place of the compound with formula F(I). 5-HT_{1A} receptor binding evaluation for compound with formula D(III) (designated simply “D-III” along the x-axis) is shown in FIG. 10E. Comparison of data acquired in +5-HT_{1A} cultures with those acquired in −5-HT_{1A} cultures suggests no receptor modulation.

### Evaluation of Metabolic Stability in Human Intestine, Liver, and Serum Fractions In Vitro.

Evaluations of metabolic stability and capacity of novel molecules to release psilocin under various in vitro conditions were performed as described in Example 1, except that the compound with formula D(III) was used in place of the compound with formula F(I) for all experiments. FIGS. 10F (i)-10F(ii) show the metabolic stability curves for compound D(III) in assays containing Buffer (Panel A), AB serum (Panel B), HIM (Panel C), HIS9 (Panel D), HLM (Panel E) and HLS9 (Panel F).

### In Vivo Evaluation of 5-HTₐ Receptor Agonism in Mice.

Evaluation of in vivo HTR was conducted as described in Example 1, except that the compound with formula D(III) was used in place of the compound with formula F(I). Elevated incidences of HTR within the defined period of monitoring was observed in (1) psilocybin-treated mice, and (2) those treated with compound D(III), relative to control mice treated with i.p. injected vehicle (0.9% NaCl). These results are illustrated in FIG. 10G, wherein compound with formula D(III) is designated “D-III”.

### In Vitro Survey of Pharmacological Interaction Profiles at Receptors, Transporters and Enzymes Linked to Targeted Health Conditions.

All assays were performed as described in Example 1, except compound D(III) was used in place of F(I). To assess assay performance and establish positive control benchmarks, ligands listed in Table 1A were evaluated alongside test derivative. Results for all assays using compound D(III) (designated “D-III”) or positive controls are shown in Table 5.

TABLE 5
Data summary table of target assays for compound D(III) (designated “D-
III”) and control ligands. Potency (EC₅₀ or IC₅₀) is provided in units of μM.

| Target | Target | Assay | EC₅₀ | IC₅₀ | EC₅₀ | IC₅₀ |
|---|---|---|---|---|---|---|
| name | type | type | (control) | (control) | (D-III) | (D-III) |
| ADRA1A | GPCR | AGN | 5.00E−05 | — | >100 | — |
| ADRA1A | GPCR | ANT | — | 9.60E−04 | — | 3.86 |
| ADRA2A | GPCR | AGN | 4.00E−05 | — | >100 | — |
| ADRA2A | GPCR | ANT | — | 3.10E−03 | — | >100 |
| AVPR1A | GPCR | AGN | 4.20E−04 | — | >100 | — |
| AVPR1A | GPCR | ANT | — | 1.60E−03 | — | >100 |
| CHRM1 | GPCR | AGN | 9.70E−03 | — | >100 | — |
| CHRM1 | GPCR | ANT | — | 6.10E−03 | — | 52.58 |
| CHRM2 | GPCR | AGN | 2.70E−02 | — | >100 | — |
| CHRM2 | GPCR | ANT | — | 3.20E−03 | — | >100 |
| CNR1 | GPCR | AGN | 1.00E−05 | — | >100 | — |
| CNR1 | GPCR | ANT | — | 6.20E−04 | — | >100 |
| DRD1 | GPCR | AGN | 9.10E−02 | — | >100 | — |
| DRD1 | GPCR | ANT | — | 7.10E−04 | — | 32.25 |
| DRD2S | GPCR | AGN | 5.10E−04 | — | 17.75 | — |
| DRD2S | GPCR | ANT | — | 9.60E−04 | — | >100 |
| HTR1A | GPCR | AGN | 1.70E−03 | — | 28.66 | — |
| HTR1A | GPCR | ANT | — | 4.60E−02 | — | >100 |
| HTR1B | GPCR | AGN | 9.00E−05 | — | 2.79E−01 | — |
| HTR1B | GPCR | ANT | — | 5.80E−03 | — | >100 |
| HTR2A | GPCR | AGN | 4.50E−03 | — | >100 | — |
| HTR2A | GPCR | ANT | — | 2.20E−03 | — | 5.87E−01 |
| HTR2B | GPCR | AGN | 6.30E−04 | — | >100 | — |
| HTR2B | GPCR | ANT | — | 4.00E−04 | — | 3.01E−01 |
| OPRD1 | GPCR | AGN | 5.00E−05 | — | >100 | — |
| OPRD1 | GPCR | ANT | — | 5.80E−04 | — | >100 |
| GABAA | Ion | OP | 6.2 | — | >100 | — |
| | channel | | | | | |
| GABAA | Ion | BL | — | 4.6 | — | >100 |
| | channel | | | | | |
| HTR3A | Ion | OP | 3.00E−01 | — | >100 | — |
| | channel | | | | | |
| HTR3A | Ion | BL | — | 1.90E−03 | — | 3.24E+01 |
| | channel | | | | | |
| MAO-A | Enzyme | IN | — | 2.90E−03 | | >100 |
| DAT | transporter | BL | — | 1.40E−03 | — | >100 |
| NET | transporter | BL | — | 6.70E−03 | — | >100 |
| SERT | transporter | BL | — | 1.80E−03 | — | >100 |
| NMDAR | Ion | BL | — | 8.00E−02 | — | >100 |
| | channel | | | | | |
| NMDAR | Ion | OP | 4.40E−01 | — | >100 | — |
| | channel | | | | | |

AGN, agonist; ANT, antagonist; OP, opener; BL, blocker; IN, inhibitor.

### Example 9—Synthesis and Analysis of a Second C₄-Polyether-Substituted Tryptamine Derivative

Referring to FIG. 11A, to a solution of 1 (51 mg, 250 μmol) in dry DMF (2 ml) under argon, potassium carbonate (34.5 mg, 250 μmol) and potassium iodide (41.5 mg, 250 μmol) were added. Notably, the synthesis of psilocin (1) has been described previously (Shirota et al., J. Nat. Prod. 2003, 66:885-887; Kargbo et al., ACS Omega 2020, 5:16959-16966). After stirring at room temperature for 10 minutes, Chloromethyl butyrate (34.1 mg, 250 μmol) was added, and the resulting mixture was stirred for 3-4 days at room temperature. The reaction was monitored by TLC. TLC showed a large new spot with higher Rf value. The reaction was quenched by addition of water. The mixture was then extracted with dichloromethane and the organic phase was washed with brine and dried over anhydrous MgSO₄. After concentration by rotavapor, the resulting residue was purified by silica chromatography (7.5%-10% methanol in DCM) on the Combiflash system to provide the compound D(IV) as a white solid (41.3 mg, yield 54.3%). Purity was determined to be 95%. NMR and HRMS characterization is as follows: ¹H NMR (400 MHz, MeOD): δ 7.46 (dd, J=8.3, 0.8 Hz, 1H), 7.36 (d, J=1.0 Hz, 1H), 7.29-7.18 (m, 1H), 6.87 (td, J=8.1, 0.9 Hz, 1H), 5.60 (s, 2H), 3.56-3.44 (m, 2H), 3.25-3.10 (m, 2H), 2.93 (d, J=4.5 Hz, 7H), 2.73 (t, J=7.3 Hz, 2H), 1.84 (h, J=7.4 Hz, 2H), 1.17 (d, J=6.2 Hz, 1H), 1.11 (t, J=7.4 Hz, 3H). HRMS (HESI, positive) m/z for C17H24N2O3 [M+H]+ calculated, 305.1865. found: 305.1860.

### Assessment of Cell Viability Upon Treatment of a Psilocin Derivative.

Cell viability was assessed as described for Example 1, except that the compound with formula D(IV) was evaluated in place of the compound with formula F(I) and additional assays were conducted using ligands psilocin and tryptophan for comparative purposes. Data acquired for the derivative having chemical formula D(IV) is displayed as “D-IV” on the x-axes of FIG. 11B and FIG. 11C.

### Radioligand Receptor Binding Assays.

Activity at 5-HT_{2A} receptor was assessed as described for Example 1, except the compound with formula D(IV) was evaluated in place of the compound with formula F(I). FIG. 11D shows radioligand competition assay results for compound with formula D(IV), depicted on the x-axis simply as “D-IV”.

### Cell Lines and Control Ligands Used to Assess Activity at 5-HT_{1A}.

Cell lines, cell line maintenance, and experimental procedures assessing modulation of 5-HT_{1A} were performed as described in Example 1, except that the compound with formula D(IV) was evaluated in place of the compound with formula F(I). 5-HT_{1A} receptor binding evaluation for compound with formula D(IV) (designated simply “D-IV” along the x-axis) is shown in FIG. 11E. Comparison of data acquired in +5-HT_{1A} cultures with those acquired in −5-HT_{1A} cultures suggests receptor modulation.

### Evaluation of Metabolic Stability in Human Intestine, Liver, and Serum Fractions In Vitro.

Evaluations of metabolic stability and capacity of novel molecules to release psilocin under various in vitro conditions were performed as described in Example 1, except that the compound with formula D(IV) was used in place of the compound with formula F(I) for all experiments. FIGS. 11F (i)-11F(ii) show the metabolic stability curves for compound D(IV) in assays containing Buffer (Panel A), AB serum (Panel B), HIM (Panel C), HIS9 (Panel D), HLM (Panel E) and HLS9 (Panel F).

### In Vivo Evaluation of 5-HT_{2A} Receptor Agonism in Mice.

Evaluation of in vivo HTR was conducted as described in Example 1, except that the compound with formula D(IV) was used in place of the compound with formula F(I). Elevated incidences of HTR within the defined period of monitoring was observed in (1) psilocybin-treated mice, and (2) those treated with compound D(IV), relative to control mice treated with i.p. injected vehicle (0.9% NaCl). These results are illustrated in FIG. 11G, wherein compound with formula D(IV) is designated “D-IV”.

### In Vitro Survey of Pharmacological Interaction Profiles at Receptors, Transporters and Enzymes Linked to Targeted Health Conditions.

All assays were performed as described in Example 1, except compound D(IV) was used in place of F(I). To assess assay performance and establish positive control benchmarks, ligands listed in Table 1A were evaluated alongside test derivative. Results for all assays using compound D(IV) (designated “D-IV”) or positive controls are shown in Table 6.

TABLE 6
Data summary table of target assays for compound D(IV) (designated “D-
IV”) and control ligands. Potency (EC₅₀ or IC₅₀) is provided in units of μM.

| Target | Target | Assay | EC₅₀ | IC₅₀ | EC₅₀ | IC₅₀ |
|---|---|---|---|---|---|---|
| name | type | type | (control) | (control) | (D-IV) | (D-IV |
| ADRA1A | GPCR | AGN | 5.00E−05 | — | >100 | — |
| ADRA1A | GPCR | ANT | — | 9.60E−04 | — | 12.87 |
| ADRA2A | GPCR | AGN | 4.00E−05 | — | >100 | — |
| ADRA2A | GPCR | ANT | — | 3.10E−03 | — | >100 |
| AVPR1A | GPCR | AGN | 4.20E−04 | — | >100 | — |
| AVPR1A | GPCR | ANT | — | 1.60E−03 | — | >100 |
| CHRM1 | GPCR | AGN | 9.70E−03 | — | >100 | — |
| CHRM1 | GPCR | ANT | — | 6.10E−03 | — | >100 |
| CHRM2 | GPCR | AGN | 2.70E−02 | — | >100 | — |
| CHRM2 | GPCR | ANT | — | 3.20E−03 | — | >100 |
| CNR1 | GPCR | AGN | 1.00E−05 | — | >100 | — |
| CNR1 | GPCR | ANT | — | 6.20E−04 | — | >100 |
| DRD1 | GPCR | AGN | 9.10E−02 | — | >100 | — |
| DRD1 | GPCR | ANT | — | 7.10E−04 | — | 49.49 |
| DRD2S | GPCR | AGN | 5.10E−04 | — | 27.36 | — |
| DRD2S | GPCR | ANT | — | 9.60E−04 | — | >100 |
| HTR1A | GPCR | AGN | 1.70E−03 | — | 18 | — |
| HTR1A | GPCR | ANT | — | 4.60E−02 | — | >100 |
| HTR1B | GPCR | AGN | 9.00E−05 | — | 6.38E−01 | — |
| HTR1B | GPCR | ANT | — | 5.80E−03 | — | >100 |
| HTR2A | GPCR | AGN | 4.50E−03 | — | >100 | — |
| HTR2A | GPCR | ANT | — | 2.20E−03 | — | 1.27E+00 |
| HTR2B | GPCR | AGN | 6.30E−04 | — | >100 | — |
| HTR2B | GPCR | ANT | — | 4.00E−04 | — | 5.12E−01 |
| OPRD1 | GPCR | AGN | 5.00E−05 | — | 53.2 | — |
| OPRD1 | GPCR | ANT | — | 5.80E−04 | — | >100 |
| GABAA | Ion | OP | 6.2 | — | >100 | — |
| | channel | | | | | |
| GABAA | Ion | BL | — | 4.6 | — | >100 |
| | channel | | | | | |
| HTR3A | Ion | OP | 3.00E−01 | — | >100 | — |
| | channel | | | | | |
| HTR3A | Ion | BL | — | 1.90E−03 | — | >100 |
| | channel | | | | | |
| MAO-A | Enzyme | IN | — | 2.90E−03 | — | >100 |
| DAT | transporter | BL | — | 1.40E−03 | — | >100 |
| NET | transporter | BL | — | 6.70E−03 | — | >100 |
| SERT | transporter | BL | — | 1.80E−03 | — | >100 |
| NMDAR | Ion | BL | — | 8.00E−02 | — | >100 |
| | channel | | | | | |
| NMDAR | Ion | OP | 4.40E−01 | — | >100 | — |
| | channel | | | | | |

AGN, agonist; ANT, antagonist; OP, opener; BL, blocker; IN, inhibitor.

### Example 10—Synthesis and Analysis of a Third C₄-Polyether-Substituted Tryptamine Derivative

Referring to FIG. 12A, a dry round-bottom flask was charged with psilocin 1 (100 mg, 0.49 mmol, 1.0 eq) and dry DMF (2 mL) under argon. Notably, the synthesis of psilocin (1) has been described previously (Shirota et al., J. Nat. Prod. 2003, 66:885-887; Kargbo et al., ACS Omega 2020, 5:16959-16966). To the flask, Et₃N (69 μL, 1.0 eq) followed by chloromethyl isopropyl carbonate (71 mg, 0.95 eq) were added, and the resulting mixture was stirred at 70° C. for 16 h. Upon completion (TLC), the reaction mixture was diluted with water (20 mL) and extracted with DCM (3×15 mL). The combined organic layers were washed with brine, dried over anhydrous MgSO₄, filtered and concentrated. The crude residue was purified by flash chromatography using silica gel (MeOH/DCM 0:100 to 20:80, gradient) to yield the compound D(XIII) as a colourless oil (33 mg, 15%). Purity was determined to be 95%. MS and NMR characterization is as follows: MS-HESI: calculated: 437.2282; observed: 437.2275 m/z [M+H]⁺. ¹H NMR (400 MHz, Methanol-d4): δ 7.46 (dd, J=8.3, 0.8 Hz, 1H), 7.34 (s, 1H), 7.24 (t, J=8.1 Hz, 1H), 7.06 (dd, J=7.8, 0.7 Hz, 1H), 5.58 (s, 2H), 5.42 (s, 2H), 5.09-4.93 (m, 2H), 3.66-3.59 (m, 2H), 3.35-3.30 (m, 2H), 3.28 (s, 6H), 1.40 (d, J=6.3 Hz, 6H), 1.36 (d, J=6.3 Hz, 6H).

### Assessment of Cell Viability Upon Treatment of a Psilocin Derivative.

Cell viability was assessed as described for Example 1, except that the compound with formula D(XIII) was evaluated in place of the compound with formula F(I). Data acquired for the derivative having chemical formula D(XIII) is displayed as “D-XIII” on the x-axes of FIG. 12B and FIG. 12C.

### Radioligand Receptor Binding Assays.

Activity at 5-HT_{2A} receptor was assessed as described for Example 1, except the compound with formula D(XIII) was evaluated in place of the compound with formula F(I). FIG. 12D shows radioligand competition assay results for compound with formula D(XIII), depicted on the x-axis simply as “D-XIII”.

### Cell Lines and Control Ligands Used to Assess Activity at 5-HT_{1A}.

Cell lines, cell line maintenance, and experimental procedures assessing modulation of 5-HT_{1A} were performed as described in Example 1, except that the compound with formula D(XIII) was evaluated in place of the compound with formula F(I). 5-HT_{1A} receptor binding evaluation for compound with formula D(XIII) (designated simply “D-XIII” along the x-axis) is shown in FIG. 12E. Comparison of data acquired in +5-HT_{1A} cultures with those acquired in −5-HT_{1A} cultures suggests receptor modulation at higher ligand concentrations.

### Evaluation of Metabolic Stability in Human Intestine, Liver, and Serum Fractions In Vitro.

Evaluations of metabolic stability and capacity of novel molecules to release psilocin under various in vitro conditions were performed as described in Example 1, except that the compound with formula D(XIII) was used in place of the compound with formula F(I) for all experiments. FIGS. 12F (i)-12F(ii) show the metabolic stability curves for compound D(XIII) in assays containing Buffer (Panel A), AB serum (Panel B), HIM (Panel C), HIS9 (Panel D), HLM (Panel E) and HLS9 (Panel F).

### In Vivo Evaluation of 5-HT_{2A} Receptor Agonism in Mice.

Evaluation of in vivo HTR was conducted as described in Example 1, except that the compound with formula D(XIII) was used in place of the compound with formula F(I). Elevated incidences of HTR within the defined period of monitoring was observed in (1) psilocybin-treated mice, and (2) those treated with compound D(XIII), relative to control mice treated with i.p. injected vehicle (0.9% NaCl). These results are illustrated in FIG. 12G, wherein compound with formula D(XIII) is designated “D-XIII”.

### In Vitro Survey of Pharmacological Interaction Profiles at Receptors, Transporters and Enzymes Linked to Targeted Health Conditions.

All assays were performed as described in Example 1, except compound D(XIII) was used in place of F(I). To assess assay performance and establish positive control benchmarks, ligands listed in Table 1A were evaluated alongside test derivative. Results for all assays using compound D(XIII) (designated “D-XIII”) or positive controls are shown in Table 7.

TABLE 7
Data summary table of target assays for compound D(XIII) (designated “D-
XIII”) and control ligands. Potency (EC₅₀ or IC₅₀) is provided in units of μM.

| Target | Target | Assay | EC₅₀ | IC₅₀ | EC₅₀ | IC₅₀ |
|---|---|---|---|---|---|---|
| name | type | type | (control) | (control) | (D-XIII) | (D-XIII) |
| ADRA1A | GPCR | AGN | 5.00E−05 | — | >100 | — |
| ADRA1A | GPCR | ANT | — | 9.60E−04 | — | >100 |
| ADRA2A | GPCR | AGN | 4.00E−05 | — | >100 | — |
| ADRA2A | GPCR | ANT | — | 3.10E−03 | — | >100 |
| AVPR1A | GPCR | AGN | 4.20E−04 | — | >100 | — |
| AVPR1A | GPCR | ANT | — | 1.60E−03 | — | >100 |
| CHRM1 | GPCR | AGN | 9.70E−03 | — | >100 | — |
| CHRM1 | GPCR | ANT | — | 6.10E−03 | — | >100 |
| CHRM2 | GPCR | AGN | 2.70E−02 | — | >100 | — |
| CHRM2 | GPCR | ANT | — | 3.20E−03 | — | 66.5 |
| CNR1 | GPCR | AGN | 1.00E−05 | — | >100 | — |
| CNR1 | GPCR | ANT | — | 6.20E−04 | — | >100 |
| DRD1 | GPCR | AGN | 9.10E−02 | — | >100 | — |
| DRD1 | GPCR | ANT | — | 7.10E−04 | — | >100 |
| DRD2S | GPCR | AGN | 5.10E−04 | — | 77.01 | — |
| DRD2S | GPCR | ANT | — | 9.60E−04 | — | >100 |
| HTR1A | GPCR | AGN | 1.70E−03 | — | >100 | — |
| HTR1A | GPCR | ANT | — | 4.60E−02 | — | >100 |
| HTR1B | GPCR | AGN | 9.00E−05 | — | 9.32E+00 | — |
| HTR1B | GPCR | ANT | — | 5.80E−03 | — | >100 |
| HTR2A | GPCR | AGN | 4.50E−03 | — | >100 | — |
| HTR2A | GPCR | ANT | — | 2.20E−03 | — | 1.63E+01 |
| HTR2B | GPCR | AGN | 6.30E−04 | — | >100 | — |
| HTR2B | GPCR | ANT | — | 4.00E−04 | — | 5.14E+00 |
| OPRD1 | GPCR | AGN | 5.00E−05 | — | >100 | — |
| OPRD1 | GPCR | ANT | — | 5.80E−04 | — | >100 |
| GABAA | Ion | OP | 6.2 | — | >100 | — |
| | channel | | | | | |
| GABAA | Ion | BL | — | 4.6 | — | >100 |
| | channel | | | | | |
| HTR3A | Ion | OP | 3.00E−01 | — | >100 | — |
| | channel | | | | | |
| HTR3A | Ion | BL | — | 1.90E−03 | — | 1.67E+01 |
| | channel | | | | | |
| MAO-A | Enzyme | IN | — | 2.90E−03 | — | >100 |
| DAT | transporter | BL | — | 1.40E−03 | — | >100 |
| NET | transporter | BL | — | 6.70E−03 | — | >100 |
| SERT | transporter | BL | — | 1.80E−03 | — | >100 |
| NMDAR | Ion | BL | | 8.00E−02 | — | >100 |
| | channel | | | | | |
| NMDAR | Ion | OP | 4.40E−01 | — | >100 | — |
| | channel | | | | | |

AGN, agonist; ANT, antagonist; OP, opener; BL, blocker; IN, inhibitor.

### Example 11—Synthesis and Analysis of a Fourth C₄-Polyether-Substituted Tryptamine Derivative

Referring to FIG. 13A, to a solution of 1 (101 mg, 494 μmol) in dry DMF (1.00 mL) under argon, potassium carbonate (68.3 mg, 494 μmol) and potassium iodide (82.1 mg, 494 μmol) were added. Notably, the synthesis of psilocin (1) has been described previously (Shirota et al., J. Nat. Prod. 2003, 66:885-887; Kargbo et al., ACS Omega 2020, 5:16959-16966). After stirring at room temperature for 10 minutes, chloromethyl propionate (55.1 μL, 494 μmol) in dry DMF (500 μL) was added, and the resulting mixture was stirred overnight at 70° C. The mixture was diluted with water (10 mL) and washed with brine (10 mL). The brine was extracted with DCM (3×10 mL). There was a large emulsion that formed during extraction that was subsequently filtered off. The organic layer was dried with magnesium sulfate, and solvent removed under reduced pressure. The crude mixture was purified via flash column chromatography on 12 g normal-phase silica using a 0 to 10% methanol-dichloromethane gradient to yield a mixture of products. The mixture was further purified by flash column chromatography on 4 g normal-phase silica using a 2.5 to 5% methanol-dichloromethane gradient to yield the compound D(XIX) (6 mg, 4%) as a colourless oil. Purity was determined to be 95%. MS and NMR characterization is as follows: MS-HESI: calculated: 347.1965; observed: 347.1963 m/z [M+H]⁺. ¹H NMR (400 MHz, MeOD) δ 7.42 (dd, J=8.3, 0.8 Hz, 1H), 7.24-7.17 (m, 2H), 6.81 (dd, J=7.8, 0.8 Hz, 1H), 6.14 (s, 2H), 2.93-2.87 (m, 2H), 2.76 (q, J=7.5 Hz, 2H), 2.69-2.62 (m, 2H), 2.35 (s, 6H), 1.29 (t, J=7.5 Hz, 3H), 1.09 (t, J=7.5 Hz, 3H).

### Assessment of Cell Viability Upon Treatment of a Psilocin Derivative.

Cell viability was assessed as described for Example 1, except that the compound with formula D(XIX) was evaluated in place of the compound with formula F(I) and additional assays were conducted using ligands psilocin and tryptophan for comparative purposes. Data acquired for the derivative having chemical formula D(XIX) is displayed as “D-XIX” on the x-axes of FIG. 13B and FIG. 13C.

### Radioligand Receptor Binding Assays.

Activity at 5-HT_{2A} receptor was assessed as described for Example 1, except the compound with formula D(XIX) was evaluated in place of the compound with formula F(I). FIG. 13D shows radioligand competition assay results for compound with formula D(XIX), depicted on the x-axis simply as “D-XIX”.

### Cell Lines and Control Ligands Used to Assess Activity at 5-HT_{1A}.

Cell lines, cell line maintenance, and experimental procedures assessing modulation of 5-HT_{1A} were performed as described in Example 1, except that the compound with formula D(XIX) was evaluated in place of the compound with formula F(I). 5-HT_{1A} receptor binding evaluation for compound with formula D(XIX) (designated simply “D-XIX” along the x-axis) is shown in FIG. 13E. Comparison of data acquired in +5-HT_{1A} cultures with those acquired in −5-HT_{1A} cultures suggests significant receptor modulation.

### Evaluation of Metabolic Stability in Human Intestine, Liver, and Serum Fractions In Vitro.

Evaluations of metabolic stability and capacity of novel molecules to release psilocin under various in vitro conditions were performed as described in Example 1, except that the compound with formula D(XIX) was used in place of the compound with formula F(I) for all experiments. FIGS. 13F (i)-13F(ii) show the metabolic stability curves for compound D(XIX) in assays containing Buffer (Panel A), AB serum (Panel B), HIM (Panel C), HIS9 (Panel D), HLM (Panel E) and HLS9 (Panel F).

### In Vivo Evaluation of 5-HT_{2A} Receptor Agonism in Mice.

Evaluation of in vivo HTR was conducted as described in Example 1, except that the compound with formula D(XIX) was used in place of the compound with formula F(I). Elevated incidences of HTR within the defined period of monitoring was observed in (1) psilocybin-treated mice, and (2) those treated with compound D(XIX), relative to control mice treated with i.p. injected vehicle (0.9% NaCl). These results are illustrated in FIG. 13G, wherein compound with formula D(XIX) is designated “D-XIX”. Results for control mice injected with vehicle are not shown in FIG. 13G but are the same as those in Example 1 (FIG. 3P) since HTR experiments were run with the same control cohorts.

### Example 12—Synthesis and Analysis of a Second C₄-Phosphate-Substituted Tryptamine Derivative

Referring to FIG. 14A, Compound 1 (101 mg, 0.49 mmol, 1.0 eq) was suspended in anhydrous dichloromethane (1 mL) under argon atmosphere. Notably, the synthesis of psilocin (1) has been described previously (Shirota et al., J. Nat. Prod. 2003, 66:885-887; Kargbo et al., ACS Omega 2020, 5:16959-16966). Triethylamine (0.14 mL, 0.98 mmol, 2.0 eq) was added, followed by 2-chloro-5,5-dimethyl-1,3,2-dioxaphosphorinane-2-oxide (182 mg, 0.98 mmol, 2.0 eq) dissolved in anhydrous dichloromethane (0.5 mL) was added. The resulting mixture was stirred at room temperature for 18 hours and monitored by TLC (20% methanol-dichloromethane). The mixture was diluted with dichloromethane (10 mL) and washed with saturated sodium bicarbonate (10 mL) and brine (2×10 mL). The organic phase was dried with magnesium sulphate, and solvent was removed under reduced pressure to yield an amber oil. The crude material was purified by flash column chromatography on 12 g normal-phase silica using 0 to 40% methanol-dichloromethane as eluent to yield the compound F(II) (55 mg, 29%) as a white solid. Purity was determined to be 95%. MS and NMR characterization is as follows: MS-ESI: calculated: 353.1625; observed: 353.1624 m/z [M+H]⁺. ¹H NMR (400 MHz, Chloroform-d) δ 9.41 (s, 1H), 7.08-7.02 (m, 1H), 7.00-6.94 (m, 2H), 6.75 (d, J=2.4 Hz, 1H), 4.31 (ddd, J=11.5, 2.6, 1.4 Hz, 2H), 4.10-3.93 (m, 2H), 3.12-2.98 (m, 2H), 2.73-2.58 (m, 2H), 2.35 (s, 6H), 1.34 (s, 3H), 0.87 (s, 3H).

### Assessment of Cell Viability Upon Treatment of a Psilocin Derivative.

Cell viability was assessed as described for Example 1, except that the compound with formula F(II) was evaluated in place of the compound with formula F(I) and additional assays were conducted using ligands psilocin and psilocybin for comparative purposes. Data acquired for the derivative having chemical formula F(II) is displayed as “F-II” on the x-axes of FIG. 14B and FIG. 14C.

### Radioligand Receptor Binding Assays.

Activity at 5-HT_{2A} receptor was assessed as described for Example 1, except the compound with formula F(II) was evaluated in place of the compound with formula F(I). FIG. 14D shows radioligand competition assay results for compound with formula F(II), depicted on the x-axis simply as “F-II”.

### Cell Lines and Control Ligands Used to Assess Activity at 5-HT_{1A}.

Cell lines, cell line maintenance, and experimental procedures assessing modulation of 5-HT_{1A} were performed as described in Example 1, except that the compound with formula F(II) was evaluated in place of the compound with formula F(I). 5-HT_{1A} receptor binding evaluation for compound with formula F(II) (designated “F-II” along the x-axis) is shown in FIG. 14E. Comparison of data acquired in +5-HT_{1A} cultures with those acquired in −5-HT_{1A} cultures reveals receptor modulation.

### Evaluation of Metabolic Stability in Human Intestine, Liver, and Serum Fractions In Vitro.

Evaluations of metabolic stability and capacity of novel molecules to release psilocin under various in vitro conditions were performed as described in Example 1, except that the compound with formula F(II) was used in place of the compound with formula F(I) for all experiments. FIGS. 14F (i)-14F(ii) show the metabolic stability curves for compound F(II) in assays containing Buffer (Panel A), AB serum (Panel B), HIM (Panel C), HIS9 (Panel D), HLM (Panel E) and HLS9 (Panel F).

### In Vivo Evaluation of 5-HT_{2A} Receptor Agonism in Mice.

Evaluation of in vivo HTR was conducted as described in Example 1, except that the compound with formula F(II) was used in place of the compound with formula F(I). Elevated incidences of HTR within the defined period of monitoring was observed in (1) psilocybin-treated mice, and (2) those treated with compound F(II), relative to control mice treated with i.p. injected vehicle (0.9% NaCl). These results are illustrated in FIG. 14G, wherein compound with formula F(II) is designated “F-II”.

### Example 13—Synthesis and Analysis of a Third C₄-Ether-Substituted Tryptamine Derivative

Referring to FIG. 15A, compound 1 (100 mg, 0.49 mmol, 1.0 eq) and imidazole (100 mg, 1.5 mmol, 3.0 eq) were dissolved in anhydrous dimethylformamide (1.5 mL) under argon atmosphere, to which tert-butyl(chloro)dimethylsilane (89 mg, 0.59 mmol, 1.2 eq) was added. Notably, the synthesis of psilocin (1) has been described previously (Shirota et al., J. Nat. Prod. 2003, 66:885-887; Kargbo et al., ACS Omega 2020, 5:16959-16966). The solution was allowed to stir for 18 hours until complete as determined by TLC (20% methanol-dichloromethane). The solvent was removed under reduced pressure at 80° C. to dryness, then the residue was dissolved in ethyl acetate (15 mL), washed with water (3×10 mL), dried with magnesium sulphate, and concentrated under vacuum. Purification by flash column chromatography on 12 g normal-phase silica using a 0 to 10% methanol-dichloromethane gradient as eluent yielded the crude product contaminated with imidazole. The crude was dissolved in dichloromethane (15 mL), washed with brine (3×10 mL), dried with magnesium sulphate, and solvent removed under reduced pressure to yield the compound A(IV) (80 mg, 80%) as a white solid. Purity was determined to be 95%. MS and NMR characterization is as follows: MS-HESI: calculated: 319.2200; observed: 319.2195 m/z [M+H]⁺. ¹H NMR (400 MHz, Methanol-d4) δ 7.00-6.88 (m, 3H), 6.45 (dd, J=7.3, 1.1 Hz, 1H), 3.22-3.14 (m, 2H), 2.92 (dd, J=8.5, 6.8 Hz, 2H), 2.45 (s, 6H), 1.06 (s, 9H), 0.35 (s, 6H).

### Assessment of Cell Viability Upon Treatment of a Psilocin Derivative.

Cell viability was assessed as described for Example 1, except that the compound with formula A(IV) was evaluated in place of the compound with formula F(I). Data acquired for the derivative having chemical formula A(IV) is displayed as “A-IV” on the x-axes of FIG. 15B and FIG. 15C.

### Radioligand Receptor Binding Assays.

Activity at 5-HT_{2A} receptor was assessed as described for Example 1, except the compound with formula A(IV) was evaluated in place of the compound with formula F(I). FIG. 15D shows radioligand competition assay results for compound with formula A(IV), depicted on the x-axis simply as “A-IV”.

### Cell Lines and Control Ligands Used to Assess Activity at 5-HT_{1A}.

Cell lines, cell line maintenance, and experimental procedures assessing modulation of 5-HT_{1A} were performed as described in Example 1, except that the compound with formula A(IV) was evaluated in place of the compound with formula F(I). 5-HT_{1A} receptor binding evaluation for compound with formula A(IV) (designated “A-IV” along the x-axis) is shown in FIG. 15E. Comparison of data acquired in +5-HT_{1A} cultures with those acquired in −5-HT_{1A} cultures reveals receptor modulation.

### Evaluation of Metabolic Stability in Human Intestine, Liver, and Serum Fractions In Vitro.

Evaluations of metabolic stability and capacity of novel molecules to release psilocin under various in vitro conditions were performed as described in Example 1, except that the compound with formula A(IV) was used in place of the compound with formula F(I) for all experiments. FIGS. 15F (i)-15F(ii) show the metabolic stability curves for compound A(IV) in assays containing Buffer (Panel A), AB serum (Panel B), HIM (Panel C), HIS9 (Panel D), HLM (Panel E) and HLS9 (Panel F).

### In Vivo Evaluation of 5-HT_{2A} Receptor Agonism in Mice.

Evaluation of in vivo HTR was conducted as described in Example 1, except that the compound with formula A(IV) was used in place of the compound with formula F(I). Elevated incidences of HTR within the defined period of monitoring was observed in (1) psilocybin-treated mice, and (2) those treated with compound A(IV), relative to control mice treated with i.p. injected vehicle (0.9% NaCl). These results are illustrated in FIG. 15G, wherein compound with formula A(IV) is designated “A-IV”.

### In Vitro Survey of Pharmacological Interaction Profiles at Receptors, Transporters and Enzymes Linked to Targeted Health Conditions.

All assays were performed as described in Example 1, except compound A(IV) was used in place of F(I). To assess assay performance and establish positive control benchmarks, ligands listed in Table 1A were evaluated alongside test derivative. Results for all assays using compound A(IV) (designated “A-IV”) or positive controls are shown in Table 8.

TABLE 8
Data summary table of target assays for compound A(IV) (designated “A-
IV”) and control ligands. Potency (EC₅₀ or IC₅₀) is provided in units of μM.

| Target | Target | Assay | EC₅₀ | IC₅₀ | EC₅₀ | IC₅₀ |
|---|---|---|---|---|---|---|
| name | type | type | (control) | (control) | (A-IV) | (A-IV) |
| ADRA1A | GPCR | AGN | 5.00E−05 | — | >100 | — |
| ADRA1A | GPCR | ANT | — | 9.60E−04 | — | 18.48 |
| ADRA2A | GPCR | AGN | 4.00E−05 | — | >100 | — |
| ADRA2A | GPCR | ANT | — | 3.10E−03 | — | 15.98 |
| AVPR1A | GPCR | AGN | 4.20E−04 | — | >100 | — |
| AVPR1A | GPCR | ANT | — | 1.60E−03 | — | 42.3 |
| CHRM1 | GPCR | AGN | 9.70E−03 | — | >100 | — |
| CHRM1 | GPCR | ANT | — | 6.10E−03 | — | 25.84 |
| CHRM2 | GPCR | AGN | 2.70E−02 | — | >100 | — |
| CHRM2 | GPCR | ANT | — | 3.20E−03 | — | >100 |
| CNR1 | GPCR | AGN | 1.00E−05 | — | >100 | — |
| CNR1 | GPCR | ANT | — | 6.20E−04 | — | >100 |
| DRD1 | GPCR | AGN | 9.10E−02 | — | >100 | — |
| DRD1 | GPCR | ANT | — | 7.10E−04 | — | 10.94 |
| DRD2S | GPCR | AGN | 5.10E−04 | — | 12.67 | — |
| DRD2S | GPCR | ANT | — | 9.60E−04 | — | >100 |
| HTR1A | GPCR | AGN | 1.70E−03 | — | 4.46 | — |
| HTR1A | GPCR | ANT | — | 4.60E−02 | — | >100 |
| HTR1B | GPCR | AGN | 9.00E−05 | — | 5.03E−01 | — |
| HTR1B | GPCR | ANT | — | 5.80E−03 | — | >100 |
| HTR2A | GPCR | AGN | 4.50E−03 | — | 33.97 | — |
| HTR2A | GPCR | ANT | — | 2.20E−03 | — | 8.51E−01 |
| HTR2B | GPCR | AGN | 6.30E−04 | — | >100 | — |
| HTR2B | GPCR | ANT | — | 4.00E−04 | — | 8.14E−01 |
| OPRD1 | GPCR | AGN | 5.00E−05 | — | 29.14 | — |
| OPRD1 | GPCR | ANT | — | 5.80E−04 | — | >100 |
| GABAA | Ion | OP | 6.2 | — | >100 | — |
| | channel | | | | | |
| GABAA | Ion | BL | — | 4.6 | — | 36.47 |
| | channel | | | | | |
| HTR3A | Ion | OP | 3.00E−01 | — | >100 | — |
| | channel | | | | | |
| HTR3A | Ion | BL | — | 1.90E−03 | — | 8.30E−01 |
| | channel | | | | | |
| MAO-A | Enzyme | IN | — | 2.90E−03 | — | 54.56 |
| DAT | transporter | BL | — | 1.40E−03 | — | 25.44 |
| NET | transporter | BL | — | 6.70E−03 | — | >100 |
| SERT | transporter | BL | — | 1.80E−03 | — | >100 |
| NMDAR | Ion | BL | — | 8.00E−02 | — | 12.13 |
| | channel | | | | | |
| NMDAR | Ion | OP | 4.40E−01 | — | >100 | — |
| | channel | | | | | |

AGN, agonist; ANT, antagonist; OP, opener; BL, blocker; IN, inhibitor.

What is claimed is:

1. A chemical compound having chemical formula (I): 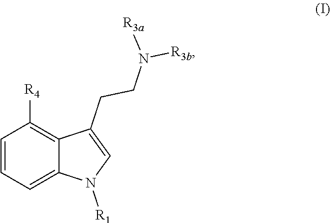
wherein R₄ is a substituent containing:
(A) an ether moiety or a derivative thereof;
(B) a carbonic ester moiety or a derivative thereof;
(C) a carboxylic acid moiety or a derivative thereof;
(D) a polyether or a derivative thereof;
(E) a carbonothioate moiety or a derivative thereof; or
(F) a phosphate moiety or a derivative thereof;
wherein R₁ is a hydrogen atom or a moiety containing a carbonic ester or derivative thereof, or a ketone or ketone derivative; and
wherein R₃ₐ and R_{3b} are each independently a hydrogen atom or an alkyl group.

2. A chemical compound according to claim 1, wherein R₄ is a substituent selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof.

3. A chemical compound according to claim 2, wherein in the compound having chemical formula (I) wherein R₄ is an ether moiety or derivative thereof, the compound having formula (I) has the chemical formula (XX): 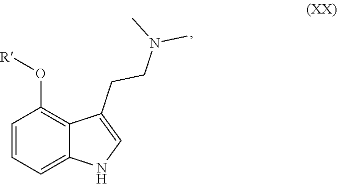
wherein
R′ is an aryl group, a heteroaryl group, a heterocyclic group, an alkyl-aryl group, an alkyl-heterocyclic group, a silyl group, or an alkyl-heteroaryl group, wherein the aryl group, heteroaryl group, alkyl-aryl group, alkyl-heterocycle group, or alkyl-heteroaryl groups are substituted or unsubstituted, and wherein the optional substituents are a halo group, an amide group, an oxo group, or an alkyl group, wherein two or more alkyl groups optionally form an additional 5-7-membered ring.

4. A chemical compound according to claim 2, wherein in the compound having chemical formula (I) wherein R₄ is an ether moiety or derivative thereof, in the compound the ether moiety or derivative thereof can be an aryl ether group, a heteroaryl ether group, a heterocyclic ether group, an alkyl-aryl ether group, an alkyl-heterocyclic group, a silyl ether group, or an alkyl-heteroaryl group, wherein the aryl group, heteroaryl group, alkyl-aryl group, alkyl-heterocycle group, or alkyl-heteroaryl groups are substituted or unsubstituted, and wherein the optional substituents are a halo group, an amide group, an oxo group, or an alkyl group, wherein two or more alkyl groups optionally form an additional 5-7-membered ring.

5.-14. (canceled)

15. A chemical compound according to claim 2, wherein in the compound having chemical formula (I) wherein R₄ is an ether moiety or derivative thereof, the compound is selected from the group consisting of A(I), A(II), A(III), A(IV), A(V), A(VI), A(VII), A(VIII), A(IX) and A(X):

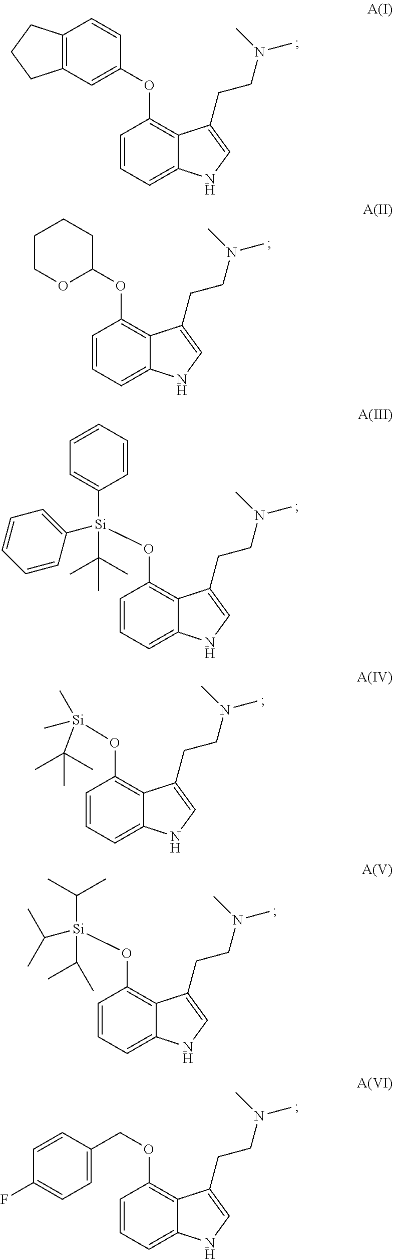

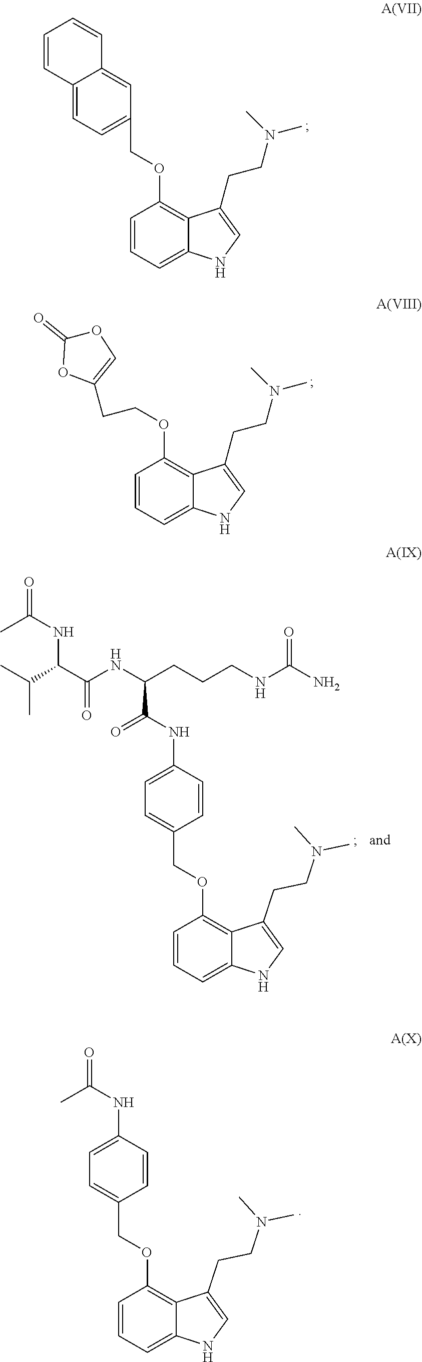

16. A chemical compound according to claim 2, wherein in the compound having chemical formula (I) wherein R₄ is a carbonic ester moiety or derivative thereof, the compound having formula (I) has the chemical formula (XIV): 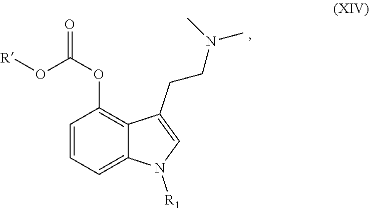
wherein
R₁ is a hydrogen atom or a carbonic ester moiety —O—C(═O)—O—(C₁-C₆)-alkyl, wherein the alkyl is optionally substituted with halo or C₆-C₁₀-aryl;
R′ is C₁-C₁₀-alkyl, wherein the C₁-C₁₀-alkyl group is optionally substituted with one or more of halo, C₆-C₁₀-aryl, C₃-C₆-cycloalkyl, or a polycyclic hydrocarbon.

17.-18. (canceled)

19. A chemical compound according to claim 2, wherein in the compound having chemical formula (I) wherein R₄ is a carbonic ester moiety or derivative thereof, the compound is selected from the group consisting of B(I), B(II), B(III), B(IV), B(V), and B(VI):

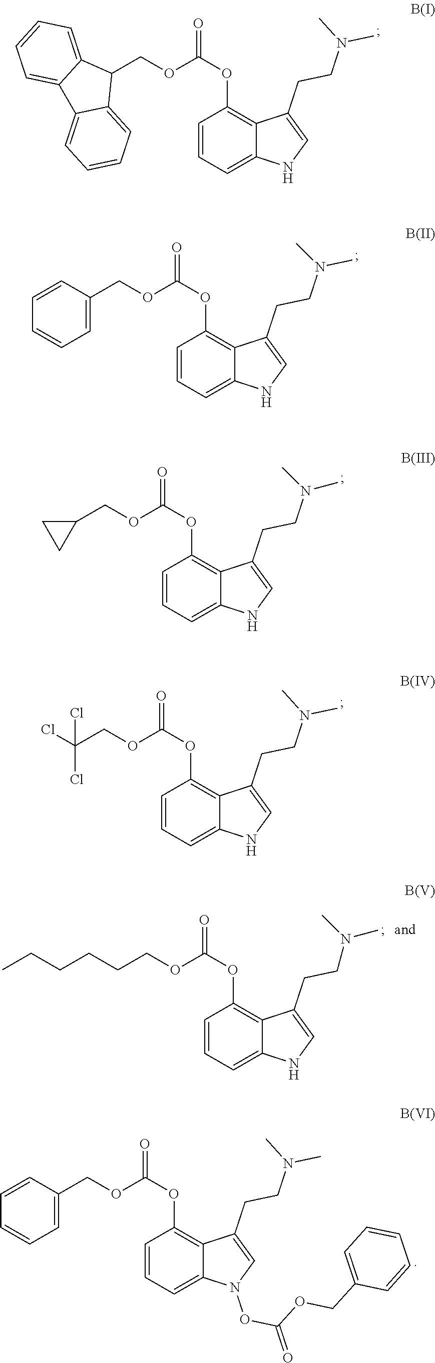

20. A chemical compound according to claim 2, wherein in the compound having chemical formula (I) wherein R₄ is a polyether moiety or derivative thereof, the polyether moiety or derivative thereof includes a carboxylic acid moiety or derivative thereof.

21. A chemical compound according to claim 2, wherein the polyether moiety or derivative thereof is selected from the group of compounds consisting of D(I), D(II), D(III), D(IV), D(V), D(VI), D(VII), D(VIII), D(IX), D(X), D(XI), D(XII), D(XIII), D(XIV), D(XV), D(XVI), D(XVII), D(XVIII), and D(XIX):

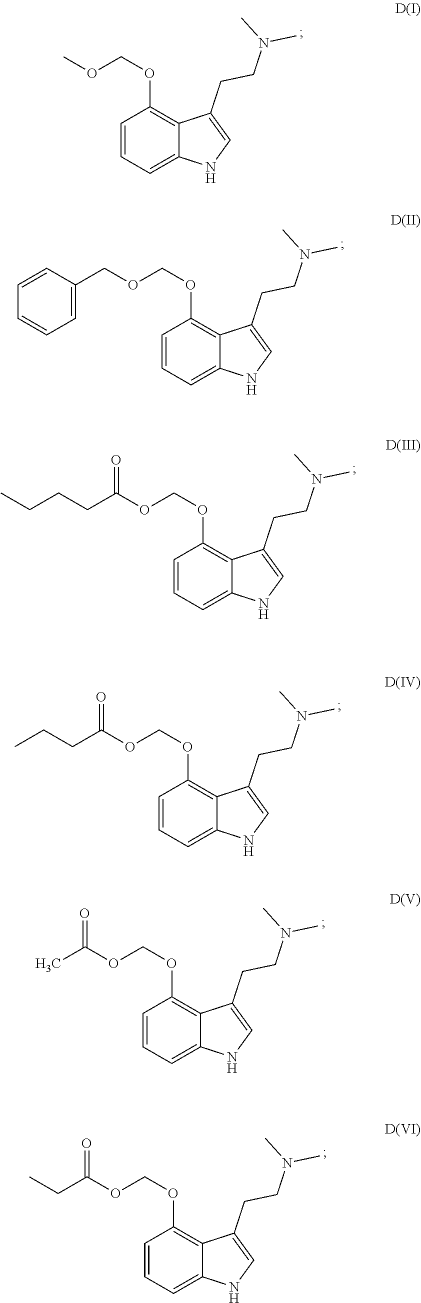

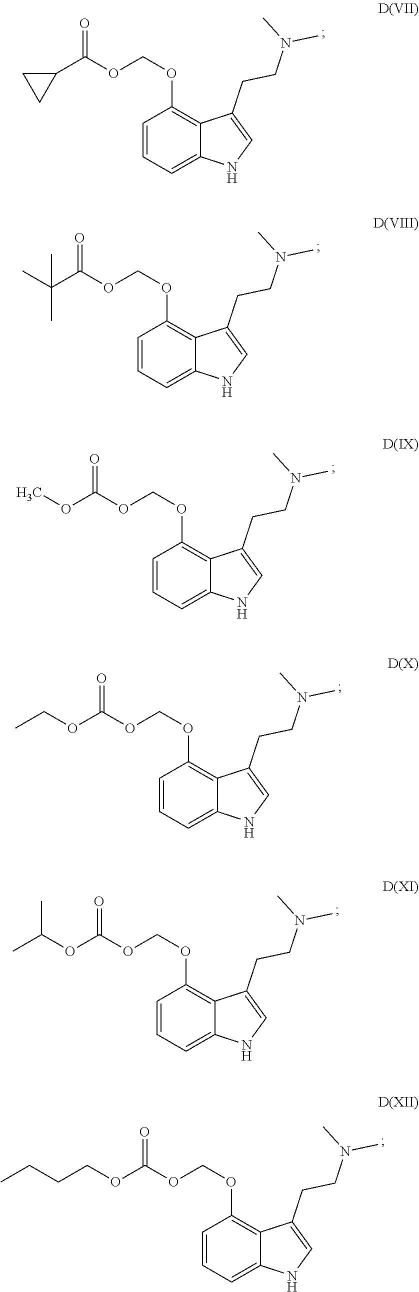

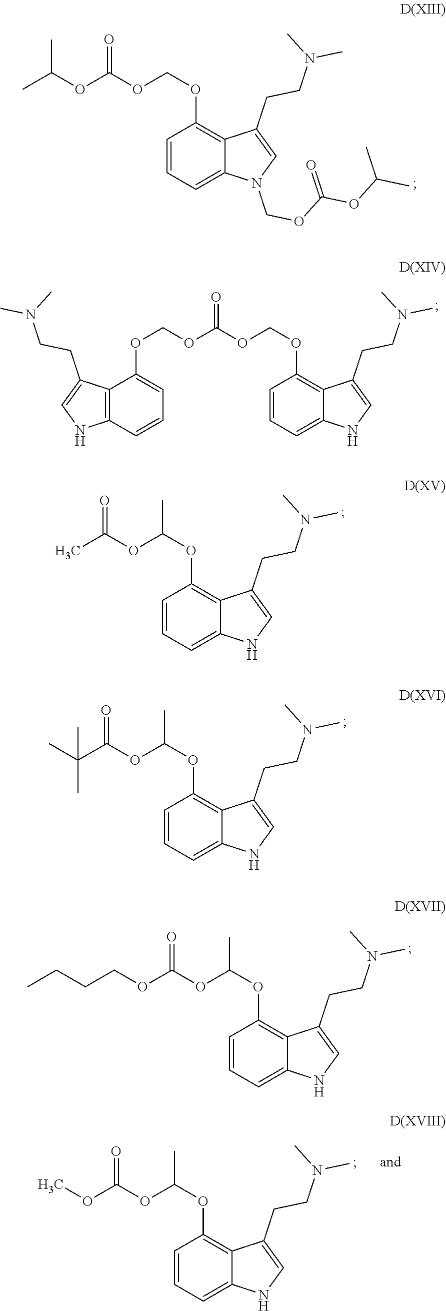

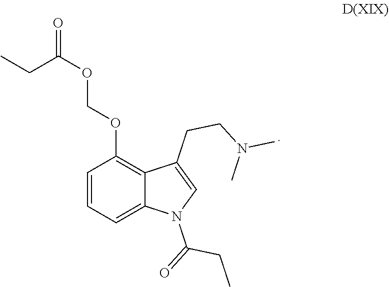 [SMILES: CCC(=O)OCOc1cccc2c1c(CCN(C)C)cn2C(=O)CC]

22.-34. (canceled)

35. A chemical compound according to claim 2, wherein in the compound having chemical formula (I) wherein R₄ is a phosphate moiety or derivative thereof, the compound having formula (I) has chemical formula (XVI):
wherein, 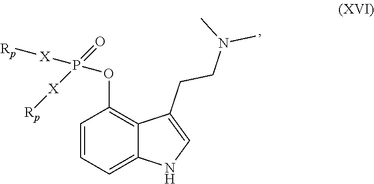
X is O or NH;
each Rₚ is independently or simultaneously alkyl, aryl, or each Rₚ is joined together to form an optionally substituted 4-7-membered heterocyclic ring.

36. A chemical compound according to claim 2, wherein in the compound having chemical formula (I) wherein R₄ is a phosphate moiety or derivative thereof, the compound is selected from the group consisting of F(I), F(II), F(III), F(IV), F(V), F(VI), F(VII), F(VIII), F(IX), F(X), F(XI), F(XII), F(XIII), F(XIV), and F(XV):

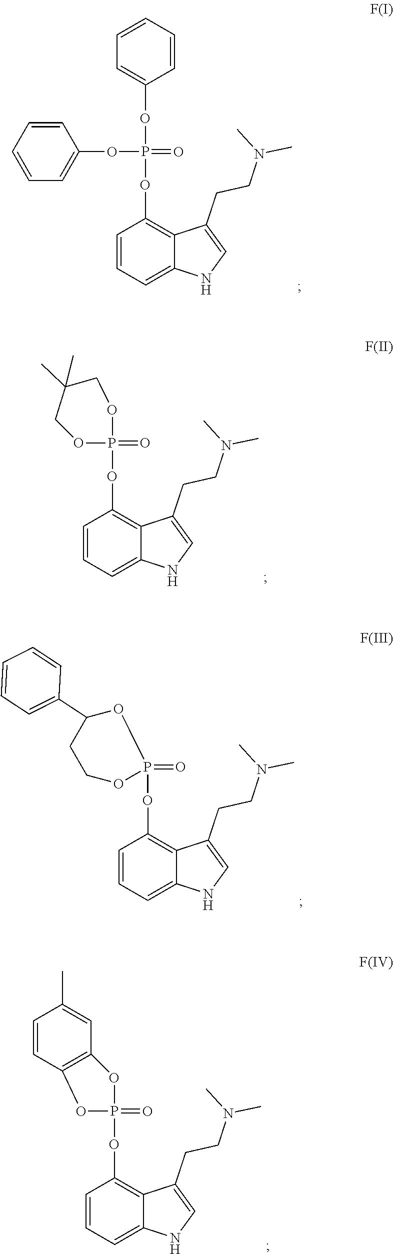

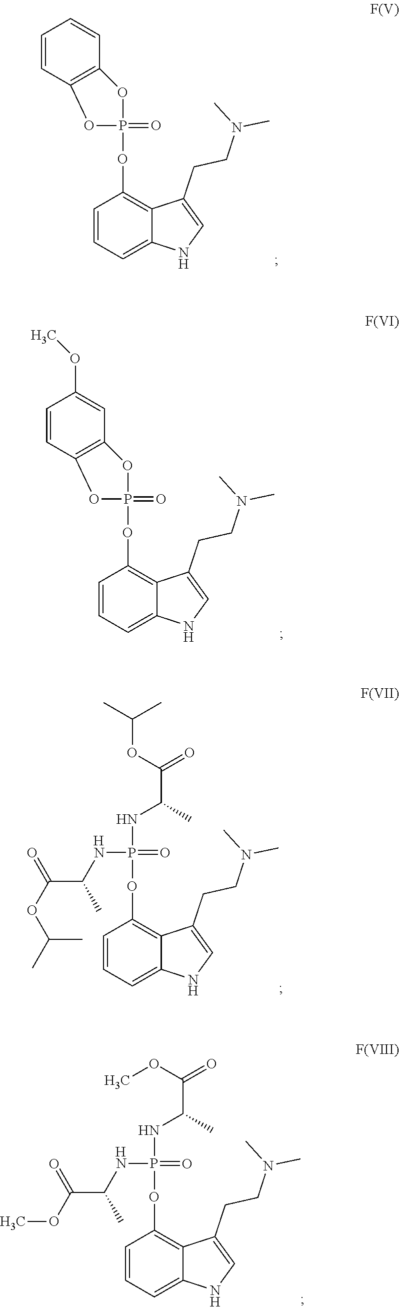

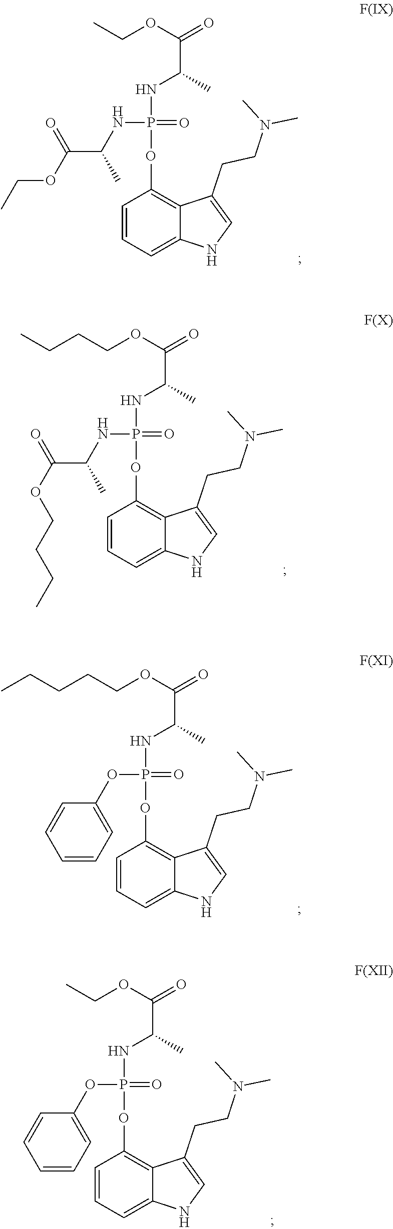

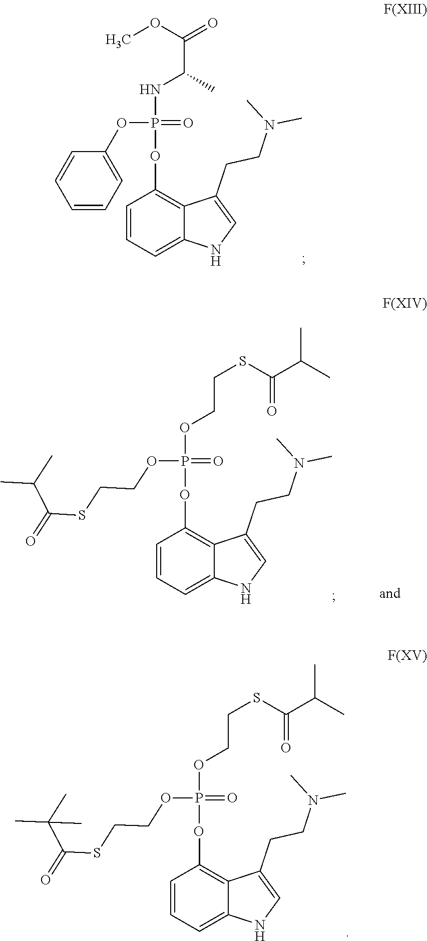

37.-42. (canceled)

43. A chemical compound according to claim 2, wherein R₁ is a carbonic ester having chemical formula (V) or (XIII):

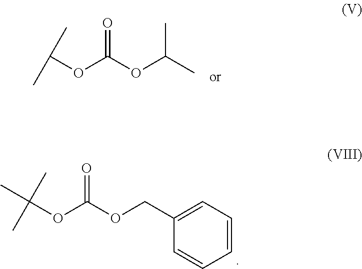

44.-45. (canceled)

46. A chemical compound according to claim 1, wherein R₄ is a substituent selected from:
(i) (A) an ether moiety or a derivative thereof;
(ii) (B) a carbonic ester moiety or a derivative thereof;
(iii) (D) a polyether or a derivative thereof; and
(iv) (F) a phosphate moiety or a derivative thereof,
wherein (NR₃ₐR_{3b}) has the formula (N⁺HR₃ₐR_{3b}Z), wherein Z is a counter-balancing anion, and wherein compound (I) is a salt.

47. A chemical compound according to claim 46, wherein Z is a mono-valent counter-balancing ion (Z⁻), a di-valent counter-balancing ion (Z²⁻), or a tri-valent counter-balancing ion (Z³⁻).

48. A chemical compound according to claim 46, wherein Z is a mono-valent counter-balancing anion (Z⁻) selected from a halide ion (Cl⁻, Br⁻, F⁻, I⁻), a nitrate ion (NO₃⁻), a benzoate ion (phenyl-COO⁻), a succinate ion (HOOC—(CH₂)₂—COO⁻), a fumarate ion (trans-HOOC—(CH═CH)—COO⁻), a tartarate ion (HOOC—(CHOH)₂—COO⁻), a malate ion (HOOC—CH₂—CHOH—COO⁻), a maleate ion (cis-HOOC—(CH═CH)—COO⁻), a dibenzoyl tartarate ion (HOOC—(CHOBz)₂-COO⁻), a ditoluoyl tartarate ion (HOOC—(CHOCOTol)₂-COO⁻), a malonate ion (HOOC—CH₂—COO⁻), a dihydrogen phosphate ion (H₂PO₄⁻), and an acetate ion (CH₃—COO⁻), wherein the salt compound has the formula (Iₐ):

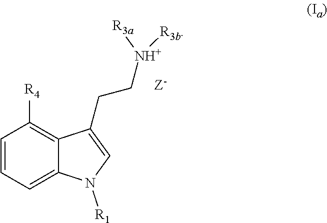

49. A chemical compound according to claim 46, wherein Z is a di-valent counter-balancing anion (Z²⁻) selected from a sulfate ion (SO₄²⁻), a hydrogen phosphate ion (HPO₄²⁻), a succinate dianion (⁻OOC—(CH₂)₂—COO⁻), a fumarate dianion (trans-⁻OOC—(CH═CH)—COO⁻), a tartarate dianion (⁻OOC—(CHOH)₂—COO⁻), a malate dianion (⁻OOC—CH₂—CHOH—COO⁻), a maleate dianion (cis-⁻OOC—(CH═CH)—COO⁻), a dibenzoyl tartarate dianion (⁻OOC—(CHOBz)₂-COO⁻), a ditoluoyl tartarate dianion (⁻OOC—(CHOCOTol)₂-COO⁻), and a malonate dianion (⁻OOC—CH₂—COO⁻), wherein the salt compound has the formula (I_{b}):

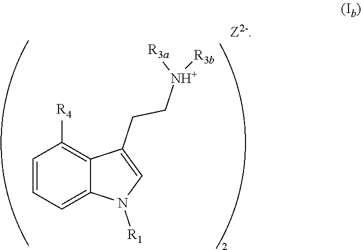

50. A chemical compound according to claim 46, wherein Z is a tri-valent counter-balancing anion (Z³⁻) selected from a phosphate ion (PO₄³⁻) and a citrate ion (⁻OOC—CH₂—C(OH)(COO⁻)—CH₂—COO⁻, and the salt compound has the formula (I_{c}):

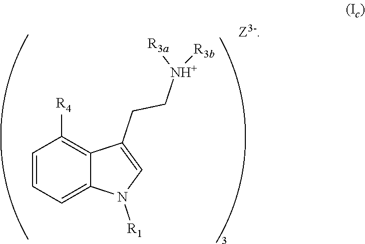

51. A pharmaceutical or recreational drug formulation comprising an effective amount of a chemical compound having a chemical formula (I) according to claim 2, together with a pharmaceutically acceptable excipient, diluent, or carrier.

52.-57. (canceled)

58. A method for treating a psychiatric disorder, the method comprising administering to a subject in need thereof a pharmaceutical formulation comprising a chemical compound having a chemical formula (I) according to claim 2, wherein the pharmaceutical formulation is administered in an effective amount to treat the psychiatric disorder in the subject.

59.-67. (canceled)

68. A method for modulating (i) a receptor selected from 5-HT_{1A} receptor, a 5-HT_{2A} receptor, a 5-HT_{1B} receptor, a 5-HT_{2B} receptor, a 5-HT_{3A} receptor, an ADRA1A receptor, an ADRA2A receptor, a CHRM1 receptor, a CHRM2 receptor, a CNR1 receptor, a DRD1 receptor, a DRD2S receptor, or an OPRD1 receptor; (ii) an enzyme, the enzyme being MOA-1; or (iii) a transmembrane transport protein selected from a dopamine active transporter (DAT), a norephedrine transporter (NET) or a serotonin transporter (SERT) transmembrane transport protein, the method comprising contacting (i) the 5-HT_{1A} receptor, the 5-HT_{2A} receptor, the 5-HT_{1B} receptor, the 5-HT_{2B} receptor, the 5-HT_{3A} receptor, the ADRA1A receptor, the ADRA2A receptor, the CHRM1 receptor, the CHRM2 receptor, the CNR1 receptor, the DRD1 receptor, the DRD2S receptor, or the OPRD1 receptor; (ii) MOA-1; or (iii) the dopamine active transporter (DAT), the norephedrine transporter (NET), or the serotonin transporter (SERT) transmembrane transport protein with a chemical compound having a chemical formula (I) according to claim 2: 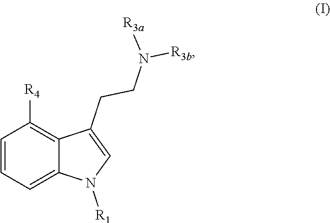
wherein R₄ is a substituent selected from:
(A) an ether moiety or a derivative thereof;
(B) a carbonic ester moiety or a derivative thereof;
(D) a polyether or a derivative thereof; and
(F) a phosphate moiety or a derivative thereof,
wherein R₁ is a hydrogen atom or a moiety containing a carbonic ester or derivative thereof, or a ketone or ketone derivative; and
wherein R₃ₐ and R_{3b} are each independently a hydrogen atom or an alkyl group, under reaction conditions sufficient to modulate (i) the 5-HT_{1A} receptor, the 5-HT_{2A} receptor, the 5-HT_{1B} receptor, the 5-HT_{2B} receptor, the 5-HT_{3A} receptor, the ADRA1A receptor, the ADRA2A receptor, the CHRM1 receptor, the CHRM2 receptor, the CNR1 receptor, the DRD1 receptor, the DRD2S receptor, or the OPRD1 receptor; (ii) MOA-1; or (iii) the dopamine active transporter (DAT), the norephedrine transporter (NET), or the serotonin transporter (SERT) transmembrane transport protein.

69.-91. (canceled)
